(19) United States
(12) Patent Application Publication
(10) Pub. No.: US 2023/0286948 A1
(43) Pub. Date: Sep. 14, 2023

(54) HALOALKYLPYRIDYL TRIAZOLE MLL1-WDR5 PROTEIN-PROTEIN INTERACTION INHIBITOR

(71) Applicant: HUYABIO International, LLC, San Diego, CA (US)

(72) Inventors: Farbod SHOJAEI, San Diego, CA (US); J. Edward SEMPLE, Lake Forest, CA (US); Mireille GILLINGS, San Diego, CA (US)

(21) Appl. No.: 18/120,326

(22) Filed: Mar. 10, 2023

Related U.S. Application Data
(60) Provisional application No. 63/319,564, filed on Mar. 14, 2022.

(51) Int. Cl. C07D 401/14; A61P 35/02; C07D 401/12; C07D 403/12; C07D 249/06

(52) U.S. Cl. CPC C07D 401/14; A61P 35/02; C07D 401/12; C07D 403/12; C07D 249/06

(57) ABSTRACT

Described herein are a haloalkylpyridyl triazole MLL1-WDR5 protein-protein interaction inhibitors, pharmaceutical compositions and methods of use.

### CROSS-REFERENCE

This application claims the benefit of U.S. Provisional Application No. 63/319,564 filed Mar. 14, 2022, the contents of which are incorporated herein by reference in their entirety.

### FIELD OF THE DISCLOSURE

The present invention relates to the field of pharmaceutical chemistry, and more particularly to haloalkylpyridyl triazole MLL1-WDR5 protein-protein interaction inhibitors, preparation and medical uses thereof.

### BACKGROUND OF DISCLOSURE

Translocation and re-arrangement of the methyl transferase mixed lineage leukemia protein-1 (MLL1) gene for histone H3K4 can lead to mixed lineage leukemia (MLL1, acute myeloid leukemia and acute lymphoid leukemia). MLL1 gene rearrangement is found in about 10% of leukemia patients. Upon re-arrangement, the MLL1 gene fuses with other chaperone genes to form fusion genes, and the carcinogenic MLL1 fusion protein is expressed. The fusion protein can interact with RNA polymerase II (Pol II) related elongation factors to form the super elongation complex (SEC). The complex can lead to abnormal expression of the Hox gene regulated by MLL1 through Pol II, which causes a series of serious consequences to induce MLL leukemia onset.

Chromosomal translocation of the MLL1 gene is monoallelic and there is a wildtype MLL1. When the wildtype MLL1 allele is knocked out, the MLL1 fusion protein alone will not lead to leukemia. Thus, specific inhibition of the enzymatic activity of the wildtype MLL1 can achieve the effect of treating leukemia.

Catalytic activity on H3K4 methylation by MLL1 alone is very weak and can only result in monomethylation; the enzyme catalytic activity improves greatly upon the formation of the MLL1 core catalytic complex, especially the catalytic activity on H3K4me2. The MLL-C-terminal WIN motif moiety is capable of binding WDR5, RbBP5, Ash2L and DPY30 to form complexes. MLL1 interacts with WDR5 directly through the C-terminal WIN motif moiety, to mediate the interaction between the catalytic domain of MLLISET and other protein complexes. When WDR5 is knocked out, the level of H3K4me2/3 decreases and the Hox gene expression is downregulated.

Thus, use of small molecule inhibitors to inhibit the protein-protein interaction of MLL1-WDR5 is an effective method to inhibit MLL1 enzymatic activity and downregulate Hox and Meis-1 gene expression to block the progression of leukemia. Previous MLL1-WDR5 protein-protein interaction inhibitors have been described in WO2019205687A1, which is herein incorporated by reference in its entirety. A need exists for improved MLL1-WDR5 protein-protein interaction inhibitors.

### BRIEF SUMMARY

Described herein are small molecule compounds that can regulate MLL1-WDR5 protein-protein interaction, and compositions and methods of using the compounds and compositions. Small molecule compound regulators of MLL1-WDR5 protein-protein interactions can inhibit the enzyme catalytic activity of MLL1 and downregulate the methylation level of H3K4 and the gene expression levels of Hox and Meis-1 genes to induce the apoptosis of leukemia cells. Therefore, the compound and compositions described herein can be used to treat cancers such as, but not limited to, leukemia.

In one aspect, described herein is a compound that has the structure of Formula (I), or a pharmaceutically acceptable salt or solvate thereof:

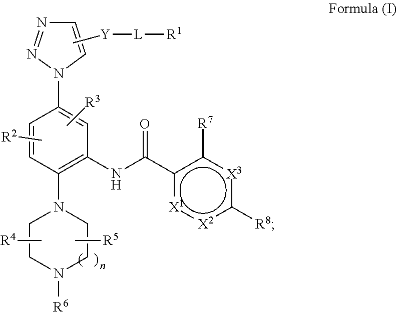

wherein:
Y is absent, —O—, —S—, —C(O)—, —CH₂O—, —NR¹⁰—, —C(O)NR¹¹— or —NR¹²C(O)—, wherein
R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, —C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶, wherein
R¹³ is hydrogen, C₁-C₄ alkyl, C₁-C₄ haloalkyl, substituted or unsubstituted phenyl,
R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring, or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X¹, X², and X³ is independently N or CR⁹, wherein one of X¹, X², or X³ is N;
each R⁷, R⁸, and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

In some embodiments, the compound has the structure of Formula (II), or a pharmaceutically acceptable salt or solvate thereof:

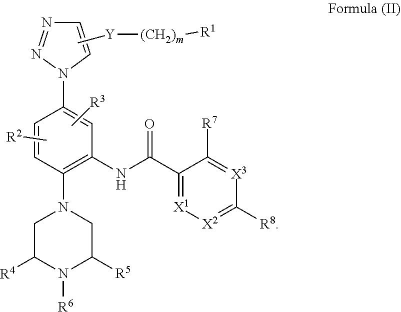

In some embodiments, n is 1 or 2. In some embodiments, L is —(CH₂)ₘ—, wherein m is an integer from 1-6. In some embodiments, m is 1, 2, 3, or 4. In some embodiments, X¹ is N; and X² and X³ are each independently CR⁹. In some embodiments, X² is N; and X¹ and X³ are each independently CR⁹. In some embodiments, X³ is N; and X¹ and X² are each independently CR⁹. In some embodiments, X¹ is N; and X² and X³ are CR⁹. In some embodiments, X¹ and X² are N; and X³ is CR⁹. In some embodiments, X¹, X², and X³ are each N.

In some embodiments, the compound has the structure of Formula (IIIA), or a pharmaceutically acceptable salt or solvate thereof:

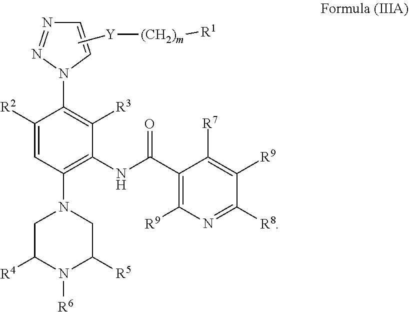

In some embodiments, each R⁹ is independently hydrogen, halogen, C₁-C₆ alkyl, C₁-C₆ alkoxy, trifluoromethyl, difluoromethyl, trifluoromethoxy, difluoromethoxy, amino, nitro, or cyano. In some embodiments, each R⁹ is independently hydrogen, chloro, fluoro, bromo, amino, cyano, methyl, methoxy, trifluoromethyl, difluoromethyl, or trifluoromethyl. In some embodiments, each R⁷ and R⁸ is independently hydrogen, halogen, C₁-C₆ alkyl, C₁-C₆ alkoxy, trifluoromethyl, difluoromethyl, trifluoromethoxy, difluoromethoxy, nitro or cyano. In some embodiments, R⁷ is trifluoromethyl, difluoromethyl, trifluoromethoxy, or difluoromethoxy; and R⁸ is chloro, fluoro, or bromo.

In some embodiments, the compound has the structure of Formula (IV), or a pharmaceutically acceptable salt or solvate thereof:

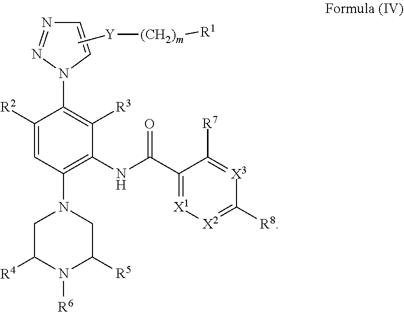

In some embodiments, Y is absent. In some embodiments, Y is —O—, —S—, —C(O)—, —CH₂O—, —NR¹⁰—, —C(O)NR¹¹— or —NR¹²C(O)—. In some embodiments, Y is —O— or —NR¹⁰—, wherein R¹⁰ is hydrogen or C₁-C₄ alkyl. In some embodiments, Y is —C(O)NR¹¹—, wherein R¹¹ is hydrogen or C₁-C₄ alkyl. In some embodiments, R¹ is hydrogen, amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring. In some embodiments, R¹ is substituted or unsubstituted nitrogen- or oxygen-containing 3 to 7 membered heterocyclic ring. In some embodiments, the 3-7 membered heterocyclic ring is piperidine, piperazine, or morpholine. In some embodiments, R¹ is —NR¹³COR¹⁴, —C(O)NR¹⁵R¹⁶ or —NR¹⁵R¹⁶. In some embodiments, R¹ is —NR¹⁵R¹⁶, wherein R¹⁵ and R¹⁶ are bonded to form a nitrogen- or oxygen-containing 3 to 7 membered heterocyclic ring. In some embodiments, R⁴ and R⁵ are each independently hydrogen or C₁-C₆ alkyl. In some embodiments, R⁴ and R⁵ are each methyl. In some embodiments, R⁴ and R⁵ are each hydrogen. In some embodiments, R⁴ is hydrogen and R⁵ is C₁-C₆ alkyl. In some embodiments, R⁴ is C₁-C₆ alkyl and R⁵ is hydrogen. In some embodiments, R⁶ is hydrogen or C₁-C₆ alkyl. In some embodiments, R⁶ is methyl. In some embodiments, R² is halogen or hydrogen; and R³ is hydrogen.

In one aspect, described herein is a compound that has the structure of Formula (V), or a pharmaceutically acceptable salt or solvate thereof:

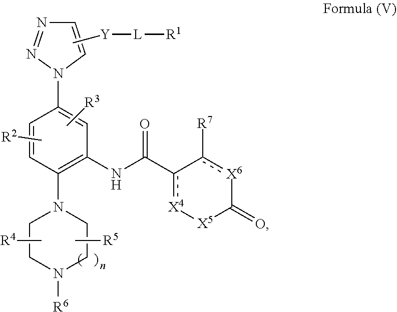

wherein:
Y is absent, —O—, —S—, —C(O)—, —CH₂O—, —NR¹⁰—, —C(O)NR¹¹— or —NR¹²C(O)—, wherein
R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, —C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶, wherein
R¹³ is hydrogen, C₁-C₄ alkyl, C₁-C₄ haloalkyl, substituted or unsubstituted phenyl,
R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring, or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X⁴, X⁵, and X⁶ is independently NR^{9A} or CR⁹; wherein one of X⁴, X⁵, or X⁶ is NR^{9A};
each R^{9A} is independently hydrogen or C₁-C₆ alkyl;
each R⁷ and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

In some embodiments, the compound has the structure of Formula (VI), or a pharmaceutically acceptable salt or solvate thereof:

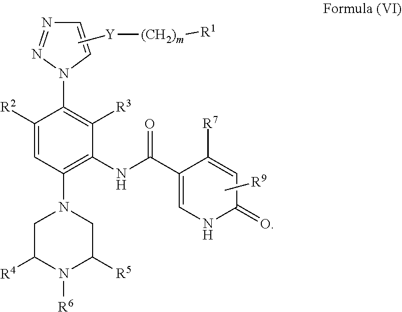

In some embodiments, n is 1 or 2. In some embodiments, L is —(CH₂)ₘ—, wherein m is an integer from 1-6. In some embodiments, X² is NH; and X¹ and X³ are each independently CR⁹. In some embodiments, each R⁷ and R⁹ is independently hydrogen, halogen, C₁-C₆ alkyl, C₁-C₆ alkoxy, trifluoromethyl, difluoromethyl, trifluoromethoxy, difluoromethoxy, amino, nitro, or cyano. In some embodiments, Y is absent. In some embodiments, Y is —O—, —S—, —C(O)—, —CH₂O—, —NR¹⁰—, —C(O)NR¹¹— or —NR¹²C(O)—. In some embodiments, Y is —O— or —NR¹⁰—, wherein R¹⁰ is hydrogen or C₁-C₄ alkyl. In some embodiments, Y is —C(O)NR¹¹—, wherein R¹¹ is hydrogen or C₁-C₄ alkyl. In some embodiments, R¹ is hydrogen, amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring. In some embodiments, R¹ is —NR¹⁵R¹⁶, wherein R¹⁵ and R¹⁶ are bonded to form a nitrogen- or oxygen-containing 3 to 7 membered heterocyclic ring. In some embodiments, R⁴ and R⁵ are each independently hydrogen or C₁-C₆ alkyl. In some embodiments, R⁶ is hydrogen or C₁-C₆ alkyl. In some embodiments, R² is halogen or hydrogen; and R³ is hydrogen. In some embodiments, the compound is a compound described herein or a pharmaceutically acceptable salt or solvate thereof.

Embodiments of compounds of Formula (I), Formula (II), Formula (IIIA), Formula (IV), Formula (V) and Formula (VI) are inhibitors of the MLL1-WDR5 protein-protein interaction.

In another aspect described herein are pharmaceutical compositions comprising a compound as described herein, or a pharmaceutically acceptable salt or solvate thereof, and one or more pharmaceutically acceptable carriers, diluents and excipients.

Another aspect described herein is a method for the treatment or prevention of acute leukemia in a patient in need thereof, comprising administering to the patient a therapeutically acceptable dose of a compound described herein, or a pharmaceutically acceptable salt or solvate thereof. Another aspect described herein is a method for the treatment or prevention of acute leukemia in a patient in need thereof, comprising administering to the patient a compound or pharmaceutical composition as described herein. In some embodiments, the acute leukemia is acute leukemia with MLL1 gene rearrangement.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

Any combination of the groups described above or below for the various variables is contemplated herein. Throughout the specification, groups and substituents thereof are chosen by one skilled in the field to provide stable moieties and compounds.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The haloalkylpyridyl triazole compounds as described herein have strong inhibitory activity against MLL1-WDR5 protein-protein interaction, can reduce the MLL1 catalytic activity of MLL1 at cellular level, downregulate the expression of Hox and Meis-1 genes and induce apoptosis of leukemia cells. Additionally, the compounds described herein exhibit good water solubility and pharmaceutical safety, and can be used for the treatment of cancers, such as but not limited to leukemia.

### Compounds

In one aspect, described herein is a compound of Formula (I), or a pharmaceutically acceptable salt or solvate thereof:

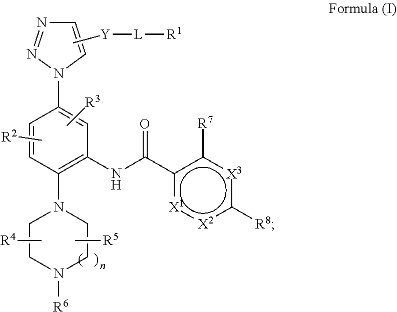

wherein:
Y is absent, —O—, —S—, —C(O)—, —CH₂O—, —NR¹⁰—, —C(O)NR¹¹— or —NR¹²C(O)—, wherein
   R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶, wherein
   R¹³ is hydrogen, C₁-C₄ alkyl, C₁-C₄ haloalkyl, substituted or unsubstituted phenyl,
   R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
   R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring,
   or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X¹, X², and X³ is independently N or CR⁹, wherein one of X¹, X², or X³ is N;
each R⁷, R⁸, and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

For any and all of the embodiments, substituents are selected from among a subset of the listed alternatives.

In some embodiments, the compound comprises a substituted or unsubstituted 6-membered monocyclic heteroaryl, substituted or unsubstituted with R⁷, R⁸, and R⁹. In some embodiments, the 6-membered monocyclic heteroaryl comprises one, two or three N atoms. In some embodiments, the 6-membered monocyclic heteroaryl comprises one N atom. In some embodiments, the 6-membered monocyclic heteroaryl comprises two N atoms. In some embodiments, the 6-membered monocyclic heteroaryl is pyridine, pyrazine, pyrimidine, pyridazine, or 1,2,4-triazine. In some embodiments, the heteroaryl is pyridine. In some embodiments, the heteroaryl is pyrimidine. In some embodiments, the heteroaryl is pyrazine. In some embodiments, the heteroaryl is pyridazine. In some embodiments, the heteroaryl is 1,2,4-triazine. In some embodiments, the heteroaryl is pyridin-2(1H)-one.

Embodiments of compounds of Formula (I) are inhibitors of the MLL1-WDR5 protein-protein interaction.

In some embodiments, the compound of Formula (I) has the structure of Formula (II), or a pharmaceutically acceptable salt or solvate thereof:

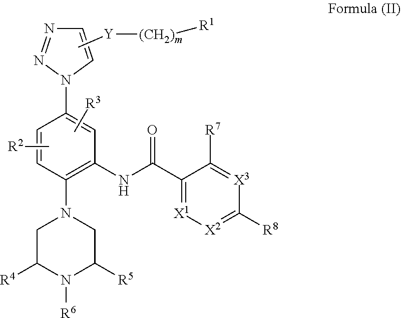

wherein, unless otherwise defined herein, the variable groups have the definitions provided in Formula (I).

In some embodiments, each X¹, X², and X³ is independently N or CR⁹, wherein one of X¹, X², or X³ is N. In some embodiments, one of X¹, X², or X³ is N. In some embodiments, each X¹, X², and X³ cannot simultaneously be CR⁹.

In some embodiments, X¹ is N; and X² and X³ are each independently CR⁹.

In some embodiments, X² is N; and X¹ and X³ are each independently CR⁹.

In some embodiments, X³ is N; and X¹ and X² are each independently CR⁹.

In some embodiments, X¹ is N; and X² and X³ are CR⁹.

In some embodiments, X¹ and X² are N; and X³ is CR⁹.

In some embodiments, X¹, X², and X³ are each N.

Embodiments of compounds of Formula (II) are inhibitors of the MLL1-WDR5 protein-protein interaction.

In some embodiments, the compound of Formula (I) has the structure of Formula (IIIA), or a pharmaceutically acceptable salt or solvate thereof:

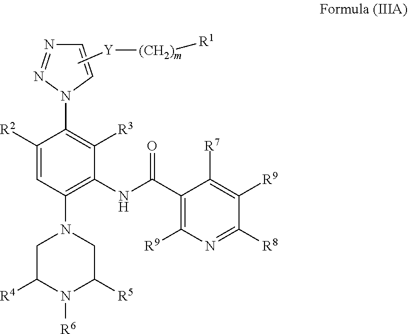

wherein, unless otherwise defined herein, the variable groups have the definitions provided in Formula (I).

In some embodiments, the compound of Formula (I) has the structure of Formula (IIIB), or a pharmaceutically acceptable salt or solvate thereof:

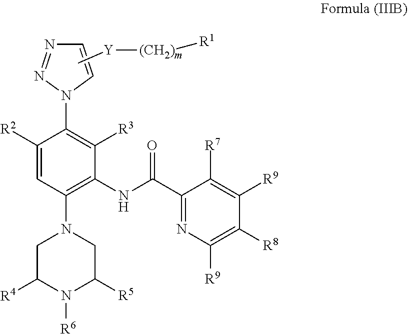

wherein, unless otherwise defined herein, the variable groups have the definitions provided in Formula (I).

In some embodiments, the compound of Formula (I) has the structure of Formula (IIIC), or a pharmaceutically acceptable salt or solvate thereof:

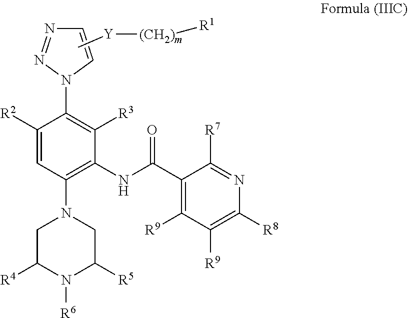

wherein, unless otherwise defined herein, the variable groups have the definitions provided in Formula (I).

In some embodiments, the compound of Formula (I) has the structure of Formula (IIID), or a pharmaceutically acceptable salt or solvate thereof:

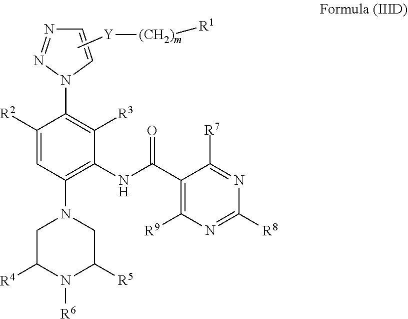

wherein, unless otherwise defined herein, the variable groups have the definitions provided in Formula (I).

In some embodiments, the compound of Formula (I) has the structure of Formula (IIIE), or a pharmaceutically acceptable salt or solvate thereof:

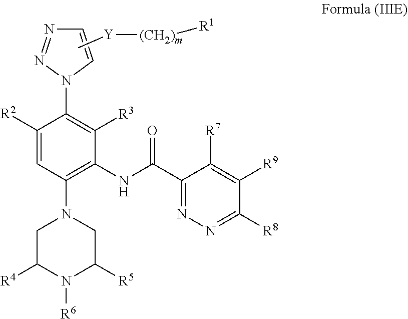

wherein, unless otherwise defined herein, the variable groups have the definitions provided in Formula (I).

In some embodiments, the compound of Formula (I) has the structure of Formula (IIIF), or a pharmaceutically acceptable salt or solvate thereof:

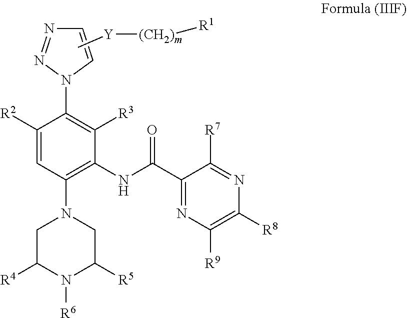

wherein, unless otherwise defined herein, the variable groups have the definitions provided in Formula (I).

In some embodiments, the compound of Formula (I) has the structure of Formula (IIIG), or a pharmaceutically acceptable salt or solvate thereof:

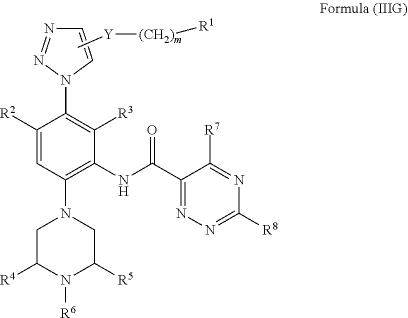

wherein, unless otherwise defined herein, the variable groups have the definitions provided in Formula (I).

In some embodiments of the compounds of Formulas (IIIA), (IIIB), (IIIC), (IIID), (IIIE), (IIIF) and (IIIG), each R⁹ is independently hydrogen, halogen, C₁-C₆ alkyl, C₁-C₆ alkoxy, trifluoromethyl, difluoromethyl, trifluoromethoxy, difluoromethoxy, amino, nitro, or cyano. In some embodiments, each R⁹ is independently hydrogen, chloro, fluoro, bromo, amino, cyano, methyl, methoxy, trifluoromethyl, difluoromethyl, or trifluoromethyl. In some embodiments, each R⁹ is independently —Cl, —F, —OH, —CF₃, —CH₃, or —OCH₃. In some embodiments, each R⁹ is independently —Cl or —F. In some embodiments, each R⁹ is independently —CF₃. In some embodiments, each R⁹ is independently hydrogen.

In some embodiments of the compounds of Formulas (IIIA), (IIIB), (IIIC), (IIID), (IIIE), (IIIF) and (IIIG), each R⁷ and R⁸ is independently hydrogen, halogen, C₁-C₆ alkyl, C₁-C₆ alkoxy, trifluoromethyl, difluoromethyl, trifluoromethoxy, difluoromethoxy, nitro or cyano. In some embodiments, each R⁷ and R⁸ is independently hydrogen, chloro, fluoro, bromo, amino, cyano, methyl, methoxy, trifluoromethyl, difluoromethyl, or trifluoromethyl. In some embodiments, each R⁷ and R⁸ is independently —Cl, —F, —OH, —CF₃, —CH₃, or —OCH₃.

In some embodiments of the compounds of Formulas (IIIA), (IIIB), (IIIC), (IIID), (IIIE), (IIIF) and (IIIG), R⁷ is trifluoromethyl, difluoromethyl, trifluoromethoxy, or difluoromethoxy; and R⁸ is hydrogen, chloro, fluoro, or bromo. In some embodiments, R⁷ is —CF₃; and R⁸ is hydrogen, —Cl, or F. In some embodiments, R⁷ is —CF₃; and R⁸ is —Cl.

In some embodiments of the compounds of Formulas (IIIA), (IIIB), (IIIC), (IIID), (IIIE), (IIIF) and (IIIG) are inhibitors of the MLL1-WDR5 protein-protein interaction.

In some embodiments, the compound of Formula (I) has the structure of Formula (IV), or a pharmaceutically acceptable salt or solvate thereof:

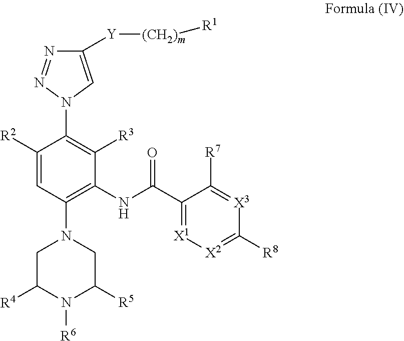

wherein, unless otherwise defined herein, the variable groups have the definitions provided in Formula (I).

In some embodiments, the compounds of Formula (IV) are inhibitors of the MLL1-WDR5 protein-protein interaction.

In another aspect described herein is a compound having the structure of Formula (V), or a pharmaceutically acceptable salt or solvate thereof:

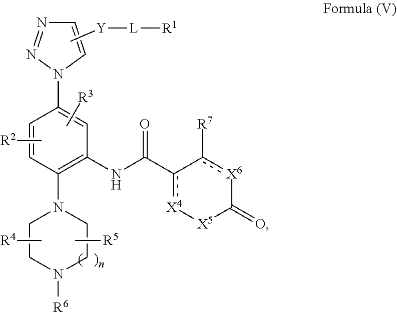

wherein:
Y is absent, —O—, —S—, —C(O)—, —CH₂O—, —NR¹⁰—, —C(O)NR¹¹— or —NR¹²C(O)—, wherein
R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶, wherein
R¹³ is hydrogen, C₁-C₄ alkyl, C₁-C₄ haloalkyl, substituted or unsubstituted phenyl,
R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring, or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X⁴, X⁵, and X⁶ is independently NR^{9A} or CR⁹; wherein one of X⁴, X⁵, or X⁶ is NR^{9A};
each R^{9A} is independently hydrogen or C₁-C₆ alkyl;
each R⁷ and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

In some embodiments, the compound of Formula (V), or a pharmaceutically acceptable salt or solvate thereof, comprises a pyridin-2(1H)-one, substituted or unsubstituted with R⁷ and R⁹.

In some embodiments, X³ is NR^{9A}; and X⁴ and X⁵ are each independently CR⁹. In some embodiments, X³ is NH; and X⁴ and X⁵ are each independently CR⁹. In some embodiments, X⁴ is NR^{9A}; and X³ and X⁵ are each independently CR⁹. In some embodiments, X⁴ is NH; and X³ and X⁵ are each independently CR⁹. In some embodiments, X⁵ is NR^{9A}; and X³ and X⁴ are each independently CR⁹. In some embodiments, X⁵ is NH; and X³ and X⁴ are each independently CR⁹.

In some embodiments, the compounds of Formula (V) are inhibitors of the MLL1-WDR5 protein-protein interaction.

In some embodiments, the compound of Formula (I) has the structure of Formula (VI), or a pharmaceutically acceptable salt or solvate thereof:

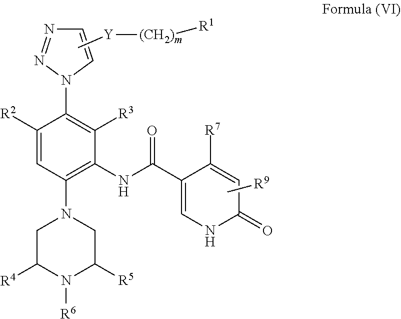

wherein, unless otherwise defined herein, the variables have the definitions provided in Formula (I).

In some embodiments, each R⁹ is independently halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy. In some embodiments, each R⁹ is independently chloro, fluoro, bromo, —CH₃, —OCH₃, or —CF₃. In some embodiments, each R⁹ is independently hydrogen.

In some embodiments, R⁷ is halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy. In some embodiments, R⁷ is chloro, fluoro, bromo, —CH₃, —OCH₃, or —CF₃. In some embodiments, R⁷ is —Cl, —F, or —Br. In some embodiments, R⁷ is —CF₃. In some embodiments, R⁷ is hydrogen.

In some embodiments, m is 1, 2, 3, 4, or 5. In some embodiments, m is 1, 2, 3, or 4. In some embodiments, m is 1, 2, or 3. In some embodiments, m is 1. In some embodiments, m is 2. In some embodiments, m is 3. In some embodiments, m is 4. In some embodiments, m is 5. In some embodiments, m is 6.

In some embodiments, n is 1 or 2. In some embodiments, n is 1. In some embodiments, n is 2. In some embodiments, n is 0.

In some embodiments, Y is —O—, —S—, —C(O)—, —CH₂O—, —NR¹⁰—, —C(O)NR¹¹— or —NR¹²C(O)—. In some embodiments, Y is —O— or —NR¹⁰—. In some embodiments, Y is —O— or —NR¹⁰—, wherein R¹⁰ is hydrogen or C₁-C₄ alkyl. In some embodiments, Y is —O—. In some embodiments, Y is —NR¹⁰—. In some embodiments, Y is —NH—. In some embodiments, Y is —NCH₃—. In some embodiments, Y is —S—. In some embodiments, Y is —C(O)—. In some embodiments, Y is —CH₂O—.

In some embodiments, Y is —C(O)NR¹¹. In some embodiments, Y is —C(O)NR¹¹—, wherein R¹¹ is hydrogen or C₁-C₄ alkyl. In some embodiments, Y is —C(O)NH—. In some embodiments, Y is C(O)N(CH₃)—. In some embodiments, Y is —NR¹²C(O)—. In some embodiments, Y is —NR¹²C(O)—, wherein R¹¹ is hydrogen or C₁-C₄ alkyl. In some embodiments, Y is —NHC(O)—. In some embodiments, Y is —N(CH₃)C(O)—.

In some embodiments, Y is absent.

In some embodiments, R¹ is amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring. In some embodiments, R¹ is hydrogen. In some embodiments, R¹ is hydroxyl, thiol, carboxyl, cyano, C₁-C₄ alkyl, or C₁-C₆ alkoxy. In some embodiments, R¹ is —OH, —SH, —CN, —CH₃, or —OCH₃. In some embodiments, R¹ is phenyl.

In some embodiments, R¹ is a substituted or unsubstituted nitrogen- or oxygen-containing 3 to 7 membered heterocyclic ring. In some embodiments, the nitrogen- or oxygen-containing 3 to 7 membered heterocyclic ring is pyrrolidine, piperidine, piperazine, or morpholine. In some embodiments, the nitrogen- or oxygen-containing 3-7 membered heterocyclic ring is pyrrolidine. In some embodiments, the 3 to 7 membered ring is piperidine. In some embodiments, the 3 to 7 membered ring is piperazine. In some embodiments, the 3 to 7 membered ring is morpholine.

In some embodiments, R¹ is —NR¹³COR¹⁴, —C(O)NR¹⁵R¹⁶ or —NR¹⁵R¹⁶. In some embodiments, R¹ is —NR¹³COR¹⁴. In some embodiments, R¹ is —C(O)NR¹⁵R¹⁶. In some embodiments, R¹ is —NR¹⁵R¹⁶.

In some embodiments, R¹ is —NR¹⁵R¹⁶, wherein R¹⁵ and R¹⁶ are bonded together with the nitrogen to which they are attached to form a nitrogen- or oxygen-containing 3 to 7 membered heterocyclic ring. In some embodiments, the 3 to 7 membered ring is piperazine, or morpholine. In some embodiments, the 3 to 7 membered ring is piperazine. In some embodiments, the 3 to 7 membered ring is morpholine.

In some embodiments, R⁴ and R⁵ are each independently C₃-C₆ cycloalkyl. In some embodiments, R⁴ and R⁵ are each independently cyclopropyl, cyclobutyl, cyclopentyl, or cyclohexyl.

In some embodiments, R⁴ and R⁵ are each independently hydrogen or C₁-C₆ alkyl. In some embodiments, R⁴ and R⁵ are each independently C₁-C₆ alkyl. In some embodiments, R⁴ and R⁵ are each independently methyl, ethyl, or isopropyl. In some embodiments, R⁴ and R⁵ are each methyl. In some embodiments, R⁴ and R⁵ are each hydrogen.

In some embodiments, R⁴ is hydrogen; and R⁵ is C₃-C₆ cycloalkyl or C₁-C₆ alkyl. In some embodiments, R⁴ is hydrogen and R⁵ is C₁-C₆ alkyl. In some embodiments, R⁴ is hydrogen; and R⁵ is methyl, ethyl or isopropyl. In some embodiments, R⁴ is hydrogen; and R⁵ is methyl. In some embodiments, R⁴ is C₃-C₆ cycloalkyl or C₁-C₆ alkyl; and R⁵ is hydrogen. In some embodiments, R⁴ is C₁-C₆ alkyl; and R⁵ is hydrogen. In some embodiments, R⁴ is methyl, ethyl, or isopropyl; and R⁵ is hydrogen. In some embodiments, R⁴ is methyl; and R⁵ is hydrogen.

In some embodiments, R⁶ is C₃-C₆ cycloalkyl. In some embodiments, R⁶ is cyclopropyl, cyclobutyl, cyclopentyl, or cyclohexyl. In some embodiments, R⁶ is cyclopropyl. In some embodiments, R⁶ is cyclobutyl. In some embodiments, R⁶ is cyclopentyl. In some embodiments, R⁶ is cyclohexyl.

In some embodiments, R⁶ is hydrogen or C₁-C₆ alkyl. In some embodiments, R⁶ is C₁-C₆ alkyl. In some embodiments, R⁶ is methyl. In some embodiments, R⁶ is methyl, ethyl, propyl, isopropyl, sec-butyl, iso-butyl or tert-butyl. In some embodiments, R⁶ is methyl. In some embodiments, R⁶ is ethyl. In some embodiments, R⁶ is tert-butyl. In some embodiments, R⁶ is hydrogen.

In some embodiments, R² and R³ are independently hydrogen, halogen, methyl, or methoxy. In some embodiments, R² and R³ are independently hydrogen, chloro, fluoro, bromo, iodo, methyl, or methoxy. In some embodiments, R² and R³ are independently hydrogen, chloro, fluoro, or methyl. In some embodiments, R² and R³ are independently difluoromethoxy or trifluoromethoxy.

In some embodiments, R² and R³ are each hydrogen, halogen, or methyl. In some embodiments, R² and R³ are each hydrogen. In some embodiments, R² and R³ are each halogen. In some embodiments, R² and R³ are each methyl.

In some embodiments, R² is halogen or methyl; and R³ is hydrogen. In some embodiments, R² is choro, fluoro, or methyl; and R³ is hydrogen. In some embodiments, R² is hydrogen; and R³ is halogen or methyl. In some embodiments, R² is hydrogen; and R³ is chloro, fluoro, or methyl.

In some embodiments, the compounds of Formula (VI) are inhibitors of the MLL1-WDR5 protein-protein interaction.

Any combination of the groups described above for the various variables is contemplated herein. Throughout the specification, groups and substituents thereof are chosen by one skilled in the field to provide stable moieties and compounds.

In some embodiments, compounds described herein include, but are not limited to the compounds of Tables 1, 2, or 3, or a pharmaceutically acceptable salt or solvate thereof.

TABLE 1
Compounds of the disclosure.

| Compound. | |
|---|---|
| No. | Structure |
| 1 | 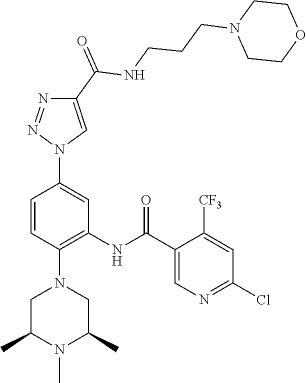 [SMILES: C[C@@H]1CN(c2ccc(-n3cc(C(=O)NCCCN4CCOCC4)nn3)cc2NC(=O)c2cnc(Cl)cc2C(F)(F)F)C[C@H](C)N1C] |
| 2 | 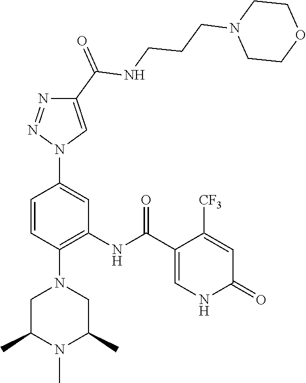 [SMILES: C[C@@H]1CN(c2ccc(-n3cc(C(=O)NCCCN4CCOCC4)nn3)cc2NC(=O)c2c[nH]c(=O)cc2C(F)(F)F)C[C@H](C)N1C] |
| 3 | 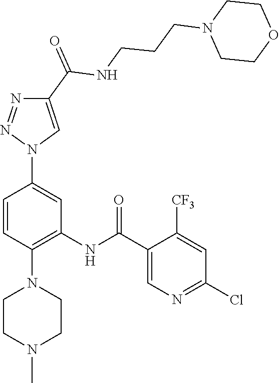 [SMILES: CN1CCN(c2ccc(-n3cc(C(=O)NCCCN4CCOCC4)nn3)cc2NC(=O)c2cnc(Cl)cc2C(F)(F)F)CC1] |
| 4 | 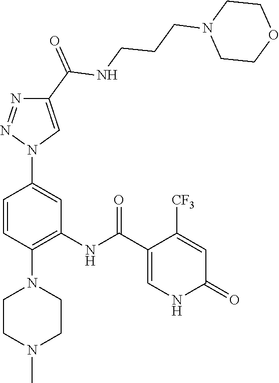 [SMILES: CN1CCN(c2ccc(-n3cc(C(=O)NCCCN4CCOCC4)nn3)cc2NC(=O)c2c[nH]c(=O)cc2C(F)(F)F)CC1] |
| 5 | 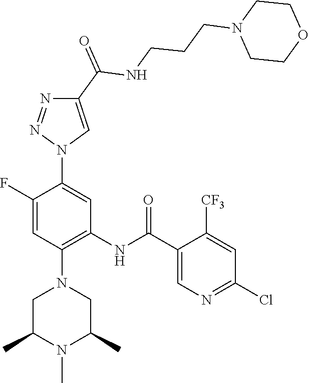 [SMILES: C[C@@H]1CN(c2cc(F)c(-n3cc(C(=O)NCCCN4CCOCC4)nn3)cc2NC(=O)c2cnc(Cl)cc2C(F)(F)F)C[C@H](C)N1C] |
| 6 | 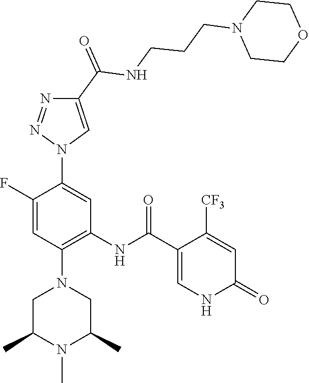 [SMILES: C[C@@H]1CN(c2cc(F)c(-n3cc(C(=O)NCCCN4CCOCC4)nn3)cc2NC(=O)c2c[nH]c(=O)cc2C(F)(F)F)C[C@H](C)N1C] |
| 7 | 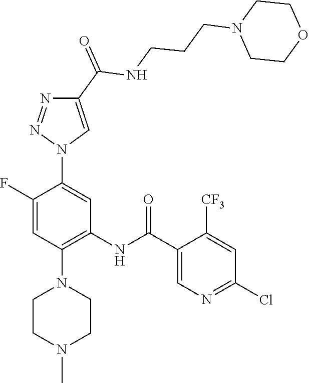 [SMILES: CN1CCN(c2cc(F)c(-n3cc(C(=O)NCCCN4CCOCC4)nn3)cc2NC(=O)c2cnc(Cl)cc2C(F)(F)F)CC1] |
| 8 | 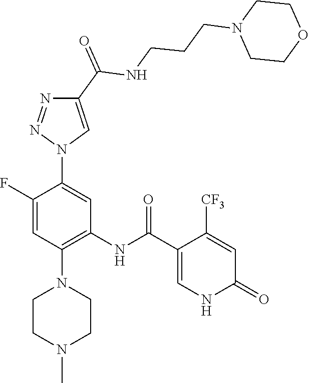 [SMILES: CN1CCN(c2cc(F)c(-n3cc(C(=O)NCCCN4CCOCC4)nn3)cc2NC(=O)c2c[nH]c(=O)cc2C(F)(F)F)CC1] |
| 9 | 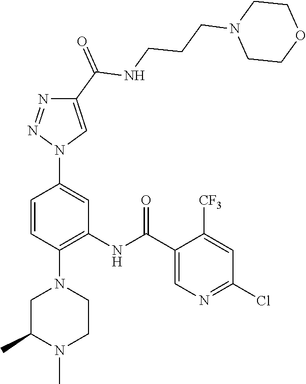 [SMILES: C[C@H]1CN(c2ccc(-n3cc(C(=O)NCCCN4CCOCC4)nn3)cc2NC(=O)c2cnc(Cl)cc2C(F)(F)F)CCN1C] |
| 10 | 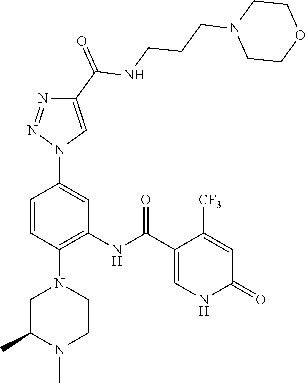 [SMILES: C[C@H]1CN(c2ccc(-n3cc(C(=O)NCCCN4CCOCC4)nn3)cc2NC(=O)c2c[nH]c(=O)cc2C(F)(F)F)CCN1C] |
| 11 | 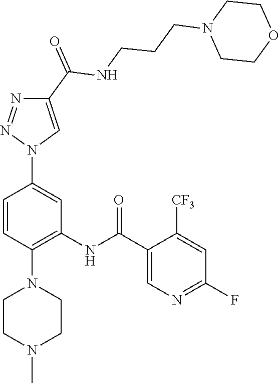 [SMILES: CN1CCN(c2ccc(-n3cc(C(=O)NCCCN4CCOCC4)nn3)cc2NC(=O)c2cnc(F)cc2C(F)(F)F)CC1] |
| 12 | 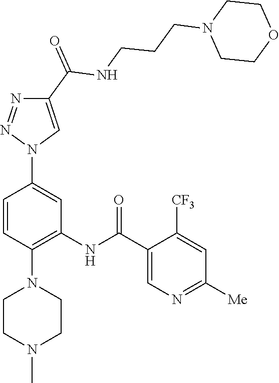 [SMILES: Cc1cc(C(F)(F)F)c(C(=O)Nc2cc(-n3cc(C(=O)NCCCN4CCOCC4)nn3)ccc2N2CCN(C)CC2)cn1] |
| 13 | 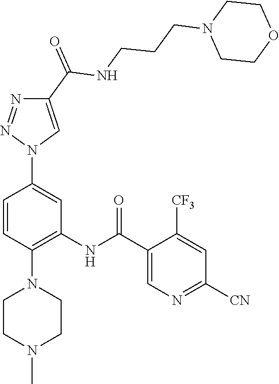 [SMILES: CN1CCN(c2ccc(-n3cc(C(=O)NCCCN4CCOCC4)nn3)cc2NC(=O)c2cnc(C#N)cc2C(F)(F)F)CC1] |
| 14 | 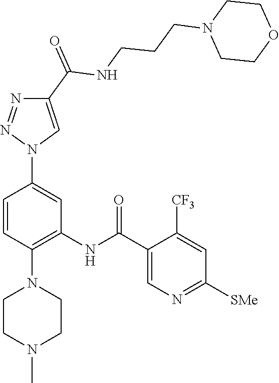 [SMILES: CSc1cc(C(F)(F)F)c(C(=O)Nc2cc(-n3cc(C(=O)NCCCN4CCOCC4)nn3)ccc2N2CCN(C)CC2)cn1] |
| 15 | 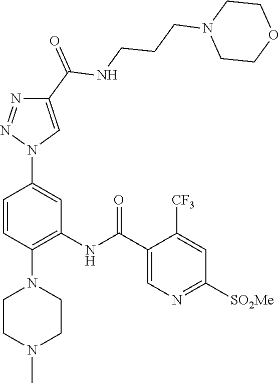 [SMILES: CN1CCN(c2ccc(-n3cc(C(=O)NCCCN4CCOCC4)nn3)cc2NC(=O)c2cnc(S(C)(=O)=O)cc2C(F)(F)F)CC1] |
| 16 | 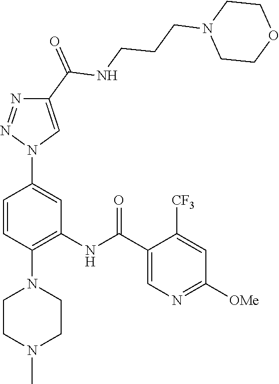 [SMILES: COc1cc(C(F)(F)F)c(C(=O)Nc2cc(-n3cc(C(=O)NCCCN4CCOCC4)nn3)ccc2N2CCN(C)CC2)cn1] |
| 17 | 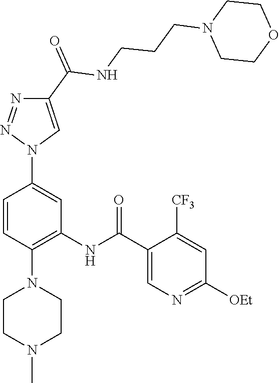 [SMILES: CCOc1cc(C(F)(F)F)c(C(=O)Nc2cc(-n3cc(C(=O)NCCCN4CCOCC4)nn3)ccc2N2CCN(C)CC2)cn1] |
| 18 | 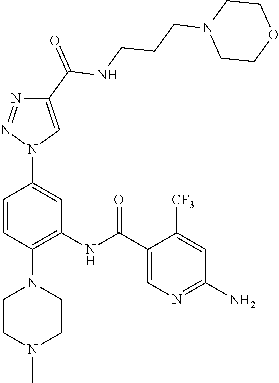 [SMILES: CN1CCN(c2ccc(-n3cc(C(=O)NCCCN4CCOCC4)nn3)cc2NC(=O)c2cnc(N)cc2C(F)(F)F)CC1] |
| 19 | 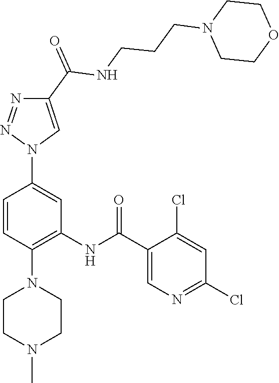 [SMILES: CN1CCN(c2ccc(-n3cc(C(=O)NCCCN4CCOCC4)nn3)cc2NC(=O)c2cnc(Cl)cc2Cl)CC1] |
| 20 | 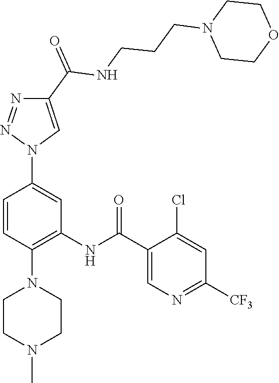 [SMILES: CN1CCN(c2ccc(-n3cc(C(=O)NCCCN4CCOCC4)nn3)cc2NC(=O)c2cnc(C(F)(F)F)cc2Cl)CC1] |
| 21 | 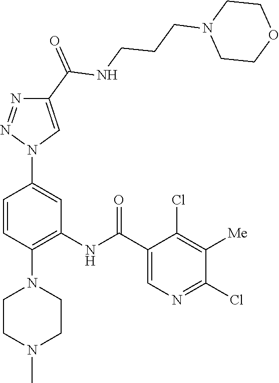 [SMILES: Cc1c(Cl)ncc(C(=O)Nc2cc(-n3cc(C(=O)NCCCN4CCOCC4)nn3)ccc2N2CCN(C)CC2)c1Cl] |
| 22 | 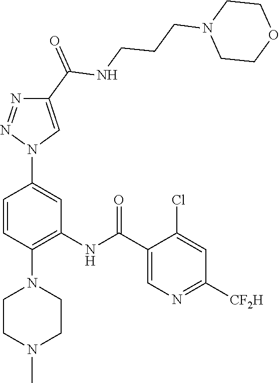 [SMILES: CN1CCN(c2ccc(-n3cc(C(=O)NCCCN4CCOCC4)nn3)cc2NC(=O)c2cnc(C(F)F)cc2Cl)CC1] |
| 23 | 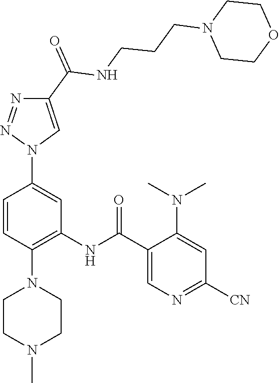 [SMILES: CN1CCN(c2ccc(-n3cc(C(=O)NCCCN4CCOCC4)nn3)cc2NC(=O)c2cnc(C#N)cc2N(C)C)CC1] |
| 24 | 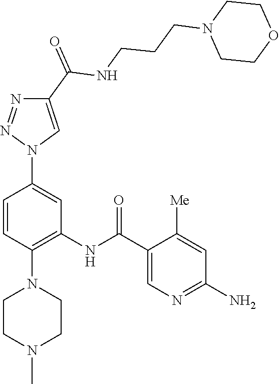 [SMILES: Cc1cc(N)ncc1C(=O)Nc1cc(-n2cc(C(=O)NCCCN3CCOCC3)nn2)ccc1N1CCN(C)CC1] |
| 25 | 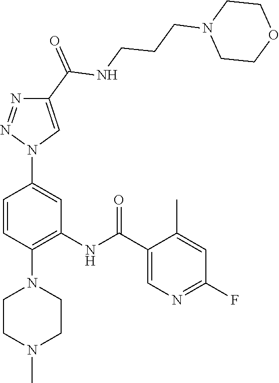 [SMILES: Cc1cc(F)ncc1C(=O)Nc1cc(-n2cc(C(=O)NCCCN3CCOCC3)nn2)ccc1N1CCN(C)CC1] |
| 26 | 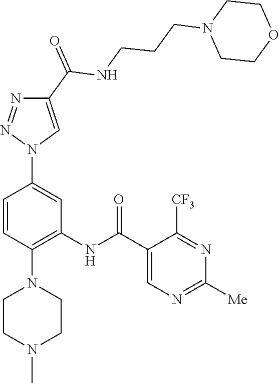 [SMILES: Cc1ncc(C(=O)Nc2cc(-n3cc(C(=O)NCCCN4CCOCC4)nn3)ccc2N2CCN(C)CC2)c(C(F)(F)F)n1] |
| 27 | 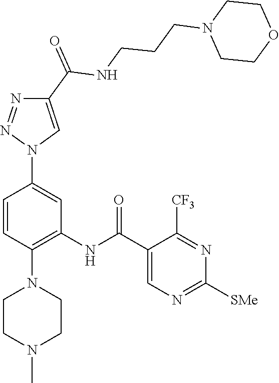 [SMILES: CSc1ncc(C(=O)Nc2cc(-n3cc(C(=O)NCCCN4CCOCC4)nn3)ccc2N2CCN(C)CC2)c(C(F)(F)F)n1] |
| 28 | 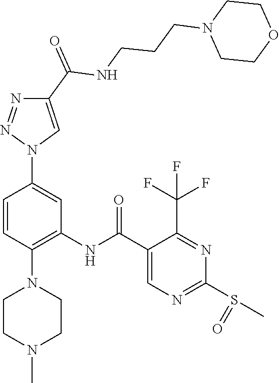 [SMILES: CN1CCN(c2ccc(-n3cc(C(=O)NCCCN4CCOCC4)nn3)cc2NC(=O)c2cnc(S(C)=O)nc2C(F)(F)F)CC1] |
| 29 | 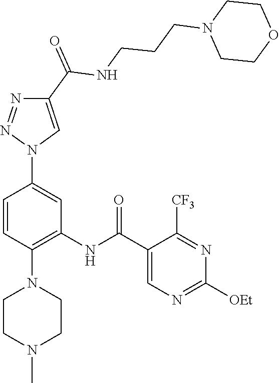 [SMILES: CCOc1ncc(C(=O)Nc2cc(-n3cc(C(=O)NCCCN4CCOCC4)nn3)ccc2N2CCN(C)CC2)c(C(F)(F)F)n1] |
| 30 | 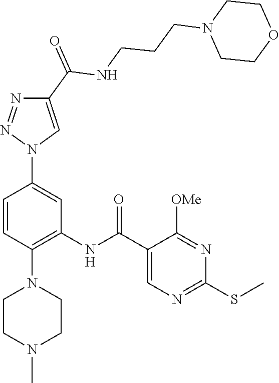 [SMILES: COc1nc(SC)ncc1C(=O)Nc1cc(-n2cc(C(=O)NCCCN3CCOCC3)nn2)ccc1N1CCN(C)CC1] |
| 31 | 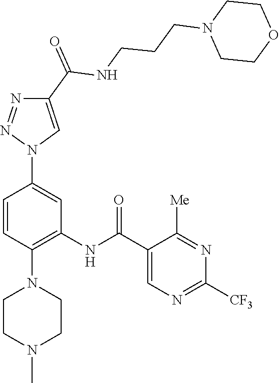 [SMILES: Cc1nc(C(F)(F)F)ncc1C(=O)Nc1cc(-n2cc(C(=O)NCCCN3CCOCC3)nn2)ccc1N1CCN(C)CC1] |
| 32 | 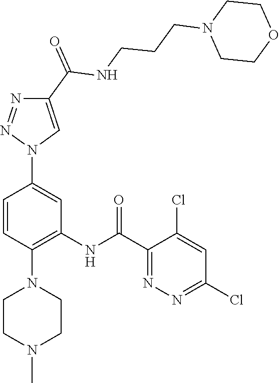 [SMILES: CN1CCN(c2ccc(-n3cc(C(=O)NCCCN4CCOCC4)nn3)cc2NC(=O)c2nnc(Cl)cc2Cl)CC1] |
| 33 | 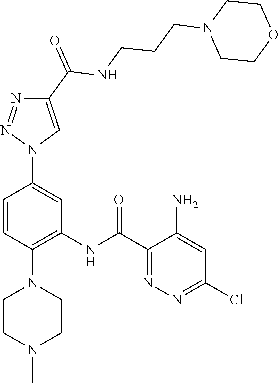 [SMILES: CN1CCN(c2ccc(-n3cc(C(=O)NCCCN4CCOCC4)nn3)cc2NC(=O)c2nnc(Cl)cc2N)CC1] |
| 34 | 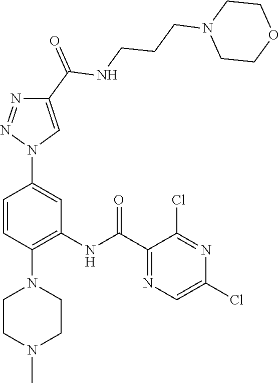 [SMILES: CN1CCN(c2ccc(-n3cc(C(=O)NCCCN4CCOCC4)nn3)cc2NC(=O)c2ncc(Cl)nc2Cl)CC1] |
| 35 | 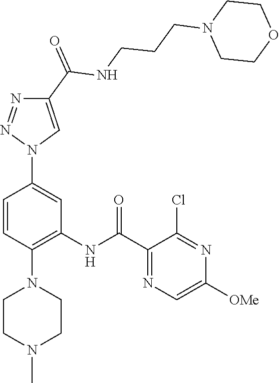 [SMILES: COc1cnc(C(=O)Nc2cc(-n3cc(C(=O)NCCCN4CCOCC4)nn3)ccc2N2CCN(C)CC2)c(Cl)n1] |
| 36 | 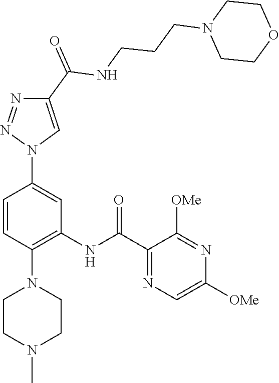 [SMILES: COc1cnc(C(=O)Nc2cc(-n3cc(C(=O)NCCCN4CCOCC4)nn3)ccc2N2CCN(C)CC2)c(OC)n1] |
| 37 | 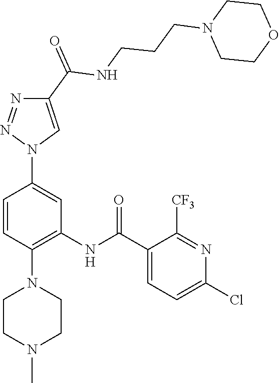 [SMILES: CN1CCN(c2ccc(-n3cc(C(=O)NCCCN4CCOCC4)nn3)cc2NC(=O)c2ccc(Cl)nc2C(F)(F)F)CC1] |
| 38 | 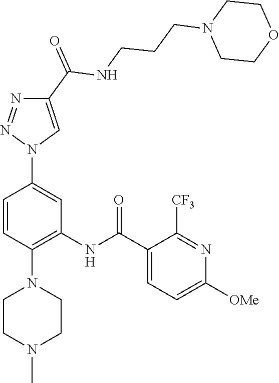 [SMILES: COc1ccc(C(=O)Nc2cc(-n3cc(C(=O)NCCCN4CCOCC4)nn3)ccc2N2CCN(C)CC2)c(C(F)(F)F)n1] |
| 39 | 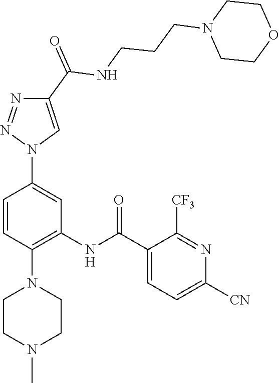 [SMILES: CN1CCN(c2ccc(-n3cc(C(=O)NCCCN4CCOCC4)nn3)cc2NC(=O)c2ccc(C#N)nc2C(F)(F)F)CC1] |
| 40 | 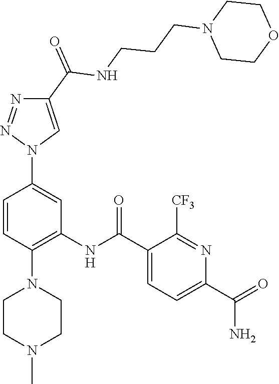 [SMILES: CN1CCN(c2ccc(-n3cc(C(=O)NCCCN4CCOCC4)nn3)cc2NC(=O)c2ccc(C(N)=O)nc2C(F)(F)F)CC1] |
| 41 | 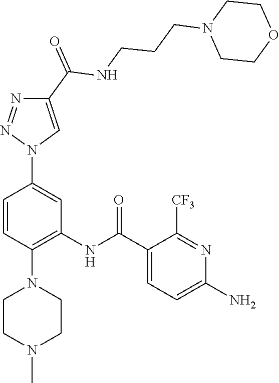 [SMILES: CN1CCN(c2ccc(-n3cc(C(=O)NCCCN4CCOCC4)nn3)cc2NC(=O)c2ccc(N)nc2C(F)(F)F)CC1] |
| 42 | 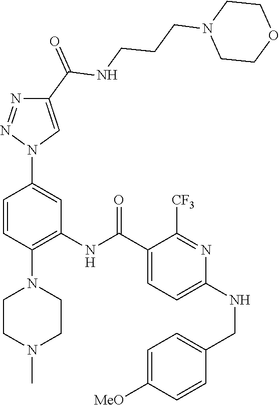 [SMILES: COc1ccc(CNc2ccc(C(=O)Nc3cc(-n4cc(C(=O)NCCCN5CCOCC5)nn4)ccc3N3CCN(C)CC3)c(C(F)(F)F)n2)cc1] |
| 43 | 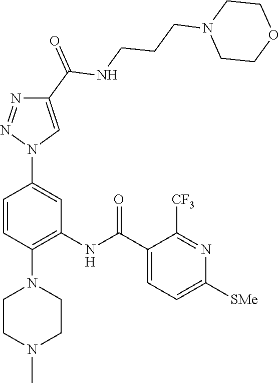 [SMILES: CSc1ccc(C(=O)Nc2cc(-n3cc(C(=O)NCCCN4CCOCC4)nn3)ccc2N2CCN(C)CC2)c(C(F)(F)F)n1] |
| 44 | 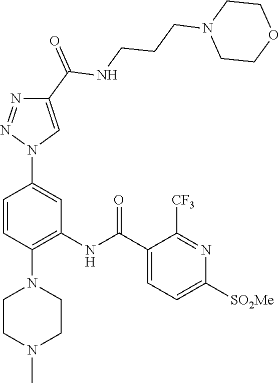 [SMILES: CN1CCN(c2ccc(-n3cc(C(=O)NCCCN4CCOCC4)nn3)cc2NC(=O)c2ccc(S(C)(=O)=O)nc2C(F)(F)F)CC1] |
| 45 | 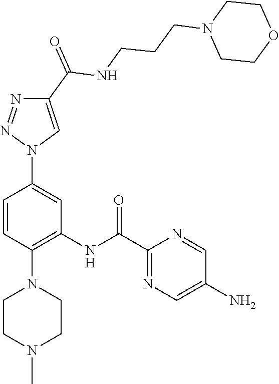 [SMILES: CN1CCN(c2ccc(-n3cc(C(=O)NCCCN4CCOCC4)nn3)cc2NC(=O)c2ncc(N)cn2)CC1] |
| 46 | 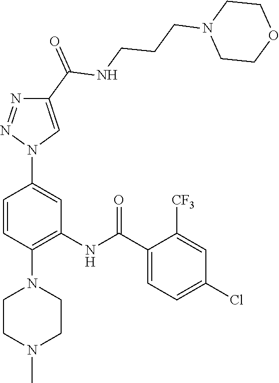 [SMILES: CN1CCN(c2ccc(-n3cc(C(=O)NCCCN4CCOCC4)nn3)cc2NC(=O)c2ccc(Cl)cc2C(F)(F)F)CC1] |
| 47 | 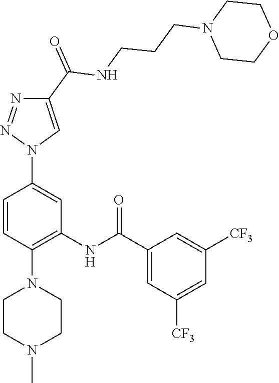 [SMILES: CN1CCN(c2ccc(-n3cc(C(=O)NCCCN4CCOCC4)nn3)cc2NC(=O)c2cc(C(F)(F)F)cc(C(F)(F)F)c2)CC1] |
| 48 | 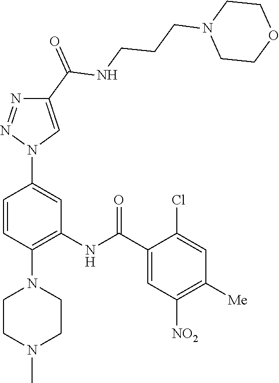 [SMILES: Cc1cc(Cl)c(C(=O)Nc2cc(-n3cc(C(=O)NCCCN4CCOCC4)nn3)ccc2N2CCN(C)CC2)cc1[N+](=O)[O-]] |
| 49 | 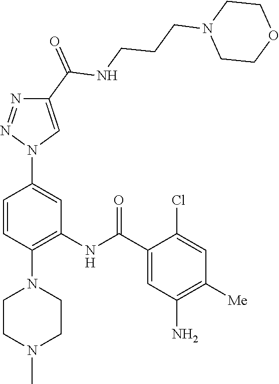 [SMILES: Cc1cc(Cl)c(C(=O)Nc2cc(-n3cc(C(=O)NCCCN4CCOCC4)nn3)ccc2N2CCN(C)CC2)cc1N] |
| 50 | 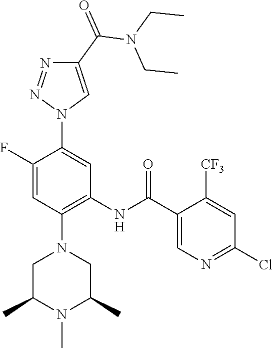 [SMILES: CCN(CC)C(=O)c1cn(-c2cc(NC(=O)c3cnc(Cl)cc3C(F)(F)F)c(N3C[C@@H](C)N(C)[C@@H](C)C3)cc2F)nn1] |
| 51 | 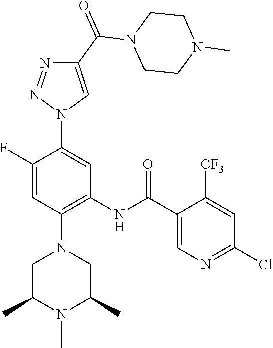 [SMILES: C[C@@H]1CN(c2cc(F)c(-n3cc(C(=O)N4CCN(C)CC4)nn3)cc2NC(=O)c2cnc(Cl)cc2C(F)(F)F)C[C@H](C)N1C] |
| 52 | 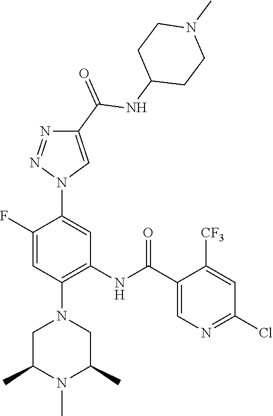 [SMILES: C[C@@H]1CN(c2cc(F)c(-n3cc(C(=O)NC4CCN(C)CC4)nn3)cc2NC(=O)c2cnc(Cl)cc2C(F)(F)F)C[C@H](C)N1C] |
| 53 | 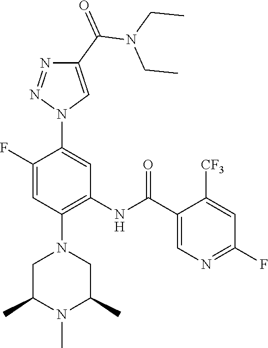 [SMILES: CCN(CC)C(=O)c1cn(-c2cc(NC(=O)c3cnc(F)cc3C(F)(F)F)c(N3C[C@@H](C)N(C)[C@@H](C)C3)cc2F)nn1] |
| 54 | 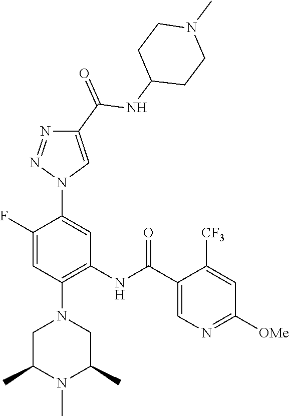 [SMILES: COc1cc(C(F)(F)F)c(C(=O)Nc2cc(-n3cc(C(=O)NC4CCN(C)CC4)nn3)c(F)cc2N2C[C@@H](C)N(C)[C@@H](C)C2)cn1] |
| 55 | 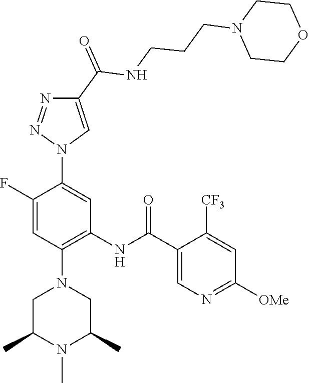 [SMILES: COc1cc(C(F)(F)F)c(C(=O)Nc2cc(-n3cc(C(=O)NCCCN4CCOCC4)nn3)c(F)cc2N2C[C@@H](C)N(C)[C@@H](C)C2)cn1] |
| 56 | 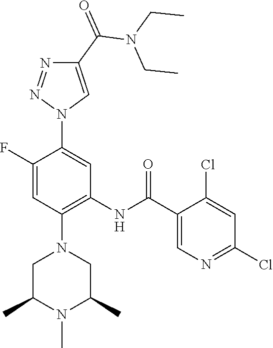 [SMILES: CCN(CC)C(=O)c1cn(-c2cc(NC(=O)c3cnc(Cl)cc3Cl)c(N3C[C@@H](C)N(C)[C@@H](C)C3)cc2F)nn1] |
| 57 | 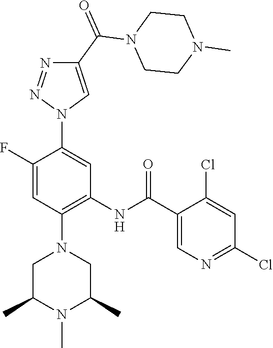 [SMILES: C[C@@H]1CN(c2cc(F)c(-n3cc(C(=O)N4CCN(C)CC4)nn3)cc2NC(=O)c2cnc(Cl)cc2Cl)C[C@H](C)N1C] |
| 58 | 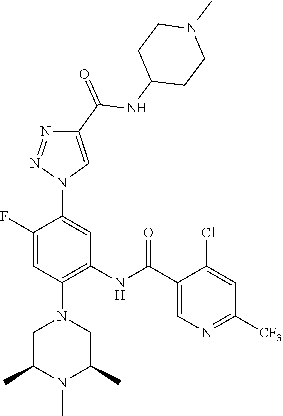 [SMILES: C[C@@H]1CN(c2cc(F)c(-n3cc(C(=O)NC4CCN(C)CC4)nn3)cc2NC(=O)c2cnc(C(F)(F)F)cc2Cl)C[C@H](C)N1C] |
| 59 | 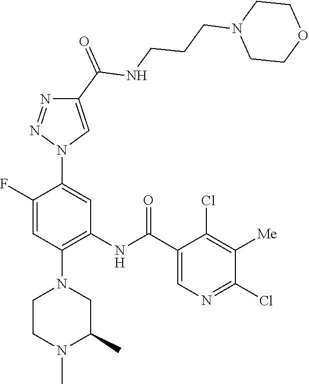 [SMILES: Cc1c(Cl)ncc(C(=O)Nc2cc(-n3cc(C(=O)NCCCN4CCOCC4)nn3)c(F)cc2N2CCN(C)[C@H](C)C2)c1Cl] |

TABLE 2
Compounds of the disclosure.

| Compound | |
|---|---|
| No. | Structure |
| 60 | 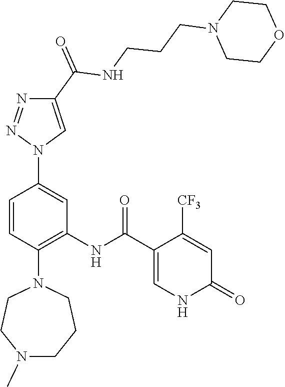 [SMILES: CN1CCCN(c2ccc(-n3cc(C(=O)NCCCN4CCOCC4)nn3)cc2NC(=O)c2c[nH]c(=O)cc2C(F)(F)F)CC1] |
| 61 | 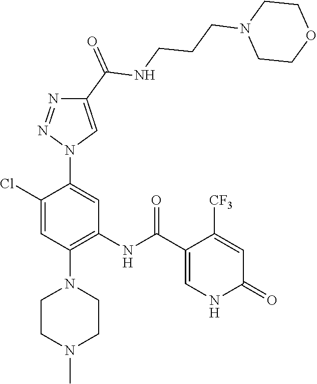 [SMILES: CN1CCN(c2cc(Cl)c(-n3cc(C(=O)NCCCN4CCOCC4)nn3)cc2NC(=O)c2c[nH]c(=O)cc2C(F)(F)F)CC1] |
| 62 | 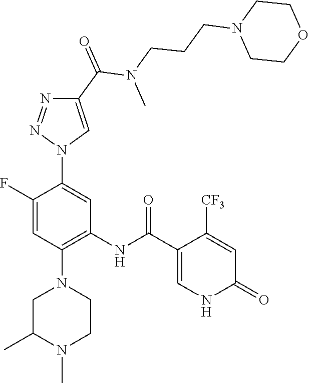 [SMILES: CC1CN(c2cc(F)c(-n3cc(C(=O)N(C)CCCN4CCOCC4)nn3)cc2NC(=O)c2c[nH]c(=O)cc2C(F)(F)F)CCN1C] |
| 63 | 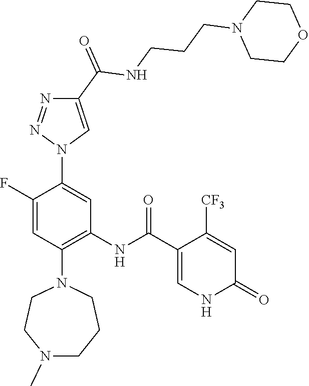 [SMILES: CN1CCCN(c2cc(F)c(-n3cc(C(=O)NCCCN4CCOCC4)nn3)cc2NC(=O)c2c[nH]c(=O)cc2C(F)(F)F)CC1] |
| 64 | 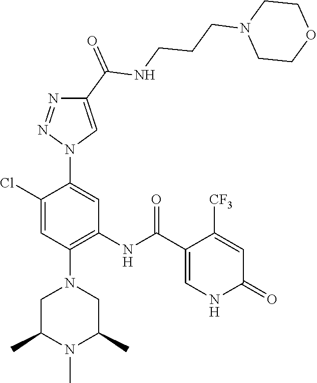 [SMILES: C[C@@H]1CN(c2cc(Cl)c(-n3cc(C(=O)NCCCN4CCOCC4)nn3)cc2NC(=O)c2c[nH]c(=O)cc2C(F)(F)F)C[C@H](C)N1C] |
| 65 | 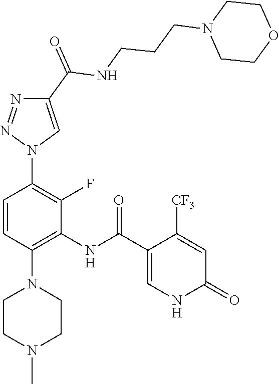 [SMILES: CN1CCN(c2ccc(-n3cc(C(=O)NCCCN4CCOCC4)nn3)c(F)c2NC(=O)c2c[nH]c(=O)cc2C(F)(F)F)CC1] |
| 66 | 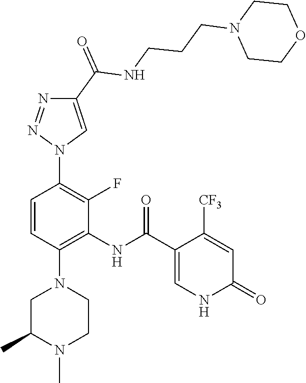 [SMILES: C[C@H]1CN(c2ccc(-n3cc(C(=O)NCCCN4CCOCC4)nn3)c(F)c2NC(=O)c2c[nH]c(=O)cc2C(F)(F)F)CCN1C] |
| 67 | 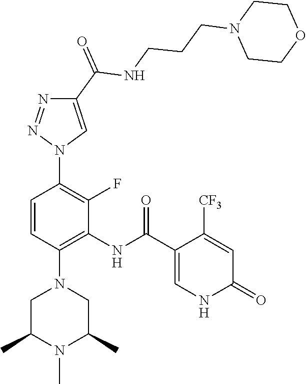 [SMILES: C[C@@H]1CN(c2ccc(-n3cc(C(=O)NCCCN4CCOCC4)nn3)c(F)c2NC(=O)c2c[nH]c(=O)cc2C(F)(F)F)C[C@H](C)N1C] |
| 68 | 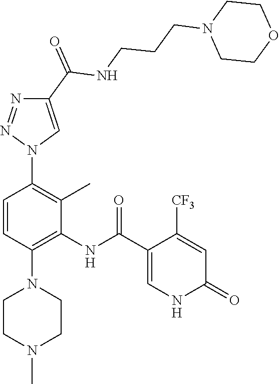 [SMILES: Cc1c(-n2cc(C(=O)NCCCN3CCOCC3)nn2)ccc(N2CCN(C)CC2)c1NC(=O)c1c[nH]c(=O)cc1C(F)(F)F] |
| 69 | 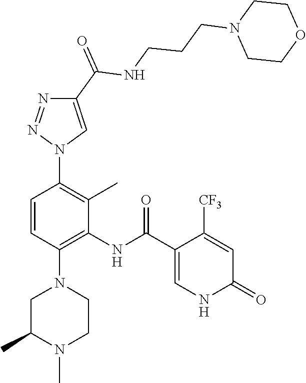 [SMILES: Cc1c(-n2cc(C(=O)NCCCN3CCOCC3)nn2)ccc(N2CCN(C)[C@@H](C)C2)c1NC(=O)c1c[nH]c(=O)cc1C(F)(F)F] |
| 70 | 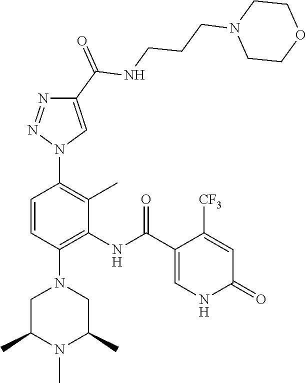 [SMILES: Cc1c(-n2cc(C(=O)NCCCN3CCOCC3)nn2)ccc(N2C[C@@H](C)N(C)[C@@H](C)C2)c1NC(=O)c1c[nH]c(=O)cc1C(F)(F)F] |
| 71 | 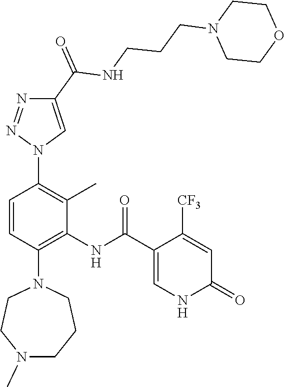 [SMILES: Cc1c(-n2cc(C(=O)NCCCN3CCOCC3)nn2)ccc(N2CCCN(C)CC2)c1NC(=O)c1c[nH]c(=O)cc1C(F)(F)F] |
| 72 | 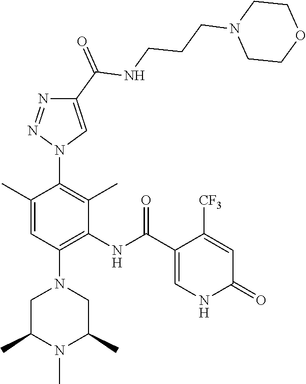 [SMILES: Cc1cc(N2C[C@@H](C)N(C)[C@@H](C)C2)c(NC(=O)c2c[nH]c(=O)cc2C(F)(F)F)c(C)c1-n1cc(C(=O)NCCCN2CCOCC2)nn1] |
| 73 | 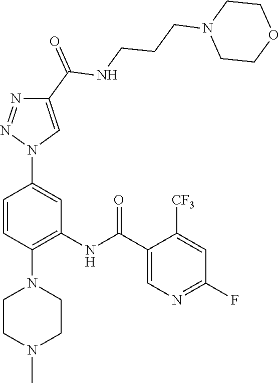 [SMILES: CN1CCN(c2ccc(-n3cc(C(=O)NCCCN4CCOCC4)nn3)cc2NC(=O)c2cnc(F)cc2C(F)(F)F)CC1] |
| 74 | 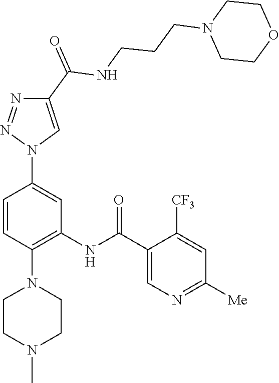 [SMILES: Cc1cc(C(F)(F)F)c(C(=O)Nc2cc(-n3cc(C(=O)NCCCN4CCOCC4)nn3)ccc2N2CCN(C)CC2)cn1] |
| 75 | 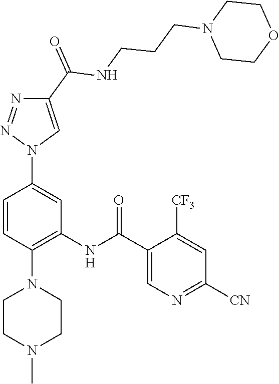 [SMILES: CN1CCN(c2ccc(-n3cc(C(=O)NCCCN4CCOCC4)nn3)cc2NC(=O)c2cnc(C#N)cc2C(F)(F)F)CC1] |
| 76 | 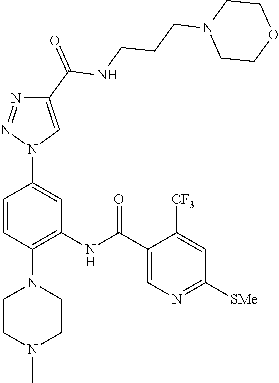 [SMILES: CSc1cc(C(F)(F)F)c(C(=O)Nc2cc(-n3cc(C(=O)NCCCN4CCOCC4)nn3)ccc2N2CCN(C)CC2)cn1] |
| 77 | 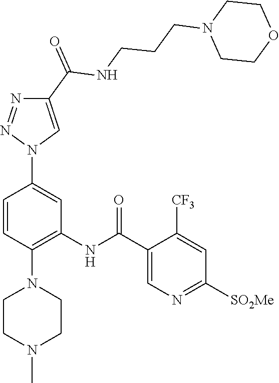 [SMILES: CN1CCN(c2ccc(-n3cc(C(=O)NCCCN4CCOCC4)nn3)cc2NC(=O)c2cnc(S(C)(=O)=O)cc2C(F)(F)F)CC1] |
| 78 | 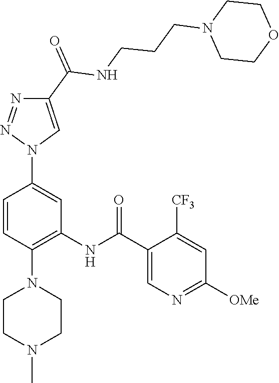 [SMILES: COc1cc(C(F)(F)F)c(C(=O)Nc2cc(-n3cc(C(=O)NCCCN4CCOCC4)nn3)ccc2N2CCN(C)CC2)cn1] |
| 79 | 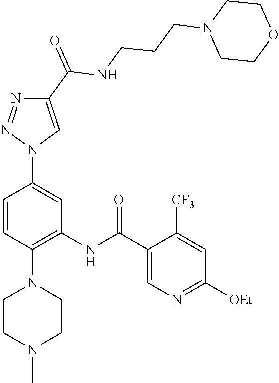 [SMILES: CCOc1cc(C(F)(F)F)c(C(=O)Nc2cc(-n3cc(C(=O)NCCCN4CCOCC4)nn3)ccc2N2CCN(C)CC2)cn1] |
| 80 | 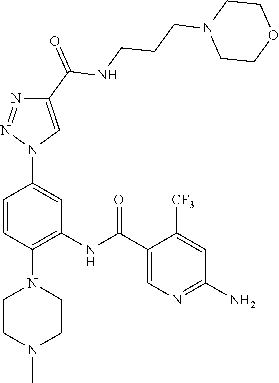 [SMILES: CN1CCN(c2ccc(-n3cc(C(=O)NCCCN4CCOCC4)nn3)cc2NC(=O)c2cnc(N)cc2C(F)(F)F)CC1] |
| 81 | 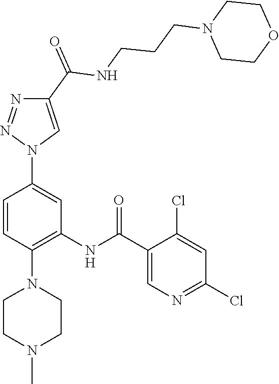 [SMILES: CN1CCN(c2ccc(-n3cc(C(=O)NCCCN4CCOCC4)nn3)cc2NC(=O)c2cnc(Cl)cc2Cl)CC1] |
| 82 | 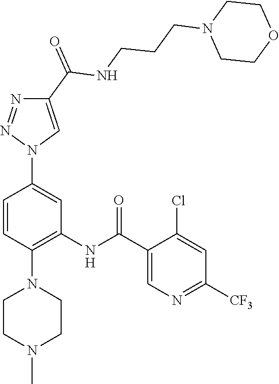 [SMILES: CN1CCN(c2ccc(-n3cc(C(=O)NCCCN4CCOCC4)nn3)cc2NC(=O)c2cnc(C(F)(F)F)cc2Cl)CC1] |
| 83 | 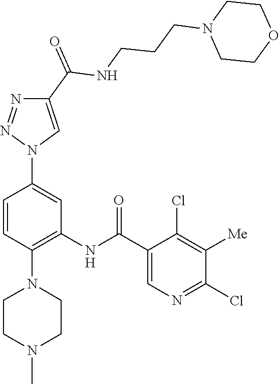 [SMILES: Cc1c(Cl)ncc(C(=O)Nc2cc(-n3cc(C(=O)NCCCN4CCOCC4)nn3)ccc2N2CCN(C)CC2)c1Cl] |
| 84 | 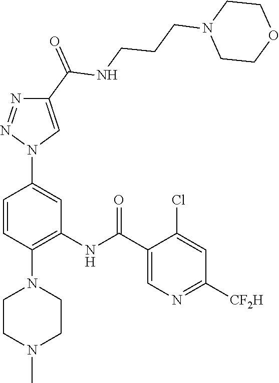 [SMILES: CN1CCN(c2ccc(-n3cc(C(=O)NCCCN4CCOCC4)nn3)cc2NC(=O)c2cnc(C(F)F)cc2Cl)CC1] |
| 85 | 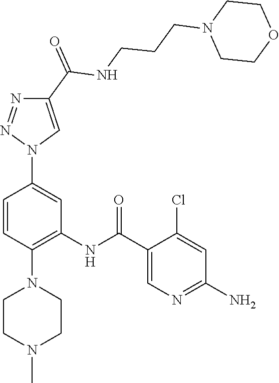 [SMILES: CN1CCN(c2ccc(-n3cc(C(=O)NCCCN4CCOCC4)nn3)cc2NC(=O)c2cnc(N)cc2Cl)CC1] |
| 86 | 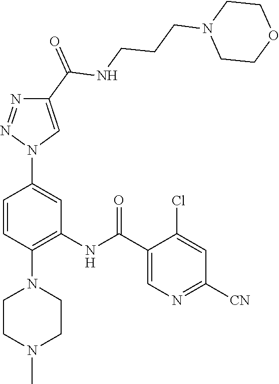 [SMILES: CN1CCN(c2ccc(-n3cc(C(=O)NCCCN4CCOCC4)nn3)cc2NC(=O)c2cnc(C#N)cc2Cl)CC1] |
| 87 | 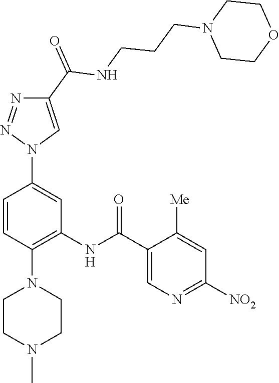 [SMILES: Cc1cc([N+](=O)[O-])ncc1C(=O)Nc1cc(-n2cc(C(=O)NCCCN3CCOCC3)nn2)ccc1N1CCN(C)CC1] |
| 88 | 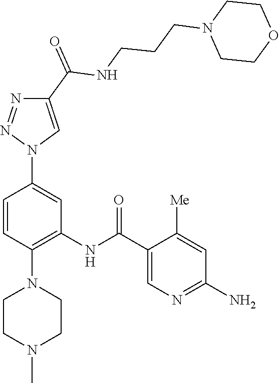 [SMILES: Cc1cc(N)ncc1C(=O)Nc1cc(-n2cc(C(=O)NCCCN3CCOCC3)nn2)ccc1N1CCN(C)CC1] |
| 89 | 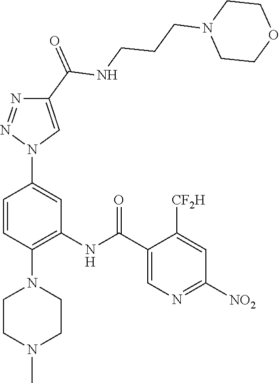 [SMILES: CN1CCN(c2ccc(-n3cc(C(=O)NCCCN4CCOCC4)nn3)cc2NC(=O)c2cnc([N+](=O)[O-])cc2C(F)F)CC1] |
| 90 | 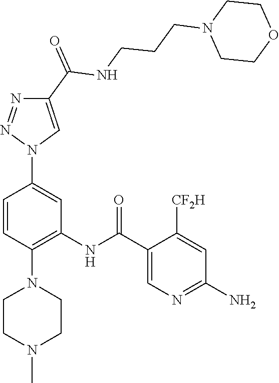 [SMILES: CN1CCN(c2ccc(-n3cc(C(=O)NCCCN4CCOCC4)nn3)cc2NC(=O)c2cnc(N)cc2C(F)F)CC1] |
| 91 | 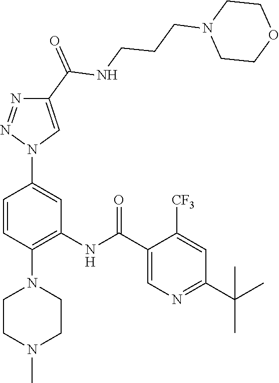 [SMILES: CN1CCN(c2ccc(-n3cc(C(=O)NCCCN4CCOCC4)nn3)cc2NC(=O)c2cnc(C(C)(C)C)cc2C(F)(F)F)CC1] |
| 92 | 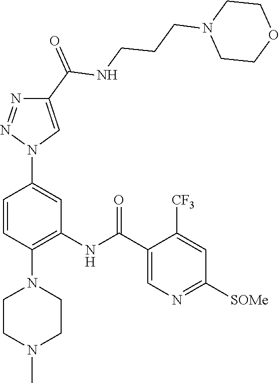 |
| 93 | 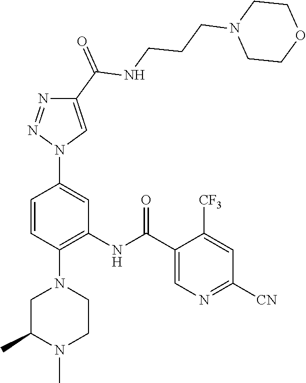 [SMILES: C[C@H]1CN(c2ccc(-n3cc(C(=O)NCCCN4CCOCC4)nn3)cc2NC(=O)c2cnc(C#N)cc2C(F)(F)F)CCN1C] |
| 94 | 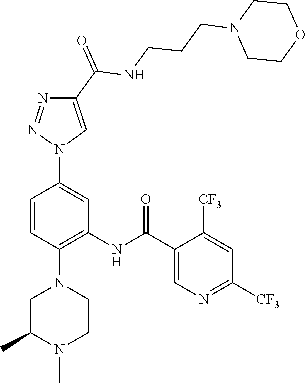 [SMILES: C[C@H]1CN(c2ccc(-n3cc(C(=O)NCCCN4CCOCC4)nn3)cc2NC(=O)c2cnc(C(F)(F)F)cc2C(F)(F)F)CCN1C] |
| 95 | 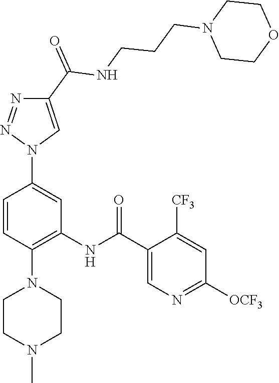 [SMILES: CN1CCN(c2ccc(-n3cc(C(=O)NCCCN4CCOCC4)nn3)cc2NC(=O)c2cnc(OC(F)(F)F)cc2C(F)(F)F)CC1] |
| 96 | 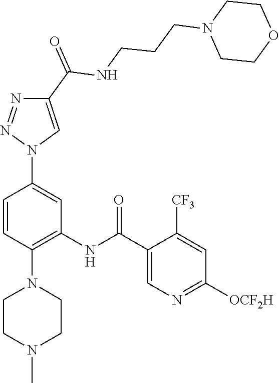 |
| 97 | 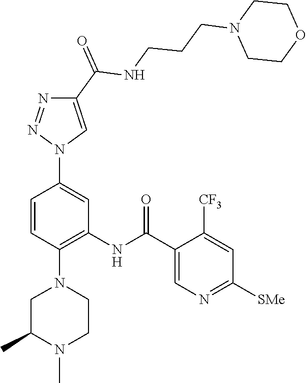 [SMILES: CSc1cc(C(F)(F)F)c(C(=O)Nc2cc(-n3cc(C(=O)NCCCN4CCOCC4)nn3)ccc2N2CCN(C)[C@@H](C)C2)cn1] |
| 98 | 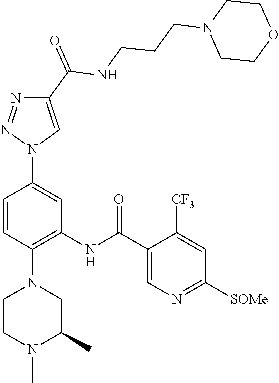 |
| 99 | 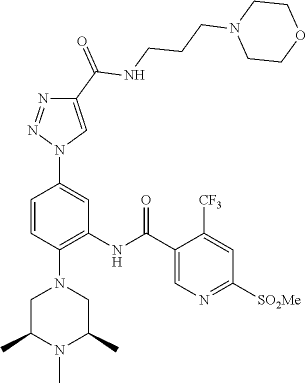 [SMILES: C[C@@H]1CN(c2ccc(-n3cc(C(=O)NCCCN4CCOCC4)nn3)cc2NC(=O)c2cnc(S(C)(=O)=O)cc2C(F)(F)F)C[C@H](C)N1C] |
| 100 | 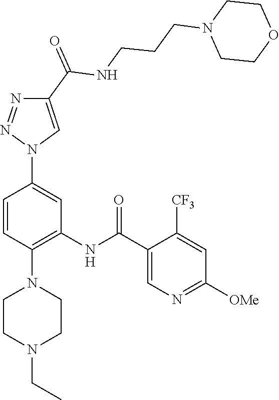 [SMILES: CCN1CCN(c2ccc(-n3cc(C(=O)NCCCN4CCOCC4)nn3)cc2NC(=O)c2cnc(OC)cc2C(F)(F)F)CC1] |
| 101 | 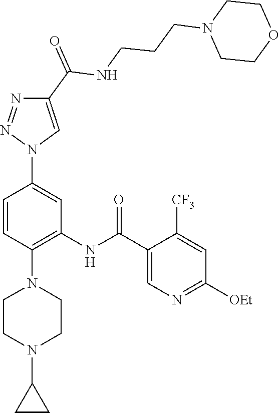 [SMILES: CCOc1cc(C(F)(F)F)c(C(=O)Nc2cc(-n3cc(C(=O)NCCCN4CCOCC4)nn3)ccc2N2CCN(C3CC3)CC2)cn1] |
| 102 | 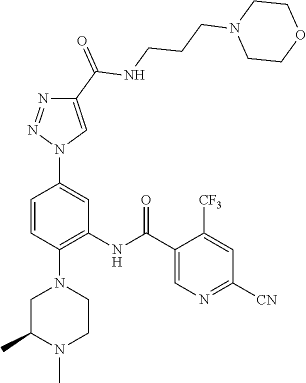 [SMILES: C[C@H]1CN(c2ccc(-n3cc(C(=O)NCCCN4CCOCC4)nn3)cc2NC(=O)c2cnc(C#N)cc2C(F)(F)F)CCN1C] |
| 103 | 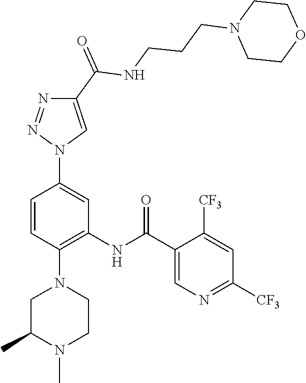 [SMILES: C[C@H]1CN(c2ccc(-n3cc(C(=O)NCCCN4CCOCC4)nn3)cc2NC(=O)c2cnc(C(F)(F)F)cc2C(F)(F)F)CCN1C] |
| 104 | 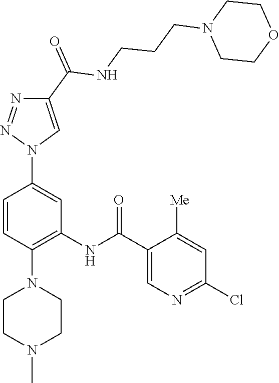 [SMILES: Cc1cc(Cl)ncc1C(=O)Nc1cc(-n2cc(C(=O)NCCCN3CCOCC3)nn2)ccc1N1CCN(C)CC1] |
| 105 | 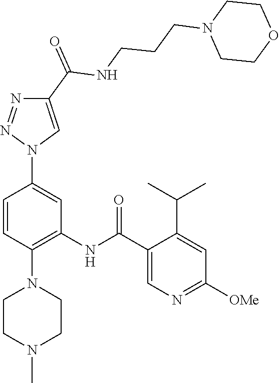 [SMILES: COc1cc(C(C)C)c(C(=O)Nc2cc(-n3cc(C(=O)NCCCN4CCOCC4)nn3)ccc2N2CCN(C)CC2)cn1] |
| 106 | 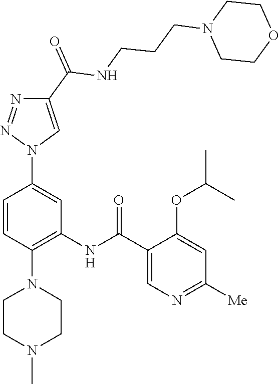 [SMILES: Cc1cc(OC(C)C)c(C(=O)Nc2cc(-n3cc(C(=O)NCCCN4CCOCC4)nn3)ccc2N2CCN(C)CC2)cn1] |
| 107 | 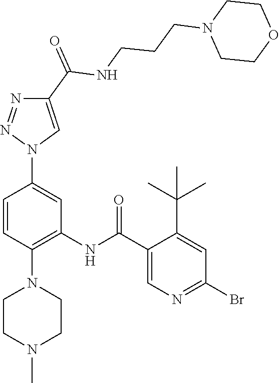 [SMILES: CN1CCN(c2ccc(-n3cc(C(=O)NCCCN4CCOCC4)nn3)cc2NC(=O)c2cnc(Br)cc2C(C)(C)C)CC1] |
| 108 | 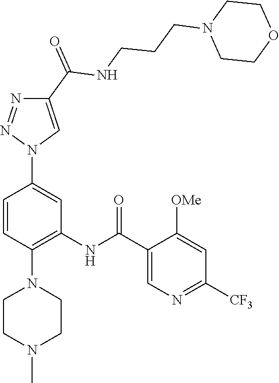 [SMILES: COc1cc(C(F)(F)F)ncc1C(=O)Nc1cc(-n2cc(C(=O)NCCCN3CCOCC3)nn2)ccc1N1CCN(C)CC1] |
| 109 | 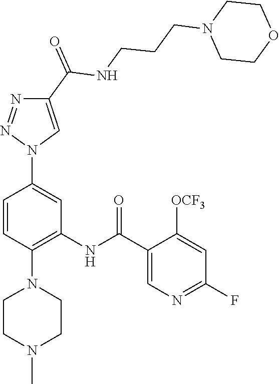 [SMILES: CN1CCN(c2ccc(-n3cc(C(=O)NCCCN4CCOCC4)nn3)cc2NC(=O)c2cnc(F)cc2OC(F)(F)F)CC1] |
| 110 | 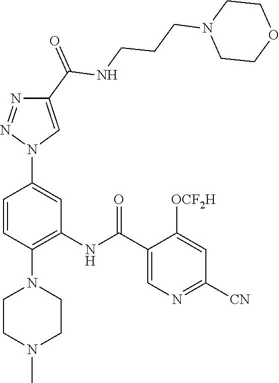 |
| 111 | 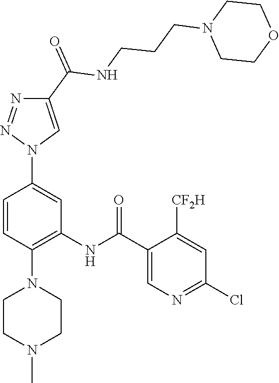 [SMILES: CN1CCN(c2ccc(-n3cc(C(=O)NCCCN4CCOCC4)nn3)cc2NC(=O)c2cnc(Cl)cc2C(F)F)CC1] |
| 112 | 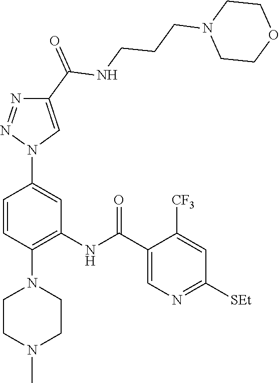 |
| 113 | 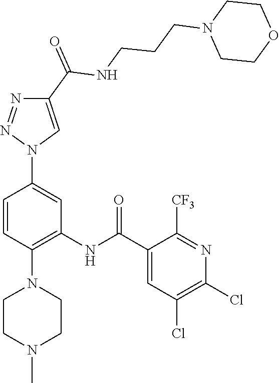 [SMILES: CN1CCN(c2ccc(-n3cc(C(=O)NCCCN4CCOCC4)nn3)cc2NC(=O)c2cc(Cl)c(Cl)nc2C(F)(F)F)CC1] |
| 114 | 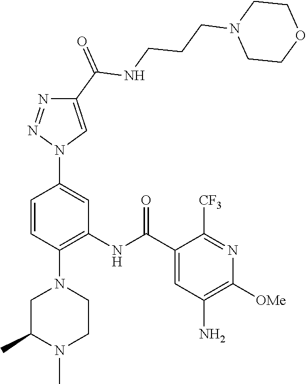 [SMILES: COc1nc(C(F)(F)F)c(C(=O)Nc2cc(-n3cc(C(=O)NCCCN4CCOCC4)nn3)ccc2N2CCN(C)[C@@H](C)C2)cc1N] |
| 115 | 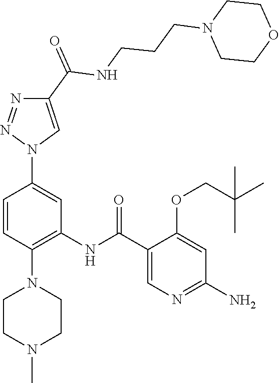 [SMILES: CN1CCN(c2ccc(-n3cc(C(=O)NCCCN4CCOCC4)nn3)cc2NC(=O)c2cnc(N)cc2OCC(C)(C)C)CC1] |
| 116 | 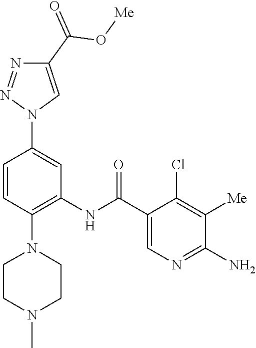 [SMILES: COC(=O)c1cn(-c2ccc(N3CCN(C)CC3)c(NC(=O)c3cnc(N)c(C)c3Cl)c2)nn1] |
| 117 | 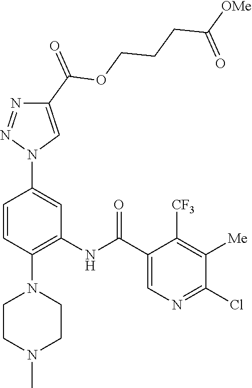 [SMILES: COC(=O)CCCOC(=O)c1cn(-c2ccc(N3CCN(C)CC3)c(NC(=O)c3cnc(Cl)c(C)c3C(F)(F)F)c2)nn1] |
| 118 | 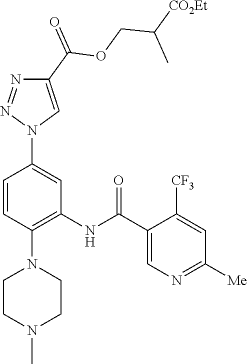 [SMILES: CCOC(=O)C(C)COC(=O)c1cn(-c2ccc(N3CCN(C)CC3)c(NC(=O)c3cnc(C)cc3C(F)(F)F)c2)nn1] |
| 119 | 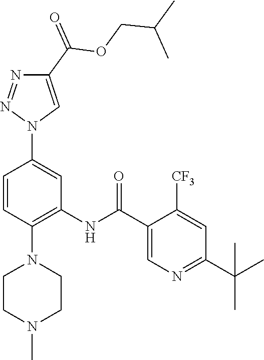 [SMILES: CC(C)COC(=O)c1cn(-c2ccc(N3CCN(C)CC3)c(NC(=O)c3cnc(C(C)(C)C)cc3C(F)(F)F)c2)nn1] |
| 120 | 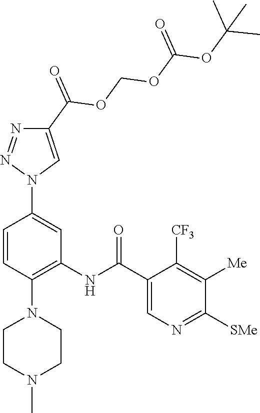 [SMILES: CSc1ncc(C(=O)Nc2cc(-n3cc(C(=O)OCOC(=O)OC(C)(C)C)nn3)ccc2N2CCN(C)CC2)c(C(F)(F)F)c1C] |
| 121 | 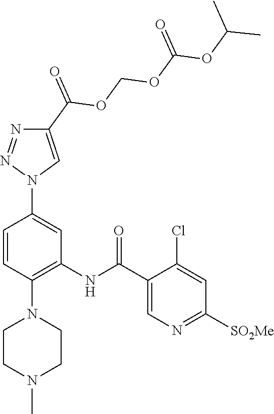 [SMILES: CC(C)OC(=O)OCOC(=O)c1cn(-c2ccc(N3CCN(C)CC3)c(NC(=O)c3cnc(S(C)(=O)=O)cc3Cl)c2)nn1] |
| 122 | 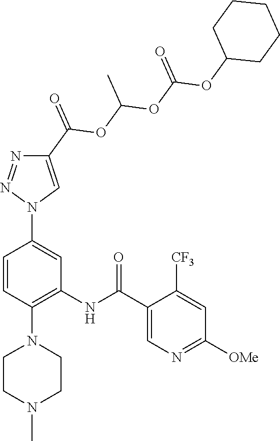 [SMILES: COc1cc(C(F)(F)F)c(C(=O)Nc2cc(-n3cc(C(=O)OC(C)OC(=O)OC4CCCCC4)nn3)ccc2N2CCN(C)CC2)cn1] |
| 123 | 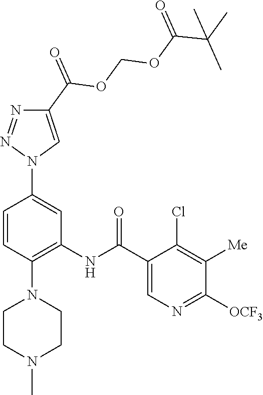 [SMILES: Cc1c(OC(F)(F)F)ncc(C(=O)Nc2cc(-n3cc(C(=O)OCOC(=O)C(C)(C)C)nn3)ccc2N2CCN(C)CC2)c1Cl] |
| 124 | 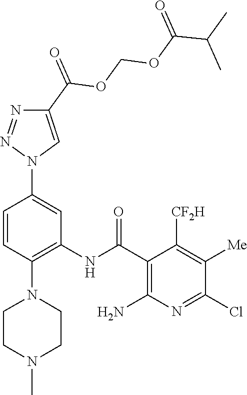 [SMILES: Cc1c(Cl)nc(N)c(C(=O)Nc2cc(-n3cc(C(=O)OCOC(=O)C(C)C)nn3)ccc2N2CCN(C)CC2)c1C(F)F] |
| 125 | 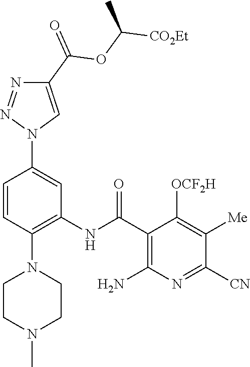 |
| 126 | 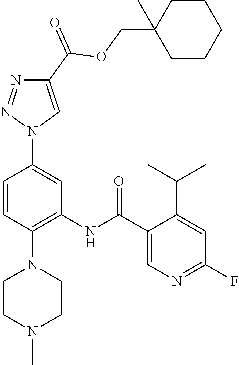 [SMILES: CC(C)c1cc(F)ncc1C(=O)Nc1cc(-n2cc(C(=O)OCC3(C)CCCCC3)nn2)ccc1N1CCN(C)CC1] |
| 127 | 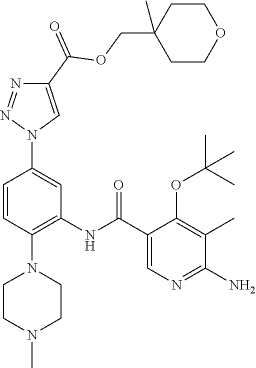 [SMILES: Cc1c(N)ncc(C(=O)Nc2cc(-n3cc(C(=O)OCC4(C)CCOCC4)nn3)ccc2N2CCN(C)CC2)c1OC(C)(C)C] |
| 128 | 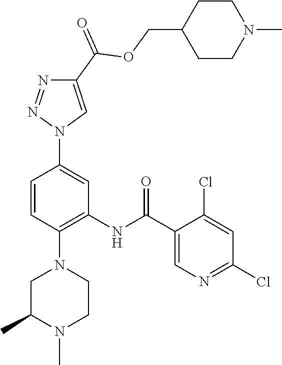 |
| 129 | 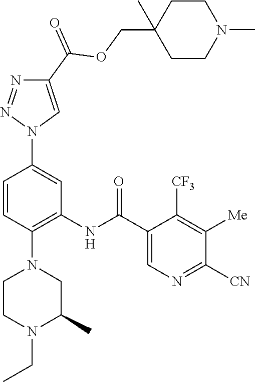 [SMILES: CCN1CCN(c2ccc(-n3cc(C(=O)OCC4(C)CCN(C)CC4)nn3)cc2NC(=O)c2cnc(C#N)c(C)c2C(F)(F)F)C[C@H]1C] |
| 130 | 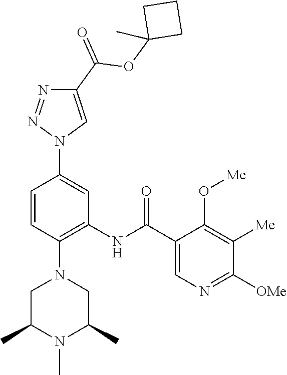 [SMILES: COc1ncc(C(=O)Nc2cc(-n3cc(C(=O)OC4(C)CCC4)nn3)ccc2N2C[C@@H](C)N(C)[C@@H](C)C2)c(OC)c1C] |
| 131 | 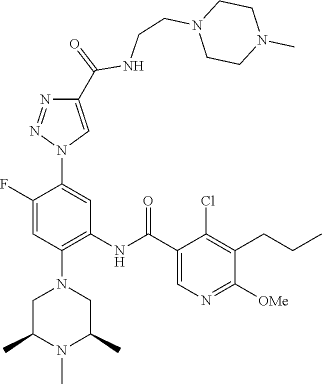 [SMILES: CCCc1c(OC)ncc(C(=O)Nc2cc(-n3cc(C(=O)NCCN4CCN(C)CC4)nn3)c(F)cc2N2C[C@@H](C)N(C)[C@@H](C)C2)c1Cl] |
| 132 | 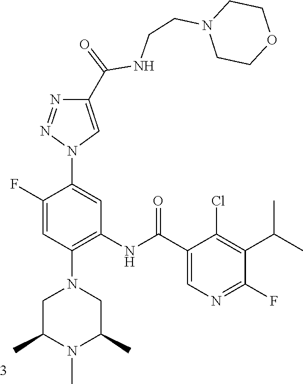 [SMILES: CC(C)c1c(F)ncc(C(=O)Nc2cc(-n3cc(C(=O)NCCN4CCOCC4)nn3)c(F)cc2N2C[C@@H](C)N(C)[C@@H](C)C2)c1Cl] |
| 133 | 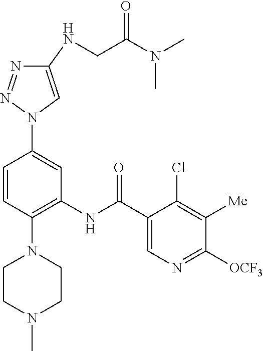 [SMILES: Cc1c(OC(F)(F)F)ncc(C(=O)Nc2cc(-n3cc(NCC(=O)N(C)C)nn3)ccc2N2CCN(C)CC2)c1Cl] |
| 134 | 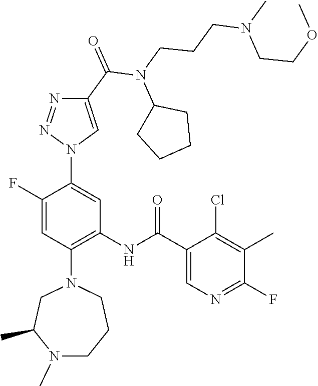 [SMILES: COCCN(C)CCCN(C(=O)c1cn(-c2cc(NC(=O)c3cnc(F)c(C)c3Cl)c(N3CCCN(C)[C@@H](C)C3)cc2F)nn1)C1CCCC1] |
| 135 | 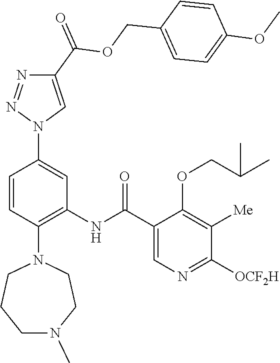 |
| 136 | 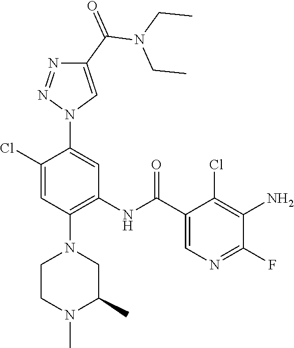 [SMILES: CCN(CC)C(=O)c1cn(-c2cc(NC(=O)c3cnc(F)c(N)c3Cl)c(N3CCN(C)[C@H](C)C3)cc2Cl)nn1] |
| 137 | 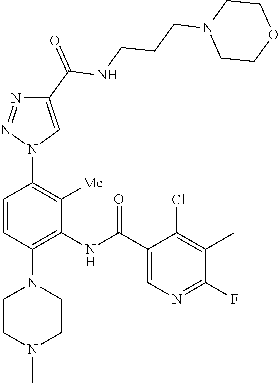 [SMILES: Cc1c(F)ncc(C(=O)Nc2c(N3CCN(C)CC3)ccc(-n3cc(C(=O)NCCCN4CCOCC4)nn3)c2C)c1Cl] |
| 138 | 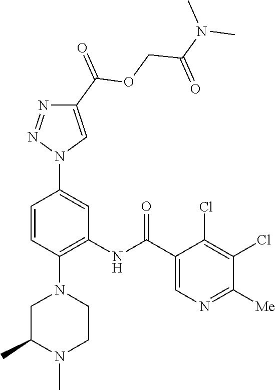 [SMILES: Cc1ncc(C(=O)Nc2cc(-n3cc(C(=O)OCC(=O)N(C)C)nn3)ccc2N2CCN(C)[C@@H](C)C2)c(Cl)c1Cl] |
| 139 | 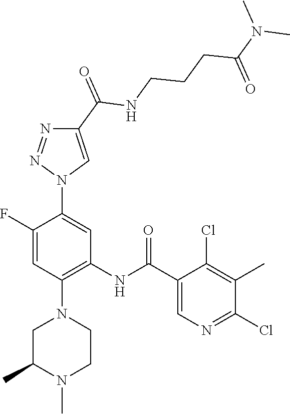 |
| 140 | 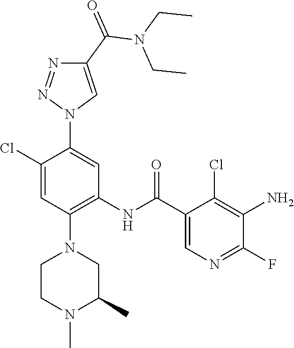 [SMILES: CCN(CC)C(=O)c1cn(-c2cc(NC(=O)c3cnc(F)c(N)c3Cl)c(N3CCN(C)[C@H](C)C3)cc2Cl)nn1] |
| 141 | 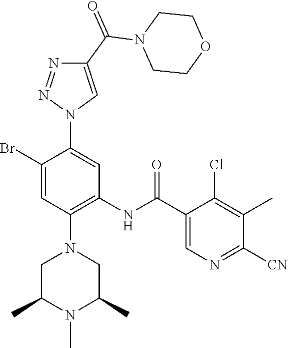 [SMILES: Cc1c(C#N)ncc(C(=O)Nc2cc(-n3cc(C(=O)N4CCOCC4)nn3)c(Br)cc2N2C[C@@H](C)N(C)[C@@H](C)C2)c1Cl] |
| 142 | 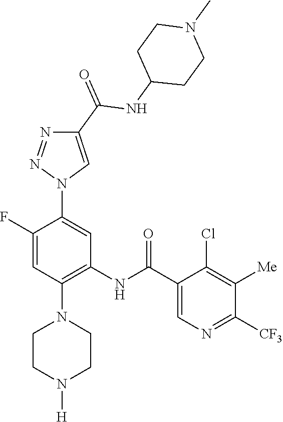 [SMILES: Cc1c(C(F)(F)F)ncc(C(=O)Nc2cc(-n3cc(C(=O)NC4CCN(C)CC4)nn3)c(F)cc2N2CCNCC2)c1Cl] |
| 143 | 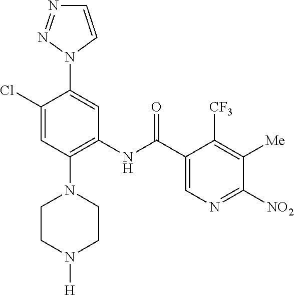 [SMILES: Cc1c([N+](=O)[O-])ncc(C(=O)Nc2cc(-n3ccnn3)c(Cl)cc2N2CCNCC2)c1C(F)(F)F] |
| 144 | 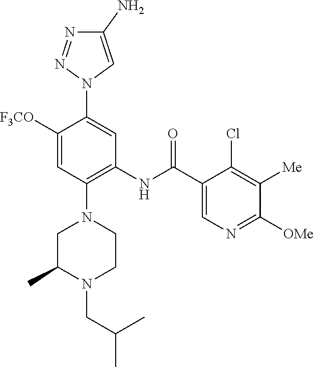 [SMILES: COc1ncc(C(=O)Nc2cc(-n3cc(N)nn3)c(OC(F)(F)F)cc2N2CCN(CC(C)C)[C@@H](C)C2)c(Cl)c1C] |
| 145 | 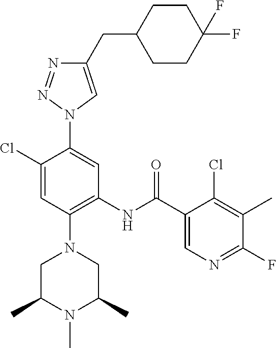 [SMILES: Cc1c(F)ncc(C(=O)Nc2cc(-n3cc(CC4CCC(F)(F)CC4)nn3)c(Cl)cc2N2C[C@@H](C)N(C)[C@@H](C)C2)c1Cl] |
| 146 | 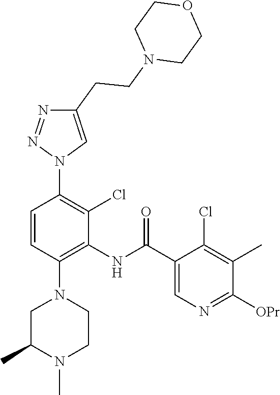 |
| 147 | 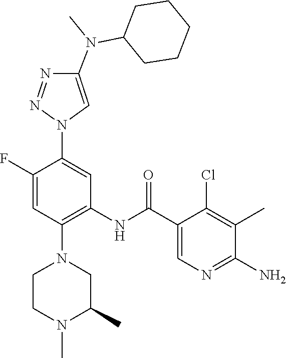 [SMILES: Cc1c(N)ncc(C(=O)Nc2cc(-n3cc(N(C)C4CCCCC4)nn3)c(F)cc2N2CCN(C)[C@H](C)C2)c1Cl] |
| 148 | 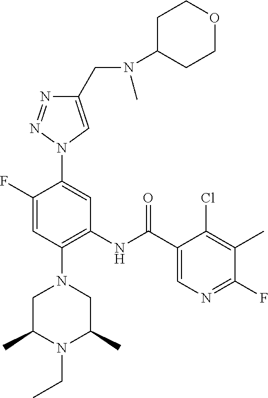 [SMILES: CCN1[C@H](C)CN(c2cc(F)c(-n3cc(CN(C)C4CCOCC4)nn3)cc2NC(=O)c2cnc(F)c(C)c2Cl)C[C@@H]1C] |
| 149 | 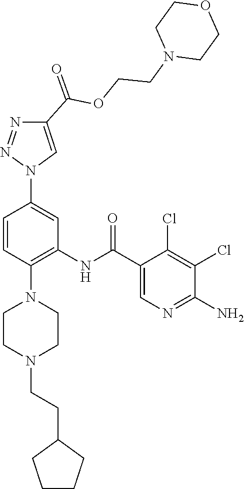 [SMILES: Nc1ncc(C(=O)Nc2cc(-n3cc(C(=O)OCCN4CCOCC4)nn3)ccc2N2CCN(CCC3CCCC3)CC2)c(Cl)c1Cl] |
| 150 | 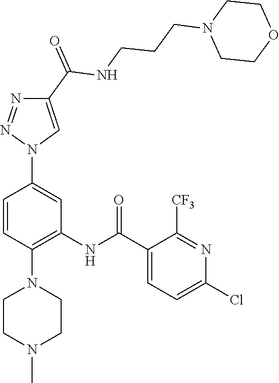 [SMILES: CN1CCN(c2ccc(-n3cc(C(=O)NCCCN4CCOCC4)nn3)cc2NC(=O)c2ccc(Cl)nc2C(F)(F)F)CC1] |
| 151 | 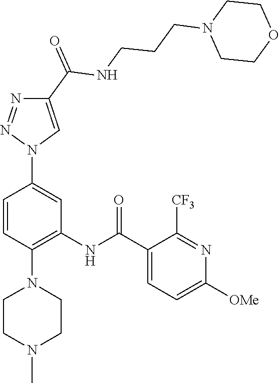 [SMILES: COc1ccc(C(=O)Nc2cc(-n3cc(C(=O)NCCCN4CCOCC4)nn3)ccc2N2CCN(C)CC2)c(C(F)(F)F)n1] |
| 152 | 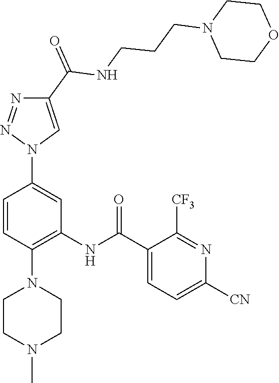 [SMILES: CN1CCN(c2ccc(-n3cc(C(=O)NCCCN4CCOCC4)nn3)cc2NC(=O)c2ccc(C#N)nc2C(F)(F)F)CC1] |
| 153 | 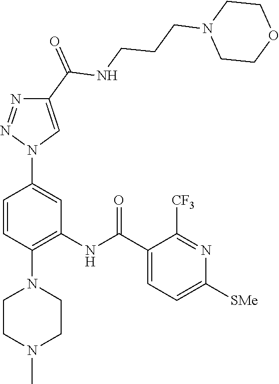 [SMILES: CSc1ccc(C(=O)Nc2cc(-n3cc(C(=O)NCCCN4CCOCC4)nn3)ccc2N2CCN(C)CC2)c(C(F)(F)F)n1] |
| 154 | 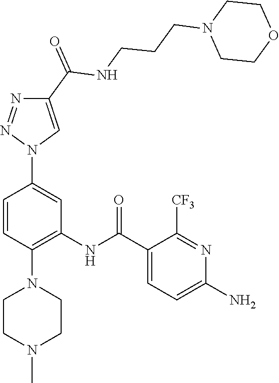 [SMILES: CN1CCN(c2ccc(-n3cc(C(=O)NCCCN4CCOCC4)nn3)cc2NC(=O)c2ccc(N)nc2C(F)(F)F)CC1] |
| 155 | 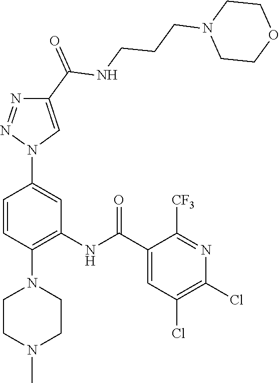 [SMILES: CN1CCN(c2ccc(-n3cc(C(=O)NCCCN4CCOCC4)nn3)cc2NC(=O)c2cc(Cl)c(Cl)nc2C(F)(F)F)CC1] |
| 156 | 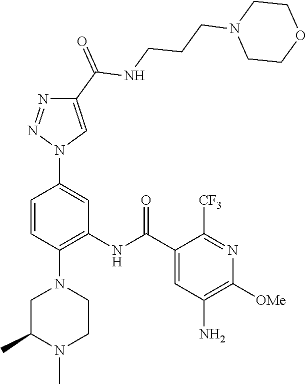 [SMILES: COc1nc(C(F)(F)F)c(C(=O)Nc2cc(-n3cc(C(=O)NCCCN4CCOCC4)nn3)ccc2N2CCN(C)[C@@H](C)C2)cc1N] |
| 157 | 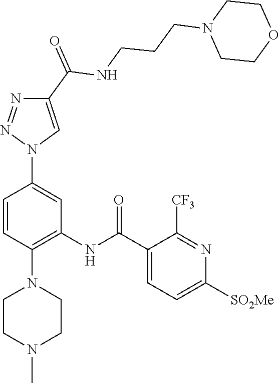 [SMILES: CN1CCN(c2ccc(-n3cc(C(=O)NCCCN4CCOCC4)nn3)cc2NC(=O)c2ccc(S(C)(=O)=O)nc2C(F)(F)F)CC1] |
| 158 | 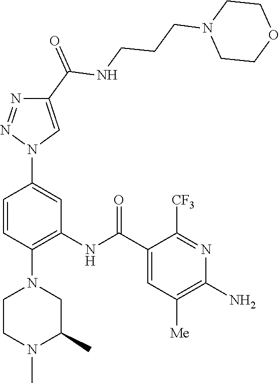 [SMILES: Cc1cc(C(=O)Nc2cc(-n3cc(C(=O)NCCCN4CCOCC4)nn3)ccc2N2CCN(C)[C@H](C)C2)c(C(F)(F)F)nc1N] |
| 159 | 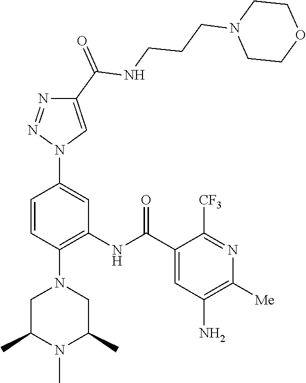 [SMILES: Cc1nc(C(F)(F)F)c(C(=O)Nc2cc(-n3cc(C(=O)NCCCN4CCOCC4)nn3)ccc2N2C[C@@H](C)N(C)[C@@H](C)C2)cc1N] |
| 160 | 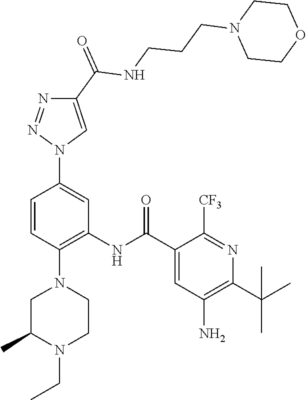 [SMILES: CCN1CCN(c2ccc(-n3cc(C(=O)NCCCN4CCOCC4)nn3)cc2NC(=O)c2cc(N)c(C(C)(C)C)nc2C(F)(F)F)C[C@@H]1C] |
| 161 | 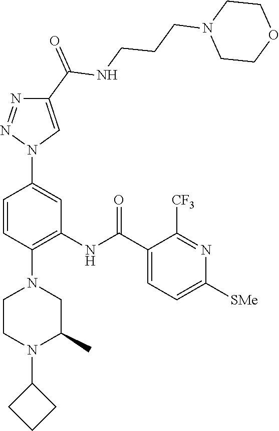 [SMILES: CSc1ccc(C(=O)Nc2cc(-n3cc(C(=O)NCCCN4CCOCC4)nn3)ccc2N2CCN(C3CCC3)[C@H](C)C2)c(C(F)(F)F)n1] |
| 162 | 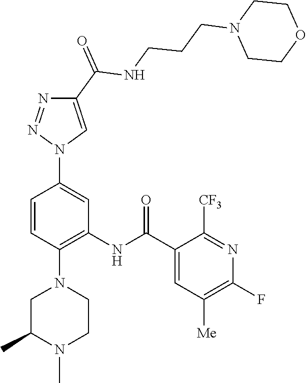 [SMILES: Cc1cc(C(=O)Nc2cc(-n3cc(C(=O)NCCCN4CCOCC4)nn3)ccc2N2CCN(C)[C@@H](C)C2)c(C(F)(F)F)nc1F] |
| 163 | 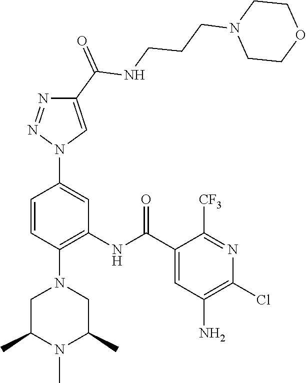 [SMILES: C[C@@H]1CN(c2ccc(-n3cc(C(=O)NCCCN4CCOCC4)nn3)cc2NC(=O)c2cc(N)c(Cl)nc2C(F)(F)F)C[C@H](C)N1C] |
| 164 | 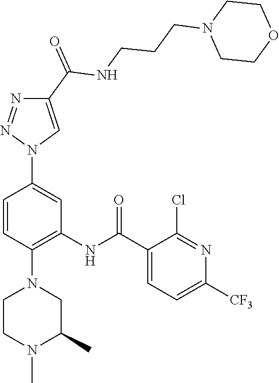 [SMILES: C[C@@H]1CN(c2ccc(-n3cc(C(=O)NCCCN4CCOCC4)nn3)cc2NC(=O)c2ccc(C(F)(F)F)nc2Cl)CCN1C] |
| 165 | 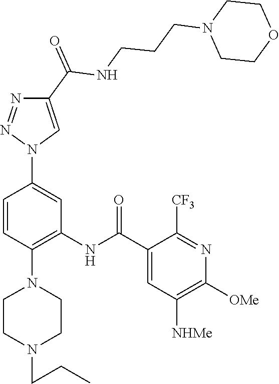 [SMILES: CCCN1CCN(c2ccc(-n3cc(C(=O)NCCCN4CCOCC4)nn3)cc2NC(=O)c2cc(NC)c(OC)nc2C(F)(F)F)CC1] |
| 166 | 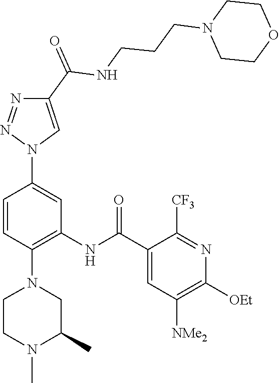 [SMILES: CCOc1nc(C(F)(F)F)c(C(=O)Nc2cc(-n3cc(C(=O)NCCCN4CCOCC4)nn3)ccc2N2CCN(C)[C@H](C)C2)cc1N(C)C] |
| 167 | 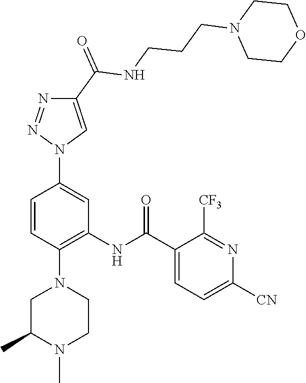 [SMILES: C[C@H]1CN(c2ccc(-n3cc(C(=O)NCCCN4CCOCC4)nn3)cc2NC(=O)c2ccc(C#N)nc2C(F)(F)F)CCN1C] |
| 168 | 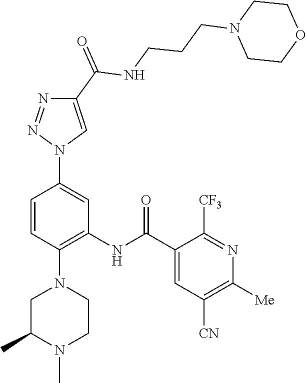 [SMILES: Cc1nc(C(F)(F)F)c(C(=O)Nc2cc(-n3cc(C(=O)NCCCN4CCOCC4)nn3)ccc2N2CCN(C)[C@@H](C)C2)cc1C#N] |
| 169 | 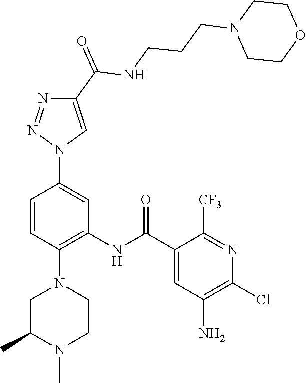 [SMILES: C[C@H]1CN(c2ccc(-n3cc(C(=O)NCCCN4CCOCC4)nn3)cc2NC(=O)c2cc(N)c(Cl)nc2C(F)(F)F)CCN1C] |
| 170 | 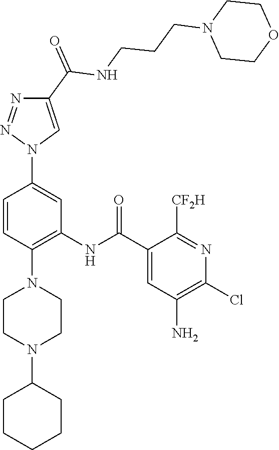 [SMILES: Nc1cc(C(=O)Nc2cc(-n3cc(C(=O)NCCCN4CCOCC4)nn3)ccc2N2CCN(C3CCCCC3)CC2)c(C(F)F)nc1Cl] |
| 171 | 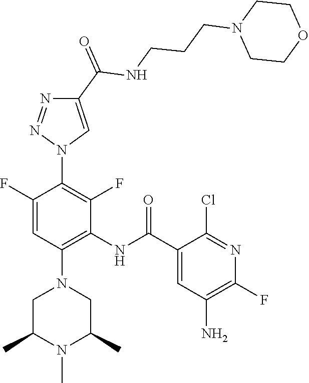 [SMILES: C[C@@H]1CN(c2cc(F)c(-n3cc(C(=O)NCCCN4CCOCC4)nn3)c(F)c2NC(=O)c2cc(N)c(F)nc2Cl)C[C@H](C)N1C] |
| 172 | 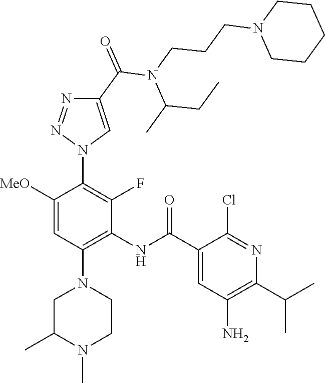 [SMILES: CCC(C)N(CCCN1CCCCC1)C(=O)c1cn(-c2c(OC)cc(N3CCN(C)C(C)C3)c(NC(=O)c3cc(N)c(C(C)C)nc3Cl)c2F)nn1] |
| 173 | 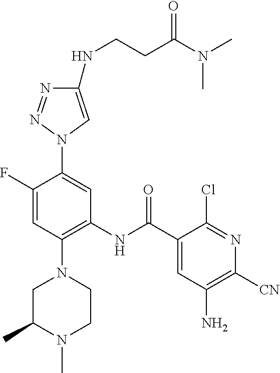 [SMILES: C[C@H]1CN(c2cc(F)c(-n3cc(NCCC(=O)N(C)C)nn3)cc2NC(=O)c2cc(N)c(C#N)nc2Cl)CCN1C] |
| 174 | 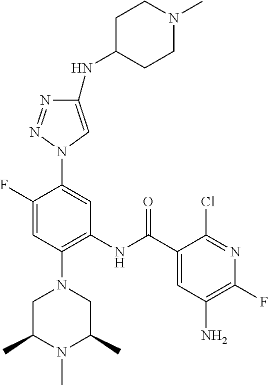 [SMILES: C[C@@H]1CN(c2cc(F)c(-n3cc(NC4CCN(C)CC4)nn3)cc2NC(=O)c2cc(N)c(F)nc2Cl)C[C@H](C)N1C] |
| 175 | 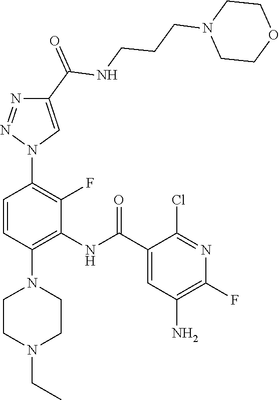 [SMILES: CCN1CCN(c2ccc(-n3cc(C(=O)NCCCN4CCOCC4)nn3)c(F)c2NC(=O)c2cc(N)c(F)nc2Cl)CC1] |
| 176 | 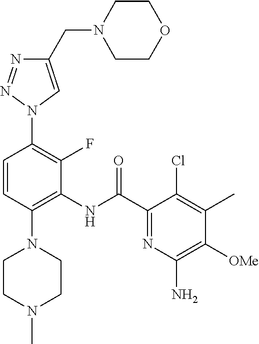 [SMILES: COc1c(N)nc(C(=O)Nc2c(N3CCN(C)CC3)ccc(-n3cc(CN4CCOCC4)nn3)c2F)c(Cl)c1C] |
| 177 | 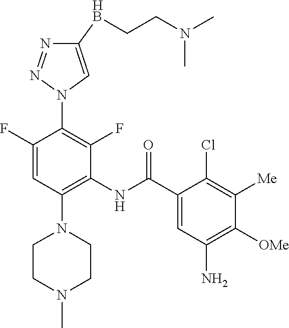 |
| 178 | 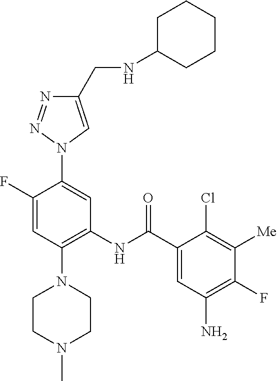 [SMILES: Cc1c(F)c(N)cc(C(=O)Nc2cc(-n3cc(CNC4CCCCC4)nn3)c(F)cc2N2CCN(C)CC2)c1Cl] |
| 179 | 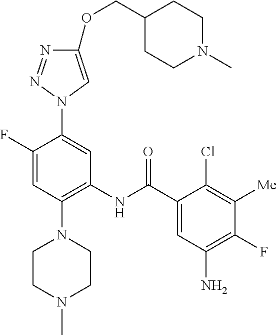 [SMILES: Cc1c(F)c(N)cc(C(=O)Nc2cc(-n3cc(OCC4CCN(C)CC4)nn3)c(F)cc2N2CCN(C)CC2)c1Cl] |
| 180 | 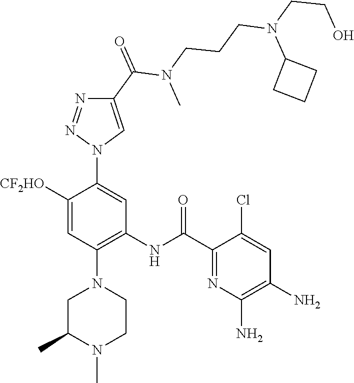 |

TABLE 3
Compounds of the disclosure.

| Compound | |
|---|---|
| No. | Structure |
| 181 | 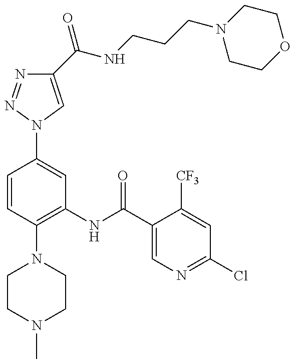 [SMILES: CN1CCN(c2ccc(-n3cc(C(=O)NCCCN4CCOCC4)nn3)cc2NC(=O)c2cnc(Cl)cc2C(F)(F)F)CC1] |
| 182 | 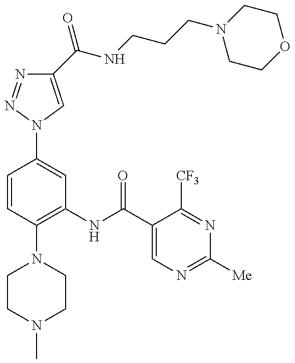 [SMILES: Cc1ncc(C(=O)Nc2cc(-n3cc(C(=O)NCCCN4CCOCC4)nn3)ccc2N2CCN(C)CC2)c(C(F)(F)F)n1] |
| 183 | 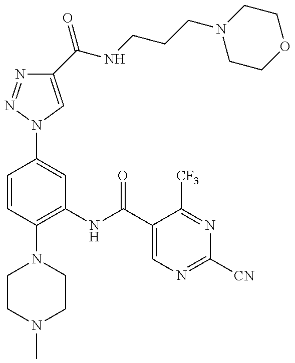 [SMILES: CN1CCN(c2ccc(-n3cc(C(=O)NCCCN4CCOCC4)nn3)cc2NC(=O)c2cnc(C#N)nc2C(F)(F)F)CC1] |
| 184 | 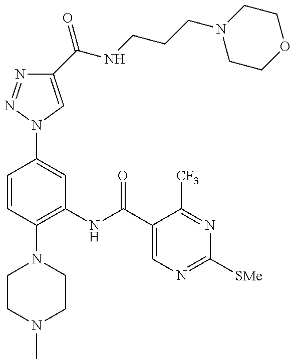 [SMILES: CSc1ncc(C(=O)Nc2cc(-n3cc(C(=O)NCCCN4CCOCC4)nn3)ccc2N2CCN(C)CC2)c(C(F)(F)F)n1] |
| 185 | 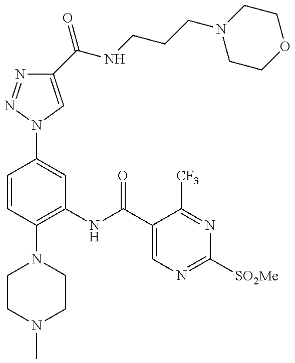 [SMILES: CN1CCN(c2ccc(-n3cc(C(=O)NCCCN4CCOCC4)nn3)cc2NC(=O)c2cnc(S(C)(=O)=O)nc2C(F)(F)F)CC1] |
| 186 | 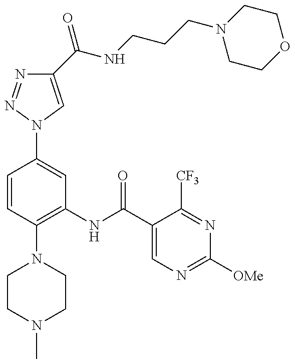 [SMILES: COc1ncc(C(=O)Nc2cc(-n3cc(C(=O)NCCCN4CCOCC4)nn3)ccc2N2CCN(C)CC2)c(C(F)(F)F)n1] |
| 187 | 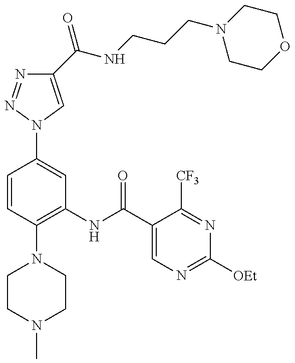 [SMILES: CCOc1ncc(C(=O)Nc2cc(-n3cc(C(=O)NCCCN4CCOCC4)nn3)ccc2N2CCN(C)CC2)c(C(F)(F)F)n1] |
| 188 | 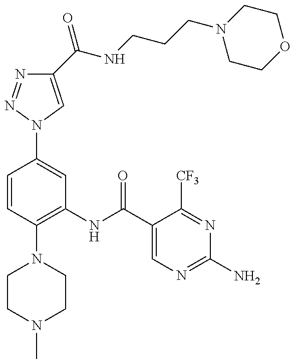 [SMILES: CN1CCN(c2ccc(-n3cc(C(=O)NCCCN4CCOCC4)nn3)cc2NC(=O)c2cnc(N)nc2C(F)(F)F)CC1] |
| 189 | 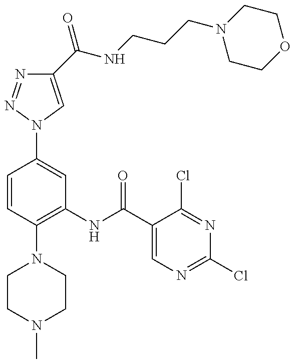 [SMILES: CN1CCN(c2ccc(-n3cc(C(=O)NCCCN4CCOCC4)nn3)cc2NC(=O)c2cnc(Cl)nc2Cl)CC1] |
| 190 | 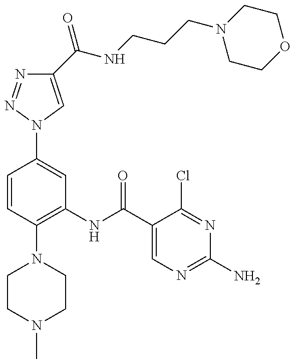 [SMILES: CN1CCN(c2ccc(-n3cc(C(=O)NCCCN4CCOCC4)nn3)cc2NC(=O)c2cnc(N)nc2Cl)CC1] |
| 191 | 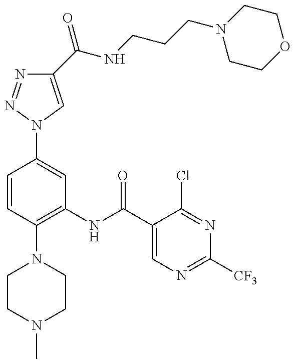 [SMILES: CN1CCN(c2ccc(-n3cc(C(=O)NCCCN4CCOCC4)nn3)cc2NC(=O)c2cnc(C(F)(F)F)nc2Cl)CC1] |
| 192 | 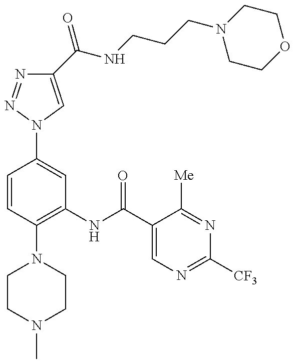 [SMILES: Cc1nc(C(F)(F)F)ncc1C(=O)Nc1cc(-n2cc(C(=O)NCCCN3CCOCC3)nn2)ccc1N1CCN(C)CC1] |
| 193 | 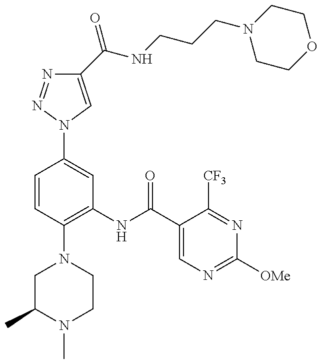 [SMILES: COc1ncc(C(=O)Nc2cc(-n3cc(C(=O)NCCCN4CCOCC4)nn3)ccc2N2CCN(C)[C@@H](C)C2)c(C(F)(F)F)n1] |
| 194 | 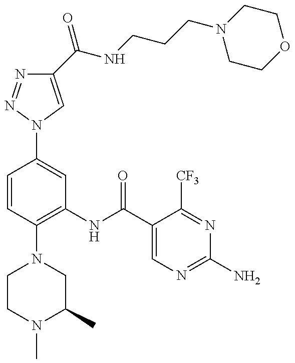 [SMILES: C[C@@H]1CN(c2ccc(-n3cc(C(=O)NCCCN4CCOCC4)nn3)cc2NC(=O)c2cnc(N)nc2C(F)(F)F)CCN1C] |
| 195 | 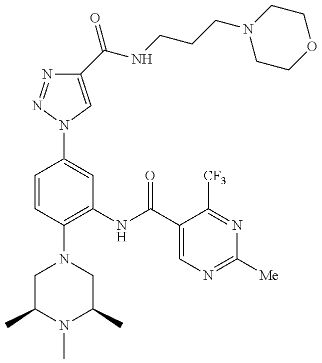 [SMILES: Cc1ncc(C(=O)Nc2cc(-n3cc(C(=O)NCCCN4CCOCC4)nn3)ccc2N2C[C@@H](C)N(C)[C@@H](C)C2)c(C(F)(F)F)n1] |
| 196 | 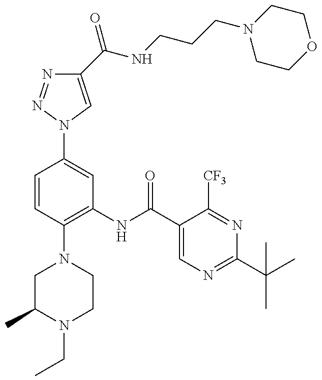 [SMILES: CCN1CCN(c2ccc(-n3cc(C(=O)NCCCN4CCOCC4)nn3)cc2NC(=O)c2cnc(C(C)(C)C)nc2C(F)(F)F)C[C@@H]1C] |
| 197 | 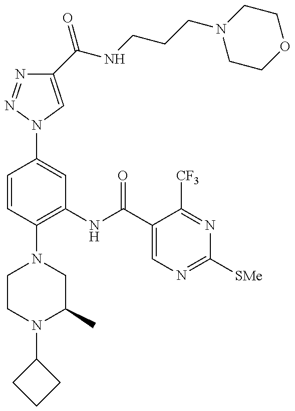 [SMILES: CSc1ncc(C(=O)Nc2cc(-n3cc(C(=O)NCCCN4CCOCC4)nn3)ccc2N2CCN(C3CCC3)[C@H](C)C2)c(C(F)(F)F)n1] |
| 198 | 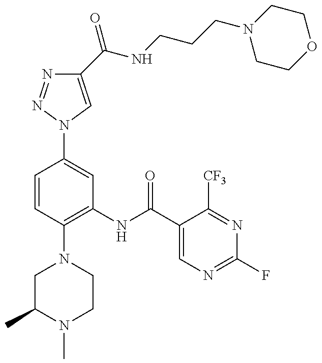 [SMILES: C[C@H]1CN(c2ccc(-n3cc(C(=O)NCCCN4CCOCC4)nn3)cc2NC(=O)c2cnc(F)nc2C(F)(F)F)CCN1C] |
| 199 | 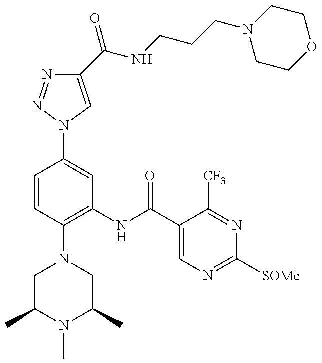 |
| 200 | 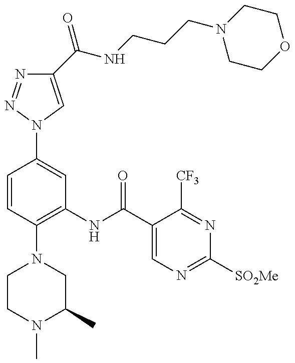 [SMILES: C[C@@H]1CN(c2ccc(-n3cc(C(=O)NCCCN4CCOCC4)nn3)cc2NC(=O)c2cnc(S(C)(=O)=O)nc2C(F)(F)F)CCN1C] |
| 201 | 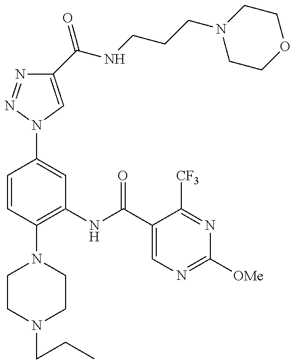 [SMILES: CCCN1CCN(c2ccc(-n3cc(C(=O)NCCCN4CCOCC4)nn3)cc2NC(=O)c2cnc(OC)nc2C(F)(F)F)CC1] |
| 202 | 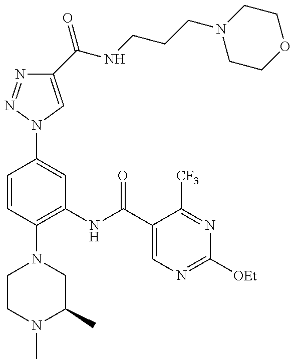 [SMILES: CCOc1ncc(C(=O)Nc2cc(-n3cc(C(=O)NCCCN4CCOCC4)nn3)ccc2N2CCN(C)[C@H](C)C2)c(C(F)(F)F)n1] |
| 203 | 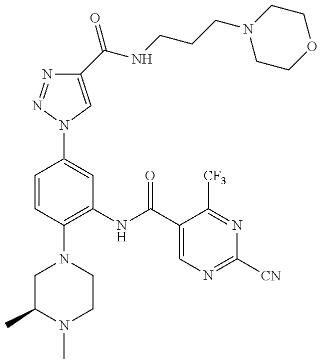 [SMILES: C[C@H]1CN(c2ccc(-n3cc(C(=O)NCCCN4CCOCC4)nn3)cc2NC(=O)c2cnc(C#N)nc2C(F)(F)F)CCN1C] |
| 204 | 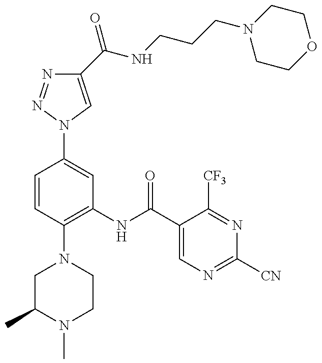 [SMILES: C[C@H]1CN(c2ccc(-n3cc(C(=O)NCCCN4CCOCC4)nn3)cc2NC(=O)c2cnc(C#N)nc2C(F)(F)F)CCN1C] |
| 205 | 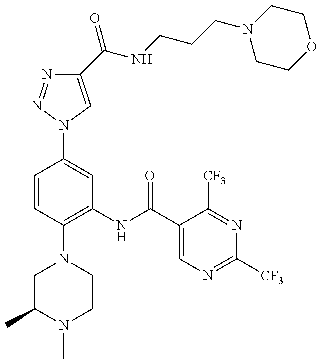 [SMILES: C[C@H]1CN(c2ccc(-n3cc(C(=O)NCCCN4CCOCC4)nn3)cc2NC(=O)c2cnc(C(F)(F)F)nc2C(F)(F)F)CCN1C] |
| 206 | 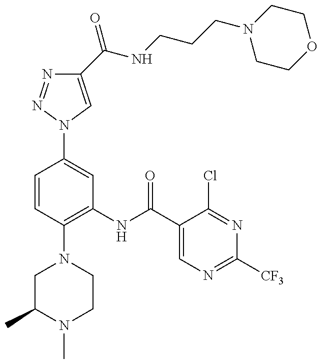 [SMILES: C[C@H]1CN(c2ccc(-n3cc(C(=O)NCCCN4CCOCC4)nn3)cc2NC(=O)c2cnc(C(F)(F)F)nc2Cl)CCN1C] |
| 207 | 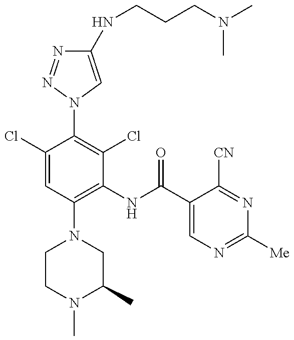 [SMILES: Cc1ncc(C(=O)Nc2c(N3CCN(C)[C@H](C)C3)cc(Cl)c(-n3cc(NCCCN(C)C)nn3)c2Cl)c(C#N)n1] |
| 208 | 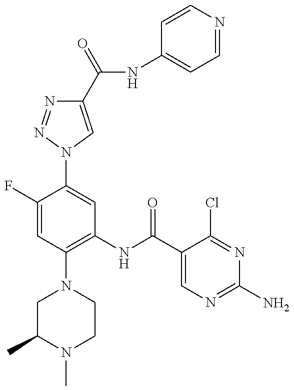 [SMILES: C[C@H]1CN(c2cc(F)c(-n3cc(C(=O)Nc4ccncc4)nn3)cc2NC(=O)c2cnc(N)nc2Cl)CCN1C] |
| 209 | 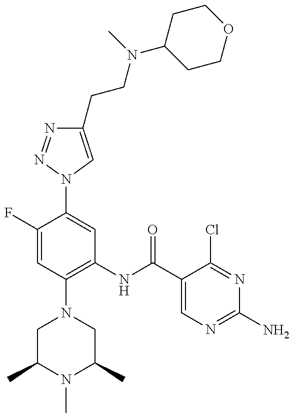 [SMILES: C[C@@H]1CN(c2cc(F)c(-n3cc(CCN(C)C4CCOCC4)nn3)cc2NC(=O)c2cnc(N)nc2Cl)C[C@H](C)N1C] |
| 210 | 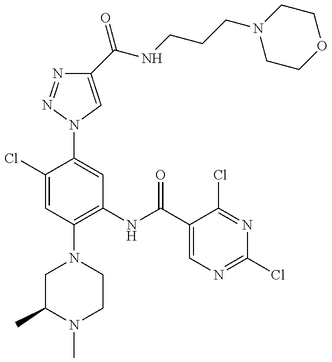 [SMILES: C[C@H]1CN(c2cc(Cl)c(-n3cc(C(=O)NCCCN4CCOCC4)nn3)cc2NC(=O)c2cnc(Cl)nc2Cl)CCN1C] |
| 211 | 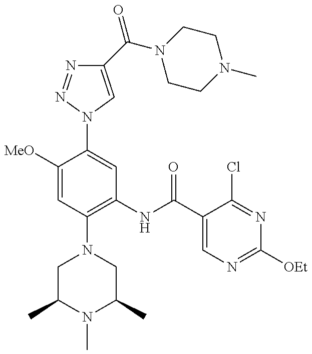 [SMILES: CCOc1ncc(C(=O)Nc2cc(-n3cc(C(=O)N4CCN(C)CC4)nn3)c(OC)cc2N2C[C@@H](C)N(C)[C@@H](C)C2)c(Cl)n1] |
| 212 | 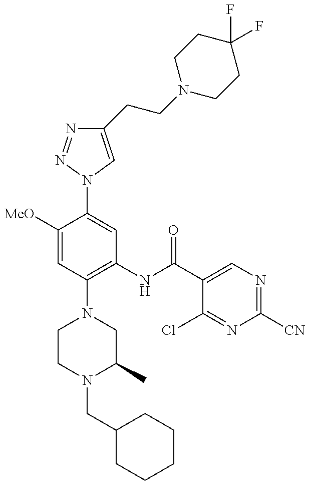 [SMILES: COc1cc(N2CCN(CC3CCCCC3)[C@H](C)C2)c(NC(=O)c2cnc(C#N)nc2Cl)cc1-n1cc(CCN2CCC(F)(F)CC2)nn1] |
| 213 | 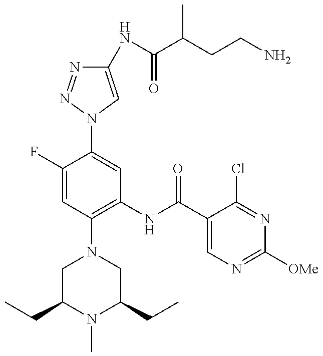 [SMILES: CC[C@@H]1CN(c2cc(F)c(-n3cc(NC(=O)C(C)CCN)nn3)cc2NC(=O)c2cnc(OC)nc2Cl)C[C@H](CC)N1C] |
| 214 | 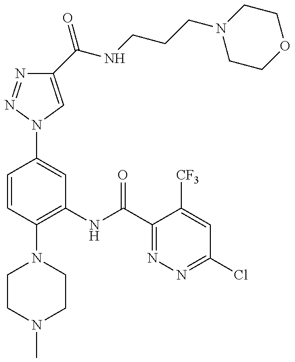 [SMILES: CN1CCN(c2ccc(-n3cc(C(=O)NCCCN4CCOCC4)nn3)cc2NC(=O)c2nnc(Cl)cc2C(F)(F)F)CC1] |
| 215 | 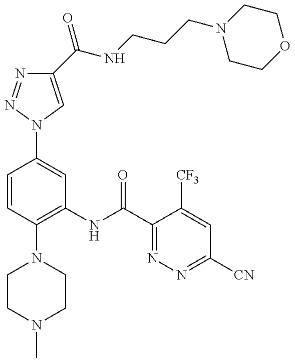 [SMILES: CN1CCN(c2ccc(-n3cc(C(=O)NCCCN4CCOCC4)nn3)cc2NC(=O)c2nnc(C#N)cc2C(F)(F)F)CC1] |
| 216 | 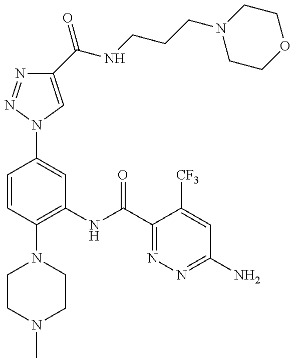 [SMILES: CN1CCN(c2ccc(-n3cc(C(=O)NCCCN4CCOCC4)nn3)cc2NC(=O)c2nnc(N)cc2C(F)(F)F)CC1] |
| 217 | 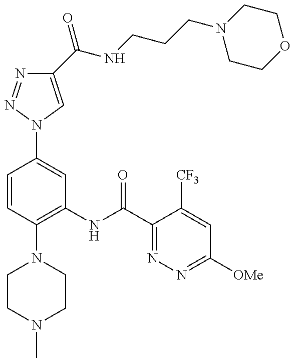 [SMILES: COc1cc(C(F)(F)F)c(C(=O)Nc2cc(-n3cc(C(=O)NCCCN4CCOCC4)nn3)ccc2N2CCN(C)CC2)nn1] |
| 218 | 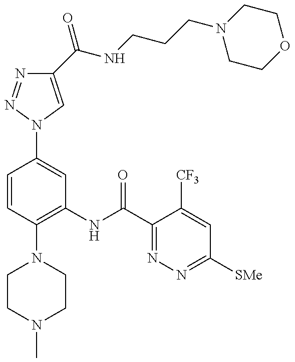 [SMILES: CSc1cc(C(F)(F)F)c(C(=O)Nc2cc(-n3cc(C(=O)NCCCN4CCOCC4)nn3)ccc2N2CCN(C)CC2)nn1] |
| 219 | 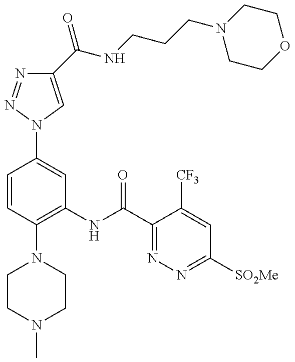 [SMILES: CN1CCN(c2ccc(-n3cc(C(=O)NCCCN4CCOCC4)nn3)cc2NC(=O)c2nnc(S(C)(=O)=O)cc2C(F)(F)F)CC1] |
| 220 | 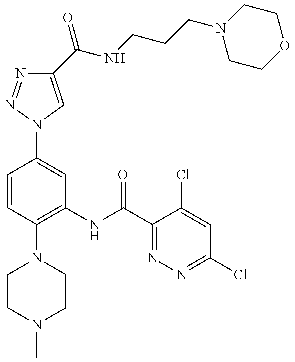 [SMILES: CN1CCN(c2ccc(-n3cc(C(=O)NCCCN4CCOCC4)nn3)cc2NC(=O)c2nnc(Cl)cc2Cl)CC1] |
| 221 | 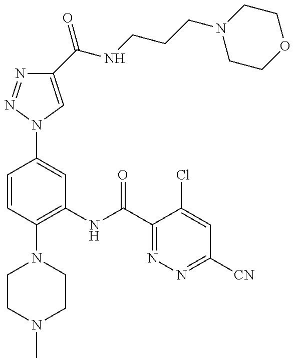 [SMILES: CN1CCN(c2ccc(-n3cc(C(=O)NCCCN4CCOCC4)nn3)cc2NC(=O)c2nnc(C#N)cc2Cl)CC1] |
| 222 | 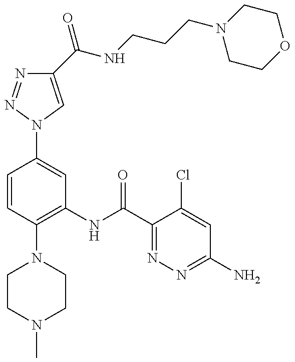 [SMILES: CN1CCN(c2ccc(-n3cc(C(=O)NCCCN4CCOCC4)nn3)cc2NC(=O)c2nnc(N)cc2Cl)CC1] |
| 223 | 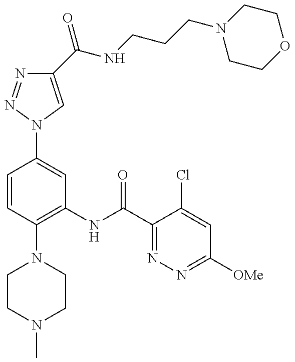 [SMILES: COc1cc(Cl)c(C(=O)Nc2cc(-n3cc(C(=O)NCCCN4CCOCC4)nn3)ccc2N2CCN(C)CC2)nn1] |
| 224 | 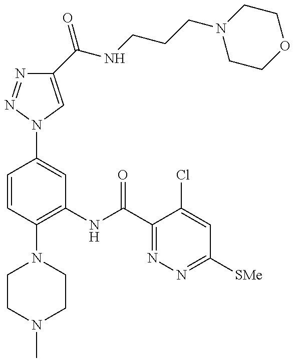 [SMILES: CSc1cc(Cl)c(C(=O)Nc2cc(-n3cc(C(=O)NCCCN4CCOCC4)nn3)ccc2N2CCN(C)CC2)nn1] |
| 225 | 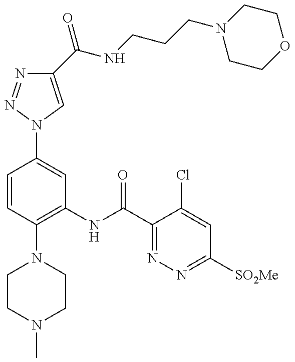 [SMILES: CN1CCN(c2ccc(-n3cc(C(=O)NCCCN4CCOCC4)nn3)cc2NC(=O)c2nnc(S(C)(=O)=O)cc2Cl)CC1] |
| 226 | 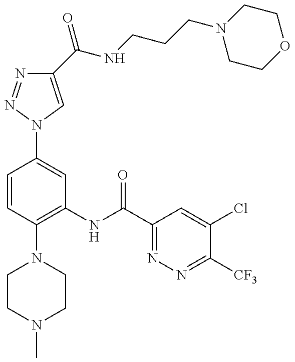 [SMILES: CN1CCN(c2ccc(-n3cc(C(=O)NCCCN4CCOCC4)nn3)cc2NC(=O)c2cc(Cl)c(C(F)(F)F)nn2)CC1] |
| 227 | 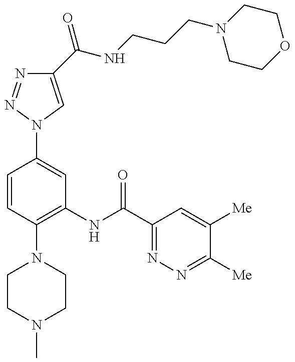 [SMILES: Cc1cc(C(=O)Nc2cc(-n3cc(C(=O)NCCCN4CCOCC4)nn3)ccc2N2CCN(C)CC2)nnc1C] |
| 228 | 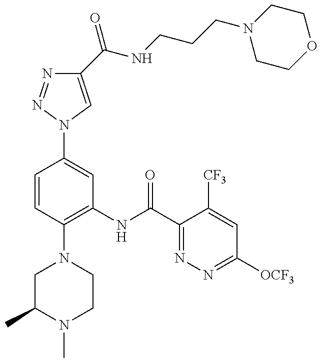 [SMILES: C[C@H]1CN(c2ccc(-n3cc(C(=O)NCCCN4CCOCC4)nn3)cc2NC(=O)c2nnc(OC(F)(F)F)cc2C(F)(F)F)CCN1C] |
| 229 | 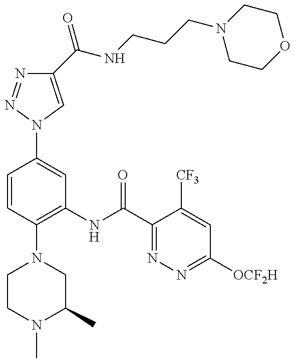 |
| 230 | 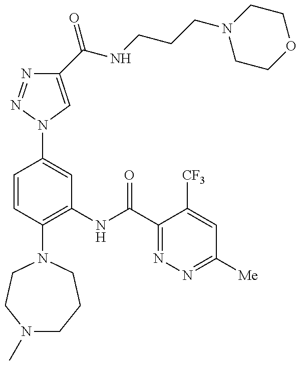 [SMILES: Cc1cc(C(F)(F)F)c(C(=O)Nc2cc(-n3cc(C(=O)NCCCN4CCOCC4)nn3)ccc2N2CCCN(C)CC2)nn1] |
| 231 | 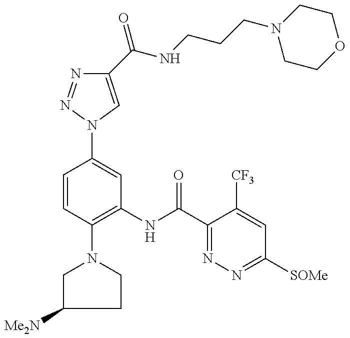 |
| 232 | 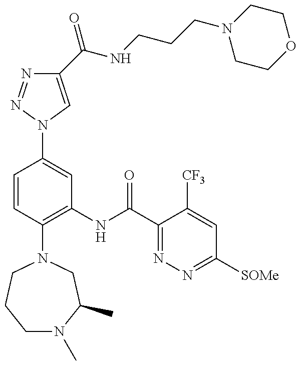 |
| 233 | 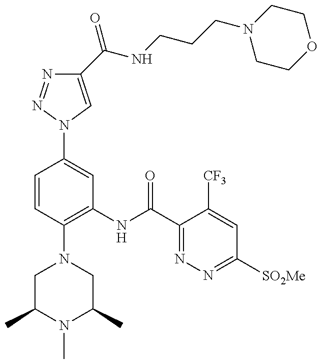 [SMILES: C[C@@H]1CN(c2ccc(-n3cc(C(=O)NCCCN4CCOCC4)nn3)cc2NC(=O)c2nnc(S(C)(=O)=O)cc2C(F)(F)F)C[C@H](C)N1C] |
| 234 | 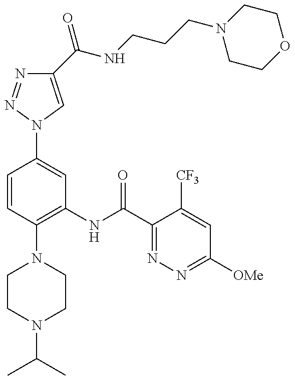 [SMILES: COc1cc(C(F)(F)F)c(C(=O)Nc2cc(-n3cc(C(=O)NCCCN4CCOCC4)nn3)ccc2N2CCN(C(C)C)CC2)nn1] |
| 235 | 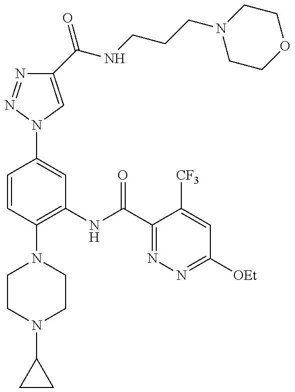 [SMILES: CCOc1cc(C(F)(F)F)c(C(=O)Nc2cc(-n3cc(C(=O)NCCCN4CCOCC4)nn3)ccc2N2CCN(C3CC3)CC2)nn1] |
| 236 | 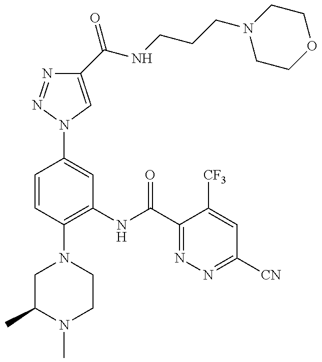 [SMILES: C[C@H]1CN(c2ccc(-n3cc(C(=O)NCCCN4CCOCC4)nn3)cc2NC(=O)c2nnc(C#N)cc2C(F)(F)F)CCN1C] |
| 237 | 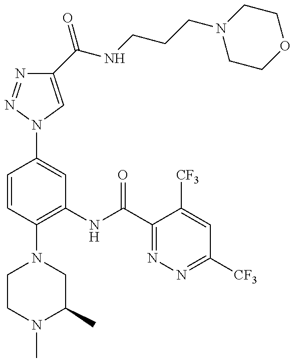 [SMILES: C[C@@H]1CN(c2ccc(-n3cc(C(=O)NCCCN4CCOCC4)nn3)cc2NC(=O)c2nnc(C(F)(F)F)cc2C(F)(F)F)CCN1C] |
| 238 | 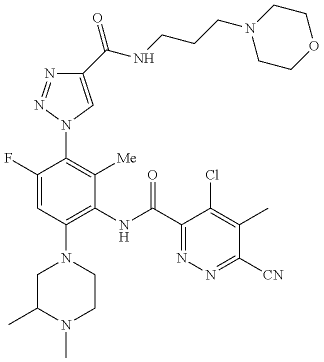 [SMILES: Cc1c(C#N)nnc(C(=O)Nc2c(N3CCN(C)C(C)C3)cc(F)c(-n3cc(C(=O)NCCCN4CCOCC4)nn3)c2C)c1Cl] |
| 239 | 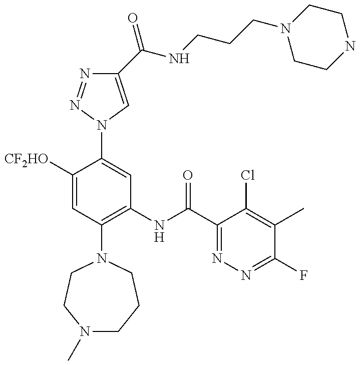 |
| 240 | 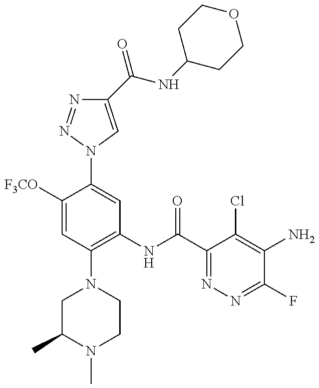 [SMILES: C[C@H]1CN(c2cc(OC(F)(F)F)c(-n3cc(C(=O)NC4CCOCC4)nn3)cc2NC(=O)c2nnc(F)c(N)c2Cl)CCN1C] |
| 241 | 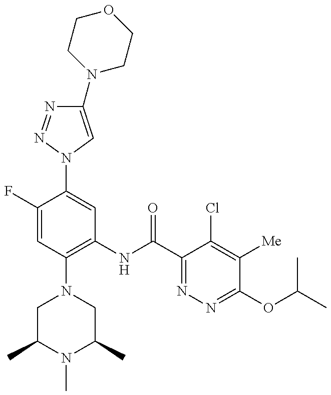 [SMILES: Cc1c(OC(C)C)nnc(C(=O)Nc2cc(-n3cc(N4CCOCC4)nn3)c(F)cc2N2C[C@@H](C)N(C)[C@@H](C)C2)c1Cl] |
| 242 | 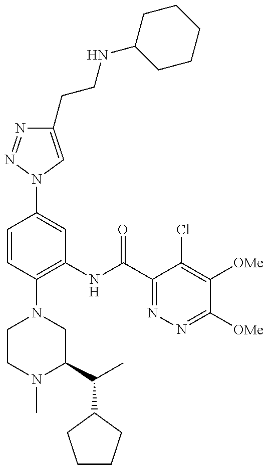 [SMILES: COc1nnc(C(=O)Nc2cc(-n3cc(CCNC4CCCCC4)nn3)ccc2N2CCN(C)[C@H]([C@H](C)C3CCCC3)C2)c(Cl)c1OC] |
| 243 | 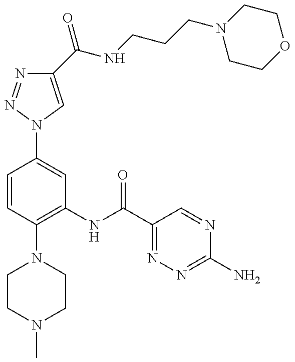 [SMILES: CN1CCN(c2ccc(-n3cc(C(=O)NCCCN4CCOCC4)nn3)cc2NC(=O)c2cnc(N)nn2)CC1] |
| 244 | 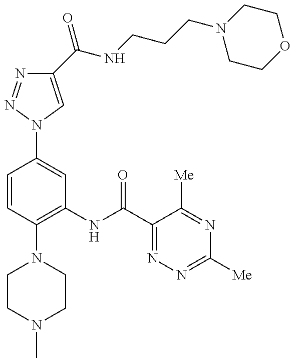 [SMILES: Cc1nnc(C(=O)Nc2cc(-n3cc(C(=O)NCCCN4CCOCC4)nn3)ccc2N2CCN(C)CC2)c(C)n1] |
| 245 | 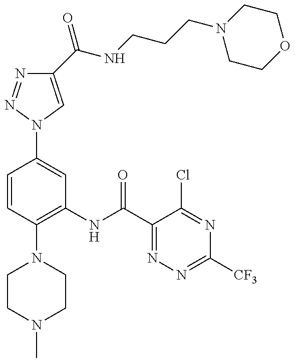 [SMILES: CN1CCN(c2ccc(-n3cc(C(=O)NCCCN4CCOCC4)nn3)cc2NC(=O)c2nnc(C(F)(F)F)nc2Cl)CC1] |
| 246 | 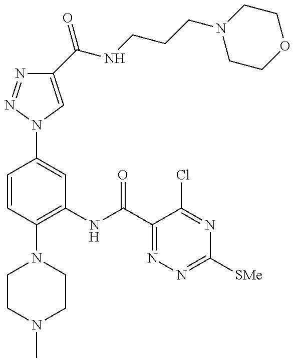 [SMILES: CSc1nnc(C(=O)Nc2cc(-n3cc(C(=O)NCCCN4CCOCC4)nn3)ccc2N2CCN(C)CC2)c(Cl)n1] |
| 247 | 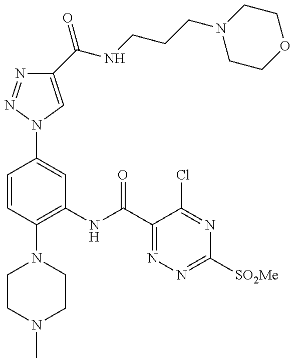 [SMILES: CN1CCN(c2ccc(-n3cc(C(=O)NCCCN4CCOCC4)nn3)cc2NC(=O)c2nnc(S(C)(=O)=O)nc2Cl)CC1] |
| 248 | 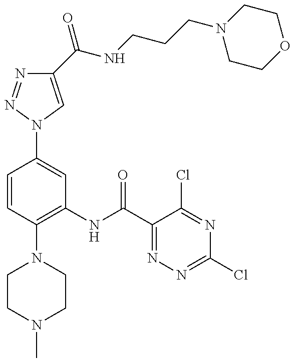 [SMILES: CN1CCN(c2ccc(-n3cc(C(=O)NCCCN4CCOCC4)nn3)cc2NC(=O)c2nnc(Cl)nc2Cl)CC1] |
| 249 | 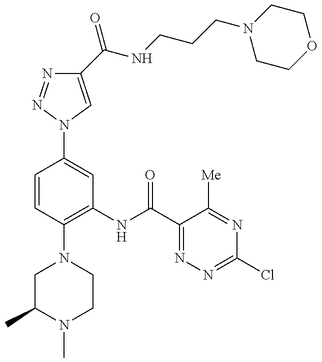 [SMILES: Cc1nc(Cl)nnc1C(=O)Nc1cc(-n2cc(C(=O)NCCCN3CCOCC3)nn2)ccc1N1CCN(C)[C@@H](C)C1] |
| 250 | 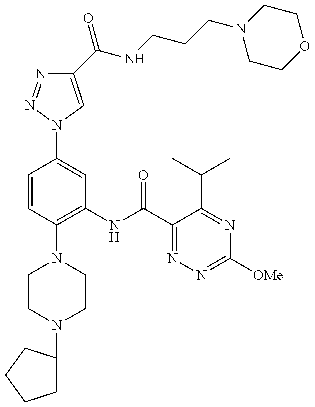 [SMILES: COc1nnc(C(=O)Nc2cc(-n3cc(C(=O)NCCCN4CCOCC4)nn3)ccc2N2CCN(C3CCCC3)CC2)c(C(C)C)n1] |
| 251 | 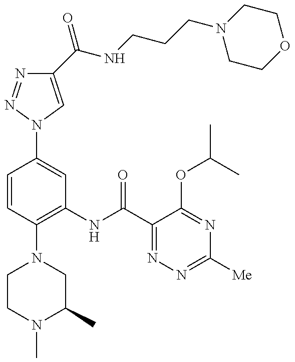 [SMILES: Cc1nnc(C(=O)Nc2cc(-n3cc(C(=O)NCCCN4CCOCC4)nn3)ccc2N2CCN(C)[C@H](C)C2)c(OC(C)C)n1] |
| 252 | 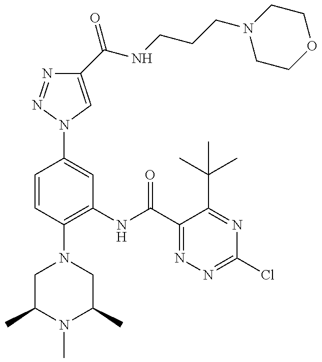 [SMILES: C[C@@H]1CN(c2ccc(-n3cc(C(=O)NCCCN4CCOCC4)nn3)cc2NC(=O)c2nnc(Cl)nc2C(C)(C)C)C[C@H](C)N1C] |
| 253 | 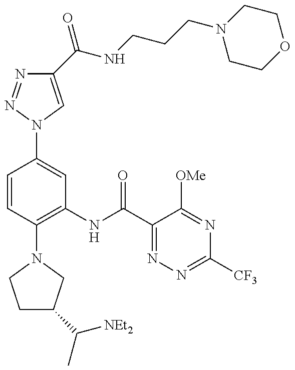 |
| 254 | 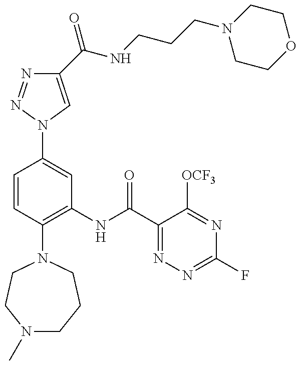 [SMILES: CN1CCCN(c2ccc(-n3cc(C(=O)NCCCN4CCOCC4)nn3)cc2NC(=O)c2nnc(F)nc2OC(F)(F)F)CC1] |
| 255 | 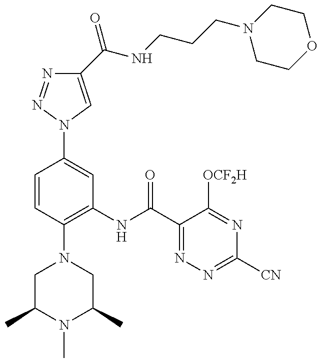 |
| 256 | 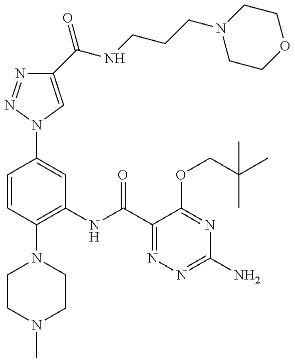 [SMILES: CN1CCN(c2ccc(-n3cc(C(=O)NCCCN4CCOCC4)nn3)cc2NC(=O)c2nnc(N)nc2OCC(C)(C)C)CC1] |
| 257 | 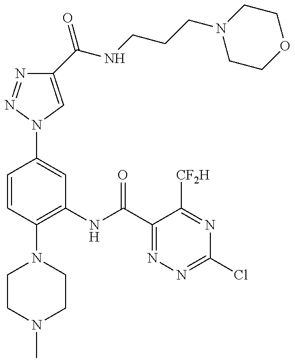 [SMILES: CN1CCN(c2ccc(-n3cc(C(=O)NCCCN4CCOCC4)nn3)cc2NC(=O)c2nnc(Cl)nc2C(F)F)CC1] |
| 258 | 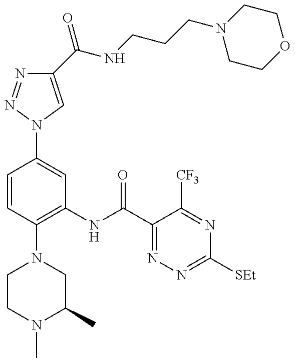 |
| 260 | 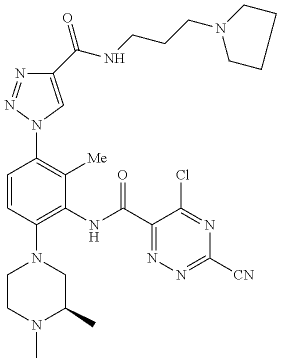 [SMILES: Cc1c(-n2cc(C(=O)NCCCN3CCCC3)nn2)ccc(N2CCN(C)[C@H](C)C2)c1NC(=O)c1nnc(C#N)nc1Cl] |
| 261 | 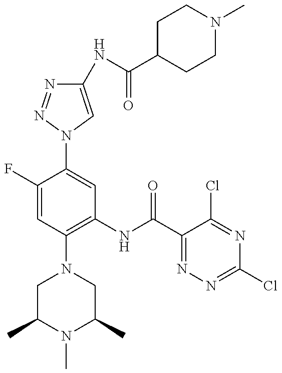 [SMILES: C[C@@H]1CN(c2cc(F)c(-n3cc(NC(=O)C4CCN(C)CC4)nn3)cc2NC(=O)c2nnc(Cl)nc2Cl)C[C@H](C)N1C] |
| 262 | 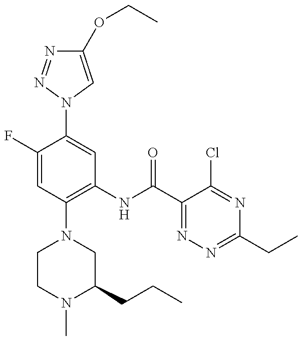 [SMILES: CCC[C@@H]1CN(c2cc(F)c(-n3cc(OCC)nn3)cc2NC(=O)c2nnc(CC)nc2Cl)CCN1C] |
| 263 | 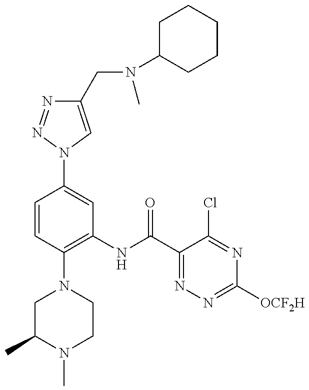 |
| 264 | 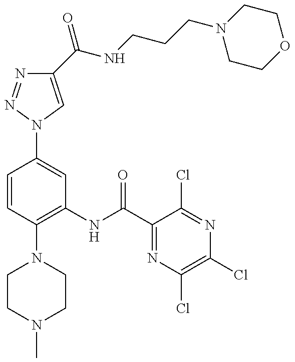 [SMILES: CN1CCN(c2ccc(-n3cc(C(=O)NCCCN4CCOCC4)nn3)cc2NC(=O)c2nc(Cl)c(Cl)nc2Cl)CC1] |
| 265 | 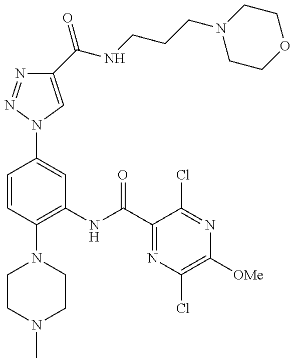 [SMILES: COc1nc(Cl)c(C(=O)Nc2cc(-n3cc(C(=O)NCCCN4CCOCC4)nn3)ccc2N2CCN(C)CC2)nc1Cl] |
| 266 | 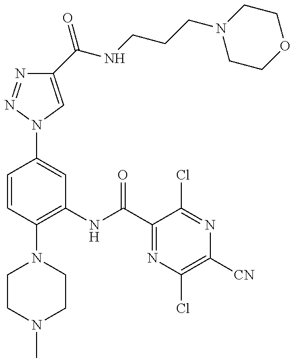 [SMILES: CN1CCN(c2ccc(-n3cc(C(=O)NCCCN4CCOCC4)nn3)cc2NC(=O)c2nc(Cl)c(C#N)nc2Cl)CC1] |
| 267 | 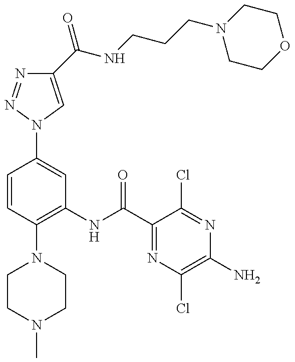 [SMILES: CN1CCN(c2ccc(-n3cc(C(=O)NCCCN4CCOCC4)nn3)cc2NC(=O)c2nc(Cl)c(N)nc2Cl)CC1] |
| 268 | 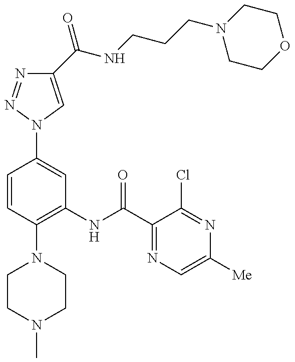 [SMILES: Cc1cnc(C(=O)Nc2cc(-n3cc(C(=O)NCCCN4CCOCC4)nn3)ccc2N2CCN(C)CC2)c(Cl)n1] |
| 269 | 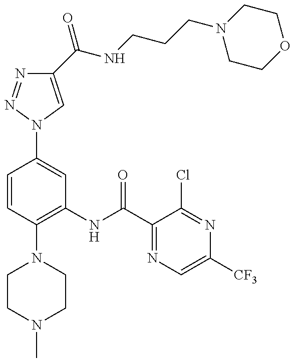 [SMILES: CN1CCN(c2ccc(-n3cc(C(=O)NCCCN4CCOCC4)nn3)cc2NC(=O)c2ncc(C(F)(F)F)nc2Cl)CC1] |
| 270 | 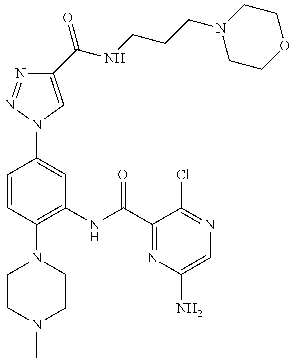 [SMILES: CN1CCN(c2ccc(-n3cc(C(=O)NCCCN4CCOCC4)nn3)cc2NC(=O)c2nc(N)cnc2Cl)CC1] |
| 280 | 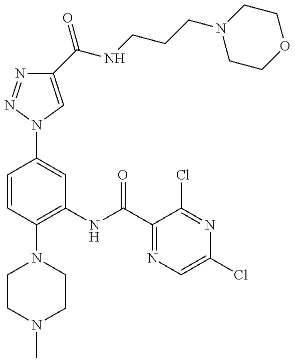 [SMILES: CN1CCN(c2ccc(-n3cc(C(=O)NCCCN4CCOCC4)nn3)cc2NC(=O)c2ncc(Cl)nc2Cl)CC1] |
| 281 | 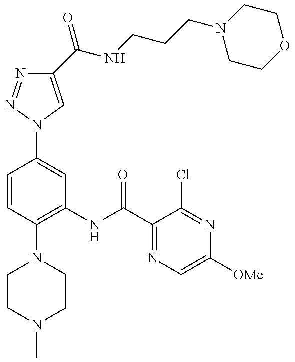 [SMILES: COc1cnc(C(=O)Nc2cc(-n3cc(C(=O)NCCCN4CCOCC4)nn3)ccc2N2CCN(C)CC2)c(Cl)n1] |
| 282 | 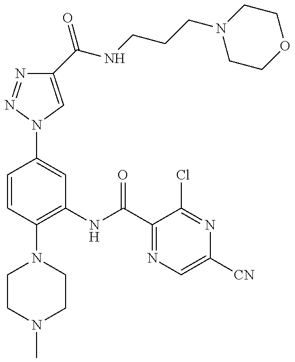 [SMILES: CN1CCN(c2ccc(-n3cc(C(=O)NCCCN4CCOCC4)nn3)cc2NC(=O)c2ncc(C#N)nc2Cl)CC1] |
| 283 | 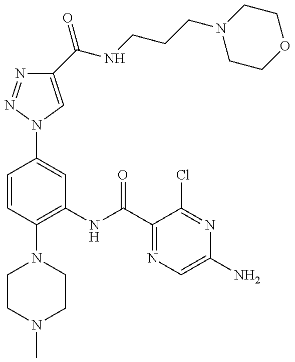 [SMILES: CN1CCN(c2ccc(-n3cc(C(=O)NCCCN4CCOCC4)nn3)cc2NC(=O)c2ncc(N)nc2Cl)CC1] |
| 284 | 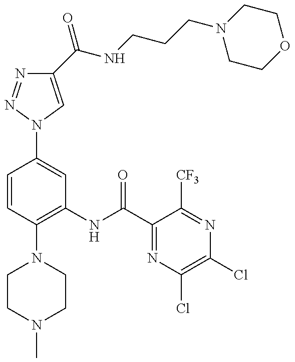 [SMILES: CN1CCN(c2ccc(-n3cc(C(=O)NCCCN4CCOCC4)nn3)cc2NC(=O)c2nc(Cl)c(Cl)nc2C(F)(F)F)CC1] |
| 285 | 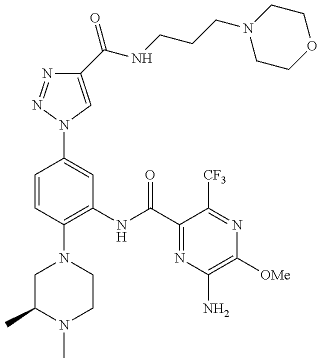 [SMILES: COc1nc(C(F)(F)F)c(C(=O)Nc2cc(-n3cc(C(=O)NCCCN4CCOCC4)nn3)ccc2N2CCN(C)[C@@H](C)C2)nc1N] |
| 286 | 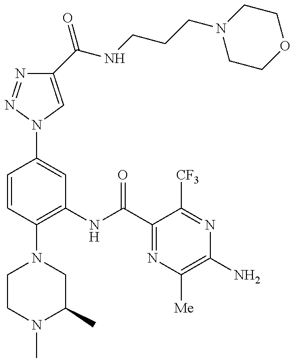 [SMILES: Cc1nc(C(=O)Nc2cc(-n3cc(C(=O)NCCCN4CCOCC4)nn3)ccc2N2CCN(C)[C@H](C)C2)c(C(F)(F)F)nc1N] |
| 287 | 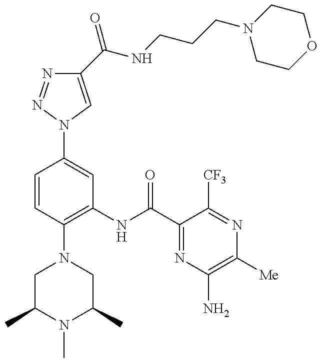 [SMILES: Cc1nc(C(F)(F)F)c(C(=O)Nc2cc(-n3cc(C(=O)NCCCN4CCOCC4)nn3)ccc2N2C[C@@H](C)N(C)[C@@H](C)C2)nc1N] |
| 288 | 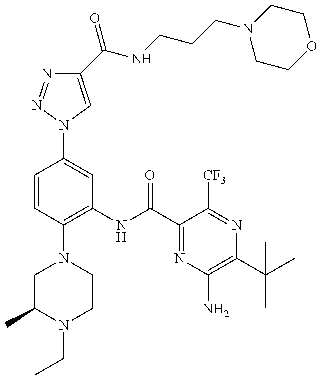 [SMILES: CCN1CCN(c2ccc(-n3cc(C(=O)NCCCN4CCOCC4)nn3)cc2NC(=O)c2nc(N)c(C(C)(C)C)nc2C(F)(F)F)C[C@@H]1C] |
| 289 | 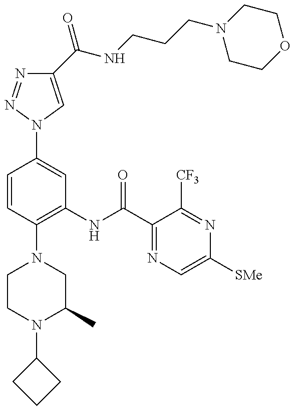 [SMILES: CSc1cnc(C(=O)Nc2cc(-n3cc(C(=O)NCCCN4CCOCC4)nn3)ccc2N2CCN(C3CCC3)[C@H](C)C2)c(C(F)(F)F)n1] |
| 290 | 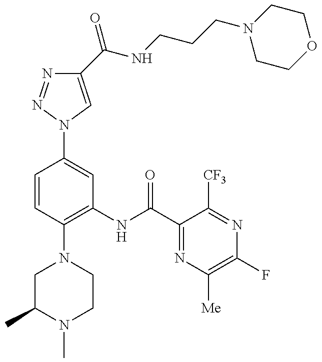 [SMILES: Cc1nc(C(=O)Nc2cc(-n3cc(C(=O)NCCCN4CCOCC4)nn3)ccc2N2CCN(C)[C@@H](C)C2)c(C(F)(F)F)nc1F] |
| 291 | 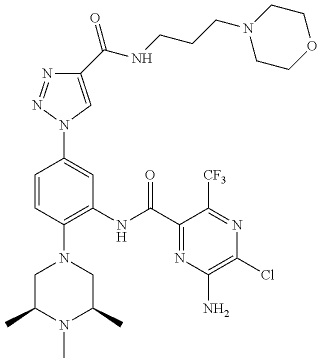 [SMILES: C[C@@H]1CN(c2ccc(-n3cc(C(=O)NCCCN4CCOCC4)nn3)cc2NC(=O)c2nc(N)c(Cl)nc2C(F)(F)F)C[C@H](C)N1C] |
| 292 | 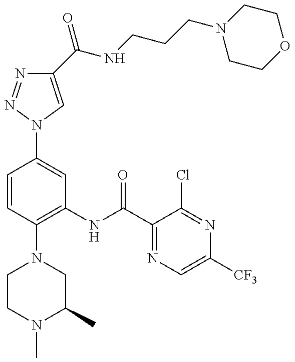 [SMILES: C[C@@H]1CN(c2ccc(-n3cc(C(=O)NCCCN4CCOCC4)nn3)cc2NC(=O)c2ncc(C(F)(F)F)nc2Cl)CCN1C] |
| 293 | 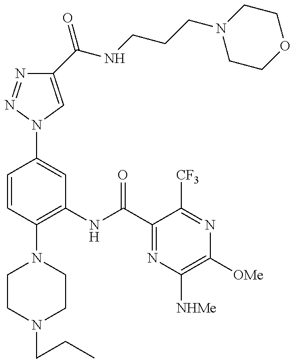 [SMILES: CCCN1CCN(c2ccc(-n3cc(C(=O)NCCCN4CCOCC4)nn3)cc2NC(=O)c2nc(NC)c(OC)nc2C(F)(F)F)CC1] |
| 294 | 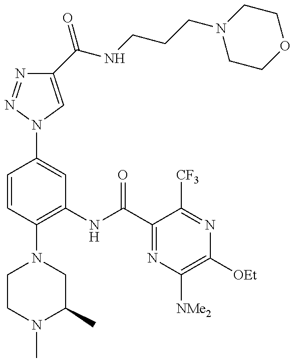 [SMILES: CCOc1nc(C(F)(F)F)c(C(=O)Nc2cc(-n3cc(C(=O)NCCCN4CCOCC4)nn3)ccc2N2CCN(C)[C@H](C)C2)nc1N(C)C] |
| 295 | 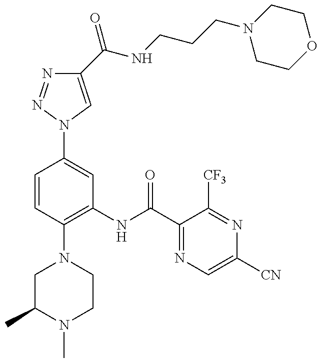 [SMILES: C[C@H]1CN(c2ccc(-n3cc(C(=O)NCCCN4CCOCC4)nn3)cc2NC(=O)c2ncc(C#N)nc2C(F)(F)F)CCN1C] |
| 296 | 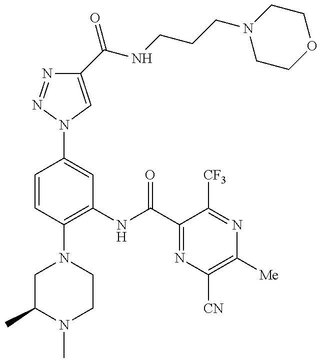 [SMILES: Cc1nc(C(F)(F)F)c(C(=O)Nc2cc(-n3cc(C(=O)NCCCN4CCOCC4)nn3)ccc2N2CCN(C)[C@@H](C)C2)nc1C#N] |
| 297 | 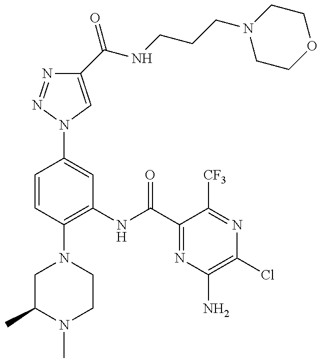 [SMILES: C[C@H]1CN(c2ccc(-n3cc(C(=O)NCCCN4CCOCC4)nn3)cc2NC(=O)c2nc(N)c(Cl)nc2C(F)(F)F)CCN1C] |
| 298 | 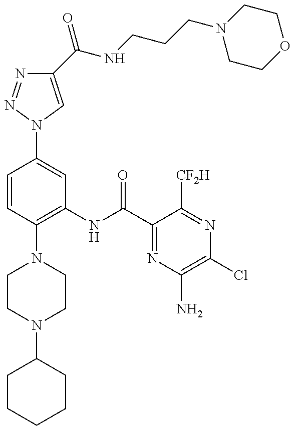 [SMILES: Nc1nc(C(=O)Nc2cc(-n3cc(C(=O)NCCCN4CCOCC4)nn3)ccc2N2CCN(C3CCCCC3)CC2)c(C(F)F)nc1Cl] |
| 299 | 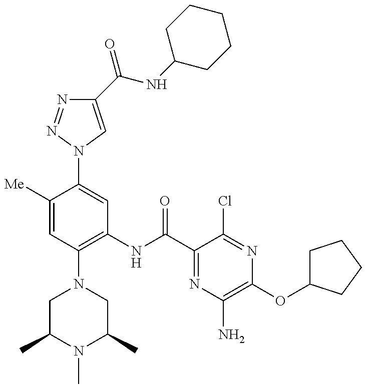 [SMILES: Cc1cc(N2C[C@@H](C)N(C)[C@@H](C)C2)c(NC(=O)c2nc(N)c(OC3CCCC3)nc2Cl)cc1-n1cc(C(=O)NC2CCCCC2)nn1] |
| 300 | 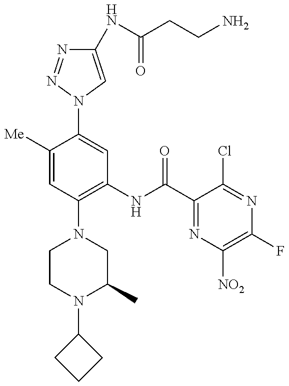 [SMILES: Cc1cc(N2CCN(C3CCC3)[C@H](C)C2)c(NC(=O)c2nc([N+](=O)[O-])c(F)nc2Cl)cc1-n1cc(NC(=O)CCN)nn1] |
| 301 | 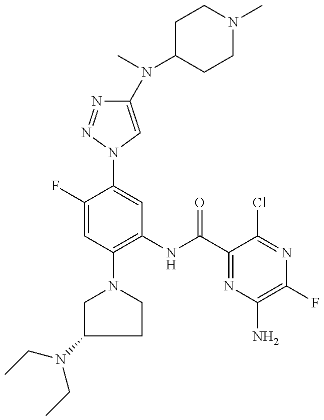 [SMILES: CCN(CC)[C@H]1CCN(c2cc(F)c(-n3cc(N(C)C4CCN(C)CC4)nn3)cc2NC(=O)c2nc(N)c(F)nc2Cl)C1] |
| 302 | 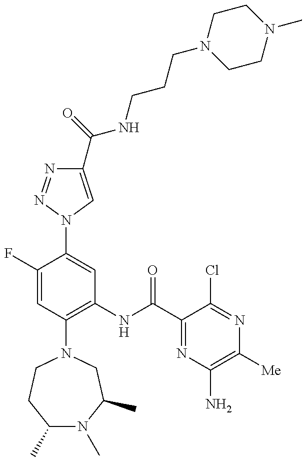 [SMILES: Cc1nc(Cl)c(C(=O)Nc2cc(-n3cc(C(=O)NCCCN4CCN(C)CC4)nn3)c(F)cc2N2CC[C@@H](C)N(C)[C@H](C)C2)nc1N] |
| 303 | 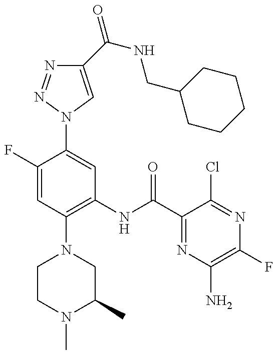 [SMILES: C[C@@H]1CN(c2cc(F)c(-n3cc(C(=O)NCC4CCCCC4)nn3)cc2NC(=O)c2nc(N)c(F)nc2Cl)CCN1C] |
| 304 | 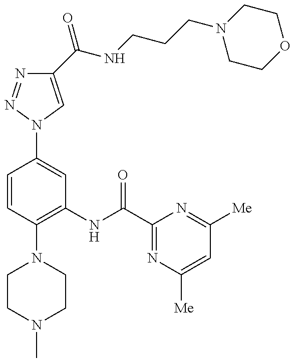 [SMILES: Cc1cc(C)nc(C(=O)Nc2cc(-n3cc(C(=O)NCCCN4CCOCC4)nn3)ccc2N2CCN(C)CC2)n1] |
| 305 | 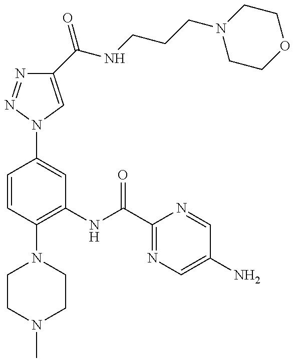 [SMILES: CN1CCN(c2ccc(-n3cc(C(=O)NCCCN4CCOCC4)nn3)cc2NC(=O)c2ncc(N)cn2)CC1] |
| 306 | 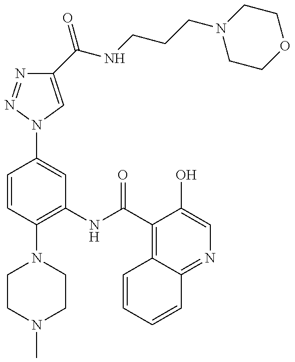 [SMILES: CN1CCN(c2ccc(-n3cc(C(=O)NCCCN4CCOCC4)nn3)cc2NC(=O)c2c(O)cnc3ccccc23)CC1] |
| 307 | 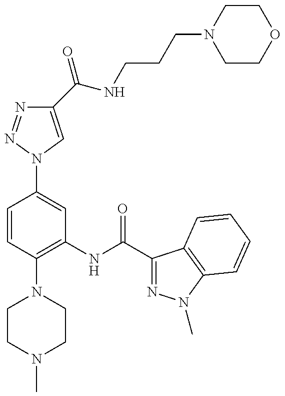 [SMILES: CN1CCN(c2ccc(-n3cc(C(=O)NCCCN4CCOCC4)nn3)cc2NC(=O)c2nn(C)c3ccccc23)CC1] |

In some embodiments, the compound is a compound of Table 1, or a pharmaceutically acceptable salt or solvate thereof. In some embodiments, the compound is a compound of Table 2, or a pharmaceutically acceptable salt or solvate thereof. In some embodiments, the compound is a compound of Table 3, or a pharmaceutically acceptable salt or solvate thereof.

### Further Forms of Compounds

In some embodiments, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by forming diastereomeric salts and separation is by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. Prodrugs may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery,” Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In some embodiments, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C ¹⁴C, ¹⁵N, ¹⁸O, ¹⁷O, ³⁵S, ¹⁸F, ³⁶Cl, and ¹²⁵I. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids. Pharmaceutically acceptable salts are also obtained by reacting a compound disclosed herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some embodiments, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In some embodiments, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compounds disclosed herein are formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin.

In some embodiments, the compounds disclosed herein are administered by inhalation.

In some embodiments, the compounds disclosed herein are formulated for intranasal administration. Such formulations include nasal sprays, nasal mists, and the like.

In some embodiments, the compounds disclosed herein are formulated as eye drops.

In any of the aforementioned embodiments are further embodiments in which an effective amount of the compounds disclosed herein are: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned embodiments are further embodiments comprising single administrations of an effective amount of the compounds disclosed herein, including further embodiments in which (i) the compounds are administered once; (ii) the compounds are administered to the mammal multiple times over the span of one day; (iii) the compounds are administered continually; or (iv) the compounds are administered continuously.

In any of the aforementioned embodiments are further embodiments comprising multiple administrations of the effective amount of the compounds disclosed herein, including further embodiments in which (i) the compounds are administered continuously or intermittently: as in a single dose; (ii) the time between multiple administrations is every 6 hours; (iii) the compounds are administered to the mammal every 8 hours; (iv) the compounds are administered to the mammal every 12 hours; (v) the compounds are administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compounds disclosed herein are administered in a local rather than systemic manner.

In some embodiments, the compounds disclosed herein are administered topically. In some embodiments, the compounds disclosed herein are administered systemically.

In some embodiments, the pharmaceutical formulation is in the form of a tablet. In other embodiments, pharmaceutical formulations of the compounds disclosed herein are in the form of a capsule.

In some embodiments, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In some embodiments, a compound disclosed herein is formulated into a pharmaceutical composition suitable for intramuscular, subcutaneous, or intravenous injection.

In some embodiments, the compounds disclosed herein are administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In some embodiments, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compounds disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 0.01 mg to about 1000 mg per day. In one embodiment, the desired dose is conveniently presented in a single dose or in divided doses.

### Methods of Treatment

Described herein is are methods for the treatment of diseases mediated by MLL1 through inhibiting MLL1-WDR5 protein-protein interaction, wherein the diseases, such as for example MLL gene fusion type leukemia can be treated through inhibition of the enzymatic activity of MLL1. In some embodiments, described herein is a method of treating a disease or condition including administering to a subject in need thereof an effective amount of a compound disclosed herein.

In some embodiments, the disease or condition being treated is a cancer comprising a solid tumor or hematologoical cancer. In some embodiments, the cancer is a blood cancer.

### Leukemia

Leukemia is characterized by an abnormal increase of white blood cells in the blood or bone marrow. Among all types of cancers, the morbidity of leukemia is the highest for patients below 35 years old. Over 70% of infant leukemia patients bear a translocation involving chromosome 11, resulting in the fusion of the MLL1 gene with other genes (Nat. Rev. Cancer., 2007, 7(11):823-833). MLL1 translocations are also found in approximately 10% of adult acute myeloid leukemia (AML) patients who were previously treated with topoisomerase II inhibitors for other types of cancers.

MLL1 enzymatic activity is determined by MLL1 and WDR5 protein-protein interaction; MLL1 enzymatic activity affects the methylation level of H3K4. The H3K4 methylation level increases abnormally in MLL fusion type leukemia, and the downstream Hox and Meis-1 gene expression levels are up-regulated abnormally. When MLL1-WDR5 protein-protein interaction is inhibited, MLL1 catalytic activity decreases, H3K4 methylation level decreases, Hox and Meis-1 gene expression levels are downregulated, inhibiting leukemia cell proliferation.

In some embodiments, the cancer is leukemia. In some embodiments, the leukemia is acute leukemia. In some embodiments, the acute leukemia is acute leukemia with MLL1 gene rearrangement.

### Acute Myeloid Leukemia (AML)

The CEBPA gene is mutated in 9% of patients with acute myeloid leukemia (AML). Selective expression of a short (30-kDa) CCAAT-enhancer binding protein-a (C/EBPa) translational isoform, termed p30, represents the most common type of CEBPA mutation in AML. The molecular mechanisms underlying p30-mediated transformation remain incompletely understood. Studies have shown that C/EBPa p30, but not the normal p42 isoform, preferentially interacts with WDR5, a key component of SET/MLL (SET-domain/mixed-lineage leukemia) histone-methyltransferase complexes. Accordingly, p30-bound genomic regions are enriched for MLL-dependent H3K4me3 marks. The p30-dependent increase in self-renewal and inhibition of myeloid differentiation required WDR5, as downregulation of the latter inhibited proliferation and restored differentiation in p30-dependent AML models. Small-molecule inhibitors of WDR5-MLL binding selectively inhibited proliferation and induced differentiation in p30-expressing human AML cells revealing the mechanism of p30-dependent transformation and establish the p30 cofactor WDR5 as a therapeutic target in CEBPA-mutant AML (Nat Chem Biol. 2015; 11(8):571-8).

In some embodiments, the leukemia is AML leukemia.

### MYCN-Amplified Neuroblastoma

MYCN gene amplification in neuroblastoma drives a gene expression program that correlates strongly with aggressive disease. Mechanistically, trimethylation of histone H3 lysine 4 (H3K4) at target gene promoters is a prerequisite for the transcriptional program to be enacted. WDR5 is a histone H3K4 presenter that has been found to have an essential role in H3K4 trimethylation. The relationship between WDR5-mediated H3K4 trimethylation and N-Myc transcriptional programs in neuroblastoma cells was investigated. N-Myc upregulated WDR5 expression in neuroblastoma cells. Gene expression analysis revealed that WDR5 target genes included those with MYC-binding elements at promoters such as MDM2. WDR5 has been shown to form a protein complex at the MDM2 promoter with N-Myc, but not p53, leading to histone H3K4 trimethylation and activation of MDM2 transcription (Cancer Res 2015; 75(23); 5143-54). RNAi-mediated attenuation of WDR5 upregulated expression of wild-type but not mutant p53, an effect associated with growth inhibition and apoptosis. Similarly, a small-molecule antagonist of WDR5 reduced N-Myc/WDR5 complex formation, N-Myc target gene expression, and cell growth in neuroblastoma cells. In MYCN-transgenic mice, WDR5 was overexpressed in precancerous ganglion and neuroblastoma cells compared with normal ganglion cells. Clinically, elevated levels of WDR5 in neuroblastoma specimens have an independent predictor of poor overall survival. WDR5 has been identified as a relevant cofactor for N-Myc-regulated transcriptional activation and tumorogenesis and as a novel therapeutic target for MYCN-amplified neuroblastomas (Cancer Res 2015; 75(23); 5143-54, Mol Cell. 2015; 58(3):440-52).

In some embodiments, the cancer is a solid tumor. In some embodiments, the cancer is a neuroblastoma.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkyl” refers to an alkyl radical as described above where one or more carbon atoms of the alkyl is replaced with a 0, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized n-electron system containing 4n+2 ?t electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

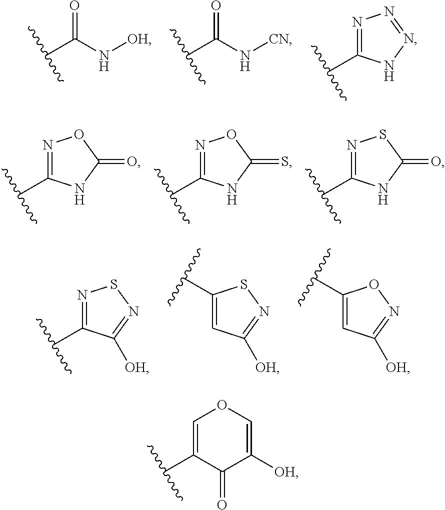

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆cycloalkyl. In some embodiments, the cycloalkyl is monocyclic, bicyclic or polycyclic. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, decalinyl and adamantyl. In some embodiments, the cycloalkyl is monocyclic. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the cycloalkyl is bicyclic. Bicyclic cycloalkyl groups include fused bicyclic cycloalkyl groups, spiro bicyclic cycloalkyl groups, and bridged bicyclic cycloalkyl groups. In some embodiments, cycloalkyl groups are selected from among spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, 3,4-dihydronaphthalen-1(2H)-one and decalinyl. In some embodiments, the cycloalkyl is polycyclic. Polycyclic radicals include, for example, adamantyl, and. In some embodiments, the polycyclic cycloalkyl is adamantyl. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro heterocycloalkyl), or polycyclic. In some embodiments, the heterocycloalkyl is monocyclic or bicyclic. In some embodiments, the heterocycloalkyl is monocyclic. In some embodiments, the heterocycloalkyl is bicyclic. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

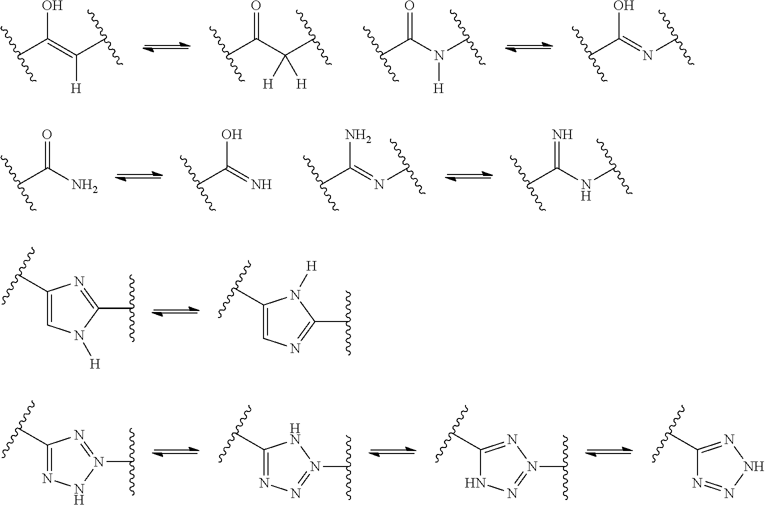

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I) and a co-agent, are both administered to a patient simultaneously in the form of a single entity or dosage. The term “non-fixed combination” means that the active ingredients, e.g., a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The term “subject” or “patient” encompasses mammals. Examples of mammals include, but are not limited to, humans. In some embodiments, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

It is understood that other analogous procedures and reagents could be used, and that the following reaction schemes are only meant as non-limiting examples.

### Examples

### Preparation of Compounds

### Abbreviations

DCM: Dichloromethane
DIEA: Diisopropylethylamine
DMF: Dimethyl formamide
DMSO: Dimethyl sulfoxide
ESI: Electrospray ionization
HPLC: High performance liquid chromatography
HRMS: High resolution mass spectrometry
h or hr(s): Hour(s)
HATU: 1-[Bis(dimethylamino)methylene]-1H-1,2,3-triazolo[4,5-b]pyridinium 3-oxid hexafluorophosphate
min(s): Minutes
m/z: Mass-to-charge ratio
¹H NMR: Proton nuclear magnetic resonance
¹³C NMR: Carbon nuclear magnetic resonance
rt: Room temperature
   The following Examples depict compounds of interest from Table 1.

### Example 1. Synthesis of 6-chloro-N-(5-(4-((3-morpholinopropyl)carbamoyl)-1H-1,2,3-triazol-1-yl)-2-((3S,5R)-3,4,5-trimethylpiperazin-1-yl)phenyl)-4-(trifluoromethyl)nicotinamide (HYBI-065A)

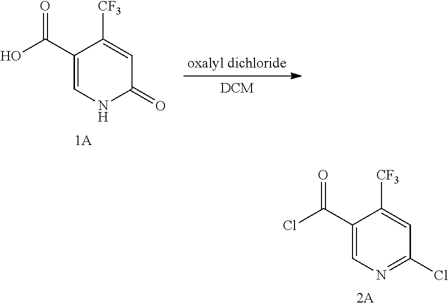 [SMILES: O=C(O)c1c[nH]c(=O)cc1C(F)(F)F ; O=C(Cl)c1cnc(Cl)cc1C(F)(F)F]

Synthesis of intermediate 2A: To a solution of 6-oxo-4-(trifluoromethyl)-1,6-dihydropyridine-3-carboxylic acid (compound 1A) (120 mg, 579.41 μmol, 1 eq.) and DMF (42 mg, 579.41 μmol, 44.58 μL, 1 eq.) in DCM (2 mL), was added (COCl)₂ (147 mg, 1.16 mmol, 101.44 μL, 2 eq.) drop-wise at 0° C. The reaction mixture was stirred at 20° C. for 1 hr. The reaction mixture was concentrated under reduced pressure to give a residue. The product was used directly to the next step without further purification. Intermediate compound 2A (140 mg, crude) was obtained as a yellow oil.

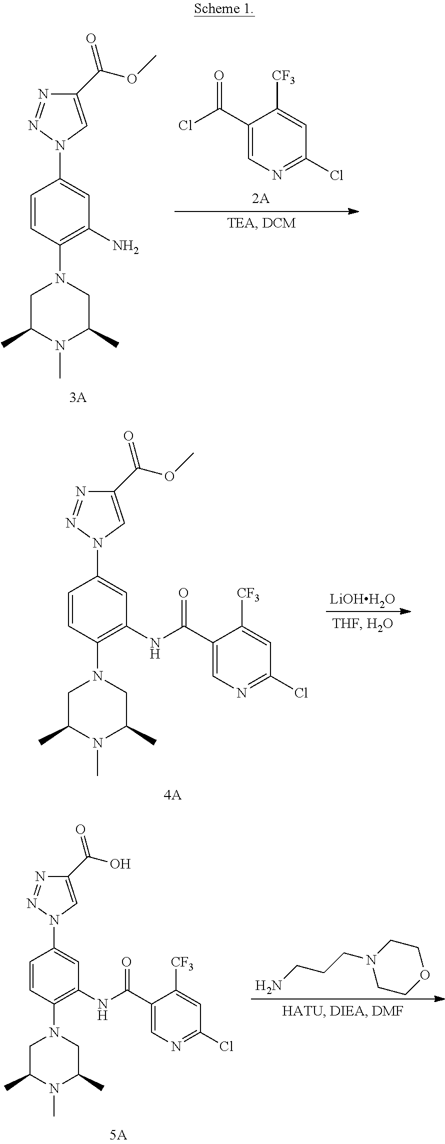

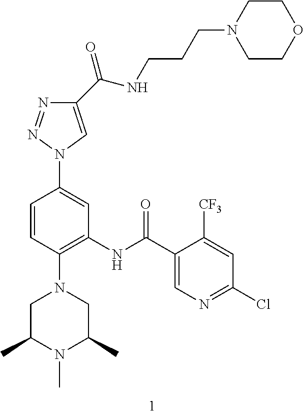 [SMILES: C[C@@H]1CN(c2ccc(-n3cc(C(=O)NCCCN4CCOCC4)nn3)cc2NC(=O)c2cnc(Cl)cc2C(F)(F)F)C[C@H](C)N1C]

Step 1: To a solution of intermediate methyl 1-(3-amino-4-((3S,5R)-3,4,5-trimethylpiperazin-1-yl)phenyl)-1H-1,2,3-triazole-4-carboxylate, intermediate 3A (135 mg, 551.67 μmol, 0.95 eq.) and intermediate compound 2A (200 mg, 580.70 μmol, 1 eq.) in DCM (10 mL) was added drop-wise TEA (294 mg, 2.90 mmol, 404.13 μL, 5 eq.) at −20° C. The reaction mixture was allowed to warm to 20° C. and stirred for 2 hr. The reaction was concentrated under reduced pressure to give a residue. The residue was purified by flash silica gel chromatography (ISCO®; 12 g SepaFlash® Silica Flash Column, Eluent of 0-17% MeOH/DCM at 30 m/min). The product 1-(3-(6-chloro-4-(trifluoromethyl)nicotinamido)-4-((3S,5R)-3,4,5-trimethylpiperazin-1-yl)phenyl)-1H-1,2,3-triazole-4-carboxylate, intermediate 4A (160 mg, 235.99 μmol, 40.64% yield) was obtained as a light yellow solid.

¹H NMR: (DMSO-d₆, 400 MHz) δ_{H}=10.09 (s, 1H), 9.47 (s, 1H), 8.87 (s, 1H), 8.60-8.52 (m, 1H), 8.16 (s, 1H), 7.80-7.74 (m, 1H), 7.40-7.32 (m, 1H), 3.90 (s, 3H), 3.33-3.29 (m, 1H), 3.10-3.00 (m, 2H), 2.58-2.54 (m, 1H), 2.45-2.35 (m, 2H), 2.25-2.15 (m, 3H), 1.04 (d, J=6.0 Hz, 6H).

Step 2: To a solution of 1-(3-(6-chloro-4-(trifluoromethyl)nicotinamido)-4-((3S,5R)-3,4,5-trimethylpiperazin-1-yl)phenyl)-1H-1,2,3-triazole-4-carboxylate, 4A (160 mg, 289.88 μmol, 1 eq.) in THF (2 mL) and H₂O (0.5 mL) was added LiOH·H₂O (24 mg, 579.76 μmol, 2 eq.). The mixture was stirred at 25° C. for 2 hrs. The reaction mixture was adjusted to pH=5 by 1N aq. HCl and concentrated under reduced pressure to give a residue. The product was used directly to the next step without further purification. 1-(3-(6-Chloro-4-(trifluoromethyl)nicotinamido)-4-((3S,5R)-3,4,5-trimethylpiperazin-1-yl)phenyl)-1H-1,2,3-triazole-4-carboxylic acid, 5A (150 mg, 249.54 μmol, 86.08% yield) was obtained as a light yellow solid.

¹H NMR: (DMSO-d₆, 400 MHz) δ_{H}=11.25 (d, J=3.6 Hz, 1H), 10.34 (s, 1H), 9.39 (s, 1H), 8.93 (s, 1H), 8.66 (d, J=2.6 Hz, 1H), 8.17 (s, 1H), 7.84-7.78 (m, 1H), 7.42 (d, J=8.8 Hz, 1H), 3.37-3.29 (m, 2H), 2.77 (d, J=4.8 Hz, 3H), 2.65-2.59 (m, 1H), 1.43-1.37 (m, 6H), 1.07 (s, 3H).

Step 3: To a solution of 1-(3-(6-chloro-4-(trifluoromethyl)nicotinamido)-4-((3S,5R)-3,4,5-trimethylpiperazin-1-yl)phenyl)-1H-1,2,3-triazole-4-carboxylic acid 5A (150 mg, 278.85 μmol, 1 eq.) and 3-morpholinopropan-1-amine (61 mg, 418.28 μmol, 61.12 μL, 1.5 eq.) in DMF (3 mL) was added HATU (212 mg, 557.70 μmol, 2 eq.) and DIEA (108 mg, 836.55 μmol, 145.71 μL, 3 eq.). The mixture was stirred at 25° C. for 12 hr. The mixture was concentrated under reduced pressure to give a residue. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 22-57% B in 14 min.). Title compound Example 1 (78.3 mg, 117.84 μmol, 42.26% yield) was obtained as a white solid. 38 mg of the product was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 0-80% B in 11 min.) to give the pure product Example 1 (14 mg, 21.87 μmol, 3.21% yield, 100% purity) as a white solid.

¹H NMR: (DMSO-d₆, 400 MHz) δ_{H}=9.95 (s, 1H), 9.23-9.10 (m, 1H), 8.92-8.73 (m, 2H), 8.59-8.46 (m, 1H), 8.30-8.09 (m, 1H), 7.74 (dd, J=2.4, 8.8 Hz, 1H), 7.33 (d, J=9.2 Hz, 1H), 3.60 (d, J=4.0 Hz, 4H), 3.36 (s, 3H), 3.03 (d, J=10.8 Hz, 2H), 2.54 (s, 4H), 2.37 (d, J=6.0 Hz, 8H), 2.18 (s, 3H), 1.70 (t, J=6.8 Hz, 2H), 1.06-0.92 (m, 6H).

HPLC: Rₜ=3.568 min in 8 min chromatography, XBridge Shield RP18, 5 μm, 2.1*50 mm, purity 99.19%. LCMS: Rₜ=2.729 min in 4 min chromatography, XBridge Shield RP18, 5 μm, 2.1*50 mm, purity 99.04%, MS ESI calcd. for 663.27 [M+H]⁺ 664.27, found 664.5.

### Example 2. Synthesis of N-(5-(4-((3-morpholinopropyl)carbamoyl)-1H-1,2,3-triazol-1-yl)-2-((3S,5R)-3,4,5-trimethylpiperazin-1-yl)phenyl)-6-oxo-4-(trifluoromethyl)-1,6-dihydropyridine-3-carboxamide (HYBI-065)

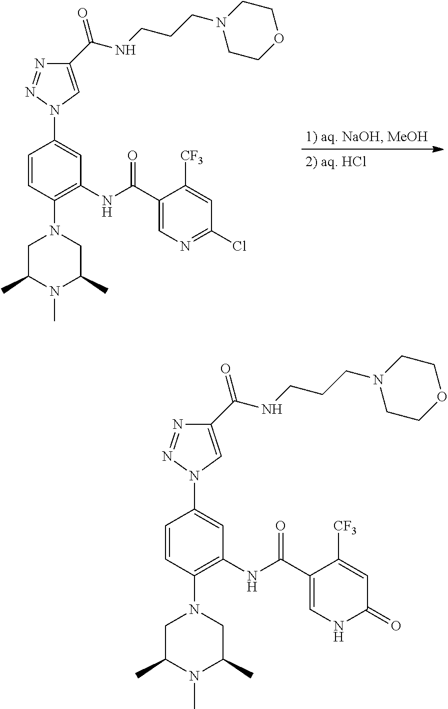 [SMILES: C[C@@H]1CN(c2ccc(-n3cc(C(=O)NCCCN4CCOCC4)nn3)cc2NC(=O)c2cnc(Cl)cc2C(F)(F)F)C[C@H](C)N1C ; C[C@@H]1CN(c2ccc(-n3cc(C(=O)NCCCN4CCOCC4)nn3)cc2NC(=O)c2c[nH]c(=O)cc2C(F)(F)F)C[C@H](C)N1C]

To a solution of compound Example 1 (40 mg, 60.23 μmol, 1 eq.) in MeOH (2 mL) and H₂O (0.5 mL) was added NaOH (2 M, 150.58 μL, 5 eq.). The mixture was stirred at 60° C. for 3 hr. Then HCl (2 M, 752.88 μL, 25 eq.) was added, the mixture was stirred at 100° C. for 2 hrs. The reaction mixture was adjusted to pH=9 by 1N aq. NaHCO₃ and concentrated under reduced pressure to give a residue. The residue was purified by prep-HPLC (Column: Phenomenex Gemini-NX C18 75*30 mm*5 μm; Mobile Phase A: purified water (0.04% NH₃H₂O+10 mM NH₄HCO₃); Mobile Phase B: acetonitrile; Gradient: 0-40% B in 13 min.). Example 2 (18 mg, 27.45 μmol, 45.57% yield, 98.45% purity) was obtained as a white solid.

¹H NMR: (DMSO-d₆, 400 MHz) δ_{H}=9.62 (s, 1H), 9.16 (s, 1H), 8.84 (t, J=5.6 Hz, 1H), 8.40 (d, J=2.8 Hz, 1H), 7.97 (s, 1H), 7.70 (dd, J=2.8, 8.8 Hz, 1H), 7.31 (d, J=8.8 Hz, 1H), 6.82 (s, 1H), 3.60 (s, 4H), 3.33-3.29 (m, 5H), 2.99 (d, J=10.8 Hz, 2H), 2.42-2.31 (m, 8H), 2.20 (s, 3H), 1.70 (quin, J=6.8 Hz, 2H), 1.01 (d, J=6.0 Hz, 6H).

HPLC: Rₜ=1.947 min in 8 min chromatography, XBridge Shield RP18, 5 μm, 2.1*50 mm, purity 98.45%. LCMS: Rₜ=1.483 min in 4 min chromatography, XBridge Shield RP18, 5 μm, 2.1*50 mm, purity 99.35%, MS ESI calcd. for 645.30 [M+H]⁺ 646.30, found 646.4.

### Example 3. Synthesis of 6-chloro-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide (HYBI-063A)

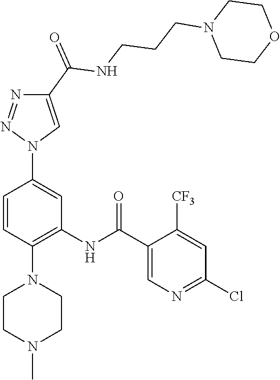 [SMILES: CN1CCN(c2ccc(-n3cc(C(=O)NCCCN4CCOCC4)nn3)cc2NC(=O)c2cnc(Cl)cc2C(F)(F)F)CC1]

Step 1: To a solution of intermediate 2A (154 mg, 632.20 μmol, 1 eq.) and methyl 1-(3-amino-4-(4-methylpiperazin-1-yl)phenyl)-1H-1,2,3-triazole-4-carboxylate (200 mg, 632.20 μmol, 1 eq.) in DCM (5 mL) was added Et₃N (320 mg, 3.16 mmol, 439.97 μL, 5 eq.) at −20° C. The reaction mixture was allowed to warm to 20° C. and stirred at 20° C. for 12 hr to give a brown mixture. Water (10 mL) was added to the reaction mixture. The resulting mixture was extracted with DCM (10 mL×3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 0-15% MeOH/DCM ether gradient at 25 mL/min). The product methyl 1-(3-(6-chloro-4-(trifluoromethyl)nicotinamido)-4-(4-methylpiperazin-1-yl)phenyl)-1H-1,2,3-triazole-4-carboxylate (180 mg, 249.65 μmol, 39.49% yield) was obtained as brown oil.

¹H NMR: (DMSO-d₆, 400 MHz) δ_{H}=10.08 (s, 1H), 9.47 (s, 1H), 8.88 (s, 1H), 8.52 (d, J=2.8 Hz, 1H), 8.16 (s, 1H), 7.77 (dd, J=2.8, 8.8 Hz, 1H), 7.39 (d, J=8.8 Hz, 1H), 3.89 (s, 3H), 3.37-3.34 (m, 4H), 2.99-2.92 (m, 4H), 2.22 (s, 3H).

Step 2: To a solution of 1-(3-(6-chloro-4-(trifluoromethyl)nicotinamido)-4-(4-methylpiperazin-1-yl)phenyl)-1H-1,2,3-triazole-4-carboxylate (170 mg, 324.49 μmol, 1 eq.) in THF (2 mL) and H₂O (0.5 mL) was added LiOH·H₂O (27 mg, 648.99 μmol, 2 eq.). The mixture was stirred at 25° C. for 2 hr. The reaction mixture was adjusted to pH=5 by 1N aq. HCl and concentrated under reduced pressure to give a residue. The product was used directly to the next step without further purification. 1-(3-(6-Chloro-4-(trifluoromethyl)nicotinamido)-4-(4-methylpiperazin-1-yl)phenyl)-1H-1,2,3-triazole-4-carboxylic acid (165 mg, 323.61 μmol, 99.73% yield) was obtained as a yellow solid.

LCMS: Rₜ=0.711 min in 1.5 min chromatography, Chromolith Flash RP-18, 5 μm, 3.0*25 mm, purity 86.19%, MS ESI calcd. for 509.12 [M+H]⁺ 510.12, found 510.0.

Step 3: To a solution of 1-(3-(6-chloro-4-(trifluoromethyl)nicotinamido)-4-(4-methylpiperazin-1-yl)phenyl)-1H-1,2,3-triazole-4-carboxylic acid (160 mg, 313.81 μmol, 1 eq.) and 3-morpholinopropan-1-amine (68 mg, 470.71 μmol, 68.78 μL, 1.5 eq) in DMF (3 mL) was added HATU (239 mg, 627.61 μmol, 2 eq.) and DIEA (122 mg, 941.42 μmol, 163.97 μL, 3 eq.), the mixture was stirred at 25° C. for 12 hr. The mixture was concentrated under reduced pressure to give a residue. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 17-57% B in 11 min.). Example 3 (70 mg, 108.39 μmol, 34.54% yield, 98.49% purity) was obtained as a white solid.

¹H NMR: (DMSO-d₆, 400 MHz) δ_{H}=10.12-9.97 (m, 1H), 9.20 (s, 1H), 8.88 (s, 1H), 8.86-8.80 (m, 1H), 8.52-8.50 (m, 1H), 8.15 (s, 1H), 7.75 (dd, J=2.8, 8.8 Hz, 1H), 7.37 (d, J=8.8 Hz, 1H), 3.64-3.57 (m, 5H), 3.38-3.34 (m, 2H), 2.99-2.87 (m, 5H), 2.40-2.31 (m, 7H), 2.22 (s, 3H), 1.76-1.65 (m, 2H).

### Example 4. Synthesis of 1-(3-(5-amino-2-chloro-4-fluoro-3-methylbenzamido)-4-((3S,5R)-3,4,5-trimethylpiperazin-1-yl)phenyl)-N-(3-morpholinopropyl)-1H-1,2,3-triazole-4-carboxamide (HYBI-063)

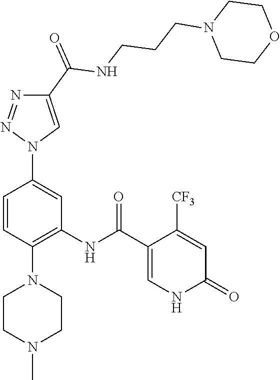 [SMILES: CN1CCN(c2ccc(-n3cc(C(=O)NCCCN4CCOCC4)nn3)cc2NC(=O)c2c[nH]c(=O)cc2C(F)(F)F)CC1]

To a solution of compound Example 3 (30 mg, 47.16 μmol, 1 eq.) in MeOH (2 mL) and H₂O (0.5 mL) was added NaOH (2 M, 117.91 μL, 5 eq.). The mixture was stirred at 60° C. for 2 hr, then HCl (2 M, 589.56 μL, 25 eq.) was added, the mixture was stirred at 85° C. for 3 hr. The reaction mixture was adjusted to pH=9 by 1N aq·NaHCO₃ and concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 6-36% B in 12 min.). Example 4 (17 mg, 27.21 μmol, 57.69% yield, 98.86% purity) was obtained as a white solid.

¹H NMR: (DMSO-d₆, 400 MHz) δ_{H}=9.59 (s, 1H), 9.17 (s, 1H), 8.83 (s, 1H), 8.44 (s, 1H), 7.99 (s, 1H), 7.71 (d, J=7.6 Hz, 1H), 7.36 (d, J=8.8 Hz, 1H), 6.82 (s, 1H), 3.60 (s, 4H), 3.42-3.36 (m, 3H), 2.92 (s, 4H), 2.48-2.44 (m, 4H), 2.36 (s, 6H), 2.23 (s, 3H), 1.71 (d, J=6.8 Hz, 2H).

HPLC: Rt=1.932 min in 8 min chromatography, XBridge Shield RP18, 5 μm, 2.1*50 mm, purity 98.86%. LCMS: Rₜ=0.971 min in 4 min chromatography, XBridge Shield RP18, 5 μm, 2.1*50 mm, purity 96.89%, MS ESI calcd. for 617.27 [M+H]⁺ 618.27, found 618.4.

### Example 5. Synthesis of 6-chloro-N-(4-fluoro-5-(4-((3-morpholinopropyl)carbamoyl)-1H-1,2,3-triazol-1-yl)-2-((3S,5R)-3,4,5-trimethylpiperazin-1-yl)phenyl)-4-(trifluoromethyl)nicotinamide (HYBI-070A)

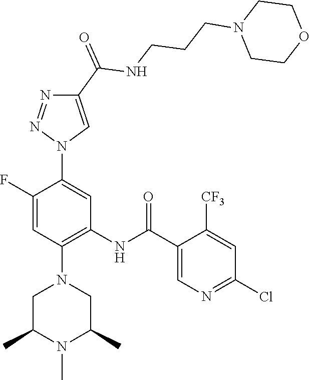 [SMILES: C[C@@H]1CN(c2cc(F)c(-n3cc(C(=O)NCCCN4CCOCC4)nn3)cc2NC(=O)c2cnc(Cl)cc2C(F)(F)F)C[C@H](C)N1C]

Step 1: To a solution of intermediate 2A (135 mg, 551.87 μmol, 1 eq.) and methyl 1-(5-amino-2-fluoro-4-((3S,5R)-3,4,5-trimethylpiperazin-1-yl)phenyl)-1H-1,2,3-triazole-4-carboxylate (200 mg, 551.87 μmol, 1 eq.) in DCM (5 mL) was added Et₃N (279 mg, 2.76 mmol, 384.07 μL, 5 eq.) at −20° C. The reaction mixture was stirred at 20° C. for 12 hrs to give a brown mixture. Water (10 mL) was added to the reaction mixture. The resulting mixture was extracted with DCM (10 mL×3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 0-15% MeOH/DCM ether gradient at 25 mL/min). The product 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylate (300 mg, 247.55 μmol, 44.86% yield) was obtained as brown oil.

LCMS: Rₜ=1.170 min in 2 min chromatography, Xtimate C18, 3 μm, 2.1*30 mm, purity 47.03%, MS ESI calcd. for 569.16 [M+H]⁺ 570.16, found 570.3.

Step 2: To a solution of 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylate (300 mg, 526.37 μmol, 1 eq.) in THF (8 mL) and H₂O (1 mL) was added LiOH·H₂O (44 mg, 1.05 mmol, 2 eq.). The mixture was stirred at 25° C. for 2 hr. The reaction mixture was adjusted to pH=5 by 1N aq. HCl and concentrated under reduced pressure to give a residue. The product was used directly to the next step without further purification. The product 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylic acid (310 mg, crude) was obtained as a yellow solid.

¹H NMR: (DMSO-d₆, 400 MHz) δ_{H}=11.14 (s, 1H), 10.42 (s, 1H), 9.15 (d, J=1.6 Hz, 1H), 8.92 (d, J=10.4 Hz, 1H), 8.35 (d, J=8.0 Hz, 1H), 8.16 (s, 1H), 8.08 (s, 1H), 2.89 (s, 2H), 2.77 (s, 2H), 2.73 (s, 2H), 1.39 (d, J=6.4 Hz, 6H), 1.28-1.22 (m, 3H).

Step 3: To a solution of 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylic acid (310 mg, 557.64 μmol, 1 eq.) and 3-morpholinopropan-1-amine (120 mg, 836.46 μmol, 122.22 μL, 1.5 eq.) in DMF (4 mL) was added HATU (424 mg, 1.12 mmol, 2 eq.) and DIEA (216 mg, 1.67 mmol, 291.39 μL, 3 eq.). The mixture was stirred at 25° C. for 3 hr. The reaction mixture was concentrated directly. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 20-60% B in 11 min.). Compound Example 5 (64 mg, 90.51 μmol, 16.23% yield, 96.47% purity) was obtained as a white solid. Take 25 mg to purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 0-60% B in 11 min.). Pure Example 5 (10.6 mg) was obtained as a white solid.

¹H NMR: (DMSO-d₆, 400 MHz) δ_{H}=10.17 (s, 1H), 8.96 (d, J=1.6 Hz, 1H), 8.85 (s, 2H), 8.25 (d, J=8 Hz, 1H), 8.14 (s, 1H), 7.32 (d, J=12.4 Hz, 1H), 3.60 (t, J=4.4 Hz, 4H), 3.39-3.35 (m, 2H), 3.11 (d, J=11.2 Hz, 2H), 2.57-2.51 (m, 2H), 2.40-2.31 (m, 8H), 2.19 (s, 3H), 1.75-1.65 (m, 2H), 1.03 (d, J=6.4 Hz, 6H).

HPLC: Rₜ=2.634 min in 8 min chromatography, XBridge Shield RP18, 5 μm, 2.1*50 mm, purity 96.4%. LCMS: Rₜ=2.112 min in 4 min chromatography, XBridge Shield RP18, 5 μm, 2.1*50 mm, purity 95.69%, MS ESI calcd. for 681.26 [M+H]⁺ 682.26, found 682.3.

### Example 6. Synthesis of N-(4-fluoro-5-(4-((3-morpholinopropyl)carbamoyl)-1H-1,2,3-triazol-1-yl)-2-((3S,5R)-3,4,5-trimethylpiperazin-1-yl)phenyl)-6-oxo-4-(trifluoromethyl)-1,6-dihydropyridine-3-carboxamide (HYBI-070)

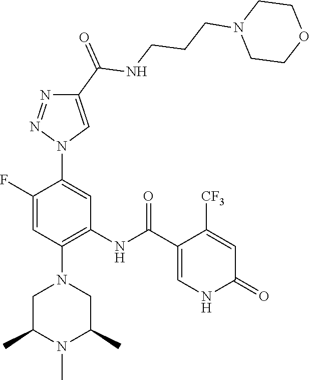 [SMILES: C[C@@H]1CN(c2cc(F)c(-n3cc(C(=O)NCCCN4CCOCC4)nn3)cc2NC(=O)c2c[nH]c(=O)cc2C(F)(F)F)C[C@H](C)N1C]

To a solution of Example 5 (38 mg, 55.71 μmol, 1 eq.) in MeOH (3 mL) and H₂O (1 mL) was added NaOH (2 M, 139.27 μL, 5 eq.). The mixture was stirred at 60° C. for 3 hr. Then HCl (2 M, 696.37 μL, 25 eq.) was added, the mixture was stirred at 100° C. for 2 hr. The reaction mixture was adjusted to pH=9 by 1N aq. NaHCO₃ and concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 0-43% B in 14 min.). Example 6 (15.8 mg, 23.31 μmol, 41.84% yield, 97.9% purity) was obtained as a white solid.

¹H NMR: (DMSO-d₆, 400 MHz) δ_{H}=9.70 (s, 1H), 8.94 (d, J=1.6 Hz, 1H), 8.83 (t, J=5.6 Hz, 1H), 8.09 (d, J=8.0 Hz, 1H), 7.94 (s, 1H), 7.27 (d, J=12.4 Hz, 1H), 6.81 (s, 1H), 3.60 (t, J=4.4 Hz, 4H), 3.39-3.36 (m, 2H), 3.09 (d, J=10.8 Hz, 2H), 2.54 (s, 2H), 2.42-2.30 (m, 9H), 2.20 (s, 3H), 1.75-1.65 (m, 2H), 1.01 (d, J=6.0 Hz, 6H).

HPLC: Rₜ=1.313 min in 8 min chromatography, XBridge Shield RP18, 5 μm, 2.1*50 mm, purity 97.91%. LCMS: Rₜ=1.075 min in 4 min chromatography, XBridge Shield RP18, 5 μm, 2.1*50 mm, purity 99.63%, MS ESI calcd. for 663.29 [M+H]⁺ 664.29, found 664.3.

### Example 7. Synthesis of 6-chloro-N-(4-fluoro-2-(4-methylpiperazin-1-yl)-5-(4-((3-morpholinopropyl)carbamoyl)-1H-1,2,3-triazol-1-yl)phenyl)-4-(trifluoromethyl)nicotinamide (HYBI-067A)

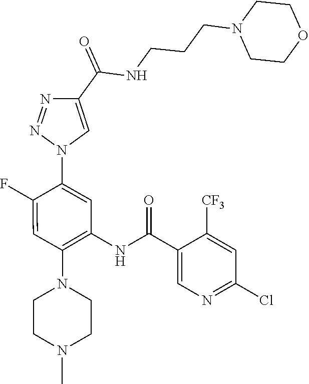 [SMILES: CN1CCN(c2cc(F)c(-n3cc(C(=O)NCCCN4CCOCC4)nn3)cc2NC(=O)c2cnc(Cl)cc2C(F)(F)F)CC1]

Step 1: To a solution of intermediate 2A (219 mg, 897.27 μmol, 1 eq.) and methyl 1-(5-amino-2-fluoro-4-(4-methylpiperazin-1-yl)phenyl)-1H-1,2,3-triazole-4-carboxylate (300 mg, 897.27 μmol, 1 eq.) in DCM (10 mL) was added drop-wise TEA (453.97 mg, 4.49 mmol, 624.45 μL, 5 . . . ) at −20° C. The reaction mixture was allowed to warm to 20° C. and stirred for 2 hrs. The reaction mixture was diluted with DCM (50 mL×2), washed with brine (20 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-17% MeOH/DCM at 30 mL/min). The product methyl 1-(5-(6-chloro-4-(trifluoromethyl)nicotinamido)-2-fluoro-4-(4-methylpiperazin-1-yl)phenyl)-1H-1,2,3-triazole-4-carboxylate (370 mg, 477.96 μmol, 53.27% yield) was obtained as a brown oil.

¹H NMR: (DMSO-d₆, 400 MHz) δ_{H}=10.14 (s, 1H), 9.22 (d, J=1.6 Hz, 1H), 8.87 (s, 1H), 8.13 (s, 1H), 7.95 (s, 1H), 7.37 (d, J=12.0 Hz, 1H), 3.90 (s, 3H), 3.17 (d, J=5.2 Hz, 2H), 3.05-2.99 (m, 2H), 2.89 (s, 4H), 2.73 (s, 3H).

Step 2: To a solution of methyl 1-(5-(6-chloro-4-(trifluoromethyl)nicotinamido)-2-fluoro-4-(4-methylpiperazin-1-yl)phenyl)-1H-1,2,3-triazole-4-carboxylate (370 mg, 682.80 μmol, 1 eq.) in THF (4 mL) and H₂O (2 mL) was added LiOH·H₂O (57 mg, 1.37 mmol, 2 eq.). The mixture was stirred at 25° C. for 2 hr. The reaction mixture was adjusted to pH=5 by 1N aq. HCl and concentrated under reduced pressure to give a residue. The product was used directly to the next step without further purification. 1-(5-(6-Chloro-4-(trifluoromethyl)nicotinamido)-2-fluoro-4-(4-methylpiperazin-1-yl)phenyl)-1H-1,2,3-triazole-4-carboxylic acid (360 mg, crude) was obtained as a yellow solid.

¹H NMR: (DMSO-d₆, 400 MHz) δ_{H}=11.34 (s, 1H), 10.37 (s, 1H), 9.15-9.12 (m, 1H), 8.95 (s, 1H), 8.35-8.29 (m, 1H), 8.17 (s, 1H), 7.95 (s, 1H), 3.60 (s, 4H), 2.89 (s, 2H), 2.73 (s, 2H), 1.76 (t, J=3.2 Hz, 3H).

Step 3: To a solution of 1-(5-(6-chloro-4-(trifluoromethyl)nicotinamido)-2-fluoro-4-(4-methylpiperazin-1-yl)phenyl)-1H-1,2,3-triazole-4-carboxylic acid (360 mg, 682.00 μmol, 1 eq.) and 3-morpholinopropan-1-amine (148 mg, 1.02 mmol, 149.47 μL, 1.5 eq.) in DMF (4 mL) was added HATU (519 mg, 1.36 mmol, 2 eq.) and DIEA (265 mg, 2.05 mmol, 356.38 μL, 3 eq.). The mixture was stirred at 25° C. for 12 hr. The reaction mixture was concentrated directly. The residue was purified by flash silica gel chromatography (ISCO®; 12 g SepaFlash® Silica Flash Column, Eluent of 0-10% MeOH/DCM at 20 mL/min). The crude product Example 7 (390 mg, 477.02 μmol, 69.94% yield, 80% purity) was obtained as a yellow oil. Example 7 (190 mg) of the product was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 0-30% B in 8 min.) to give the pure Example 7 (14 mg, 21.87 μmol, 3.21% yield, 100% purity) as a white solid.

¹H NMR: (DMSO-d₆, 400 MHz) δ_{H}=10.12 (s, 1H), 8.96 (s, 1H), 8.90-8.80 (m, 2H), 8.22 (d, J=8.0 Hz, 1H), 8.14 (s, 1H), 7.36 (d, J=12.0 Hz, 1H), 3.60 (s, 4H), 3.39-3.35 (m, 2H), 3.00 (s, 4H), 2.49-2.44 (m, 4H), 2.36 (s, 6H), 2.22 (s, 3H), 1.77-1.64 (m, 2H).

HPLC: Rₜ=3.269 min in 8 min chromatography, XBridge Shield RP18, 5 μm, 2.1*50 mm, purity 100%. LCMS: Rₜ=2.504 min in 4 min chromatography, Xtimate C18, 3 μm, 2.1*30 mm, purity 99.73%, MS ESI calcd. for 653.23 [M+H]⁺ 654.23, found 654.4.

### Example 8. Synthesis of N-(4-fluoro-2-(4-methylpiperazin-1-yl)-5-(4-((3-morpholinopropyl)carbamoyl)-1H-1,2,3-triazol-1-yl)phenyl)-6-oxo-4-(trifluoromethyl)-1,6-dihydropyridine-3-carboxamide (HYBI-064A)

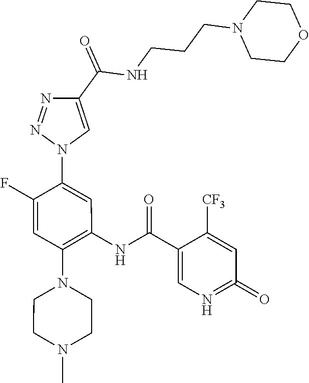

To a solution of Example 7 (200 mg, 305.78 μmol, 1 eq.) in MeOH (4 mL) and H₂O (2 mL) was added NaOH (2 M, 764.46 μL, 5 eq.). The mixture was stirred at 60° C. for 2 hr. Then HCl (2 M, 3.82 mL, 25 eq.) was added, the mixture was stirred at 100° C. for 2 hr. The reaction mixture was adjusted to pH=9 by 1N aq·NaHCO₃ concentrated under reduced pressure to give a residue. The residue was purified by prep-HPLC(Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 0-40% B in 13 min.). Example 8 (19.7 mg, 29.84 μmol, 9.76% yield, 96.29% purity) was obtained as a white solid.

¹H NMR: (DMSO-d₆, 400 MHz) δ_{H}=9.71 (s, 1H), 8.94 (d, J=1.6 Hz, 1H), 8.89-8.81 (m, 1H), 8.10 (d, J=8.0 Hz, 1H), 7.96 (s, 1H), 7.32 (d, J=12.4 Hz, 1H), 6.81 (s, 1H), 3.60 (t, J=4.8 Hz, 4H), 3.33-3.29 (m, 5H), 2.97 (s, 4H), 2.49-2.46 (m, 2H), 2.36 (d, J=6.8 Hz, 6H), 2.23 (s, 3H), 1.70 (t, J=6.8 Hz, 2H).

HPLC: Rₜ=1.907 min in 8 min chromatography, XBridge Shield RP18, 5 μm, 2.1*50 mm, purity 96.29%. LCMS: Rₜ=1.330 min in 4 min chromatography, XBridge Shield RP18, 5 μm, 2.1*50 mm, purity 95.46%, MS ESI calcd. for 635.26 [M+H]⁺ 636.26, found 636.5.

### Example 9. Synthesis of (S)-6-chloro-N-(2-(3,4-dimethylpiperazin-1-yl)-5-(4-((3-morpholinopropyl)carbamoyl)-1H-1,2,3-triazol-1-yl)phenyl)-4-(trifluoromethyl)nicotinamide (HYBI-064A)

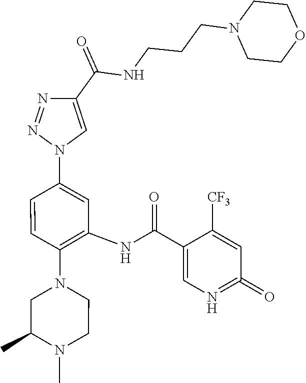

Step 1: To a solution of intermediate 2A (162 mg, 665.89 μmol, 1.1 eq.) and methyl (S)-1-(5-amino-4-(3,4-dimethylpiperazin-1-yl)-2-fluorophenyl)-1H-1,2,3-triazole-4-carboxylate (200 mg, 605.36 μmol, 1 eq.) in DCM (5 mL) was added Et₃N (306 mg, 3.03 mmol, 421.29 μL, 5 eq.) at −20° C. The reaction mixture was stirred at 20° C. for 12 hrs to give a brown mixture. Water (10 mL) was added to the reaction mixture. The resulting mixture was extracted with DCM (10 mL×3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 0-15% MeOH/DCM ether gradient at 25 mL/min). The product 1-(3-(6-chloro-4-(trifluoromethyl)nicotinamido)-4-(3,4-dimethylpiperazin-1-yl)phenyl)-1H-1,2,3-triazole-4-carboxylate (230 mg, 338.21 μmol, 55.87% yield) was obtained as brown oil.

¹H NMR: (DMSO-d₆, 400 MHz) δ_{H}=10.08 (s, 1H), 9.48 (s, 1H), 8.87 (s, 1H), 8.54 (d, J=2.8 Hz, 1H), 8.16 (s, 1H), 7.77 (dd, J=2.8, 8.8 Hz, 1H), 7.37 (d, J=8.8 Hz, 1H), 3.93-3.83 (m, 3H), 3.32 (s, 2H), 3.12-2.98 (m, 2H), 2.88-2.75 (m, 2H), 2.35-2.15 (m, 4H), 1.00 (d, J=6.0 Hz, 3H).

Step 2: To a solution of 1-(3-(6-chloro-4-(trifluoromethyl)nicotinamido)-4-(3,4-dimethylpiperazin-1-yl)phenyl)-1H-1,2,3-triazole-4-carboxylate (180 mg, 334.62 μmol, 1 eq.) in THF (2 mL) and H₂O (0.5 mL) was added LiOH·H2O (28 mg, 669.24 μmol, 2 eq.). The mixture was stirred at 25° C. for 2 hr. The reaction mixture was adjusted to pH=5 by 1N aq. HCl and concentrated under reduced pressure to give a residue. The product was used directly to the next step without further purification. (S)-1-(3-(6-Chloro-4-(trifluoromethyl)nicotinamido)-4-(3,4-dimethylpiperazin-1-yl)phenyl)-1H-1,2,3-triazole-4-carboxylic acid (175 mg, crude) was obtained as a yellow solid.

LCMS: Rₜ=1.088 min in 2 min chromatography, Xtimate C18, 3 μm, 2.1*30 mm, purity 86.19%, MS ESI calcd. for 523.13 [M+H]⁺ 524.13, found 524.2.

Step 3: To a solution of (S)-1-(3-(6-chloro-4-(trifluoromethyl)nicotinamido)-4-(3,4-dimethylpiperazin-1-yl)phenyl)-1H-1,2,3-triazole-4-carboxylic acid (170 mg, 324.49 μmol, 1 eq.) and 3-morpholinopropan-1-amine (70 mg, 486.74 μmol, 71.12 μL, 1.5 eq.) in DMF (2 mL) was added HATU (246.76 mg, 648.99 μmol, 2 eq.) and DIEA (125.82 mg, 973.48 μmol, 169.56 μL, 3 eq.) at 25° C. for 12 hr. The mixture was concentrated under reduced pressure to give a residue. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 20-60% B in 11 min.). Example 9 (72 mg, 104.75 μmol, 32.28% yield) was obtained as a white solid.

¹H NMR: (DMSO-d₆, 400 MHz) δ_{H}=10.13-9.97 (m, 1H), 9.24-9.15 (m, 1H), 8.89 (s, 1H), 8.87-8.83 (m, 1H), 8.53 (d, J=2.4 Hz, 1H), 8.16 (s, 1H), 7.79-7.73 (m, 1H), 7.37 (d, J=8.8 Hz, 1H), 3.61 (d, J=4.4 Hz, 5H), 3.10-2.98 (m, 2H), 3.10-2.97 (m, 3H), 2.37 (s, 8H), 2.21 (s, 3H), 1.74-1.66 (m, 2H), 1.00 (d, J=6.0 Hz, 3H).

### Example 10. Synthesis of (S)-N-(2-(3,4-dimethylpiperazin-1-yl)-5-(4-((3-morpholinopropyl)carbamoyl)-1H-1,2,3-triazol-1-yl)phenyl)-6-oxo-4-(trifluoromethyl)-1,6-dihydropyridine-3-carboxamide (HYBI-064)

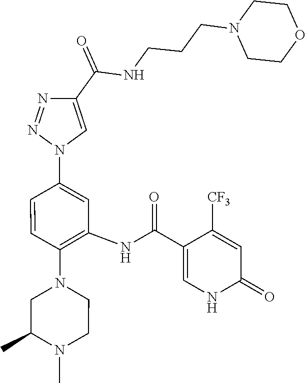 [SMILES: C[C@H]1CN(c2ccc(-n3cc(C(=O)NCCCN4CCOCC4)nn3)cc2NC(=O)c2c[nH]c(=O)cc2C(F)(F)F)CCN1C]

To a solution of compound Example 9 (40 mg, 61.53 μmol, 1 eq.) in MeOH (2 mL) and H₂O (0.5 mL) was added NaOH (2 M, 153.82 μL, 5 eq.). The mixture was stirred at 60° C. for 2 hr. Then HCl (2 M, 769.12 μL, 25 eq.) was added, the mixture was stirred at 85° C. for 12 hrs. The reaction mixture was adjusted to pH=9 by 1N aq·NaHCO₃ and concentrated under reduced pressure to give a residue. The residue was purified by prep-HPLC (Column: Phenomenex Gemini-NX C18 75*30 mm*5 μm; Mobile Phase A: purified water (0.04% NH₃H₂O+10 mM NH₄HCO₃); Mobile Phase B: acetonitrile; Gradient: 0-38% B in 15 min.). Example 10 (21 mg, 32.74 μmol, 53.21% yield, 98.48% purity) was obtained as a white solid.

¹H NMR: (DMSO-d₆, 400 MHz) δ_{H}=9.60 (s, 1H), 9.17 (s, 1H), 8.84 (t, J=5.6 Hz, 1H), 8.42 (d, J=2.8 Hz, 1H), 7.99 (s, 1H), 7.71 (dd, J=2.8, 8.8 Hz, 1H), 7.35 (d, J=8.8 Hz, 1H), 6.83 (s, 1H), 3.61 (t, J=4.8 Hz, 4H), 3.34-3.31 (m, 4H), 3.06-2.95 (m, 2H), 2.91-2.75 (m, 2H), 2.49-2.40 (m, 2H), 2.39-2.33 (m, 6H), 2.22 (s, 3H), 1.71 (quin, J=6.8 Hz, 2H), 0.99 (d, J=6.0 Hz, 3H).

HPLC: Rₜ=1.908 min in 8 min chromatography, XBridge Shield RP18, 5 μm, 2.1*50 mm, purity 98.48%. LCMS: Rₜ=0.951 min in 4 min chromatography, XBridge Shield RP18, 5 μm, 2.1*50 mm, purity 98.82%, MS ESI calcd. for 631.28 [M+H]⁺ 632.28, found 632.5.

### Example 11. Synthesis of 6-fluoro-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide

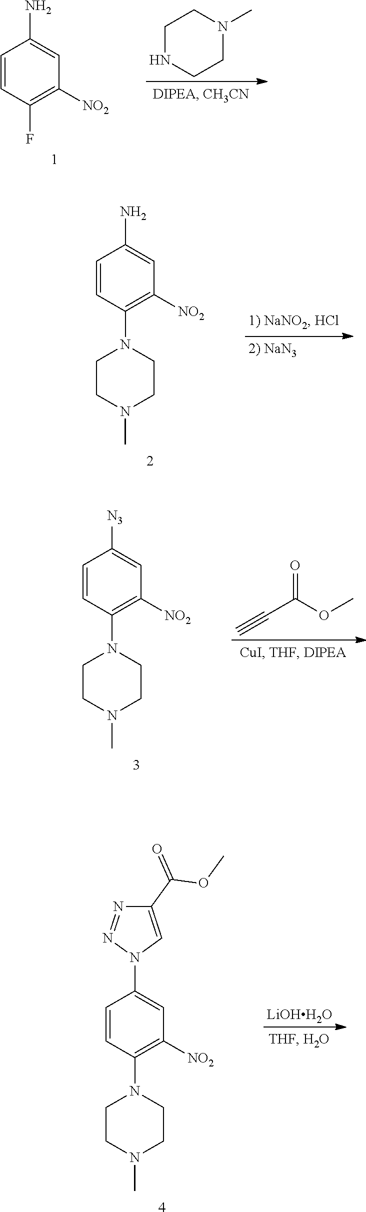

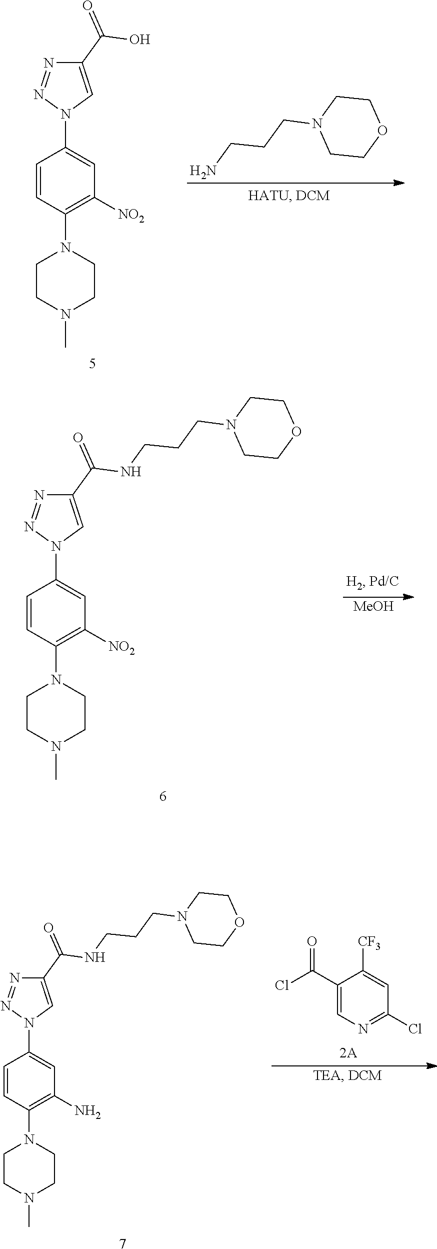

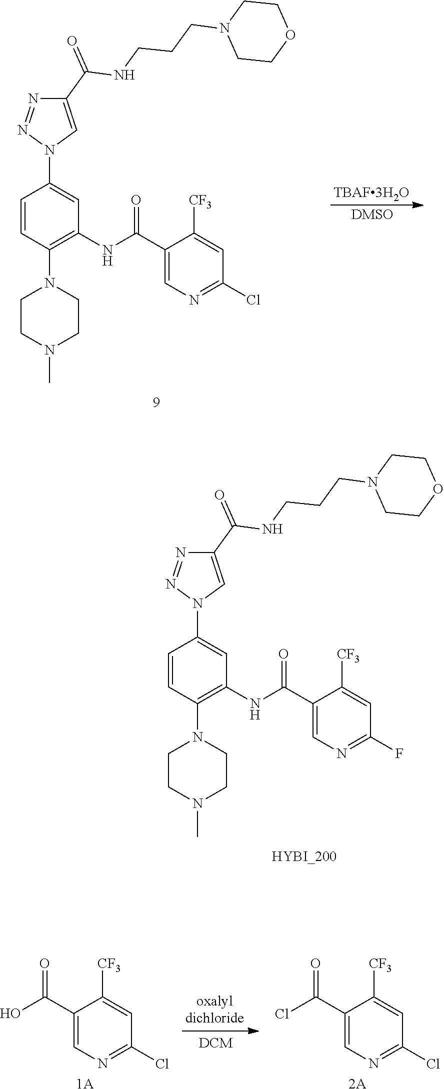

### Step 1: 4-(4-methylpiperazin-1-yl)-3-nitro-aniline (Compound 2)

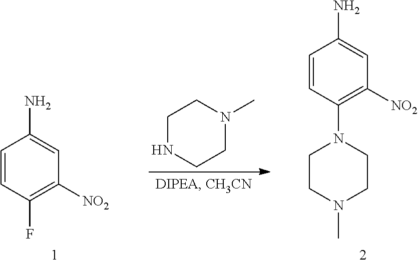

To a mixture of compound 1 (50 g, 320.28 mmol) and 1-methylpiperazine (64.16 g, 640.56 mmol, 71.05 mL) in CH₃CN (500 mL) was added DIEA (82.79 g, 640.56 mmol, 111.57 mL), and the mixture was stirred at 90° C. for 12 h. The mixture was concentrated to give the residue. The residue was diluted with DCM (200 mL), washed with brine (200 mL×3). The combined organic layer was dried over Na₂SO₄ and concentrated to give crude product. The crude product was purified by flash silica gel chromatography (Eluent of 1-10% MeOH/DCM) to give compound 2 (64 g, 270.88 mmol, 84.58% yield) as a brown solid.

¹H NMR (DMSO-d₆, 400 MHz) δ_{H}=7.15 (d, J=8.8 Hz, 1H), 6.85 (d, J=2.4 Hz, 1H), 6.78 (dd, J=8.8 Hz, 2.8 Hz, 1H), 5.43 (brs, 2H), 2.79 (t, J=4.8 Hz, 4H), 2.36 (brs, 4H), 2.18 (s, 3H).

### Step 2:1-(4-azido-2-nitro-phenyl)-4-methyl-piperazine (Compound 3)

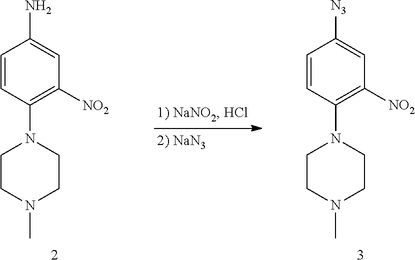

To a mixture of compound 2 (40 g, 169.30 mmol) in HCl (2 M, 1.02 L) was added a solution of NaNO₂ (17.52 g, 253.95 mmol) in H₂O (200 mL) dropwise at 0° C. After stirring for 0.5 h, a solution of NaN₃ (22.17 g, 341.02 mmol) in H₂O (200 mL) was added into the mixture at 0° C. After stirring for 0.5 hr, the mixture was warmed up to 25° C. and stirred for 0.5 hr. The mixture was basified with NaOH (2N) to pH˜9, and the mixture was filtered via a filter paper. The crude compound 3 (44 g, 167.77 mmol, 99.10% yield) was obtained as a red solid, which was used into the next step without further purification.

¹H NMR (DMSO-d₆, 400 MHz) δ_{H}=7.57 (d, J=2.4 Hz, 1H), 7.33-7.40 (m, 2H), 2.91-3.01 (m, 4H), 2.40-2.47 (m, 4H), 2.23 (s, 3H).

### Step 3: methyl 1-[4-(4-methylpiperazin-1-yl)-3-nitro-phenyl]triazole-4-carboxylate (Compound 4)

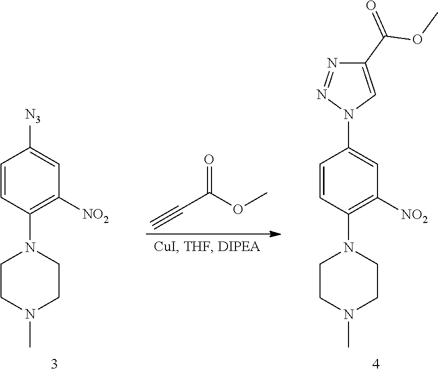

To a mixture of compound 3 (11 g, 41.94 mmol) and methyl prop-2-ynoate (3.53 g, 41.94 mmol, 3.49 mL) in THF (100 mL) was added CuI (798.78 mg, 4.19 mmol) and DIEA (16.26 g, 125.83 mmol, 21.92 mL), and the mixture was stirred at 25° C. for 1 h. The mixture was filtered through a Celite pad, and the filtrate was concentrated to give crude product. The crude product was purified by flash chromatography on silica gel (MeOH in DCM=0% to 8% to 10%) to give compound 4 (9.6 g, 27.72 mmol, 66.09% yield) as a red solid.

¹H NMR (DMSO-d₆, 400 MHz) δ_{H}=9.54 (s, 1H), 8.45 (d, J=2.8 Hz, 1H), 8.15 (dd, J=8.8, 2.8 Hz, 1H), 7.51 (d, J=8.8 Hz, 1H), 3.89 (s, 3H), 3.05-3.16 (m, 4H), 2.42-2.48 (m, 4H), 2.23 (s, 3H).

### Step 4: 1-[4-(4-methylpiperazin-1-yl)-3-nitro-phenyl]triazole-4-carboxylic acid (Compound 5)

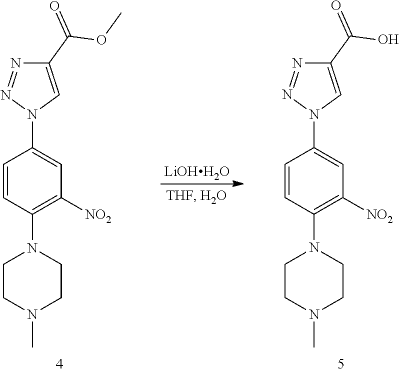

To a mixture of compound 4 (1 g, 2.89 mmol) in THF (10 mL) was added LiOH·H₂O (605.81 mg, 14.44 mmol) in H₂O (5 mL), and the mixture was stirred at 25° C. for 1 h. The mixture was concentrated to remove THF. The pH of the mixture was adjusted to around 4 with 2 N HCl. The mixture was filtered via a filter paper. The filter cake was dried under reduced pressure. The crude compound 5 (750 mg, 2.26 mmol, 78.17% yield) was obtained as a red solid, which was used into the next step without further purification.

¹H NMR (DMSO-d₆, 400 MHz) δ_{H}=9.44 (s, 1H), 8.50 (d, J=2.8 Hz, 1H), 8.20 (dd, J=8.8, 2.8 Hz, 1H), 7.59 (d, J=9.2 Hz, 1H), 3.24-3.34 (m, 4H), 2.99 (br s, 4H), 2.60 (s, 3H).

### Step 5: 1-[4-(4-methylpiperazin-1-yl)-3-nitro-phenyl]-N-(3-morpholinopropyl)triazole-4-carboxamide (Compound 6)

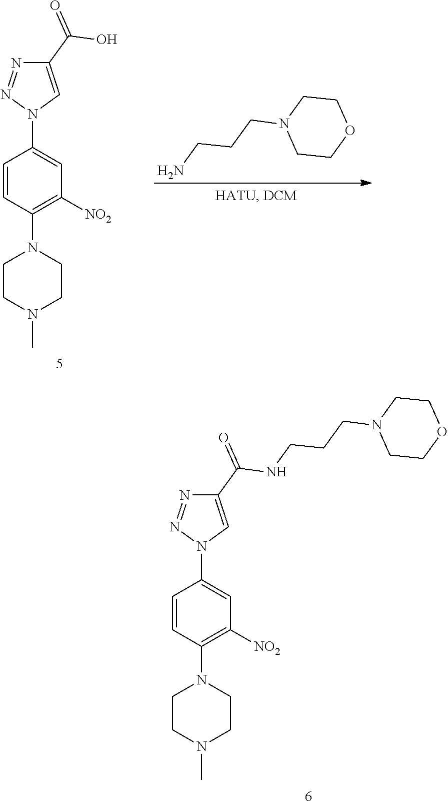

To a mixture of compound 5 (1.7 g, 5.12 mmol) and 3-morpholinopropan-1-amine (737.75 mg, 5.12 mmol, 747.46 uL) in DCM (20 mL) was added DIEA (1.98 g, 15.35 mmol, 2.67 mL) in one portion at 25° C., then HATU (2.33 g, 6.14 mmol) was added in one portion, and the mixture was stirred at 25° C. for 2 h. The mixture was concentrated to remove DCM. The crude product was triturated from MeCN (20 mL). The resulting mixture was filtered, and the filter cake was dissolved in DCM (150 mL). The DCM solvent was concentrated to dryness. The crude product was purified by flash chromatography on silica gel (MeOH in DCM=0% to 8% to 10%) to give compound 6 (630 mg, 1.37 mmol, 26.86% yield) as a yellow solid.

¹H NMR (DMSO-d₆, 400 MHz) δ_{H}=9.29 (s, 1H), 8.84 (t, J=5.6 Hz, 1H), 8.43 (d, J=2.8 Hz, 1H), 8.15 (dd, J=9.2, 2.8 Hz, 1H), 7.50 (d, J=9.2 Hz, 1H), 3.61 (t, J=4.4 Hz, 4H), 3.34 (br d, J=6.0 Hz, 2H), 3.07-3.12 (m, 4H), 2.45-2.48 (m, 4H), 2.34-2.42 (m, 6H), 2.24 (s, 3H), 1.70 (m, 2H).

### Step 6: 1-[3-amino-4-(4-methylpiperazin-1-yl)phenyl]-N-(3-morpholinopropyl)triazole-4-carboxamide (Compound 7)

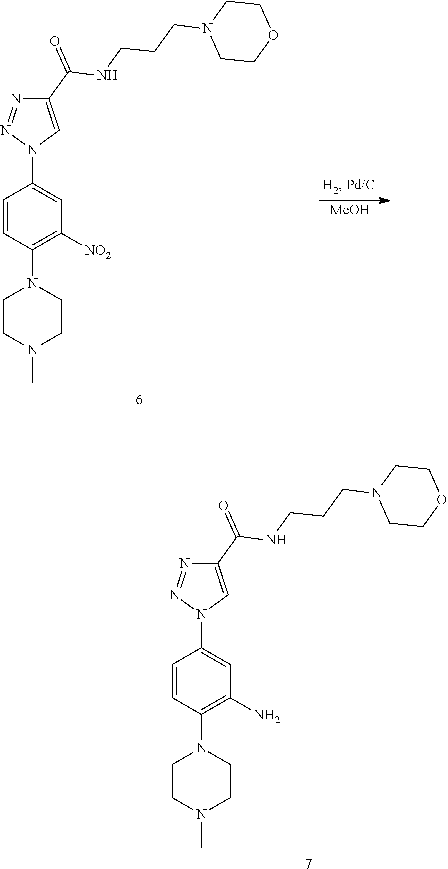 [SMILES: "MeOH" ; CN1CCN(c2ccc(-n3cc(C(=O)NCCCN4CCOCC4)nn3)cc2[N+](=O)[O-])CC1 ; "6" ; CN1CCN(c2ccc(-n3cc(C(=O)NCCCN4CCOCC4)nn3)cc2N)CC1 ; "7"]

To a mixture of compound 6 (630 mg, 1.37 mmol) in MeOH (10 mL) was added Pd/C (1.37 mmol, 10% purity). The reaction mixture was degassed and purged with H₂ for 3 times. The reaction mixture was stirred under H₂ (15 psi) for 12 hr at 30° C. to give a black mixture. The suspension was filtered through a pad of Celine or silica gel and the pad or filter cake was washed with MeOH (30 mL×2). The combined filtrates were concentrated to dryness to give a residue. Compound 7 (560 mg, 1.31 mmol, 95.11% yield) was obtained as a yellow solid, which was used into the next step without further purification.

¹H NMR (DMSO-d₆, 400 MHz) δ_{H}=9.02 (s, 1H), 8.79 (t, J=5.6 Hz, 1H), 7.26 (d, J=1.6 Hz, 1H), 7.00-7.06 (m, 2H), 5.14 (s, 2H), 3.60 (t, J=4.4 Hz, 4H), 3.33 (br d, J=6.0 Hz, 2H), 2.85 (br s, 4H), 2.51-2.59 (m, 4H), 2.31-2.39 (m, 6H), 2.25 (s, 3H), 1.69 (m, 2H).

### Step 7: 6-chloro-4-(trifluoromethyl)pyridine-3-carbonyl chloride (Compound 2A)

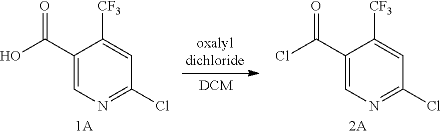

To a solution of compound 1A (500 mg, 2.22 mmol) and DMF (16.20 mg, 0.22 mmol, 0.017 mL) in DCM (5 mL) was added oxalyl dichloride (1.41 g, 11.08 mmol, 0.97 mL) dropwise at 0° C. The reaction mixture was stirred at 25° C. for 20 mins. The reaction mixture was concentrated to give compound 2A (520 mg, crude), which was used in the next step without further purification.

### Step 8: 6-chloro-N-[2-(4-methylpiperazin-1-yl)-5-[4-(3-morpholinopropylcarbamoyl)triazol-1-yl]phenyl]-4-(trifluoromethyl)pyridine-3-carboxamide (Compound 9)

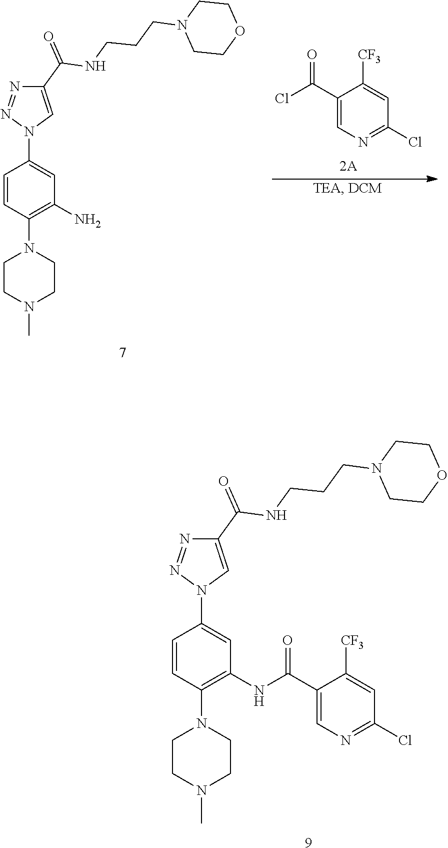 [SMILES: CN1CCN(c2ccc(-n3cc(C(=O)NCCCN4CCOCC4)nn3)cc2N)CC1 ; O=C(Cl)c1cnc(Cl)cc1C(F)(F)F ; "TEA, DCM" ; "7" ; CN1CCN(c2ccc(-n3cc(C(=O)NCCCN4CCOCC4)nn3)cc2NC(=O)c2cnc(Cl)cc2C(F)(F)F)CC1 ; "9"]

To a mixture of compound 7 (652.33 mg, 1.52 mmol) and compound 2A (520 mg, 2.13 mmol) in DCM (6 mL) was added TEA (770.18 mg, 7.61 mmol, 1.06 mL) at −10° C. The reaction mixture was stirred at 25° C. for 20 min. The mixture was concentrated to remove DCM. The crude product was purified by flash chromatography on silica gel (MeOH in DCM=0% to 1% to 9%) to give compound 9 (600 mg, 848.97 umol, 27.89% yield) was obtained as a yellow solid.

¹H NMR (CDCl3, 400 MHz) δ_{H}=9.07 (s, 1H), 8.92 (d, J=2.4 Hz, 1H), 8.77 (s, 1H), 8.56-8.36 (m, 2H), 7.74 (s, 1H), 7.64 (dd, J=2.8, 8.8 Hz, 1H), 7.44 (d, J=8.8 Hz, 1H), 3.87 (s, 4H), 3.63-3.59 (m, 2H), 3.16-2.91 (m, 6H), 2.59 (s, 8H), 2.38 (s, 3H), 1.41 (t, J=7.6 Hz, 2H).

### Step 9: 6-fluoro-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide (HYBI_200)

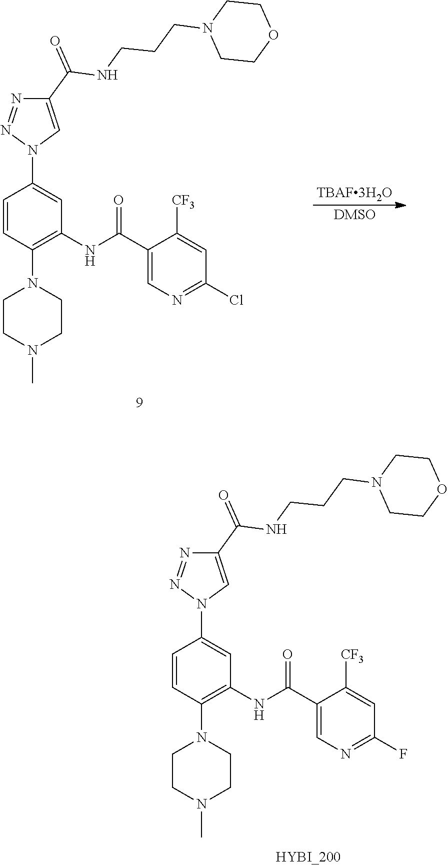

To a solution of compound 9 (110 mg, 172.94 umol, 1 eq) in DMSO (1 mL) was added TBAF·3H₂O (70.93 mg, 224.82 umol, 1.3 eq). The mixture was stirred at 100° C. for 1 h. The reaction mixture was concentrated directly. The residue was purified by prep-HPLC (column: Phenomenex Gemini-NX C18 75*30 mm*3 um; mobile phase: [water (0.05% NH₃H₂O+10 mM NH₄HCO₃)-ACN]; B %: 23%-63%, 11 min). HYBI_200 (9.4 mg, 15.11 umol, 8.73% yield, 99.57% purity) was obtained as a white solid.

¹H NMR (DMSO-d₆, 400 MHz) δ_{H}=10.03 (s, 1H), 9.17 (s, 1H), 8.82 (t, J=5.6 Hz, 1H), 8.73 (s, 1H), 8.50 (d, J=2 Hz, 1H), 7.88 (s, 1H), 7.75 (dd, J=2.4, 8.4 Hz, 1H), 7.38 (d, J=8.8 Hz, 1H), 3.61 (t, J=4.4 Hz, 4H), 3.34 (s, 2H), 2.98-2.90 (m, 4H), 2.49-2.43 (m, 4H), 2.41-2.31 (m, 6H), 2.21 (s, 3H), 1.75-1.65 (m, 2H).

HPLC Rₜ=3.524 min in 8 min chromatography, purity 99.57%.

LCMS Rₜ=1.785 min in 4 min chromatography, purity 99.01%, MS ESI calcd. for 619.26, [M+H]⁺ 620.26, found 620.3.

### Example 12. Synthesis of 6-methyl-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide

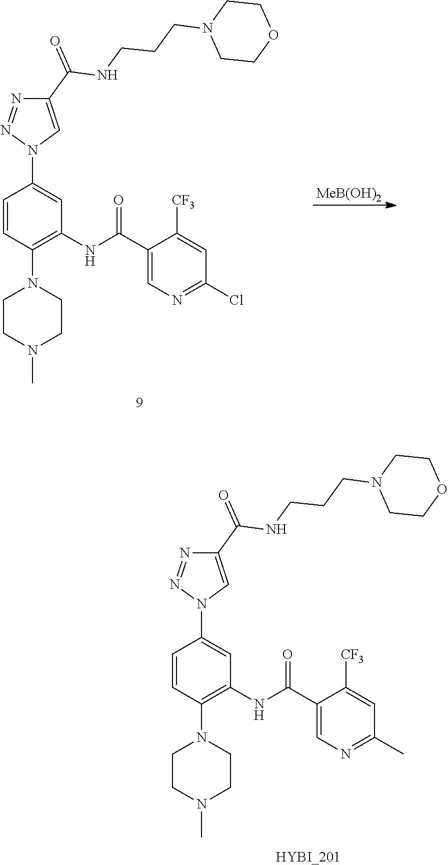

To a mixture of compound 9 (50 mg, 78.61 umol, 1 eq) in DMF (2 mL) was added MeB(OH)₂ (33 mg, 550.26 umol, 7 eq), K₂CO₃ (54 mg, 393.04 umol, 5 eq) and Pd(PPh₃)₂Cl₂ (6 mg, 7.86 umol, 0.1 eq). The mixture was stirred at 100° C. for 16 hrs. The mixture was concentrated to dryness. The mixture was purified with prep-HPLC (column: Phenomenex Gemini-NX C18 75*30 mm*3 um; mobile phase: [water(0.05% NH₃H₂O+10 mM NH₄HCO₃)-ACN]; B %: 19%-59%, 11 min). HYBI_201 (7.2 mg, 11.34 umol, 14.43% yield, 96.96% purity) was obtained as a white solid.

¹H NMR (DMSO-d₆, 400 MHz) δ_{H}=9.97 (s, 1H), 9.18 (s, 1H) 8.79-8.92 (m, 2H) 8.55-8.42 (m, 1H) 7.80 (s, 1H) 7.74 (dd, J=8.4, 2.4 Hz, 1H) 7.38 (d, J=8.4 Hz, 1H) 3.61 (t, J=1.6 Hz, 4H) 2.87-3.03 (m, 6H) 2.62-2.71 (m, 5H) 2.31-2.40 (m, 8H) 2.22 (s, 3H) 1.75-1.68 (m, 2H).

HPLC Rₜ=3.401 min in 8 min chromatography, purity 96.96%.

LCMS Rₜ=1.716 min in 4 min chromatography, XBridge Shield RP18, 5 um, 2.1*50 mm, purity 99.23%, MS ESI calcd. for 615.29 [M+H]⁺ 616.29, found 616.3.

### Example 13. Synthesis of 6-cyano-N-[2-(4-methylpiperazin-1-yl)-5-[4-(3-morpholinopropylcarbamoyl)triazol-1-yl]phenyl]-4-(trifluoromethyl)pyridine-3-carboxamide

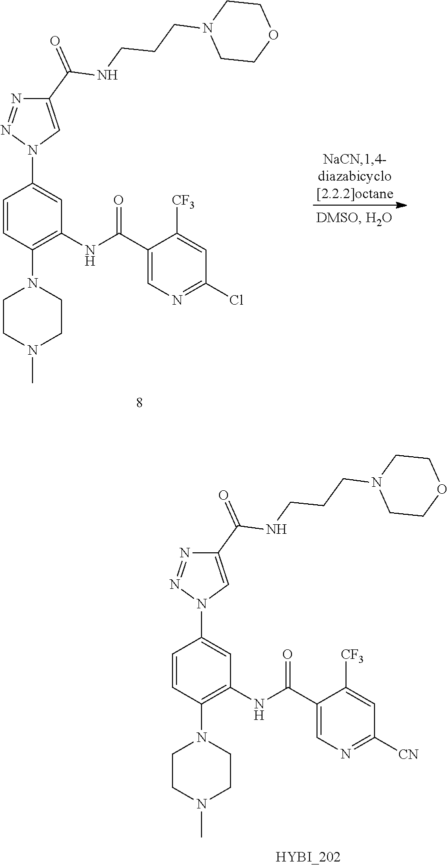

To a mixture of compound 8 (200 mg, 0.031 mmol) in DMSO (4 mL) was added 1,4-diazabicyclo[2.2.2]octane (17.64 mg, 0.16 mmol, 0.017 mL), NaCN (260 mg, 5.31 mmol) and H₂O (0.4 mL). The mixture was stirred at 100° C. for 1 h. The residue was diluted with H₂O (20 mL), and the mixture was extracted with EtOAc (20 mL×2). The combined organic phase was washed with brine (20 mL), dried over anhydrous Na₂SO₄, filtered and concentrated to give the crude product. The crude product was purified by prep-HPLC (column: Phenomenex Gemini-NX C18 75×30 mm×3 um; mobile phase: [water(10 mM NH₄HCO₃)-ACN]; B %: 25%-55%, 10 min) to give HYBI_202 (30 mg, 47.88 umol, 15.23% yield) as a yellow solid.

¹H NMR (DMSO-d₆, 400 MHz) δ_{H}=10.21 (s, 1H), 9.20 (s, 2H), 8.85 (t, J=5.6 Hz, 1H), 8.71 (s, 1H), 8.68-8.75 (m, 1H), 8.53 (d, J=2.8 Hz, 1H), 7.76 (dd, J=8.8, 2.4 Hz, 1H), 7.38 (d, J=8.8 Hz, 1H), 3.61 (t, J=4.4 Hz, 5H), 3.39-3.44 (m, 2H), 2.95 (br s, 4H), 2.45-2.49 (m, 4H), 2.33-2.40 (m, 6H), 2.21 (s, 3H), 1.66-1.75 (m, 2H).

HPLC Rₜ=2.110 min in 8 min chromatography, purity 94.83%.

LCMS Rₜ=2.194 min in 7 min chromatography, purity 94.63%, MS ESI calcd. for 626.27 [M+H]⁺ 627.27, found 627.3.

### Example 14. N-[2-(4-methylpiperazin-1-yl)-5-[4-(3-morpholinopropylcarbamoyl)triazol-1-yl]phenyl]-6-methylsulfanyl-4-(trifluoromethyl)pyridine-3-carboxamide

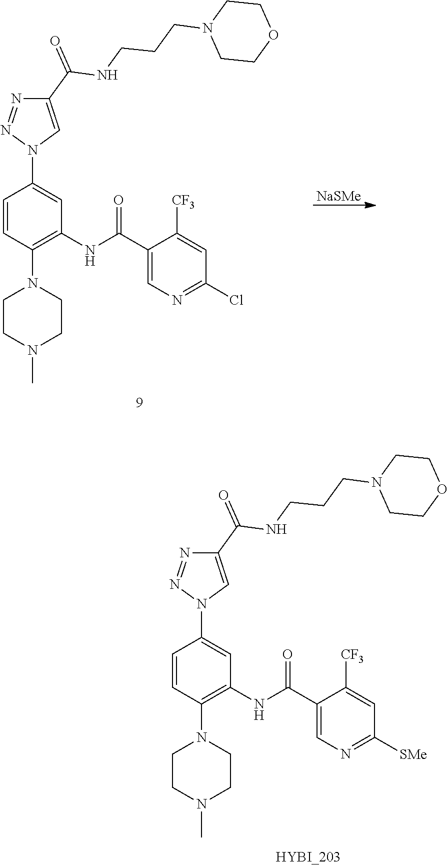

To a mixture of NaSMe (60 mg, 0.86 mmol, 0.055 mL) in DMF (1 mL) was added compound 9 (50 mg, 0.079 mmol) in DMF (1 mL), and the mixture was stirred at 20° C. for 1 h. The residue was diluted with H₂O (50 mL), and the mixture was extracted with EtOAc (30 mL×2). The combined organic phase was washed with brine (20 mL), dried over anhydrous Na₂SO₄, filtered and concentrated to give the crude product. The crude product was purified by Prep-HPLC (column: Phenomenex Gemini-NX C18 75×30 mm×3 um; mobile phase: [water(10 mM NH₄HCO₃)-ACN]; B %: 32%-62%, 10 min) to give HYBI_203 (30 mg, 46.32 umol, 58.92% yield) as a yellow solid.

¹H NMR (DMSO-d₆, 400 MHz) δ_{H}=9.92 (s, 1H), 9.19 (s, 1H), 8.78-8.86 (m, 2H), 8.50 (d, J=2.4 Hz, 1H), 7.80 (s, 1H), 7.74 (dd, J=8.8, 2.8 Hz, 1H), 7.38 (d, J=8.8 Hz, 1H), 3.61 (t, J=4.4 Hz, 4H), 3.33-3.38 (m, 2H), 2.94 (d, J=4.4 Hz, 4H), 2.64 (s, 3H), 2.52-2.55 (m, 4H), 2.36 (m, 6H), 2.22 (s, 3H), 1.70 (m, 2H).

HPLC Rₜ=1.306 min in 8 min chromatography, XBridge Shield RP18 2.1×50 mm, 5 m, purity 99.57%.

LCMS Rₜ=1.390 min in 7 min chromatography, Xtimate C18, 3 m, 2.1×30 mm, purity 99.61%, MS ESI calcd. for 647.26 [M+H]⁺ 648.26, found 648.5.

### Example 15. N-[2-(4-methylpiperazin-1-yl)-5-[4-(3-morpholinopropylcarbamoyl)triazol-1-yl]phenyl]-6-methylsulfonyl-4-(trifluoromethyl)pyridine-3-carboxamide

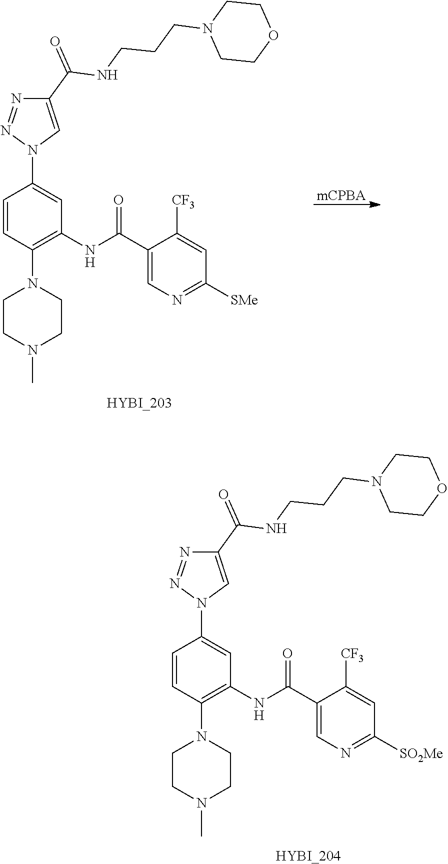

To a solution of HYBI_203 (150 mg, 231.58 umol, 1 eq) in DCM (5 mL) was added m-CPBA (85% purity, 79.93 mg, 463.17 umol, 2 eq) in portions at 0° C. Then the mixture was stirred at 20° C. for 2 hours under N₂. The mixture was added to saturated aqueous Na₂S2O₃ (10 mL) and saturated aqueous NaHCO₃ (10 mL) and the aqueous phase was extracted with DCM (3×20 mL). The combined organic phase was washed with brine (50 mL), dried over anhydrous Na₂SO₄, filtered and concentrated under vacuum to give a residue. The residue was purified by prep-HPLC (column: Phenomenex Gemini NX C18 150*40 mm*5 um; mobile phase: [water(0.05% HCl)-ACN]; B %: 1%-25%, 10 min) to give HYBI_204 (hydrochloride, 31.6 mg, 45.47 umol, 19.63% yield, 97.80% purity) as a yellow solid.

¹H NMR (DMSO-d₆, 400 MHz) δ_{H}=12.77 (br s, 1H), 12.33 (br s, 1H), 10.04 (s, 1H), 9.30 (s, 1H), 9.02-8.81 (m, 2H), 8.68 (d, J=2.0 Hz, 1H), 7.83 (s, 1H), 7.79 (dd, J=2.4, 8.8 Hz, 1H), 7.58 (d, J=8.8 Hz, 1H), 3.98-3.86 (m, 8H), 3.84-3.77 (m, 2H), 3.77-3.68 (m, 4H), 3.60 (s, 3H), 3.55-3.53 (m, 4H), 3.27 (br d, J=13.2 Hz, 2H), 2.64 (s, 3H), 2.18-2.05 (m, 2H).

LCMS Rₜ=0.730 min in 1.5 min chromatography, Agilent Pursult 5 C18 20*2.0 mm, purity 98.26%, MS ESI calcd. for 679.25 [M+H]⁺ 680.25, found 680.6.

HPLC Rₜ=1.88 min in 8 min chromatography, Xbridge Shield RP18 5 um 2.1*50 mm, purity 97.80%.

### Example 16. 6-methoxy-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide

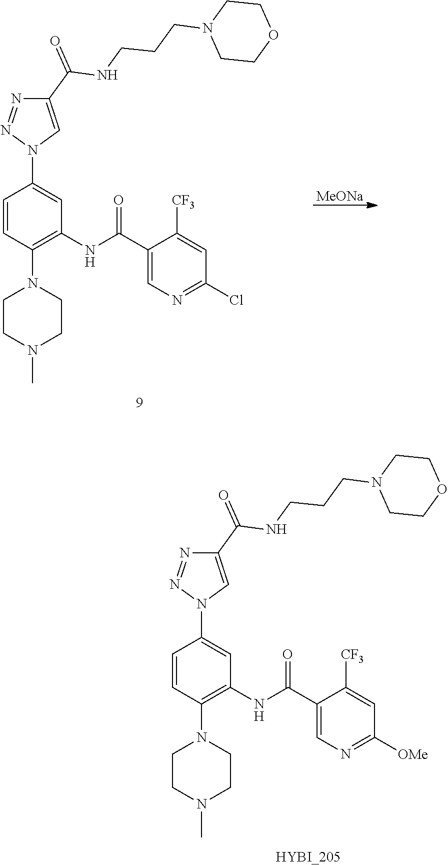

To a mixture of compound 9 (50 mg, 78.61 umol, 1 eq) in MeOH (1 mL) was added NaOMe (8 mg, 157.22 umol, 2 eq). The mixture was stirred at 25° C. for 16 hrs. Another batch of NaOMe (34 mg, 628.86 umol, 8 eq) was added into the mixture after stirring. The mixture was stirred at 40° C. for another 16 hrs. The mixture was concentrated to dryness. The mixture was purified by prep-HPLC (column: Phenomenex Gemini-NX C18 75*30 mm*3 um; mobile phase: [water (0.05% NH₃H₂O+10 mM NH₄HCO₃)-ACN]; B %: 23%-63%, 11 min) and SFC (column: DAICEL CHIRALCEL OD (250 mm*30 mm, 10 um); mobile phase: [0.1% NH₃H₂O EtOH]; B %: 50%-50%, min). HYBI_205 (11.6 mg, 17.83 umol, 22.68% yield, 97.08% purity) was obtained as a white solid.

¹H NMR (DMSO-d₆, 400 MHz) δH=9.77 (s, 1H) 9.19 (s, 1H) 8.81 (t, J=5.6 Hz, 1H) 8.52 (s, 1H) 8.10 (d, J=8.4 Hz, 1H) 7.73 (dd, J=8.8, 2.4 Hz, 1H) 7.38 (d, J=8.8 Hz, 1H) 7.29 (d, J=8.4 Hz, 1H) 3.98 (s, 3H) 3.62 (t, J=4.4 Hz, 4H) 3.34-3.39 (m, 4H) 3.29 (s, 2H) 2.86-3.03 (m, 5H) 2.33-2.42 (m, 6H) 2.22 (s, 3H) 1.67-1.79 (m, 2H).

HPLC Rₜ=3.864 min in 8 min chromatography, purity 97.08%.

LCMS Rₜ=1.942 min in 4 min chromatography, Chromolith Flash RP-18.5 um, 3.0*25 mm, purity 97.76%, MS ESI calcd. for 631.28 [M+H]⁺ 632.28, found 632.3.

### Example 17. 6-ethoxy-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide

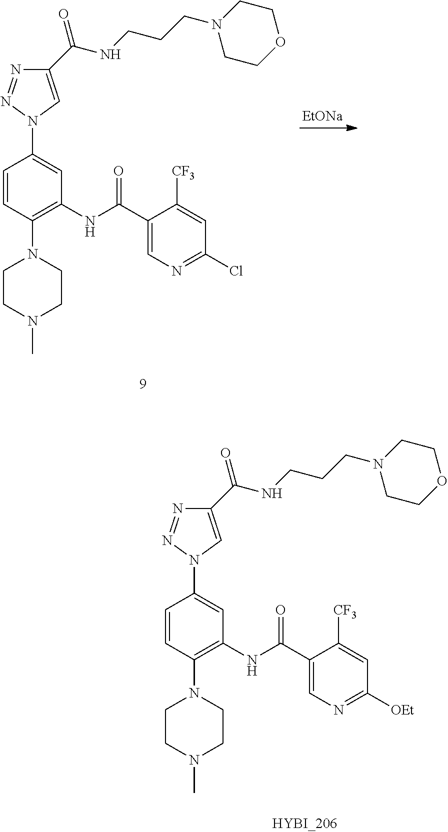 [SMILES: "EtONa" ; CN1CCN(c2ccc(-n3cc(C(=O)NCCCN4CCOCC4)nn3)cc2NC(=O)c2cnc(Cl)cc2C(F)(F)F)CC1 ; CCOc1cc(C(F)(F)F)c(C(=O)Nc2cc(-n3cc(C(=O)NCCCN4CCOCC4)nn3)ccc2N2CCN(C)CC2)cn1]

To a mixture of compound 9 (50 mg, 78.61 umol, 1 eq) in EtOH (1 mL) was added EtONa (11 mg, 157.22 umol, 2 eq). The mixture was stirred at 70° C. for 32 hrs. The mixture was concentrated to dryness. The mixture was purified by prep-HPLC (column: Phenomenex Gemini-NX C18 75*30 mm*3 um; mobile phase: [water(0.05% NH₃H₂O+10 mM NH₄HCO₃)-ACN]; B %: 23%-73%, 12 min). HYBI_206 (9.9 mg, 15.07 umol, 19.17% yield, 98.27% purity) was obtained as a white solid.

¹H NMR (DMSO-d₆, 400 MHz) δ_{H}=9.86 (s, 1H) 9.17 (s, 1H) 8.85 (t, J=5.6 Hz, 1H) 8.61 (s, 1H) 8.48 (d, J=2.4 Hz, 1H) 7.73 (dd, J=8.8, 2.4 Hz, 1H) 7.38 (d, J=8.4 Hz, 1H) 7.30 (s, 1H) 4.46 (q, J=7.2 Hz, 2H) 3.59-3.63 (m, 6H) 2.92-2.97 (m, 4H) 2.67-2.69 (m, 2H) 2.36-2.39 (m, 4H) 2.32-2.35 (m, 4H) 2.22 (s, 3H) 1.71 (t, J=6.8 Hz, 2H) 1.38 (t, J=7.2 Hz, 3H).

HPLC Rₜ=4.018 min in 8 min chromatography, Ultimate XB-C18 3.0*50 mm, 3 um, purity 98.42%.

LCMS Rₜ=2.064 min in 4 min chromatography, Chromolith Flash RP-18.5 um, 3.0*25 mm, purity 100%, MS ESI calcd. for 645.30 [M+H]⁺ 646.30, found 646.4.

### Example 18. 6-amino-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide

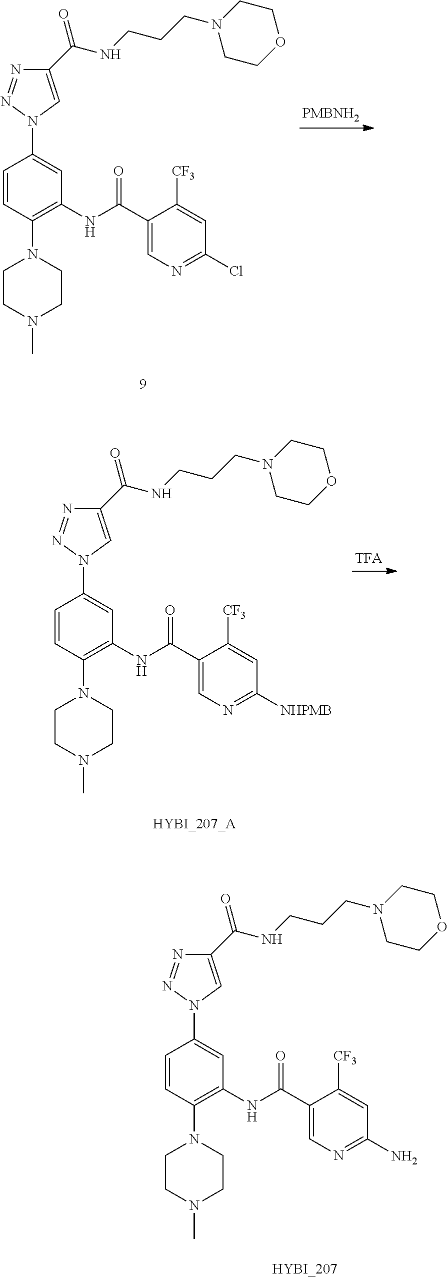

### Step 1: 6-((4-methoxybenzyl)amino)-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide (HYBI_207_A)

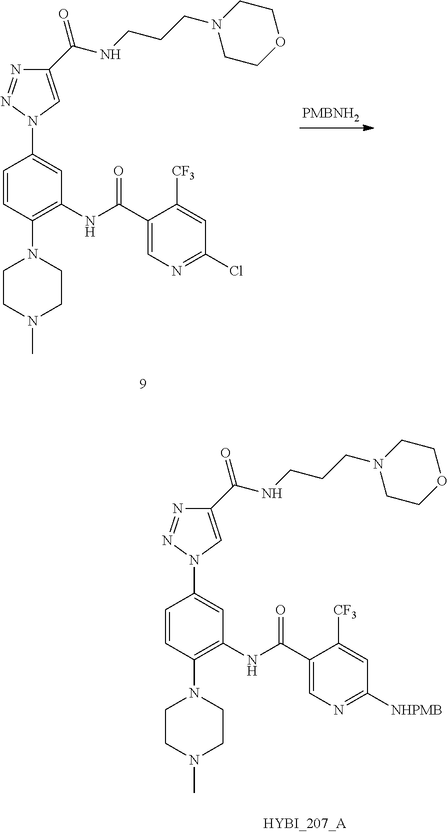

To a mixture of compound 9 (400 mg, 628.86 umol, 1 eq) in DMF (4 mL) was added PMBNH₂ (86 mg, 628.86 umol, 81.38 uL, 1 eq), DIEA (244 mg, 1.89 mmol, 328.61 uL, 3 eq) and DABCO (21 mg, 188.66 umol, 20.75 uL, 0.3 eq). The mixture was stirred at 80° C. for 16 hrs. The combined mixture was concentrated to dryness. The mixture was purified with prep-HPLC (column: Xtimate C18 150*40 mm*10 um; mobile phase: [water(10 mM NH₄HCO₃)-ACN]; B %: 35%-65%, 10 min). HYBI_207_A (33 mg, 33.59 umol, 5.34% yield, 75% purity) was obtained as a white solid.

¹H NMR (DMSO-d₆, 400 MHz) δ_{H}=9.54 (s, 1H), 9.18 (s, 1H), 8.81 (t, J=5.6 Hz, 1H), 8.53 (d, J=2.4 Hz, 1H), 8.46 (s, 1H), 7.97-8.11 (m, 1H), 7.70 (dd, J=8.4, 2.4 Hz, 1H), 7.40 (d, J=8.4 Hz, 1H), 7.30 (d, J=8.4 Hz, 2H), 6.81-7.02 (m, 3H), 4.53 (d, J=5.6 Hz, 2H), 3.74 (s, 3H), 3.61 (t, J=4.4 Hz, 4H), 3.36 (s, 2H), 2.96-2.91 (m, 4H), 2.69-2.66 (m, 2H), 2.32-2.41 (m, 8H), 2.23 (s, 3H), 1.71 (t, J=6.8 Hz, 2H).

### Step 2: 6-amino-N-(2-(4-methylpiperazin-1-yl)-5-(4-((3-morpholinopropyl)carbamoyl)-1H-1,2,3-triazol-1-yl)phenyl)-4-(trifluoromethyl)nicotinamide (HYBI_207)

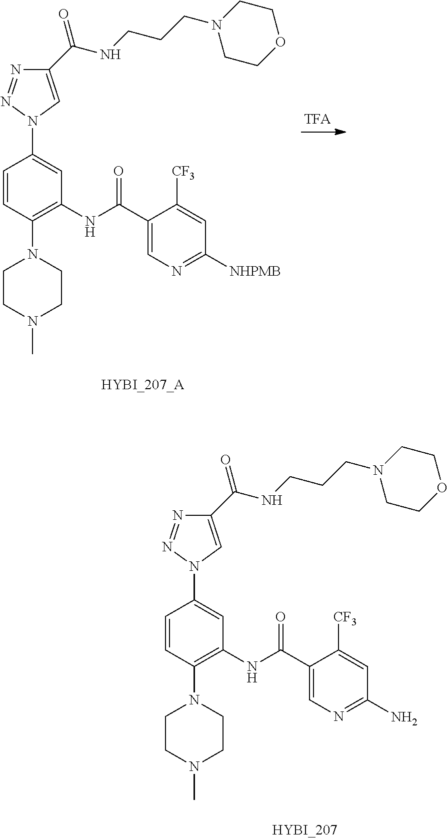

A mixture of HYBI_207_A (30 mg, 40.72 umol, 1 eq) and TFA (3 mL) was stirred at 50° C. for 1 hr. The mixture was concentrated. The mixture was adjusted with saturated aqueous NaHCO₃ to pH˜8. The mixture was filtered and the filtrate was concentrated to dryness2e. The residue was purified by prep-HPLC column: Phenomenex Gemini-NX C18 75*30 mm*3 um; mobile phase: [water(10 mM NH₄HCO₃)-ACN]; B %: 22%-42%, 7 min. HYBI_207 (10.6 mg, 17.20 umol, 42.21% yield, 97.67% purity) was obtained as a white solid.

¹H NMR (DMSO-d₆, 400 MHz) δ_{H}=9.51 (s, 1H), 9.18 (s, 1H), 8.82 (t, J=5.6 Hz, 1H), 8.55 (d, J=2.8 Hz, 1H), 8.40 (s, 1H), 7.69 (dd, J=2.4, 8.4 Hz, 1H), 7.40 (d, J=8.8 Hz, 1H), 7.05 (s, 2H), 6.84 (s, 1H), 3.61 (t, J=4.8 Hz, 4H), 2.93 (t, J=4.8 Hz, 4H), 2.53-2.52 (m, 2H), 2.49-2.46 (m, 4H), 2.39-2.32 (m, 6H), 2.22 (s, 3H), 1.70 (m, 2H).

HPLC Rₜ=3.554 min in 8 min chromatography, purity 97.67%.

LCMS Rₜ=1.492 min in 4 min chromatography, purity 95.25%, MS ESI calcd. for 616.28 [M+H]⁺ 617.28, found 617.3.

### Example 19. 4,6-dichloro-N-[2-(4-methylpiperazin-1-yl)-5-[4-(3-morpholinopropylcarbamoyl)triazol-1-yl]phenyl]pyridine-3-carboxamide

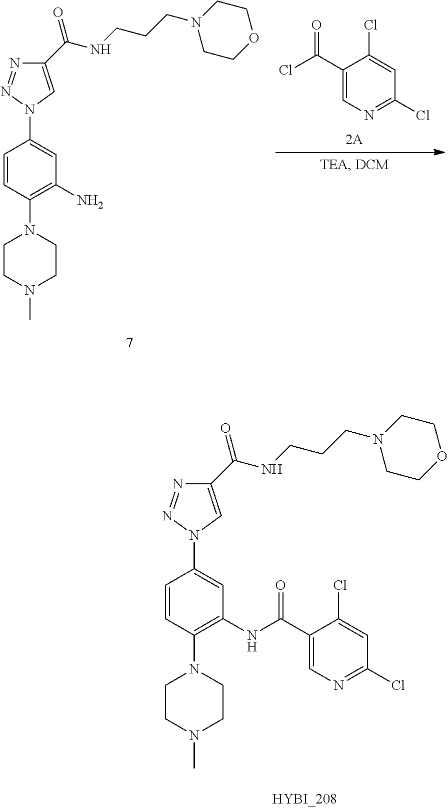 [SMILES: CN1CCN(c2ccc(-n3cc(C(=O)NCCCN4CCOCC4)nn3)cc2N)CC1 ; O=C(Cl)c1cnc(Cl)cc1Cl ; CN1CCN(c2ccc(-n3cc(C(=O)NCCCN4CCOCC4)nn3)cc2NC(=O)c2cnc(Cl)cc2Cl)CC1]

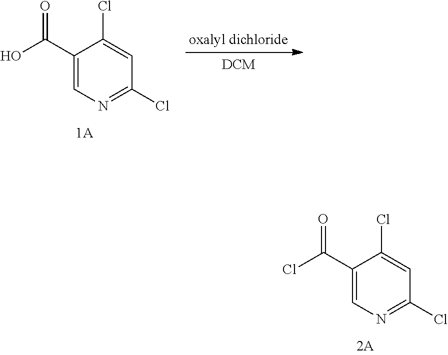 [SMILES: O=C(O)c1cnc(Cl)cc1Cl ; O=C(Cl)c1cnc(Cl)cc1Cl]

Step 1: 4,6-dichloropyridine-3-carbonyl chloride (Compound 2A)

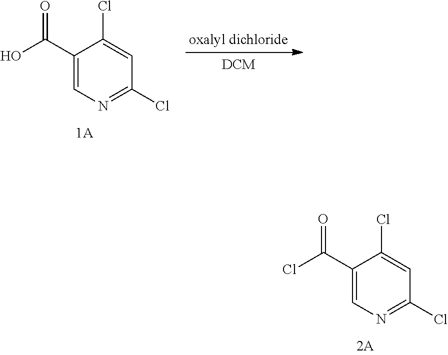 [SMILES: O=C(O)c1cnc(Cl)cc1Cl ; O=C(Cl)c1cnc(Cl)cc1Cl]

To a mixture of compound 1A (100 mg, 0.521 mmol) and DMF (one drop) in DCM (3 mL) was added oxalyl dichloride (330.54 mg, 2.60 mmol, 0.23 mL) dropwise at 0° C., The mixture was stirred at 20° C. for 30 min. The mixture was concentrated to give the residue. The crude compound 2A (100 mg, 475.18 umol, 91.23% yield) as a yellow oil, which was used into the next step without further purification.

### Step 2: 4,6-dichloro-N-[2-(4-methylpiperazin-1-yl)-5-[4-(3-morpholinopropylcarbamoyl)triazol-1-yl]phenyl]pyridine-3-carboxamide (HYBI_208)

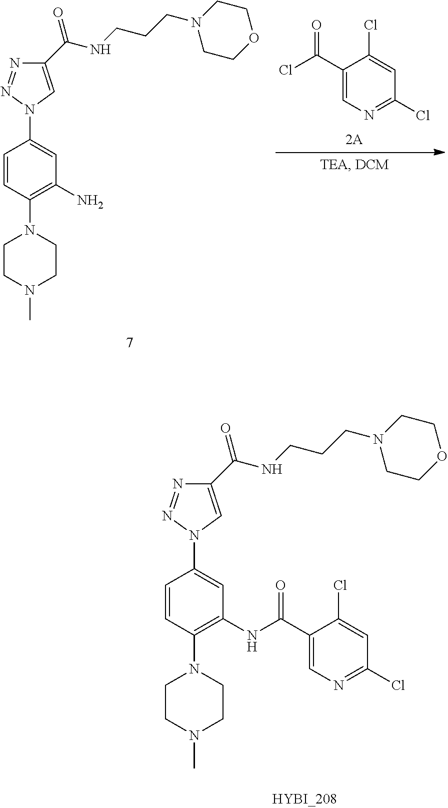 [SMILES: CN1CCN(c2ccc(-n3cc(C(=O)NCCCN4CCOCC4)nn3)cc2N)CC1 ; O=C(Cl)c1cnc(Cl)cc1Cl ; CN1CCN(c2ccc(-n3cc(C(=O)NCCCN4CCOCC4)nn3)cc2NC(=O)c2cnc(Cl)cc2Cl)CC1]

To a mixture of compound 7 (100 mg, 0.23 mmol) and compound 2A (68.75 mg, 0.33 mmol) in DCM (2 mL) at −10° C. was added TEA (236.13 mg, 2.33 mmol, 0.32 mL). The mixture was stirred at 25° C. for 10 min. The residue was diluted with H₂O (30 mL), and the mixture was extracted with DCM (30 mL×2). The combined organic phase was washed with water (20 mL) and brine (20 mL), dried over anhydrous Na₂SO₄, filtered and concentrated to give the crude product. The crude product was purified by prep-HPLC (column: Phenomenex Gemini NX C18 150×40 mm×5 um; mobile phase: [water(0.05% HCl)-ACN]; B %: 0%-35%, 10 min) and then (column: Phenomenex Gemini-NX C18 75×30 mm×3 um; mobile phase: [water(10 mM NH₄HCO₃)-ACN]; B %: 30%-50%, 7.5 min) to give HYBI_208 (20 mg, 33.19 umol, 14.22% yield) as a white solid.

¹H NMR (DMSO-d₆, 400 MHz) δ_{H}=10.01 (s, 1H), 9.19 (s, 1H), 8.83 (t, J=5.6 Hz, 1H), 8.69 (s, 1H), 8.58 (s, 1H), 8.00 (s, 1H), 7.75 (dd, J=8.8, 2.8 Hz, 1H), 7.38 (d, J=8.8 Hz, 1H), 3.61 (t, J=4.4 Hz, 4H), 3.35-3.39 (m, 2H), 2.95 (d, J=4.4 Hz, 4H), 2.52 (m, 4H), 2.36 (m, 6H), 2.22 (s, 3H), 1.70 (m, 2H).

HPLC Rₜ=0.935 min in 8 min chromatography, XBridge Shield RP18 2.1×50 mm, 5 μm, purity 99.94%.

LCMS Rₜ=0.765 min in 7 min chromatography, Xtimate C18, 3 m, 2.1×30 mm, purity 99.92%, MS ESI calcd. for 601.21 [M+H]⁺ 602.21, found 602.4.

### Example 20. 4-chloro-N-(2-(4-methylpiperazin-1-yl)-5-(4-((3-morpholinopropyl)carbamoyl)-1H-1,2,3-triazol-1-yl)phenyl)-6-(trifluoromethyl)nicotinamide

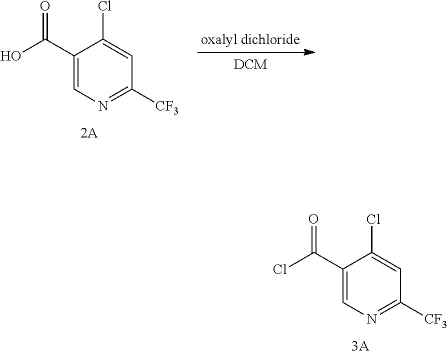 [SMILES: O=C(O)c1cnc(C(F)(F)F)cc1Cl ; O=C(Cl)c1cnc(C(F)(F)F)cc1Cl]

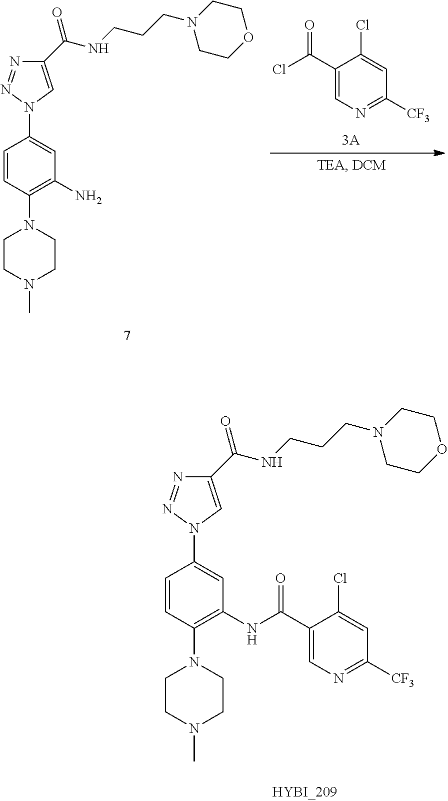 [SMILES: CN1CCN(c2ccc(-n3cc(C(=O)NCCCN4CCOCC4)nn3)cc2N)CC1 ; O=C(Cl)c1cnc(C(F)(F)F)cc1Cl ; CN1CCN(c2ccc(-n3cc(C(=O)NCCCN4CCOCC4)nn3)cc2NC(=O)c2cnc(C(F)(F)F)cc2Cl)CC1]

### Step 1: 4-chloro-6-(trifluoromethyl)nicotinoyl chloride (Compound 3A)

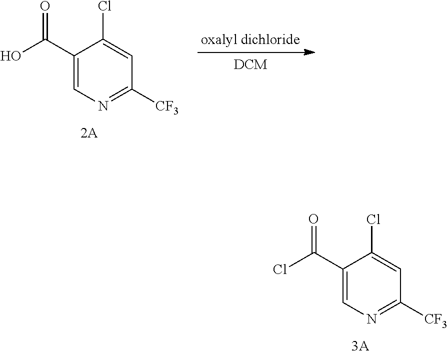 [SMILES: O=C(O)c1cnc(C(F)(F)F)cc1Cl ; O=C(Cl)c1cnc(C(F)(F)F)cc1Cl]

To a solution of compound 2A (200 mg, 886.71 umol, 1 eq) and DMF (6.48 mg, 88.67 umol, 6.82 uL, 0.1 eq) in DCM (3 mL) was added oxalyl dichloride (562.75 mg, 4.43 mmol, 388.10 uL, 5 eq) dropwise at 0° C. The reaction mixture was stirred at 25° C. for 20 min. The mixture was concentrated to remove DCM. The product was used in the next step without further purification. Compound 3A (210 mg, crude) was obtained as yellow oil.

Step 2: 4-chloro-N-(2-(4-methylpiperazin-1-yl)-5-(4-((3-morpholinopropyl)carbamoyl)-1H-1,2,3-triazol-1-yl)phenyl)-6-(trifluoromethyl)nicotinamide (HYBI_209)

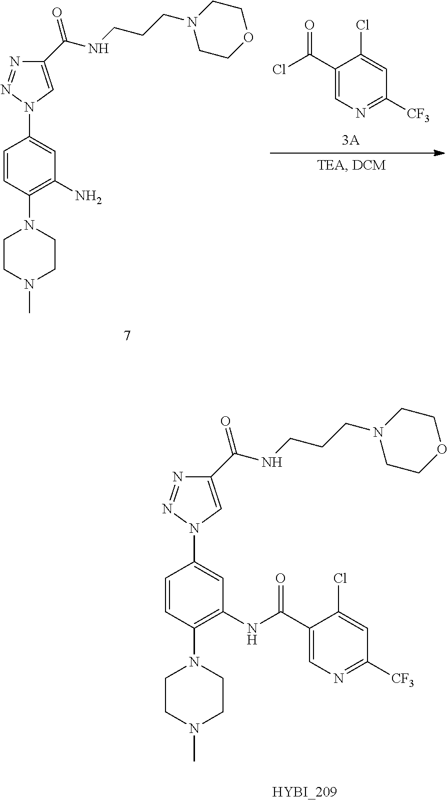 [SMILES: CN1CCN(c2ccc(-n3cc(C(=O)NCCCN4CCOCC4)nn3)cc2N)CC1 ; O=C(Cl)c1cnc(C(F)(F)F)cc1Cl ; CN1CCN(c2ccc(-n3cc(C(=O)NCCCN4CCOCC4)nn3)cc2NC(=O)c2cnc(C(F)(F)F)cc2Cl)CC1]

To a mixture of compound 7 (263.44 mg, 614.76 umol, 1 eq) and compound 3A (210 mg, 860.66 umol, 1.4 eq) in DCM (3 mL) was added TEA (311.03 mg, 3.07 mmol, 427.83 uL, 5 eq) at −10° C. The reaction mixture was stirred at 25° C. for 20 min. The mixture was concentrated to remove DCM. The residue was purified by prep-HPLC column: Phenomenex Gemini-NX C18 75*30 mm*3 um; mobile phase: [water(10 mM NH4HCO3)-ACN]; B %: 30%-60%, 10 min. HYBI_209 (27.4 mg, 42.74 umol, 6.95% yield, 99.22% purity) was obtained as a white solid.

¹H NMR (DMSO-d₆, 400 MHz) δ_{H}=10.15 (s, 1H), 9.20 (s, 1H), 9.00 (s, 1H), 8.84 (t, J=5.2 Hz, 1H), 8.59 (s, 1H), 8.33 (s, 1H), 7.79-7.73 (m, 1H), 7.37 (d, J=8.8 Hz, 1H), 3.65-3.56 (m, 4H), 3.36-3.32 (m, 2H), 2.96 (br s, 4H), 2.50 (s, 4H), 2.39-2.33 (m, 6H), 2.21 (s, 3H), 1.74-1.66 (m, 2H).

HPLC Rₜ=3.85 min in 8 min chromatography, purity 99.22%.

LCMS Rₜ=1.928 min in 4 min chromatography, purity 99.26%, MS ESI calcd. for 635.24 [M+H]⁺ 636.24, found 636.6.

### Example 21. 4,6-dichloro-5-methyl-N-(2-(4-methylpiperazin-1-yl)-5-(4-((3-morpholinopropyl)carbamoyl)-1H-1,2,3-triazol-1-yl)phenyl)nicotinamide

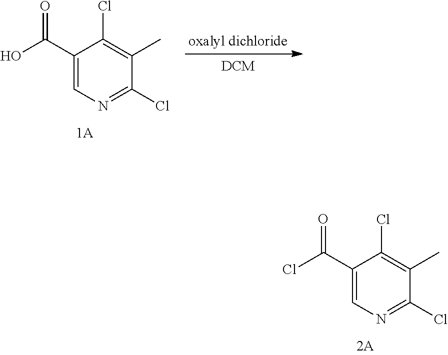

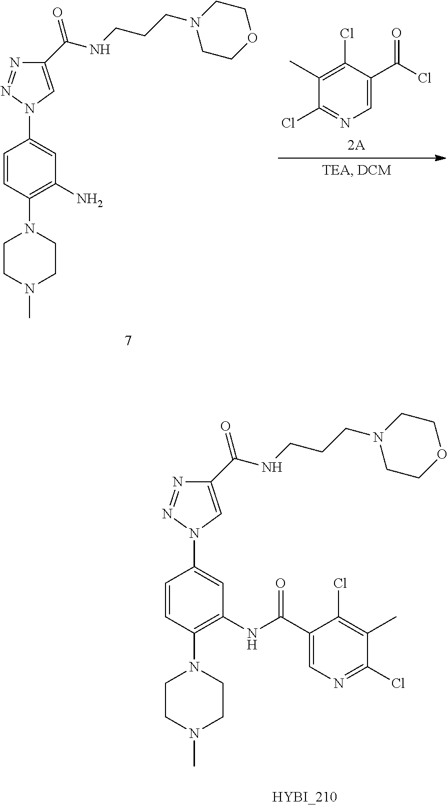

### Step 1: 4,6-dichloro-5-methylnicotinoyl chloride (Compound 2A)

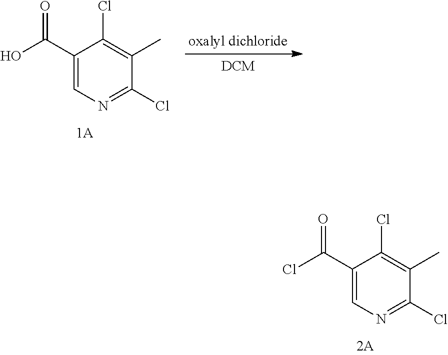

To a solution of compound 1A (200 mg, 970.75 umol, 1 eq) and DMF (7.10 mg, 97.08 umol, 7.47 uL, 0.1 eq) in DCM (3 mL) was added oxalyl dichloride (616.09 mg, 4.85 mmol, 424.89 uL, 5 eq) dropwise at 0° C. The reaction mixture was stirred at 25° C. for 20 min. The mixture was concentrated to remove DCM. The product was used in the next step without further purification. Compound 2A (210 mg, crude) was obtained as yellow oil.

### Step 2: 4, 6-dichloro-5-methyl-N-(2-(4-methylpiperazin-1-yl)-5-(4-((3-morpholinopropyl)carbamoyl)-1H-1,2,3-triazol-1-yl)phenyl)nicotinamide (HYBI_210)

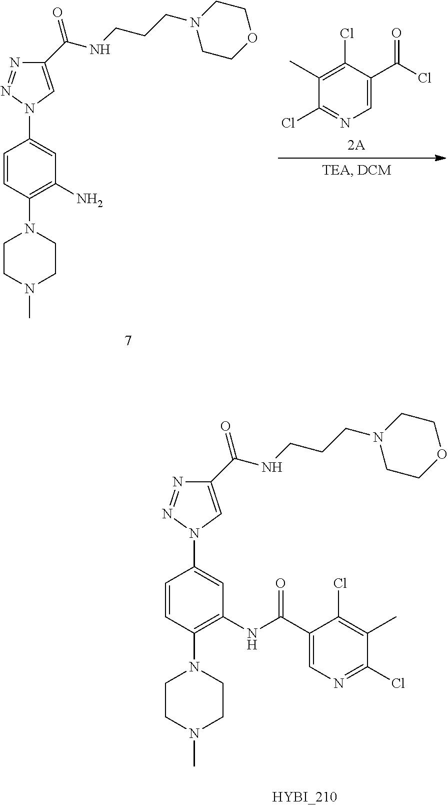

To a mixture of compound 7 (286.36 mg, 668.24 umol, 1 eq) and compound 2A (210 mg, 935.53 umol, 1.4 eq) in DCM (3 mL) was added TEA (338.09 mg, 3.34 mmol, 465.05 uL, 5 eq) at −10° C. The reaction mixture was stirred at 25° C. for 20 min. The mixture was concentrated to remove DCM. The residue was purified by prep-HPLC column: Phenomenex Gemini-NX C18 75*30 mm*3 um; mobile phase: [water(10 mM NH4HCO3)-ACN]; B %: 30%-60%, 10 min. HYBI_210 (59.3 mg, 94.73 umol, 14.18% yield, 98.49% purity) was obtained as a white solid ¹H NMR (DMSO-d₆, 400 MHz) δ_{H}=9.97 (s, 1H), 9.20 (s, 1H), 8.89-8.79 (m, 1H), 8.53 (d, J=17.6 Hz, 2H), 7.75 (dd, J=2.0, 8.8 Hz, 1H), 7.37 (d, J=8.8 Hz, 1H), 3.61 (s, 4H), 3.30-3.22 (m, 2H), 2.95 (s, 4H), 2.57-2.51 (m, 4H), 2.43-2.31 (m, 9H), 2.22 (s, 3H), 1.74-1.65 (m, 2H).

HPLC Rₜ=3.859 min in 8 min chromatography, purity 98.498%.

LCMS Rₜ=1.879 min in 4 min chromatography, purity 99.70%, MS ESI calcd. for 615.22 [M+H]⁺ 616.22, found 616.3.

### Example 22. 4-amino-6-chloro-N-(2-(4-methylpiperazin-1-yl)-5-(4-((3-morpholinopropyl)carbamoyl)-1H-1,2,3-triazol-1-yl)phenyl)nicotinamide

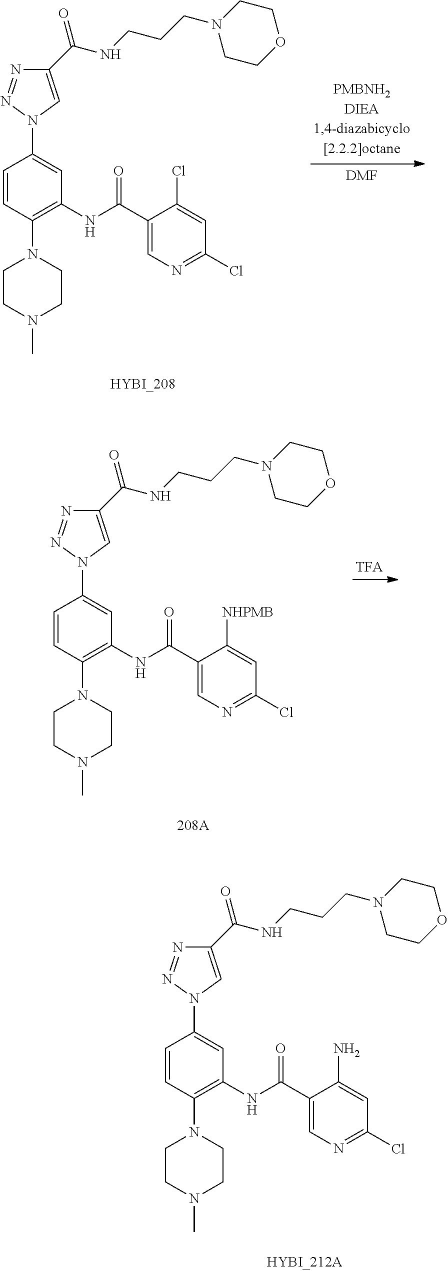

### Step 1:6-chloro-4-((4-methoxybenzyl)amino)-N-(2-(4-methylpiperazin-1-yl)-5-(4-((3-morpholinopropyl)carbamoyl)-1H-1,2,3-triazol-1-yl)phenyl)nicotinamide (Compound 208A)

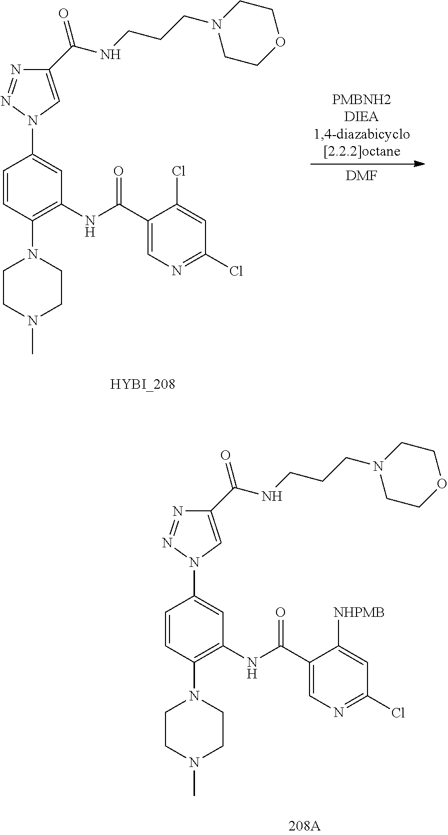

To a mixture of compound HYBI_208 (100 mg, 165.97 umol, 1 eq) in DMF (1 mL) was added PMBNH₂ (22.77 mg, 165.97 umol, 21.48 uL, 1 eq), DIEA (64.35 mg, 497.91 umol, 86.73 uL, 3 eq) and 1,4-diazabicyclo[2.2.2]octane (5.59 mg, 49.79 umol, 5.48 uL, 0.3 eq), and the mixture was stirred at 80° C. for 1 h. The residue was diluted with H₂O (5 mL), and the mixture was extracted with EtOAc (5 mL*3). The combined organic phase was washed with brine (10 mL), dried over anhydrous Na₂SO₄, filtered and concentrated. The product was used in the next step without further purification. Compound 208A (140 mg, crude) was obtained as a yellow solid.

### Step 2: 4-amino-6-chloro-N-(2-(4-methylpiperazin-1-yl)-5-(4-((3-morpholinopropyl)carbamoyl)-1H-1,2,3-triazol-1-yl)phenyl)nicotinamide (HYBI_212A)

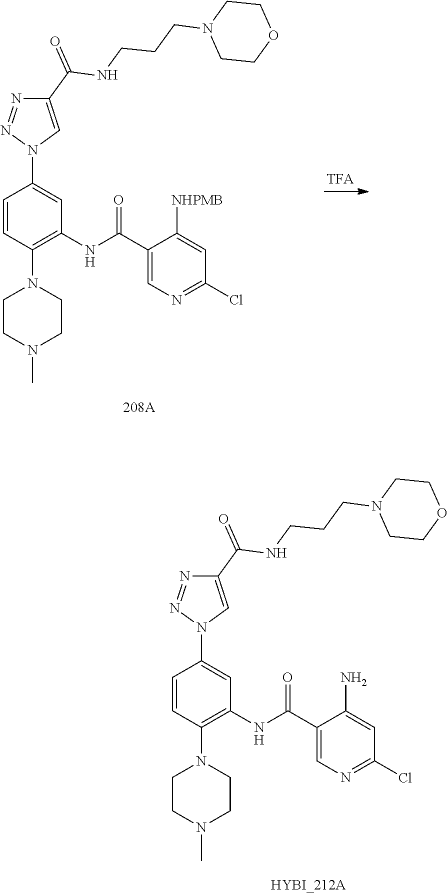

A mixture of compound 208A (140.00 mg, 199.08 umol, 1 eq) in TFA (3.08 g, 27.01 mmol, 2.00 mL, 135.68 eq) was stirred at 50° C. for 2 h. Water (5 mL) was added to the reaction mixture. The reaction mixture was then adjusted to pH=9 with aq. NaoH (1 N). The resulting mixture was extracted with DCM (5 mL*3). The combined organic phase was washed with brine (10 mL), dried over anhydrous Na₂SO₄, filtered and concentrated. The residue was purified by prep-HPLC (column: Phenomenex Gemini-NX C18 75*30 mm*3 um; mobile phase: [water(10 mM NH₄HCO₃)-ACN]; B %: 28%-48%, 7 min) and then further purified by SFC (column: DAICEL CHIRALPAK AS (250 mm*30 mm, 10 um); mobile phase: [0.1% NH₃H₂O ETOH]; B %: 30%-30%, min). HYBI_212A (5.3 mg, 8.84 umol, 4.44% yield, 97.25% purity) was obtained as a white solid.

¹H NMR (DMSO-d₆, 400 MHz) δ_{H}=9.70 (brs, 1H), 9.20-9.08 (m, 1H), 8.82 (t, J=5.6 Hz, 1H), 8.60 (d, J=2.4 Hz, 1H), 8.47 (s, 1H), 7.77-7.64 (m, 1H), 7.56-7.28 (m, 3H), 6.75 (s, 1H), 3.61 (t, J=4.4 Hz, 4H), 3.38-3.33 (m, 2H), 2.96-2.91 (m, 4H), 2.58-2.51 (m, 4H), 2.39-2.34 (m, 6H), 2.24 (s, 3H), 1.75-1.66 (m, 2H).

HPLC Rₜ=3.360 min in 8 min chromatography, purity 97.25%.

LCMS Rₜ=1.584 min in 4 min chromatography, purity 96.96%, MS ESI calcd. for 582.26, [M+H]⁺ 583.26, found 583.3.

### Example 23. 6-chloro-4-(dimethylamino)-N-[2-(4-methylpiperazin-1-yl)-5-[4-(3-morpholinopropylcarbamoyl)triazol-1-yl]phenyl]pyridine-3-carboxamide

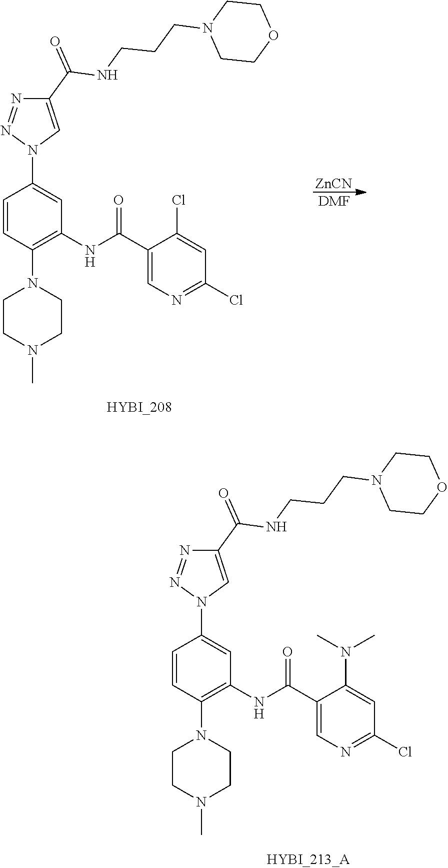

A mixture of HYBI_208 (100 mg, 0.017 mmol), (1E,4E)-1,5-diphenylpenta-1,4-dien-3-one; palladium (30.40 mg, 0.033 mmol), cyclopentyl(diphenyl)phosphane; iron (36.80 mg, 0.066 mol) and Zn(CN)₂ (80 mg, 0.68 mmol) in DMF (3 mL) was stirred at 120° C. for 1 h. The residue was diluted with H₂O (50 mL), and the mixture was extracted with EtOAc (50 mL×2). The combined organic phase was washed with water (20 mL) and brine (20 mL), dried over anhydrous Na₂SO₄, filtered and concentrated to give the crude product. The crude product was purified by Prep-HPLC (column: Phenomenex Gemini-NX C18 75×30 mm×3 um; mobile phase: [water(10 mM NH₄HCO₃)-ACN]; B %: 30%-60%, 10 min) to give HYBI_213_A (20 mg, 32.73 umol, 19.72% yield) as a yellow solid.

Note: Byproduct came from the decomposition of DMF at higher temperature.

¹H NMR (DMSO-d₆, 400 MHz) δ_{H}=9.70 (s, 1H), 9.19 (s, 1H), 8.73-8.85 (m, 2H), 8.24 (s, 1H), 7.70 (dd, J=8.8, 2.8 Hz, 1H), 7.44 (d, J=8.8 Hz, 1H), 6.93 (s, 1H), 3.61 (t, J=4.4 Hz, 4H), 3.33-3.39 (m, 2H), 2.98 (s, 6H), 2.91 (m, 4H), 2.44-2.49 (m, 4H), 2.33-2.39 (m, 6H), 2.24 (s, 3H), 1.65-1.76 (m, 2H).

HPLC Rₜ=1.884 min in 8 min chromatography, purity 96.28%.

LCMS Rₜ=1.832 min in 7 min chromatography, Xtimate C18, 3 m, 2.1×30 mm, purity 95.35%, MS ESI calcd. for 610.29 [M+H]⁺ 611.29, found 611.6.

### Example 24. 6-amino-4-methyl-N-(2-(4-methylpiperazin-1-yl)-5-(4-((3-morpholinopropyl)carbamoyl)-1H-1,2,3-triazol-1-yl)phenyl)nicotinamide

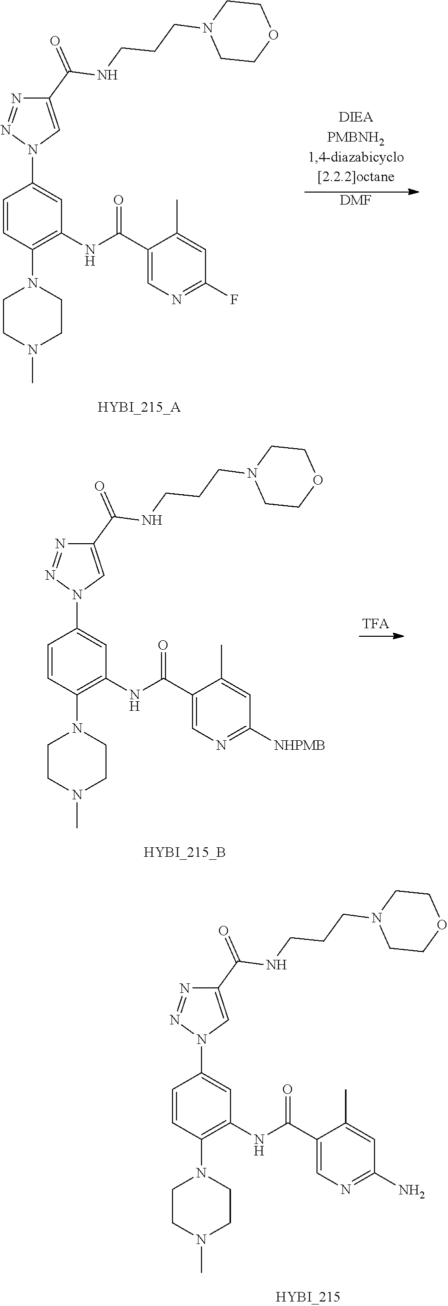

### Step 1: 6-((4-methoxybenzyl)amino)-4-methyl-N-(2-(4-methylpiperazin-1-yl)-5-(4-((3-morpholinopropyl)carbamoyl)-1H-1,2,3-triazol-1-yl)phenyl)nicotinamide (HYBI_215_B)

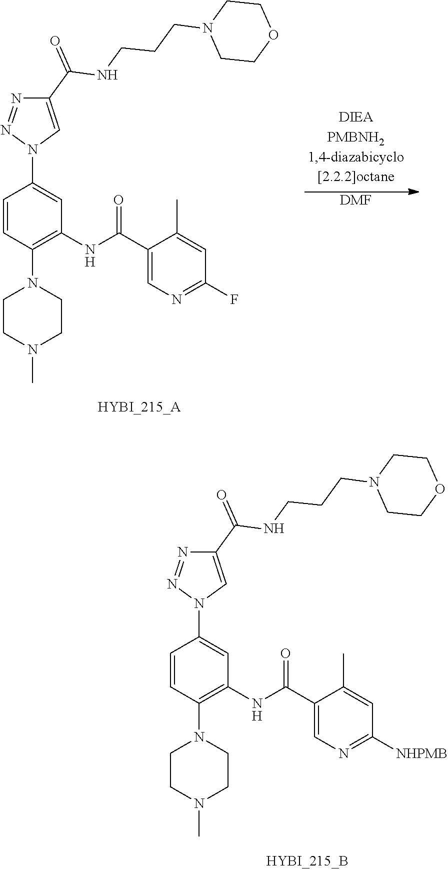

To a mixture of HYBI_215_A (210 mg, 371.26 umol, 1 eq) in DMF (3 mL) was added PMBNH₂ (50.93 mg, 371.26 umol, 48.05 uL, 1 eq), DIEA (143.95 mg, 1.11 mmol, 194.00 uL, 3 eq) and 1,4-diazabicyclo[2.2.2]octane (12.49 mg, 111.38 umol, 12.25 uL, 0.3 eq). The mixture was stirred at 80° C. for 1 h. Water (20 mL) was added to the residue. The resulting mixture was extracted with EtOAc (20 mL*3). The combined organic phase was washed with brine (20 mL), dried over anhydrous Na₂SO₄, filtered and concentrated. The crude product was purified by prep-HPLC (column: Waters Torus 2-PIC 150*19 mm*5 um; mobile phase: [Heptane-EtOH (0.1% NH3H₂O)]; B %: 0%-30%, 13 min). HYBI_215_B (30 mg, 39.50 umol, 10.64% yield, 89.9% purity) was obtained as a white solid.

LCMS Rₜ=2.028 min in 4 min chromatography, purity 89.90%, MS ESI calcd. for 682.37 [M+H]⁺ 683.37, found 683.5.

### Step 2: 6-amino-4-methyl-N-(2-(4-methylpiperazin-1-yl)-5-(4-((3-morpholinopropyl)carbamoyl)-1H-1,2,3-triazol-1-yl)phenyl)nicotinamide (HYBI_215)

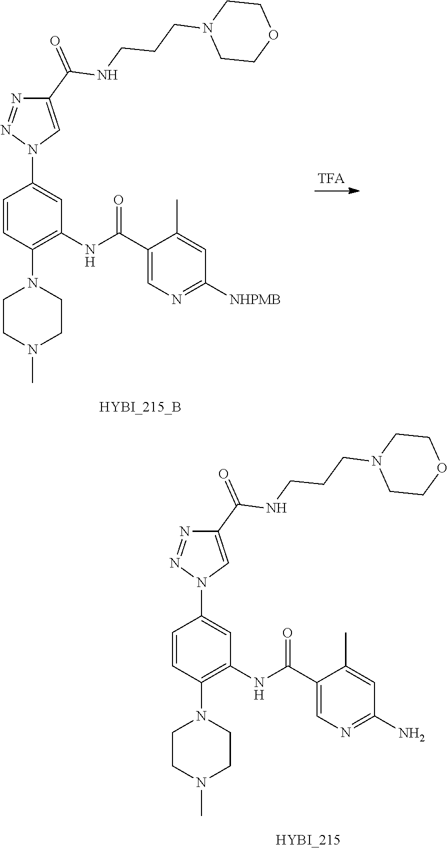

A mixture of HYBI_215_B (20 mg, 29.29 umol, 1 eq) and TFA (3.08 g, 27.01 mmol, 2.00 mL, 922.21 eq) was stirred at 50° C. for 1 h. The reaction mixture was concentrated directly. Water (2 mL) was added to the reaction mixture. The reaction mixture was then adjusted to pH˜9 by aq. NaOH (1 N) and concentrated to dryness. The residue was purified by prep-HPLC (column: Phenomenex Gemini-NX C18 75*30 mm*3 um; mobile phase: [water(10 mM NH4HCO3)-ACN]; B %: 20%-40%, 7 min). HYBI_215 (7.2 mg, 12.70 umol, 21.68% yield, 99.26% purity) was obtained as a white solid.

¹H NMR (DMSO-d₆, 400 MHz) δ_{H}=9.25 (s, 1H), 9.18 (s, 1H), 8.85-8.76 (m, 2H), 8.28 (s, 1H), 7.65 (dd, J=2.8, 8.8 Hz, 1H), 7.44 (d, J=8.8 Hz, 1H), 6.47 (s, 2H), 6.33 (s, 1H), 3.61 (t, J=4.4 Hz, 4H), 3.40-3.33 (m, 2H), 2.93 (t, J=4.4 Hz, 4H), 2.54-2.51 (m, 4H), 2.40-2.34 (m, 9H), 2.27-2.21 (m, 3H), 1.77-1.65 (m, 2H).

HPLC Rₜ=2.955 min in 8 min chromatography, purity 99.27%.

LCMS Rₜ=1.321 min in 4 min chromatography, purity 98.64%, MS ESI calcd. for 562.31, [M+H]⁺ 563.31, found 563.3.

### Example 25. 6-fluoro-4-methyl-N-(2-(4-methylpiperazin-1-yl)-5-(4-((3-morpholinopropyl)carbamoyl)-1H-1,2,3-triazol-1-yl)phenyl)nicotinamide

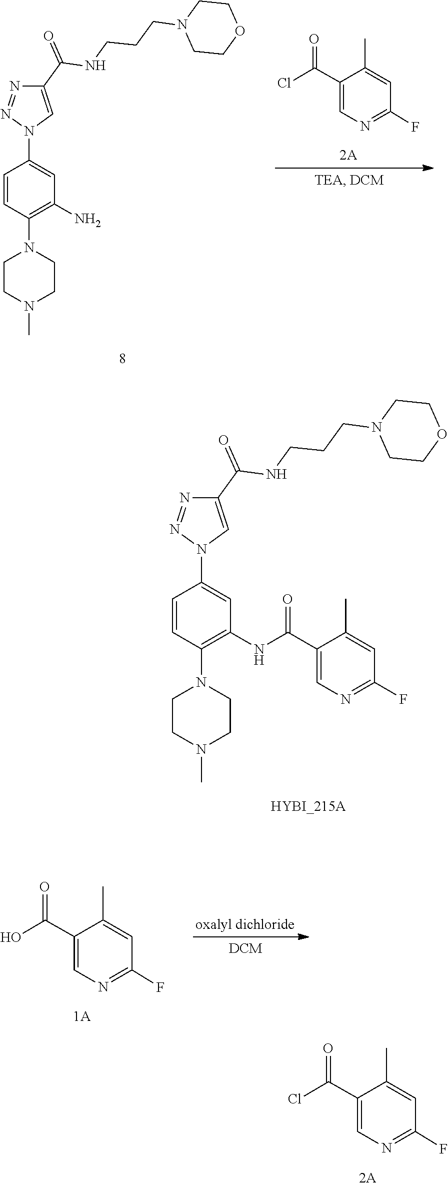 [SMILES: CN1CCN(c2ccc(-n3cc(C(=O)NCCCN4CCOCC4)nn3)cc2N)CC1 ; Cc1cc(F)ncc1C(=O)Cl ; Cc1cc(F)ncc1C(=O)Nc1cc(-n2cc(C(=O)NCCCN3CCOCC3)nn2)ccc1N1CCN(C)CC1 ; Cc1cc(F)ncc1C(=O)O ; Cc1cc(F)ncc1C(=O)Cl]

### Step 1: 6-fluoro-4-methylnicotinoyl chloride (Compound 2A)

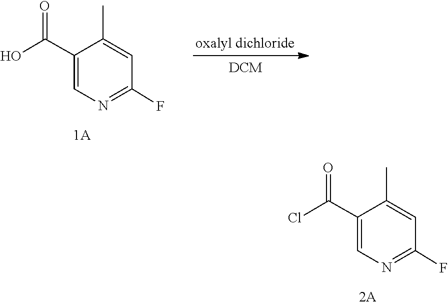

To a mixture of compound 1A (100 mg, 644.64 umol, 1 eq) in DCM (1 mL) was added DMF (5 mg, 64.46 umol, 4.96 uL, 0.1 eq). (COCl)₂ (409 mg, 3.22 mmol, 282.14 uL, 5 eq) was added into the above mixture at −10° C. The mixture was stirred at 10° C. for 1 hr. The mixture was concentrated to afford compound 2A (111 mg, 639.50 umol, 99.20% yield) as a brown solid, which was used to next step directly.

### Step 2: 6-fluoro-4-methyl-N-(2-(4-methylpiperazin-1-yl)-5-(4-((3-morpholinopropyl)carbamoyl)-1H-1,2,3-triazol-1-yl)phenyl)nicotinamide (HYBI_215A)

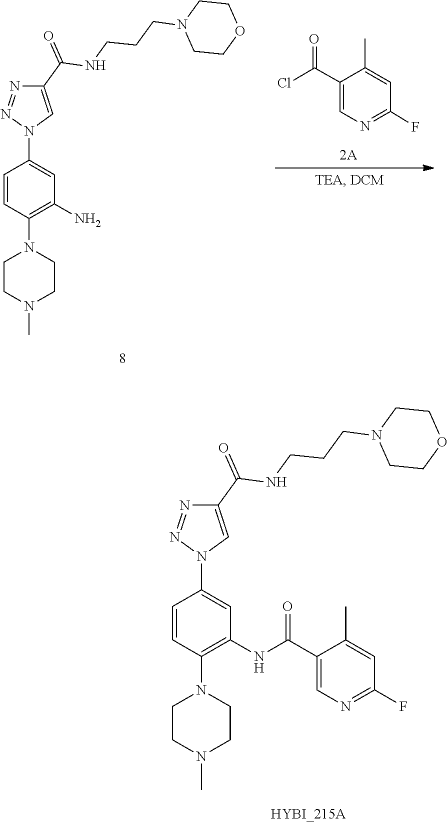 [SMILES: CN1CCN(c2ccc(-n3cc(C(=O)NCCCN4CCOCC4)nn3)cc2N)CC1 ; Cc1cc(F)ncc1C(=O)Cl ; Cc1cc(F)ncc1C(=O)Nc1cc(-n2cc(C(=O)NCCCN3CCOCC3)nn2)ccc1N1CCN(C)CC1]

To a mixture compound 2A (111 mg, 639.50 umol, 1.5 eq) in DCM (2 mL) was added compound 8 (183 mg, 426.34 umol, 1 eq) at 0° C. TEA (216 mg, 2.13 mmol, 296.71 uL, 5 eq) was added into the mixture at 0° C. The mixture was stirred at 10° C. for 1 hr. The mixture was concentrated to dryness. The mixture was purified with prep-HPLC (column: Phenomenex Gemini-NX C18 75*30 mm*3 um; mobile phase: [water (0.05% NH₃H₂O+10 mM NH₄HCO₃)-ACN]; B %: 17%-57%, 11 min). HYBI_215A (15.9 mg, 26.58 umol, 6.24% yield, 94.57% purity) was obtained as a light yellow solid.

¹H NMR (DMSO-d₆, 400 MHz) δ_{H}=9.75 (s, 1H) 9.21 (s, 1H) 8.83 (t, J=5.6 Hz, 1H) 8.52-8.65 (m, 1H) 8.44 (s, 1H) 7.76 (dd, J=8.4, 2.4 Hz, 1H) 7.42 (d, J=8.8 Hz, 1H) 7.24 (s, 1H) 3.62 (t, J=4.4 Hz, 4H) 3.30 (s, 2H) 2.96 (t, J=4.4 Hz, 4H) 2.54 (brs, 3H) 2.46-2.49 (m, 4H) 2.35-2.40 (m, 6H) 2.24 (s, 3H) 1.65-1.77 (m, 2H).

HPLC Rₜ=3.122 min in 8 min chromatography, Ultimate XB-C18 3.0*50 mm, 3 um, purity 94.57%.

LCMS Rₜ=1.610 min in 4 min chromatography, XBridge Shield RP18, 5 um, 2.1*50 mm, purity 94.31%, MS ESI calcd. for 565.29 [M+H]⁺ 566.29, found 566.3.

### Example 26. 2-methyl-N-(2-(4-methylpiperazin-1-yl)-5-(4-((3-morpholinopropyl)carbamoyl)-1H-1,2,3-triazol-1-yl)phenyl)-4-(trifluoromethyl)pyrimidine-5-carboxamide

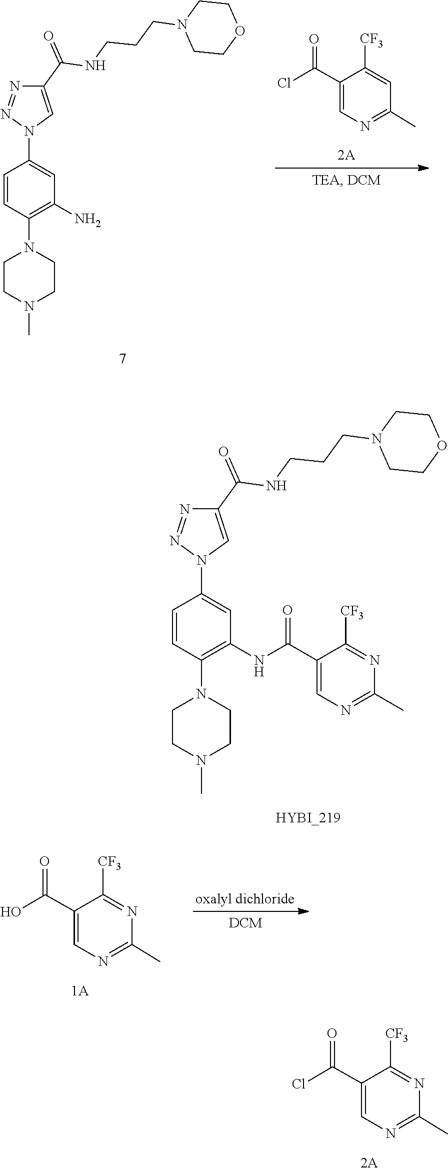 [SMILES: CN1CCN(c2ccc(-n3cc(C(=O)NCCCN4CCOCC4)nn3)cc2N)CC1 ; Cc1cc(C(F)(F)F)c(C(=O)Cl)cn1 ; Cc1ncc(C(=O)Nc2cc(-n3cc(C(=O)NCCCN4CCOCC4)nn3)ccc2N2CCN(C)CC2)c(C(F)(F)F)n1 ; Cc1ncc(C(=O)O)c(C(F)(F)F)n1 ; Cc1ncc(C(=O)Cl)c(C(F)(F)F)n1]

Note: The preparation method of compound 7 can be found in Example 1 above.

### Step 1: 2-methyl-4-(trifluoromethyl)pyrimidine-5-carbonyl chloride (Compound 2A)

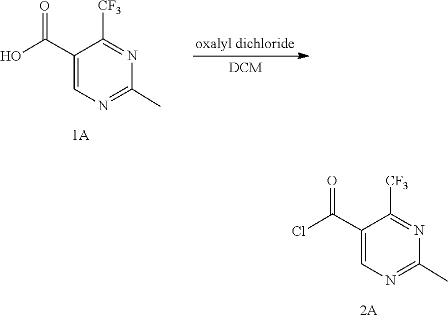

To a solution of compound 1A (200 mg, 970.30 umol, 1 eq) in DCM (2 mL) and DMF (one drop) was added oxalyl dichloride (615.78 mg, 4.85 mmol, 424.68 uL, 5 eq) at 0° C. The mixture was stirred at 20° C. for 30 min. The reaction mixture was concentrated directly. The product was used in the next step without further purification. Compound 2A (210 mg, 935.13 umol, 96.38% yield) was obtained as a brown solid.

### Step 2: 2-methyl-N-(2-(4-methylpiperazin-1-yl)-5-(4-((3-morpholinopropyl)carbamoyl)-1H-1,2,3-triazol-1-yl)phenyl)-4-(trifluoromethyl)pyrimidine-5-carboxamide (HYBI_219)

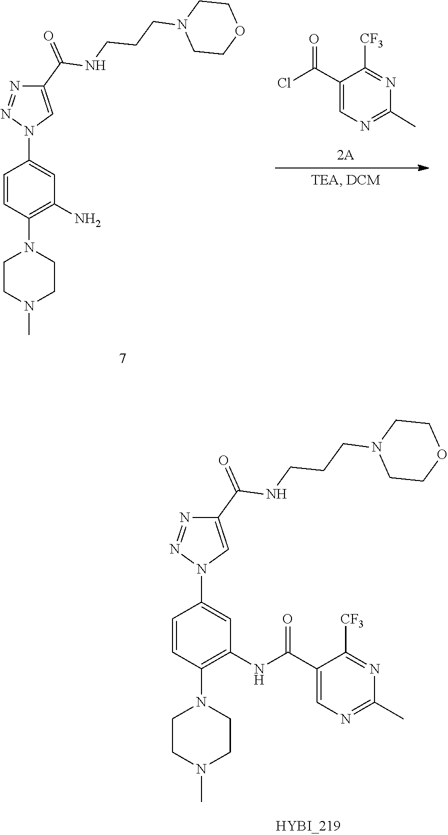 [SMILES: CN1CCN(c2ccc(-n3cc(C(=O)NCCCN4CCOCC4)nn3)cc2N)CC1 ; Cc1ncc(C(=O)Cl)c(C(F)(F)F)n1 ; Cc1ncc(C(=O)Nc2cc(-n3cc(C(=O)NCCCN4CCOCC4)nn3)ccc2N2CCN(C)CC2)c(C(F)(F)F)n1]

To a mixture of compound 7 (210 mg, 935.13 umol, 1.4 eq) in DCM (3 mL) was added TEA (337.95 mg, 3.34 mmol, 464.85 uL, 5 eq) at −10° C. The reaction mixture was stirred at 25° C. for 1 h. The reaction mixture was concentrated directly. The residue was purified by prep-HPLC (column: Phenomenex Gemini-NX C18 75*30 mm*3 um; mobile phase: [water(10 mM NH₄HCO₃)-ACN]; B %: 30%-50%, 7 min). Compound HYBI_219 (82.7 mg, 133.98 umol, 20.06% yield, 99.90% purity) was obtained as a white solid.

¹H NMR (DMSO-d₆, 400 MHz) δ_{H}=10.07 (s, 1H), 9.21 (s, 1H), 9.18 (s, 1H), 8.83 (t, J=5.6 Hz, 1H), 8.54 (d, J=2.4 Hz, 1H), 7.75 (dd, J=2.8, 8.8 Hz, 1H), 7.73 (d, J=8.8 Hz, 1H), 3.60 (t, J=4.4 Hz, 4H), 3.31-3.24 (m, 2H), 3.00-2.90 (m, 4H), 2.83 (s, 3H), 2.49-2.45 (m, 4H), 2.43-2.31 (m, 6H), 2.22 (s, 3H), 1.76-1.65 (m, 2H).

HPLC Rₜ=3.483 min in 8 min chromatography, purity 98.85%.

LCMS Rₜ=1.676 min in 4 min chromatography, purity 96.19%, MS ESI calcd. for 616.28, [M+H]⁺ 617.28, found 617.5.

### Example 27 and 28. N-(2-(4-methylpiperazin-1-yl)-5-(4-((3-morpholinopropyl)carbamoyl)-1H-1,2,3-triazol-1-yl)phenyl)-2-(methylthio)-4-(trifluoromethyl)pyrimidine-5-carboxamide (HYBI_221) and N-(2-(4-methylpiperazin-1-yl)-5-(4-((3-morpholinopropyl)carbamoyl)-1H-1,2,3-triazol-1-yl)phenyl)-2-(methylsulfinyl)-4-(trifluoromethyl)pyrimidine-5-carboxamide (HYBI_222A)

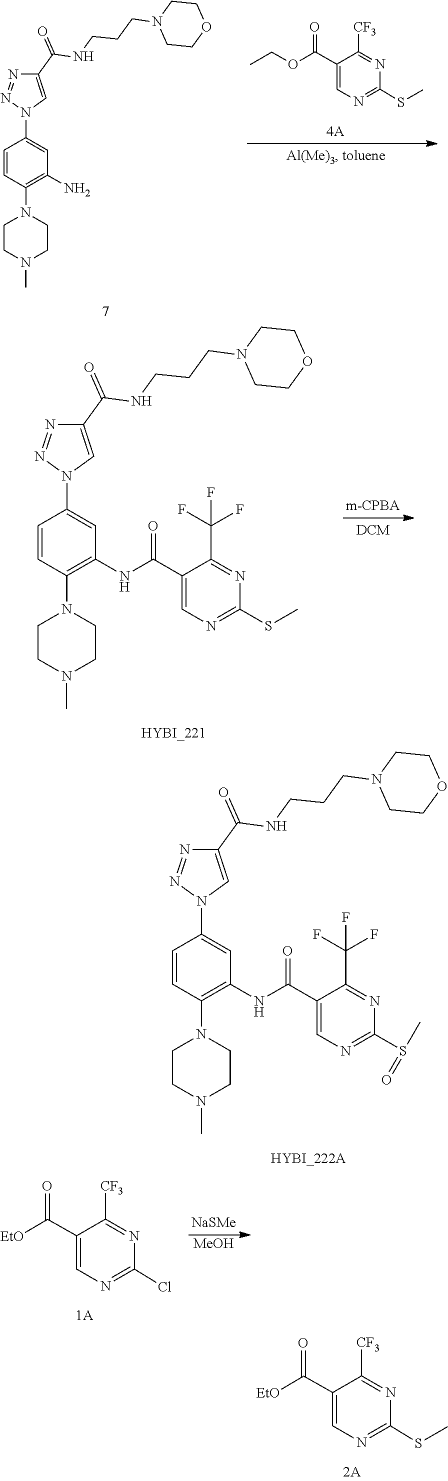

Note: The preparation method of compound 7 can be found in Example 1 above.

### Step 1: 2-methylsulfanyl-4-(trifluoromethyl)pyrimidine-5-carboxylate (Compound 2A)

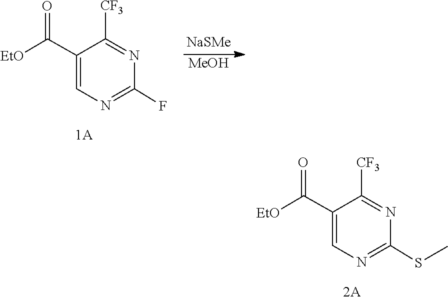 [SMILES: "MeOH" ; CCOC(=O)c1cnc(F)nc1C(F)(F)F ; CCOC(=O)c1cnc(SC)nc1C(F)(F)F ; "NaSMe"]

To the mixture of NaSMe (144.24 mg, 2.06 mmol, 131.13 uL, 1.05 eq) in MeOH (5 mL) was added compound 1A (500 mg, 1.96 mmol, 1 eq) at 15° C. The mixture was stirred at 50° C. for 1 hours. The reaction mixture was quenched by H₂O (20 mL) at 15° C., and extracted with EtOAc (10 mL*2). The combined organic layers were washed with brine (15 mL*2), dried over Na₂SO₄, filtered and concentrated under reduced pressure to give a residue. Compound 2A (500 mg, 1.88 mmol, 95.82% yield) was obtained as yellow oil.

¹H NMR (CDCl₃, 400 MHz) δ_{H}=9.07-8.97 (m, 1H), 4.44-4.39 (m, 2H), 2.64 (s, 3H), 1.42-1.37 (m, 3H)

### Step 2: N-(2-(4-methylpiperazin-1-yl)-5-(4-((3-morpholinopropyl)carbamoyl)-1H-1,2,3-triazol-1-yl)phenyl)-2-(methylthio)-4-(trifluoromethyl)pyrimidine-5-carboxamide (HYBI_221)

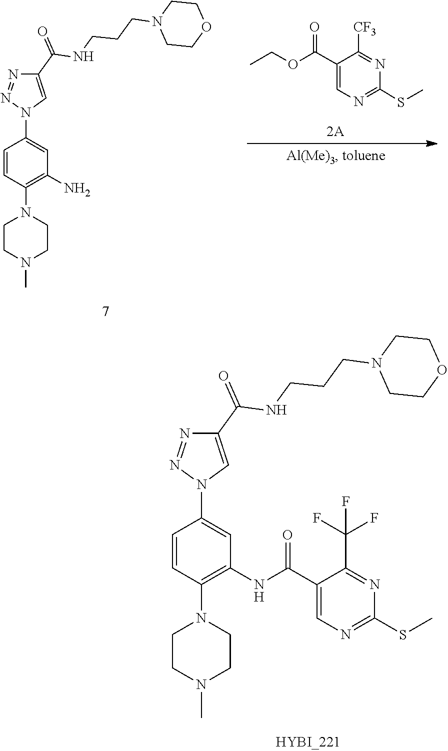 [SMILES: CN1CCN(c2ccc(-n3cc(C(=O)NCCCN4CCOCC4)nn3)cc2N)CC1 ; CCOC(=O)c1cnc(SC)nc1C(F)(F)F ; CSc1ncc(C(=O)Nc2cc(-n3cc(C(=O)NCCCN4CCOCC4)nn3)ccc2N2CCN(C)CC2)c(C(F)(F)F)n1]

To a solution of compound 7 (500 mg, 1.17 mmol, 1 eq) in toluene (4 mL) was added dropwise Al(CH₃)₃ (2 M, 1.46 mL, 2.5 eq) at 0° C. After addition, the mixture was stirred at this temperature for 30 min, and compound 2A (310.64 mg, 1.17 mmol, 1 eq) was added dropwise at 15° C. The resulting mixture was stirred at 100° C. for 16 hr. The reaction mixture was quenched by addition H₂O (1 mL) at 0° C., and then filtered. The filtrate was concentrated under reduced pressure to give a residue. The 1/5 residue was purified by Prep-HPLC (column: Phenomenex Gemini-NX C18 75*30 mm*3 um; mobile phase: [water(0.04% NH3H2O+10 mM NH4HCO3)-ACN]; B %: 44%-74%, 7 min) to give HYBI_221 (16.5 mg, 25.44 umol, 2.18% yield). The 4/5 residue was purified by flash silica gel chromatography (Silica Flash Column, Eluent of 0-10% MeOH/DCM) to give HYBI_221 (100 mg, 154.15 umol, 13.21% yield) was obtained as a white solid.

¹H NMR (CDCl₃, 400 MHz) δ_{H}=9.10 (s, 1H), 8.96-8.92 (m, 1H), 8.91 (s, 1H), 8.53 (s, 1H), 8.51-8.44 (m, 1H), 7.68-7.57 (m, 1H), 7.48-7.41 (m, 1H), 3.91-3.80 (m, 4H), 3.65-3.57 (m, 2H), 3.05-2.90 (m, 4H), 2.68 (s, 3H), 2.64-2.48 (m, 9H), 2.38 (s, 3H), 1.90-1.79 (m, 2H).

HPLC Rₜ=1.93 min in 8 min chromatography, Ultimate C18 3*50 mm 3 um, purity 99.40%.

LCMS Rₜ=1.42 min in 4 min chromatography, Xtimate C18 2.1*30 mm, 3 um, purity 99.37%, MS ESI calcd. for 648.26 [M+H]⁺ 649.26, found 649.2.

### Step 3: N-(2-(4-methylpiperazin-1-yl)-5-(4-((3-morpholinopropyl)carbamoyl)-1H-1,2,3-triazol-1-yl)phenyl)-2-(methylsulfinyl)-4-(trifluoromethyl)pyrimidine-5-carboxamide (HYBI_222A)

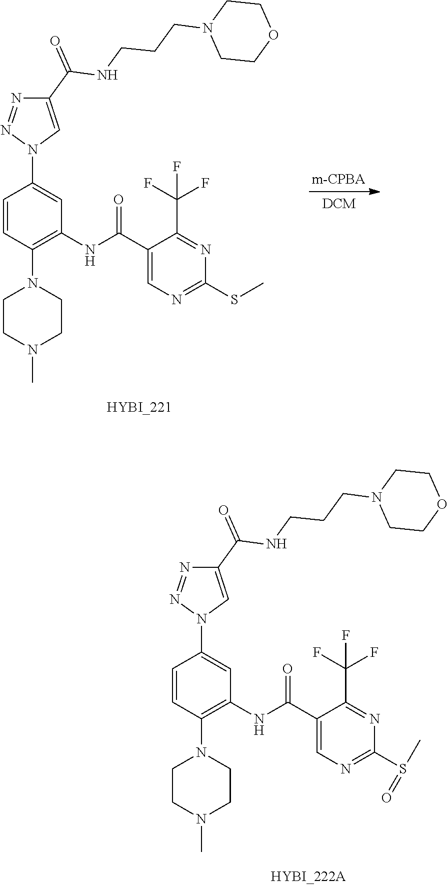 [SMILES: CSc1ncc(C(=O)Nc2cc(-n3cc(C(=O)NCCCN4CCOCC4)nn3)ccc2N2CCN(C)CC2)c(C(F)(F)F)n1 ; CN1CCN(c2ccc(-n3cc(C(=O)NCCCN4CCOCC4)nn3)cc2NC(=O)c2cnc(S(C)=O)nc2C(F)(F)F)CC1]

To a solution of HYBI_221 (50.00 mg, 77.08 umol, 1 eq) in DCM (5 mL) was added m-CPBA (31.30 mg, 154.15 umol, 85% purity, 2 eq). The mixture was stirred at 15° C. for 16 hr. The mixture was concentrated. The residue was purified by prep-HPLC (column: Phenomenex Gemini-NX C18 75*30 mm*3 um; mobile phase: [water(0.04% NH₃H₂O+10 mM NH₄HCO₃)-ACN]; B %: 28%-58%, 7 min) and prep-HPLC (column: Welch Xtimate C18 150*30 mm*5 um; mobile phase: [water(0.05% NH₃H₂O+10 mM NH₄HCO₃)-ACN]; B %: 13%-43%, 9 min) to give HYBI_222A (5 mg, 7.52 umol, 9.76% yield) was obtained as a white solid.

¹H NMR (CDCl₃, 400 MHz) δ_{H}=9.38-9.15 (m, 1H), 8.97-8.88 (m, 2H), 8.63-8.52 (m, 2H), 7.72-7.63 (m, 1H), 7.63-7.55 (m, 1H), 3.91-3.74 (m, 6H), 3.66-3.54 (m, 4H), 3.53-3.42 (m, 2H), 3.40-3.30 (m, 3H), 2.92-2.81 (m, 2H), 2.64 (s, 3H), 2.58-2.45 (m, 6H), 1.90-1.79 (m, 2H).

HPLC Rₜ=1.92 min in 8 min chromatography, Ultimate C18 3*50 mm 3 um, purity 99.12%.

LCMS Rₜ=1.42 min in 4 min chromatography, Xtimate C18 2.1*30 mm, 3 um, purity 99.56%, MS ESI calcd. for 664.25 [M+H]⁺ 665.25, found 665.1.

### Example 29. 2-ethoxy-N-(2-(4-methylpiperazin-1-yl)-5-(4-((3-morpholinopropyl)carbamoyl)-1H-1,2,3-triazol-1-yl)phenyl)-4-(trifluoromethyl)pyrimidine-5-carboxamide

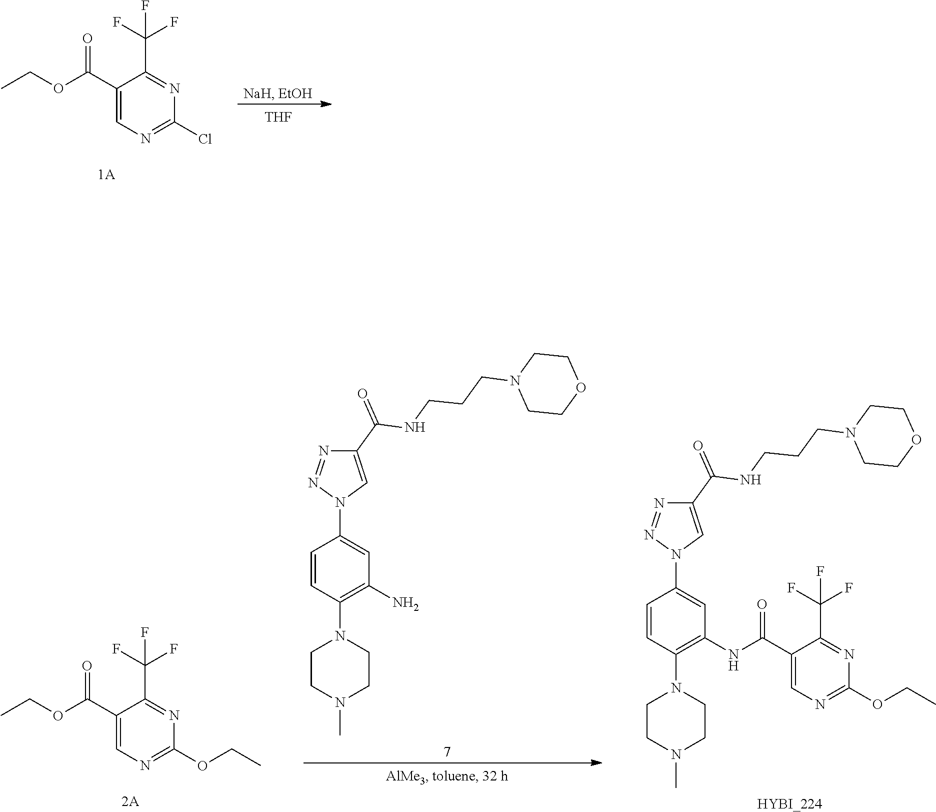

Note: The preparation method of compound 7 can be found in Example 1 above.

### Step 1: ethyl 2-ethoxy-4-(trifluoromethyl)pyrimidine-5-carboxylate (Compound 2A)

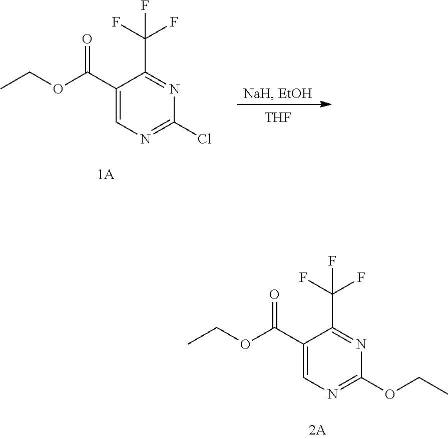

To the mixture of NaH (392.75 mg, 9.82 mmol, 60% purity, 5 eq) in THF (5 mL) was added EtOH (452.37 mg, 9.82 mmol, 572.62 uL, 5 eq). After stirred at 0° C. for 0.5 hour, 1A (500 mg, 1.96 mmol) was wadded. The mixture was stirred at 15° C. for 1 hours. The reaction mixture was quenched by addition H₂O (10 mL*2) at 15° C., and extracted with EtOAc (10 mL*2). The combined organic layers were washed with bine (5 mL*2), dried over Na₂SO₄, filtered and concentrated under reduced pressure to give 2A (380 mg, 1.44 mmol, 73.24% yield) was obtained as yellow oil.

¹H NMR (CDCl₃, 400 MHz) δ_{H}=9.08 (s, 1H), 4.60 (q, J=7.2 Hz, 2H), 4.40 (q, J=7.2 Hz, 2H), 1.48 (t, J=7.2 Hz, 3H), 1.40 (t, J=7.2 Hz, 3H)

### Step 2: 2-ethoxy-N-(2-(4-methylpiperazin-1-yl)-5-(4-((3-morpholinopropyl)carbamoyl)-1H-1,2,3-triazol-1-yl)phenyl)-4-(trifluoromethyl)pyrimidine-5-carboxamide (HYBI_224)

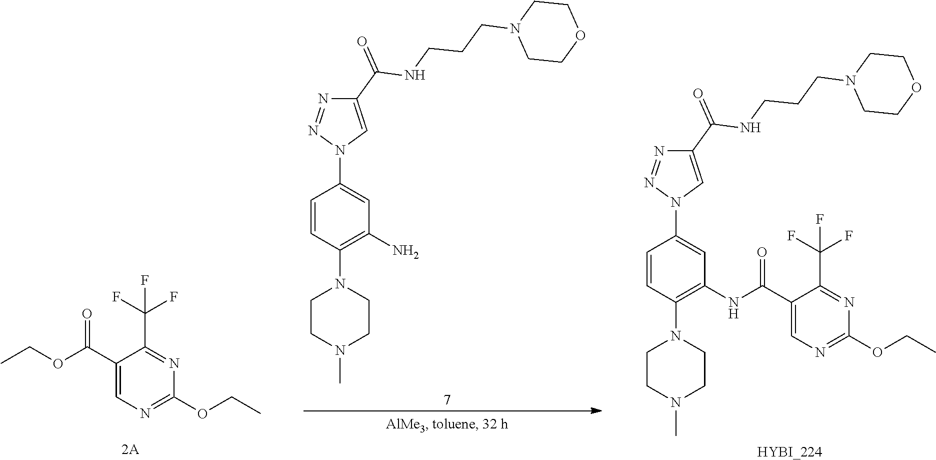

To a solution of compound 7 (145.98 mg, 340.65 umol) in toluene (2 mL) was added dropwise Al(CH₃)₃ (2 M, 170.33 uL, 1 eq) at 0° C. over 30 min. After addition, and then compound 2A (27.00 mg, 102.19 umol, 0.3 eq) was added at 0° C. The resulting mixture was stirred at 100° C. for 16 hr. To a solution was added Al(CH₃)₃ (2 M, 510.97 uL, 3 eq) and compound 2A (90.00 mg, 340.65 umol). The reaction mixture was quenched by addition H₂O (0.2 mL) at 0° C., and then filtered. The filtrate was concentrated under reduced pressure to give a residue. The residue was purified by prep-HPLC (column: Phenomenex Gemini-NX C18 75*30 mm*3 um; mobile phase: [water (0.04% NH₃H₂O+10 mM NH₄HCO₃)-ACN]; B %: 25%-55%, 7 min) to give HYBI_224 (12.9 mg, 19.35 umol, 5.68% yield, 97.% purity) was obtained as a white solid.

¹H NMR (CDCl₃, 400 MHz) δ_{H}=9.08 (brs, 1H), 8.98-8.91 (m, 2H), 8.53 (s, 1H), 7.65-7.59 (m, 1H), 7.50-7.40 (m, 1H), 4.64-4.56 (m, 2H), 3.98-3.79 (m, 4H), 3.69-3.53 (m, 2H), 3.10-2.88 (m, 4H), 2.75-2.30 (m, 13H), 1.95-1.76 (m, 2H), 1.54-1.49 (m, 3H) HPLC Rₜ=97.73 min in 15 min chromatography, UltimateLP-C18 150*4.6 mm, 5 um, purity 97.73%.

LCMS Rₜ=1.374 min in 4 min chromatography, Xtimate C18 2.1*30 mm, 3 um purity 99%, MS ESI calcd. for 646.30 [M+H]⁺ 647.30, found 647.3.

### Example 30. 4-methoxy-N-(2-(4-methylpiperazin-1-yl)-5-(4-((3-morpholinopropyl)carbamoyl)-1H-1,2,3-triazol-1-yl)phenyl)-2-(methylthio)pyrimidine-5-carboxamide

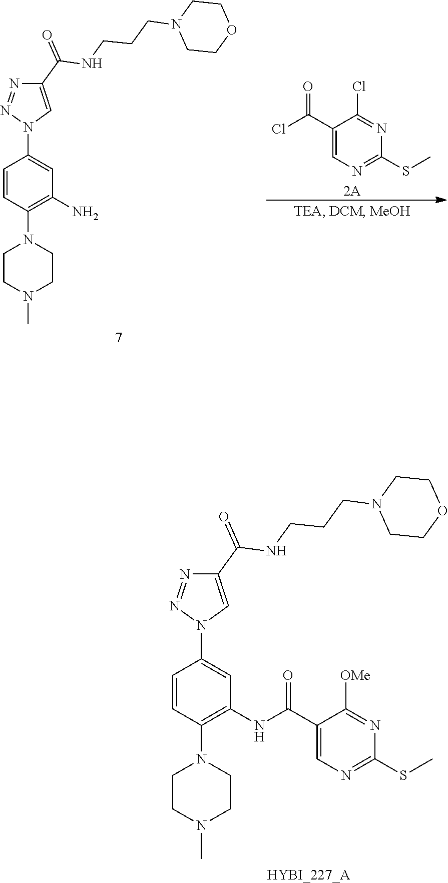

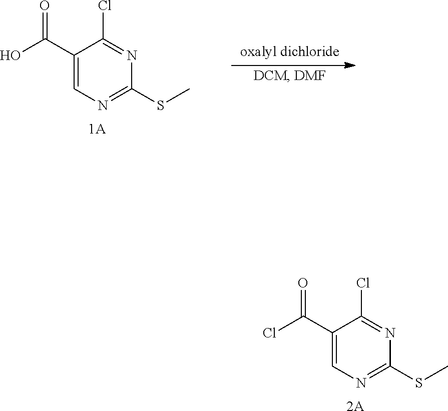

Note: The preparation method of compound 7 can be found in Example 1 above.

### Step 1: 4-chloro-2-(methylthio)pyrimidine-5-carbonyl chloride (Compound 2A)

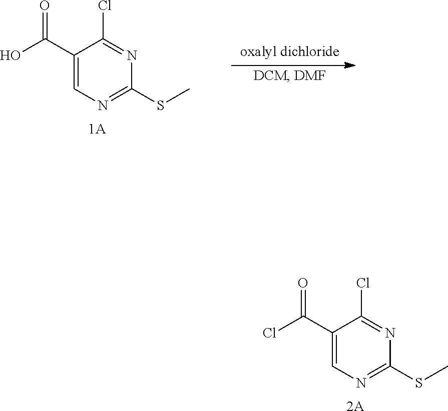

To a solution of compound 1A (400 mg, 1.95 mmol, 1 eq) and DMF (14.29 mg, 195.47 umol, 15.04 uL, 0.1 eq) in DCM (4 mL) was added oxalyl dichloride (1.24 g, 9.77 mmol, 855.56 uL, 5 eq) dropwise at 0° C. The reaction mixture was stirred at 25° C. for 20 min. The mixture was concentrated to remove DCM. The product was used in the next step without further purification. Compound 2A (430 mg, crude) was obtained as white solid.

### Step 2: 4-methoxy-N-(2-(4-methylpiperazin-1-yl)-5-(4-((3-morpholinopropyl)carbamoyl)-1H-1,2,3-triazol-1-yl)phenyl)-2-(methylthio)pyrimidine-5-carboxamide (HYBI_227_A)

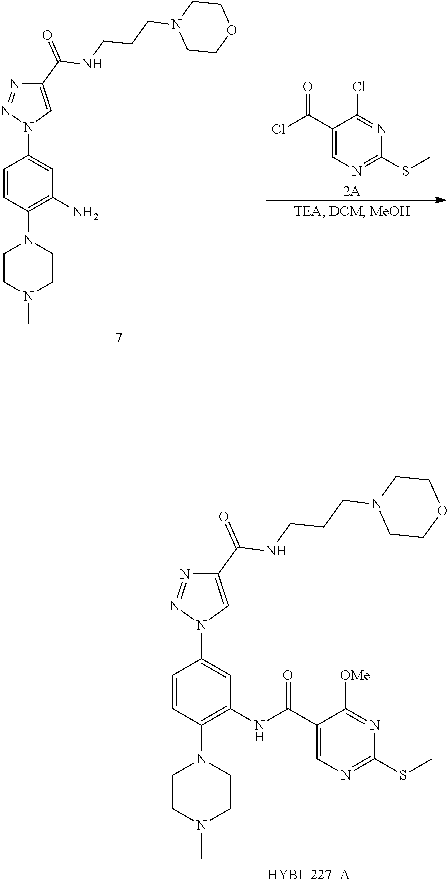

To a mixture of compound 7 (590.01 mg, 1.38 mmol, 1 eq) and compound 2A (430 mg, 1.93 mmol, 1.4 eq) in DCM (5 mL) was added TEA (696.60 mg, 6.88 mmol, 958.19 uL, 5 eq) at −10° C. The reaction mixture was stirred at 25° C. for 20 min. The mixture was concentrated to remove DCM. MeOH (5 mL) was added to the residue and the mixture was purified by prep-HPLC column: Phenomenex Gemini-NX C18 75*30 mm*3 um; mobile phase: [water(10 mM NH4HCO3)-ACN]; B %: 30%-60%, 10 min, which was further separated by SFC (condition: DAICEL CHIRALPAK AD (250 mm*30 mm, 10 um); mobile phase: [0.1% NH3H2O IPA]; B %: 55%-55%, min) and prep-HPLC column: Phenomenex Gemini-NX C18 75*30 mm*3 um; mobile phase: [water(10 mM NH4HCO3)-ACN]; B %: 30%-55%, 8 min. HYBI_227A (14.2 mg, 23.15 umol, 1.68% yield, 93.43% purity) was obtained as a white solid.

¹H NMR (DMSO-d₆, 400 MHz) δ_{H}=10.03 (s, 1H), 9.25 (s, 1H), 9.01 (d, J=2.4 Hz, 1H), 8.94 (s, 1H), 8.88 (t, J=5.6 Hz, 1H), 7.73 (dd, J=2.4, 8.4 Hz, 1H), 7.51 (d, J=8.8 Hz, 1H), 4.25 (s, 3H), 3.74 (s, 4H), 3.48-3.41 (m, 2H), 3.22-3.07 (m, 7H), 3.06-2.65 (m, 10H), 2.62 (s, 3H), 1.97-1.77 (m, 2H).

HPLC Rₜ=3.977 min in 8 min chromatography, purity 93.43%.

LCMS Rₜ=1.817 min in 4 min chromatography, purity 90.14%, MS ESI calcd. for 610.28 [M+H]⁺ 611.28, found 611.3.

### Example 31. 4-methyl-N-(2-(4-methylpiperazin-1-yl)-5-(4-((3-morpholinopropyl)carbamoyl)-1H-1,2,3-triazol-1-yl)phenyl)-6-(trifluoromethyl)nicotinamide

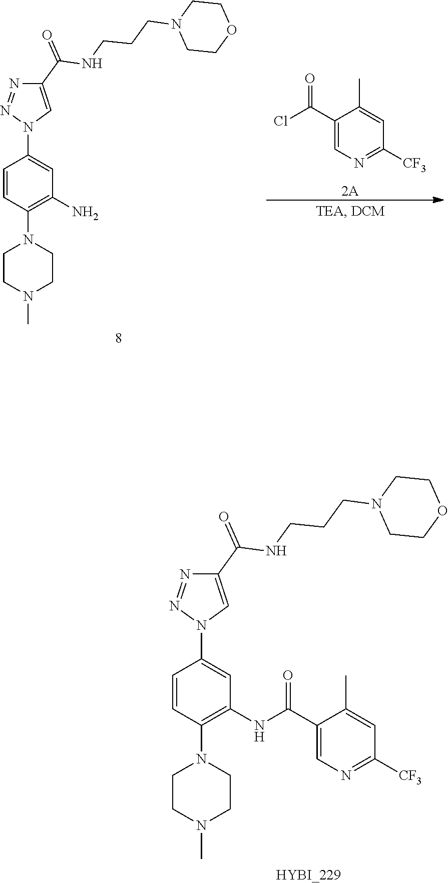

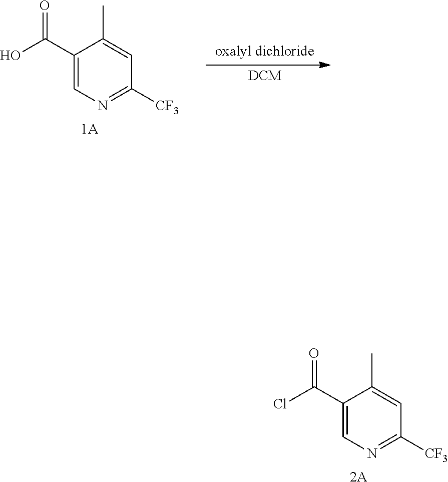

Note: The preparation method of compound 8 can be found in Example 1 above.

### Step 1: 4-methyl-6-(trifluoromethyl)nicotinoyl chloride (Compound 2A)

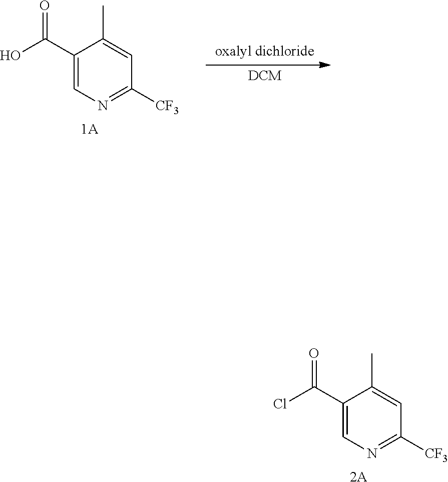

To a mixture of compound 1A (100 mg, 485.15 umol, 1 eq) in DCM (1 mL) was added DMF (35 mg, 485.15 umol, 37.33 uL, 1 eq). (COCl)₂ (308 mg, 2.43 mmol, 212.34 uL, 5 eq) was added into the above mixture at −10° C. The mixture was stirred at 10° C. for 20 mins. The mixture was concentrated to dryness. Compound 2A (108 mg, 480.92 umol, 99.13% yield) was obtained as a yellow solid.

### Step 2: 4-methyl-N-(2-(4-methylpiperazin-1-yl)-5-(4-((3-morpholinopropyl)carbamoyl)-1H-1,2,3-triazol-1-yl)phenyl)-6-(trifluoromethyl)nicotinamide (HYBI_229)

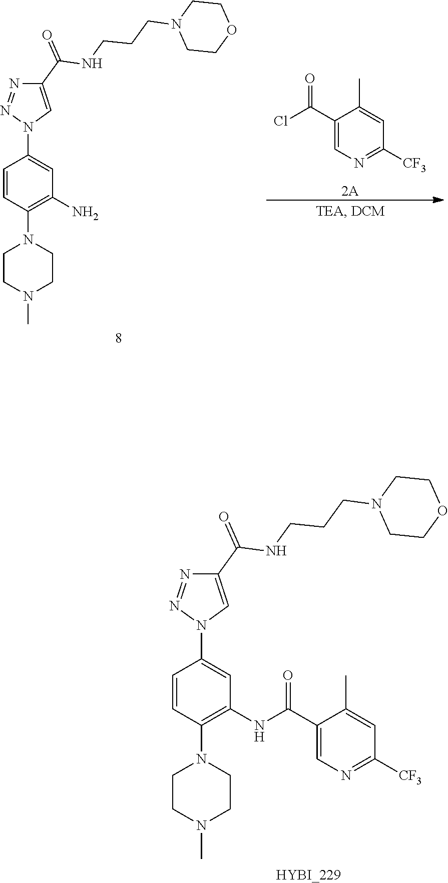

To a mixture of compound 2A (108 mg, 480.92 umol, 1.5 eq) in DCM (2 mL) was added compound 8 (138 mg, 320.62 umol, 1 eq) at 0° C. TEA (162 mg, 1.60 mmol, 223.13 uL, 5 eq) was added into the mixture at 0° C. The mixture was stirred at 10° C. for 20 mins. The mixture was concentrated to dryness. The mixture was purified with prep-HPLC (column: Phenomenex Gemini-NX C18 75*30 mm*3 um; mobile phase: [water(0.05% NH₃H₂O+10 mM NH₄HCO₃)-ACN]; B %: 26%-56%, 11 min). HYBI_229 (85.1 mg, 136.96 umol, 42.72% yield, 99.24% purity) was obtained as a yellow solid.

¹H NMR (DMSO-d₆, 400 MHz) δ_{H}=9.22 (s, 1H) 9.13 (s, 1H) 8.78-8.87 (m, 1H) 8.60 (s, 1H) 7.71-7.86 (m, 1H) 7.41 (d, J=8.8 Hz, 1H) 3.62 (t, J=4.0 Hz, 4H) 3.35-3.40 (m, 2H) 3.30-3.10 (m, 4H), 3.00-2.85 (m, 4H) 2.75 (s, 3H) 2.34-2.41 (m, 2H) 2.40-2.25 (m, 4H) 2.24 (s, 3H) 1.64-1.67-1.76 (m, 2H).

HPLC Rₜ=3.608 min in 8 min chromatography, purity 99.24%.

LCMS Rₜ=1.764 min in 4 min chromatography, Chromolith Flash RP-18.5 um, 3.0*25 mm, purity 99.68%, MS ESI calcd. for 616.28 [M+H]⁺ 617.18, found 617.3.

### Example 32. 4,6-dichloro-N-(2-(4-methylpiperazin-1-yl)-5-(4-((3-morpholinopropyl)carbamoyl)-1H-1,2,3-triazol-1-yl)phenyl)pyridazine-3-carboxamide

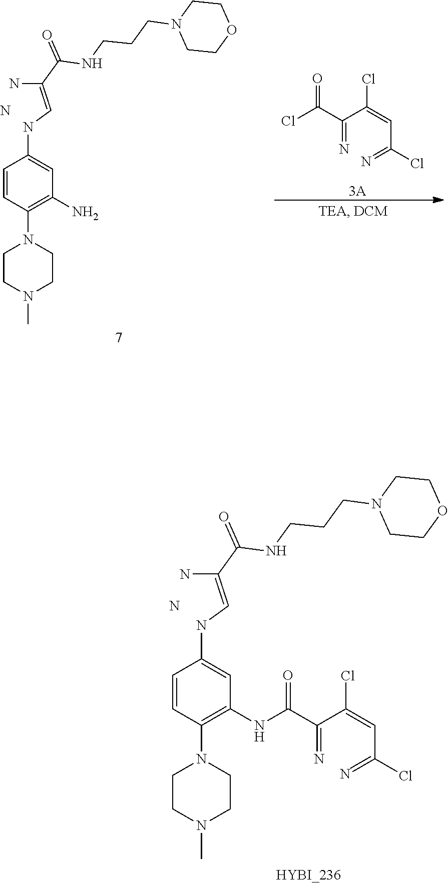

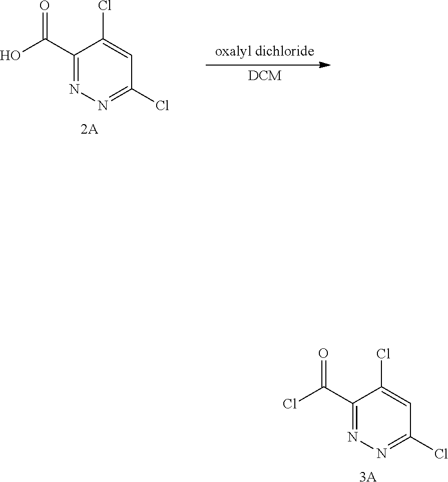

Note: The preparation method of compound 7 can be found in Example 1 above.

### Step 1: 4,6-dichloropyridazine-3-carbonyl chloride (Compound 3A)

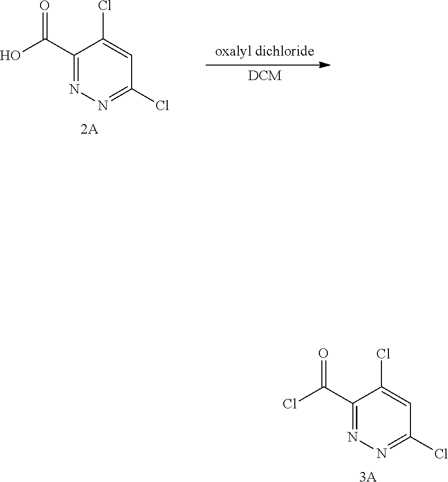

To a solution of compound 2A (500 mg, 2.59 mmol, 1 eq) in DCM (5 mL) and DMF (one drop) was added oxalyl dichloride (1.64 g, 12.95 mmol, 1.13 mL, 5 eq) at 0° C. The mixture was stirred at 20° C. for 30 min. The reaction mixture was concentrated directly. The residue was used to the next step directly. Compound 3A (540 mg, 2.55 mmol, 98.58% yield) was obtained as a yellow oil.

### Step 2: 4,6-dichloro-N-(2-(4-methylpiperazin-1-yl)-5-(4-((3-morpholinopropyl)carbamoyl)-1H-1,2,3-triazol-1-yl)phenyl)pyridazine-3-carboxamide (Compound HYBI_236)

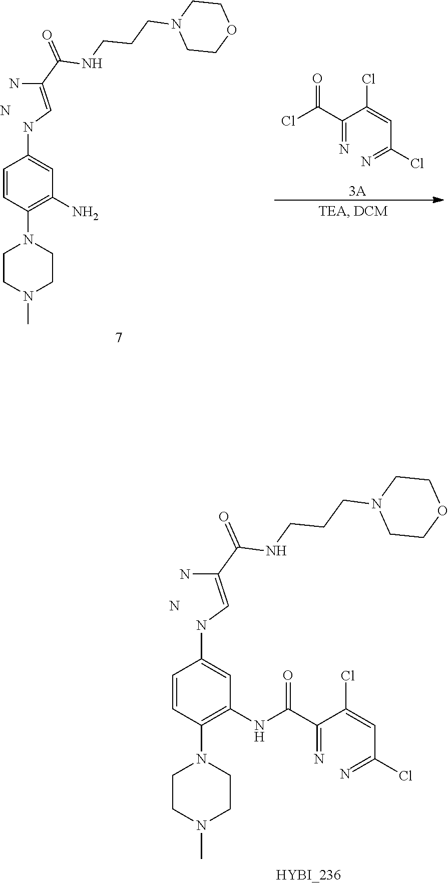

To a solution of compound 3A (540 mg, 2.55 mmol, 1.4 eq) in DCM (5 mL) was added compound 7 (781.76 mg, 1.82 mmol, 1 eq) and TEA (922.99 mg, 9.12 mmol, 1.27 mL, 5 eq) at −10° C. The mixture was stirred at 25° C. for 2 h. The reaction mixture was concentrated directly. The residue was purified by flash silica gel chromatography (eluent of 0-10% MeOH/DCM. The crude product was purified by prep-HPLC (column: Phenomenex Gemini-NX C18 75*30 mm*3 um; mobile phase: [water(10 mM NH₄HCO₃)-ACN]; B %: 32%-60%, 9 min). HYBI_236 (2100 mg, 3.32 mmol, 91.03% yield, 95.45% purity) was obtained as a white solid.

¹H NMR (DMSO-d₆, 400 MHz) δ_{H}=10.59 (s, 1H), 9.22 (s, 1H), 8.88-8.78 (m, 2H), 8.58 (s, 1H), 7.79-7.70 (m, 1H), 7.46 (d, J=8.8 Hz, 1H), 3.66-3.57 (m, 4H), 3.38-3.34 (m, 2H), 2.97 (s, 4H), 2.62-2.54 (m, 4H), 2.42-2.32 (m, 6H), 2.26 (s, 3H), 1.77-1.65 (m, 2H).

HPLC Rₜ=3.595 min in 8 min chromatography, purity 95.45%.

LCMS Rₜ=1.812 min in 4 min chromatography, purity 97.79%, MS ESI calcd. for 602.20, [M+H]⁺ 603.20 found 603.2.

### Example 33. 4-amino-6-chloro-N-(2-(4-methylpiperazin-1-yl)-5-(4-((3-morpholinopropyl)carbamoyl)-1H-1,2,3-triazol-1-yl)phenyl)pyridazine-3-carboxamide

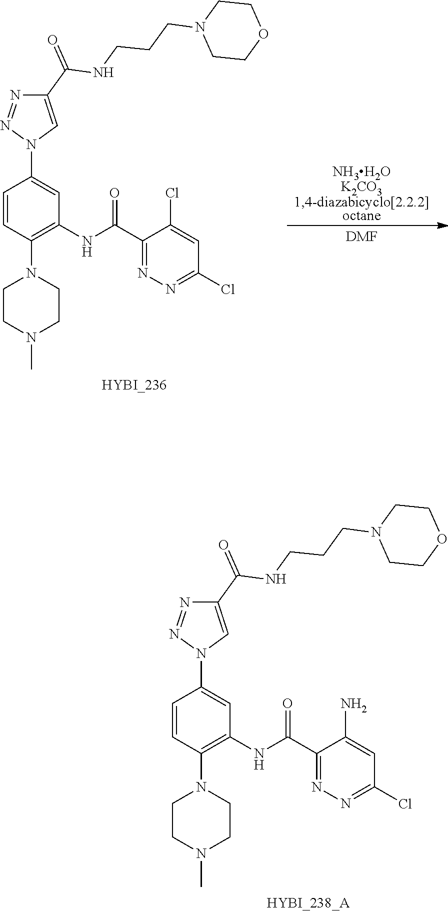

### Step 1: 4-amino-6-chloro-N-(2-(4-methylpiperazin-1-yl)-5-(4-((3-morpholinopropyl)carbamoyl)-1H-1,2,3-triazol-1-yl)phenyl)pyridazine-3-carboxamide (HYBI_238_A)

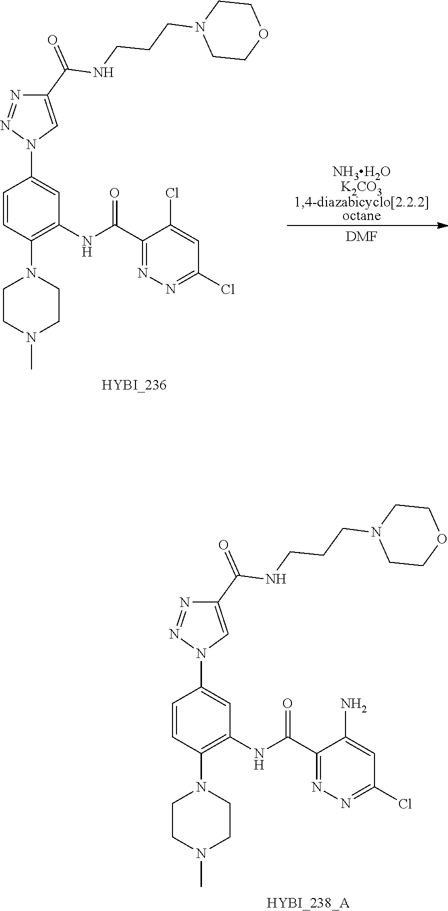

To a solution of HYBI_236 (300 mg, 497.10 umol, 1 eq) in DMF (3 mL) was added NH₃·H₂O (58.07 mg, 497.10 umol, 63.81 uL, 30% purity, 1 eq), 1,4-diazabicyclo[2.2.2]octane (16.73 mg, 149.13 umol, 16.40 uL, 0.3 eq) and K₂CO₃ (206.11 mg, 1.49 mmol, 3 eq). The mixture was stirred at 80° C. for 1 hr. The reaction mixture was filtered. The filtrate was concentrated to dryness directly. The residue was purified by prep-HPLC column: Phenomenex Gemini-NX C18 75*30 mm*3 um; mobile phase: [water(10 mM NH4HCO3)-ACN]; B %: 30%-50%, 7 min. HYBI_238_A (24.9 mg, 41.82 umol, 8.41% yield, 98.10% purity) was obtained as a white solid.

¹H NMR (DMSO-d₆, 400 MHz) δ_{H}=10.92 (s, 1H), 9.20 (s, 1H), 9.04 (d, J=2.8 Hz, 1H), 8.86 (t, J=5.6 Hz, 1H), 8.14-7.74 (m, 2H), 7.70 (dd, J=2.4, 8.8 Hz, 1H), 7.48 (d, J=8.8 Hz, 1H), 7.05 (s, 1H), 3.63 (t, J=4.8 Hz, 4H), 3.38-3.35 (m, 2H), 2.95 (t, J=4.8 Hz, 4H), 2.59 (s, 4H), 2.38 (t, J=6.8 Hz, 6H), 2.30 (s, 3H), 1.74-1.68 (m, 2H).

HPLC Rₜ=3.682 min in 8 min chromatography, purity 98.10%.

LCMS Rₜ=1.685 min in 4 min chromatography, purity 97.76%, MS ESI calcd. for 583.25 [M+H]⁺ 584.25, found 584.3.

### Example 34. 3,5-dichloro-N-[2-(4-methylpiperazin-1-yl)-5-[4-(3-morpholinopropylcarbamoyl)triazol-1-yl]phenyl]pyrazine-2-carboxamide

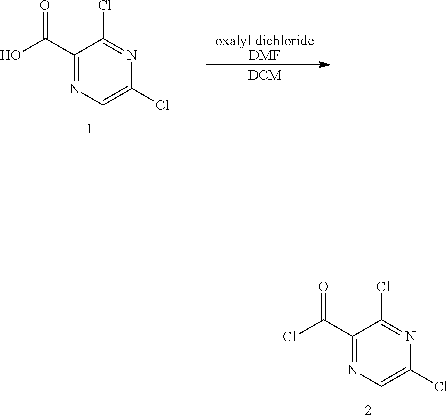

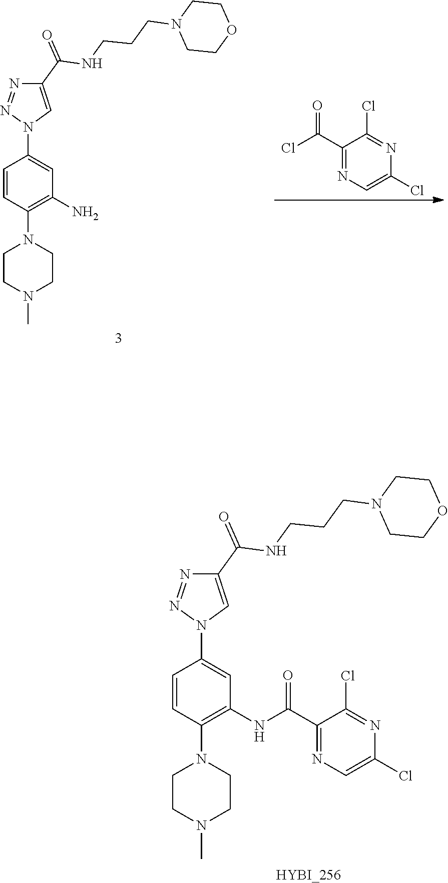

Note: The preparation method of compound 3 can be found in Example 1 above.

### Step 1: 3,5-dichloropyrazine-2-carbonyl chloride

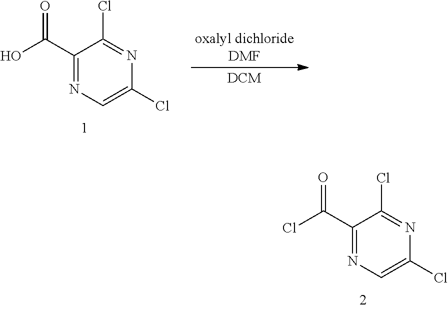 [SMILES: O=C(O)c1ncc(Cl)nc1Cl ; O=C(Cl)c1ncc(Cl)nc1Cl]

To a solution of compound 1 (200 mg, 1.04 mmol, 1 eq) in DCM (3 mL) was added DMF (38.0 mg, 519.88 umol, 0.04 mL, 5.02e-1 eq) and oxalyl dichloride (263.08 mg, 2.07 mmol, 181.43 uL, 2 eq) at 20° C. The mixture was stirred at 20° C. for 3 hours. The mixture was concentrated under reduced pressure to give an oil. Compound 2 (200 mg, crude) was obtained as a yellow oil, which was used into next step without purification.

### Step 2: 3,5-dichloro-N-[2-(4-methylpiperazin-1-yl)-5-[4-(3-morpholinopropylcarbamoyl)triazol-1-yl]phenyl]pyrazine-2-carboxamide (HYBI_256)

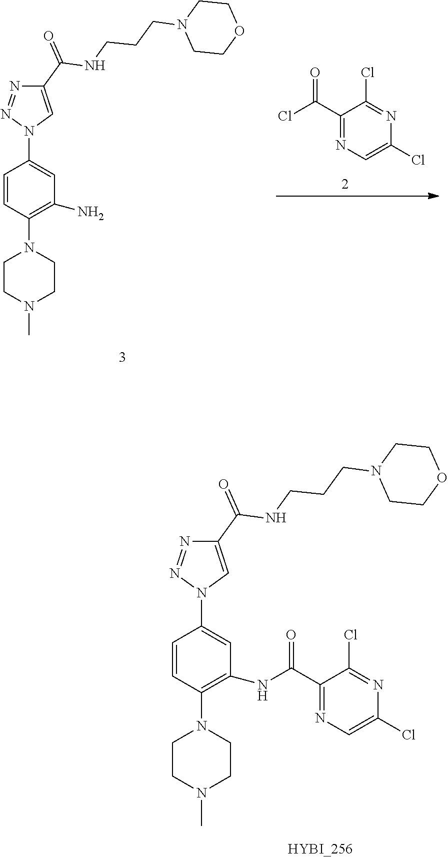 [SMILES: CN1CCN(c2ccc(-n3cc(C(=O)NCCCN4CCOCC4)nn3)cc2N)CC1 ; O=C(Cl)c1ncc(Cl)nc1Cl ; CN1CCN(c2ccc(-n3cc(C(=O)NCCCN4CCOCC4)nn3)cc2NC(=O)c2ncc(Cl)nc2Cl)CC1]

To a solution of compound 3 (200 mg, 466.71 umol, 1 eq) in DCM (2 mL) was added compound 2 (200 mg, 945.93 umol, 2.03 eq) in DCM (2 mL) at 0° C. The mixture was stirred at 20° C. for 12 hours. The mixture was concentrated under reduced pressure to give a residue. The residue was purified by prep HPLC (column: Phenomenex Gemini NX C18 150*40 mm*5 um; mobile phase: [water(0.05% HCl)-ACN]; B %: 1%-30%, 10 min) to give HYBI_256 (170 mg, 280.42 umol, 60.08% yield, 99.55% purity) as yellow solid.

¹H NMR (CDCl₃ 400 MHz) δ_{H}=11.26-11.01 (m, 1H), 10.94-10.71 (m, 1H), 10.56 (s, 1H), 9.32 (s, 1H), 9.14 (s, 1H), 8.91 (t, J=6.0 Hz, 1H), 8.85 (d, J=2.4 Hz, 1H), 7.78 (dd, J=2.4, 8.4 Hz, 1H), 7.50 (d, J=8.4 Hz, 1H), 3.95 (br d, J=10.0 Hz, 2H), 3.79 (br t, J=11.2 Hz, 2H), 3.56 (br d, J=5.2 Hz, 2H), 3.42-3.34 (m, 4H), 3.31-3.21 (m, 6H), 3.18-2.98 (m, 4H), 2.89 (t, J=2.0 Hz, 3H), 2.06-1.93 (m, 2H).

LCMS: R, =0.697 min in 1.5 min chromatography, 5-95AB, Agilent Pursult 5 C18 20*2.0 mm, purity 100.0%, LCMS ESI calcd. for C₂₆H₃₃Cl₂N₁₀O₃[M+H]⁺ 603.20, found 603.1.

HPLC: R, =3.40 min in 8 min chromatography, 10-80CD, Xbridge Shield RP18 5 um 2.1*50 mm, purity 99.55%

### Example 35. 3-chloro-5-methoxy-N-(2-(4-methylpiperazin-1-yl)-5-(4-((3-morpholinopropyl)carbamoyl)-1H-1,2,3-triazol-1-yl)phenyl)pyrazine-2-carboxamide

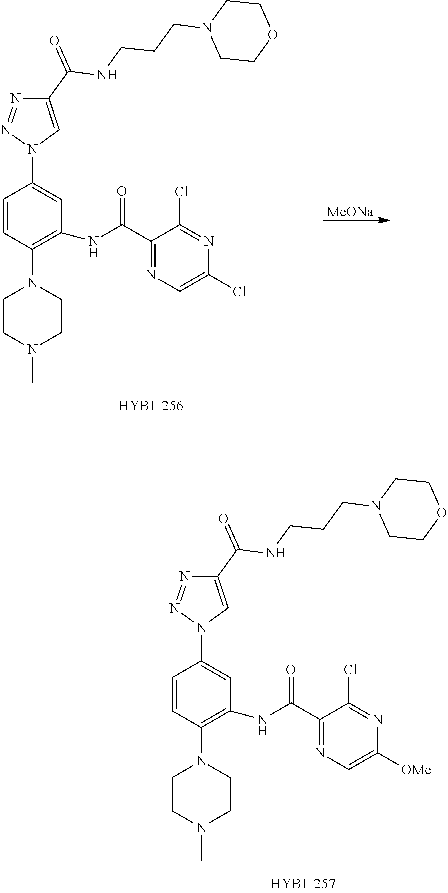 [SMILES: "MeONa" ; CN1CCN(c2ccc(-n3cc(C(=O)NCCCN4CCOCC4)nn3)cc2NC(=O)c2ncc(Cl)nc2Cl)CC1 ; COc1cnc(C(=O)Nc2cc(-n3cc(C(=O)NCCCN4CCOCC4)nn3)ccc2N2CCN(C)CC2)c(Cl)n1]

To a solution of HYBI_256 (150 mg, 248.55 umol, 1 eq) in MeOH (3 mL) was added sodium methanolate (4.48 mg, 82.85 umol, 1 eq). The mixture was stirred at 25° C. for 2 hr. The reaction mixture was filtered. The filtrate was concentrated directly. The residue was purified by prep-HPLC [column: Phenomenex Gemini-NX C18 75*30 mm*3 um; mobile phase: [water(10 mM NH4HCO3)-ACN]; B %: 33%-63%, 10 min] and further by SFC (condition: DAICEL CHIRALCEL OD (250 mm*30 mm, 10 um); mobile phase: [0.1% NH3H2O ETOH]; B %: 40%-40%, min). HYBI_257 (3.9 mg, 5.60 umol, 2.25% yield, 93.77% purity) was obtained as a white solid ¹H NMR (DMSO-d₆, 400 MHz) δ_{H}=10.73 (s, 1H), 9.30 (s, 1H), 9.04 (d, J=2.0 Hz, 1H), 8.93 (t, J=5.6 Hz, 1H), 8.60 (s, 1H), 7.76 (dd, J=2.8, 8.8 Hz, 1H), 7.55 (d, J=8.8 Hz, 1H), 4.11 (s, 3H), 3.70 (s, 4H), 3.46-3.43 (m, 2H), 3.06 (s, 4H), 2.77-2.59 (m, 4H), 2.54-2.46 (m, 6H), 2.44-2.33 (m, 3H), 1.81 (s, 2H).

HPLC Rₜ=4.218 min in 8 min chromatography, purity 93.77%.

LCMS Rₜ=1.872 min in 4 min chromatography, purity 90.73%, MS ESI calcd. for 598.25 [M+H]⁺ 599.25, found 599.3.

### Example 36. 3,5-dimethoxy-N-(2-(4-methylpiperazin-1-yl)-5-(4-((3-morpholinopropyl)carbamoyl)-1H-1,2,3-triazol-1-yl)phenyl)pyrazine-2-carboxamide

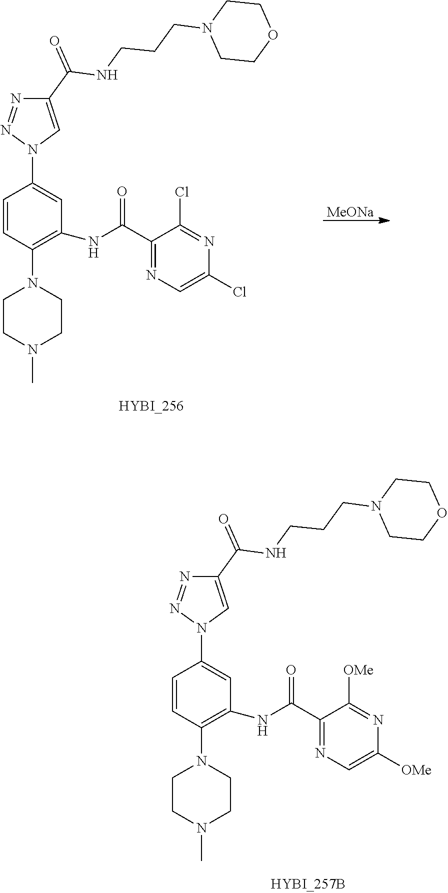 [SMILES: "MeONa" ; CN1CCN(c2ccc(-n3cc(C(=O)NCCCN4CCOCC4)nn3)cc2NC(=O)c2ncc(Cl)nc2Cl)CC1 ; COc1cnc(C(=O)Nc2cc(-n3cc(C(=O)NCCCN4CCOCC4)nn3)ccc2N2CCN(C)CC2)c(OC)n1]

To a solution of HYBI_256 (130 mg, 215.41 umol, 1 eq) in MeOH (2 mL) was added sodium methanolate (34.91 mg, 646.23 umol, 3 eq). The mixture was stirred at 25° C. for 2 hr. The reaction mixture was filtered. The filtrate was concentrated directly. The residue was purified by prep-HPLC [column: Phenomenex Gemini-NX C18 75*30 mm*3 um; mobile phase: [water(10 mM NH4HCO3)-ACN]; B %: 34%-52%, 6 min]. HYBI_257B (11.1 mg, 16.69 umol, 7.75% yield, 99.31% purity) was obtained as a white solid.

¹H NMR (DMSO-d₆, 400 MHz) δ_{H}=10.53 (s, 1H), 9.16 (s, 1H), 9.00 (d, J=2.4 Hz, 1H), 8.83 (t, J=5.6 Hz, 1H), 7.99 (s, 1H), 7.62 (dd, J=2.4, 8.4 Hz, 1H), 7.42 (d, J=8.8 Hz, 1H), 4.05 (d, J=4.4 Hz, 6H), 3.61 (t, J=4.4 Hz, 4H), 3.33-3.32 (m, 2H), 2.91 (t, J=4.4 Hz, 4H), 2.61-2.54 (m, 4H), 2.40-2.33 (m, 6H), 2.29 (s, 3H), 1.76-1.65 (m, 2H).

HPLC Rₜ=3.606 min in 8 min chromatography, purity 99.31%.

LCMS Rₜ=1.732 min in 4 min chromatography, purity 99.16%, MS ESI calcd. for 594.3 [M+H]⁺ 595.4, found 595.4.

### Example 37. 6-chloro-N-(2-(4-methylpiperazin-1-yl)-5-(4-((3-morpholinopropyl)carbamoyl)-1H-1,2,3-triazol-1-yl)phenyl)-2-(trifluoromethyl)nicotinamide

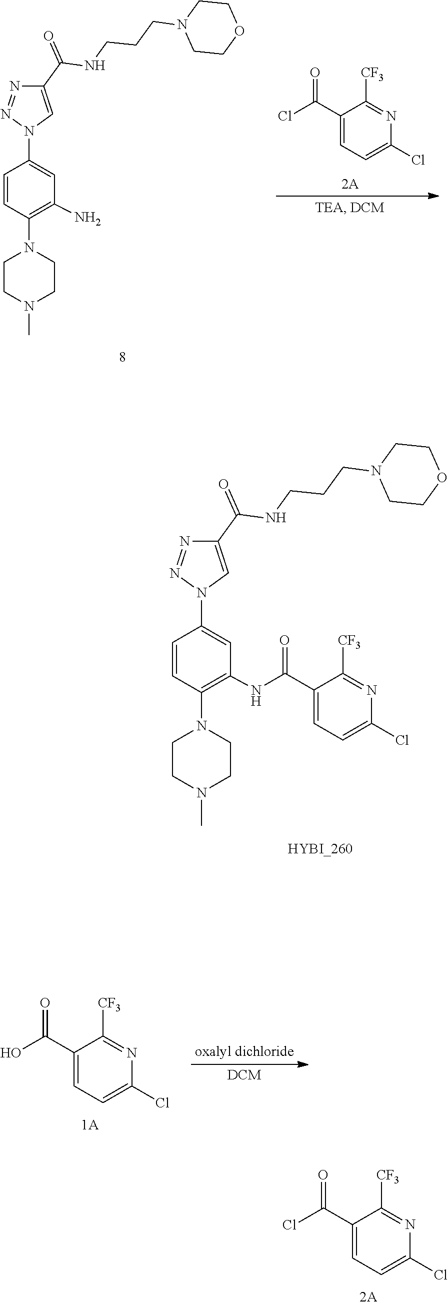 [SMILES: CN1CCN(c2ccc(-n3cc(C(=O)NCCCN4CCOCC4)nn3)cc2N)CC1 ; O=C(Cl)c1ccc(Cl)nc1C(F)(F)F ; CN1CCN(c2ccc(-n3cc(C(=O)NCCCN4CCOCC4)nn3)cc2NC(=O)c2ccc(Cl)nc2C(F)(F)F)CC1 ; O=C(O)c1ccc(Cl)nc1C(F)(F)F ; O=C(Cl)c1ccc(Cl)nc1C(F)(F)F]

Note: The preparation method of compound 8 can be found in Example 1 above.

### Step 1: 6-chloro-2-(trifluoromethyl)nicotinoyl chloride (Compound 2A)

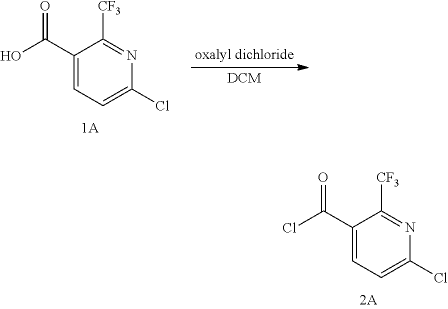 [SMILES: O=C(O)c1ccc(Cl)nc1C(F)(F)F ; O=C(Cl)c1ccc(Cl)nc1C(F)(F)F]

To a mixture of compound 2A (500 mg, 2.22 mmol, 1 eq) in DCM (5 mL) was added DMF (16 mg, 221.68 umol, 17.06 uL, 0.1 eq). (COCl)₂ (1.41 g, 11.08 mmol, 970.23 uL, 5 eq) was dropped into the mixture at −10° C. The mixture was stirred at 10° C. for 1 hr. The mixture was concentrated to dryness. Compound 2A (540 mg, 2.21 mmol, 99.84% yield) was obtained as a brown solid, which was used to next step directly.

### Step 2: 6-chloro-N-(2-(4-methylpiperazin-1-yl)-5-(4-((3-morpholinopropyl)carbamoyl)-1H-1,2,3-triazol-1-yl)phenyl)-2-(trifluoromethyl)nicotinamide (HYBI_260)

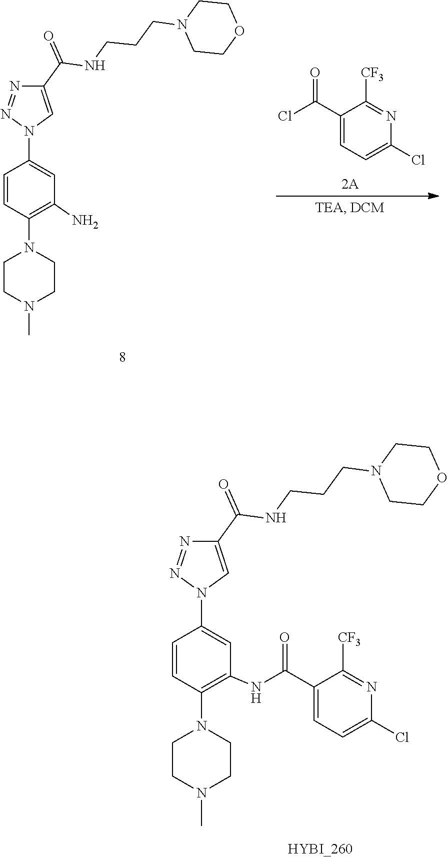 [SMILES: CN1CCN(c2ccc(-n3cc(C(=O)NCCCN4CCOCC4)nn3)cc2N)CC1 ; O=C(Cl)c1ccc(Cl)nc1C(F)(F)F ; CN1CCN(c2ccc(-n3cc(C(=O)NCCCN4CCOCC4)nn3)cc2NC(=O)c2ccc(Cl)nc2C(F)(F)F)CC1]

To a mixture of compound 8 (632.26 mg, 1.48 mmol, 1 eq) in DCM (5 mL) was added TEA (746 mg, 7.38 mmol, 1.03 mL, 5 eq). A solution of compound 2A (540 mg, 2.21 mmol, 1.5 eq) in DCM (5 mL) was dropped into the above mixture at −10° C. The mixture was stirred at 10° C. for 20 mins. The mixture was diluted with DCM (30 mL). The mixture was washed with water (25 mL×3) and brine (25 mL×2). The organic layer was dried over anhydrous Na₂SO₄, filtered and concentrated to dryness. The mixture was purified with Prep-HPLC (column: Xtimate C18 150*40 mm*10 um; mobile phase: [water (10 mM NH₄HCO₃)−ACN]; B %: 25%-55%, 10 min). HYBI_260 (300 mg, 462.64 umol, 31.36% yield, 98.09% purity) was obtained as a yellow solid.

¹H NMR (DMSO-d₆, 400 MHz) δ_{H}=9.19 (s, 1H) 8.83 (t, J=5.6 Hz, 1H) 8.52 (d, J=2.4 Hz, 1H) 8.32 (d, J=8.4 Hz, 1H) 8.07 (d, J=8.0 Hz, 1H) 7.75 (dd, J=8.8, 2.4 Hz, 1H) 7.37 (d, J=8.8 Hz, 1H) 3.61 (t, J=4.4 Hz, 4H) 3.36-3.38 (m, 2H) 3.29-3.33 (m, 2H) 2.86-3.02 (m, 4H) 2.46-2.49 (m, 2H) 2.31-2.44 (m, 6H) 2.22 (s, 3H) 1.64-1.79 (m, 2H).

HPLC Rₜ=3.740 min in 8 min chromatography, purity 98.09%.

LCMS Rₜ=1.918 min in 4 min chromatography, Chromolith Flash RP-18.5 um, 3.0*25 mm, purity 96.75%, MS ESI calcd. for 635.24 [M+H]⁺ 636.24, found 636.3.

### Example 38: 6-methoxy-N-(2-(4-methylpiperazin-1-yl)-5-(4-((3-morpholinopropyl)carbamoyl)-1H-1,2,3-triazol-1-yl)phenyl)-2-(trifluoromethyl)nicotinamide

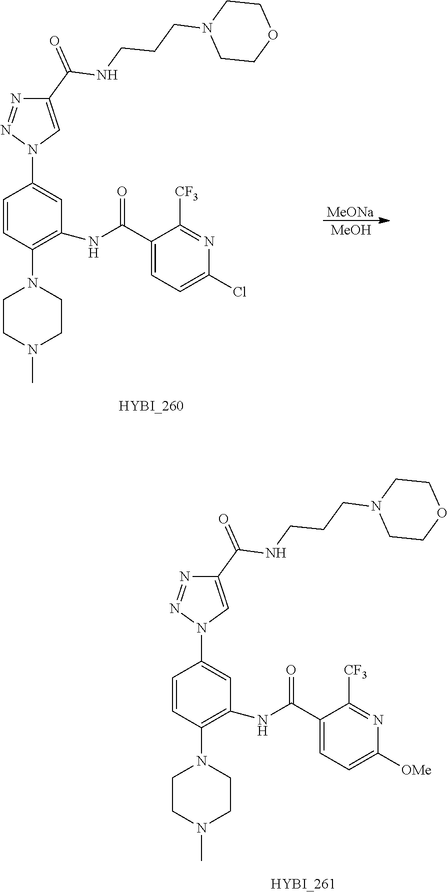 [SMILES: "MeONa" ; "MeOH" ; CN1CCN(c2ccc(-n3cc(C(=O)NCCCN4CCOCC4)nn3)cc2NC(=O)c2ccc(Cl)nc2C(F)(F)F)CC1 ; COc1ccc(C(=O)Nc2cc(-n3cc(C(=O)NCCCN4CCOCC4)nn3)ccc2N2CCN(C)CC2)c(C(F)(F)F)n1]

To a solution of HYBI_260 (50 mg, 78.61 umol, 1 eq) in MeOH (1 mL) was added MeONa (25.48 mg, 471.65 umol, 6 eq). The mixture was stirred at 25° C. for 32 hr. The mixture was concentrated to dryness. The residue was purified with prep-HPLC column: Phenomenex Gemini-NX C18 75*30 mm*3 um; mobile phase: [water(0.05% NH3H2O+10 mM NH4HCO3)-ACN]; B %: 21%-61%, 11 min. HYBI_261 (15 mg, 23.36 umol, 29.72% yield, 98.37% purity) was obtained as a white solid.

¹H NMR (DMSO-d6,400 MHz) δ_{H}=9.78 (s, 1H), 9.19 (s, 1H), 8.78 (t, J=5.6 Hz, 1H), 8.51 (s, 1H), 8.09 (d, J=8.4 Hz, 1H), 7.73 (d, J=6.8 Hz, 1H), 7.37 (d, J=8.4 Hz, 1H), 7.29 (d, J=8.4 Hz, 1H), 3.97 (s, 3H), 3.61 (t, J=4 Hz, 4H), 3.50-3.43 (m, 2H), 2.97-2.89 (s, 4H), 2.73-2.55 (m, 4H), 2.42-2.33 (m, 6H), 2.22 (s, 3H), 1.77-1.65 (m, 2H).

HPLC Rₜ=3.843 min in 8 min chromatography, purity 97.99%.

LCMS Rₜ=1.895 min in 4 min chromatography, purity 98.37%, MS ESI calcd. for 631.28 [M+H]⁺ 632.28, found 632.3.

### Example 39: 6-cyano-N-[2-(4-methylpiperazin-1-yl)-5-[4-(3-morpholinopropylcarbamoyl)triazol-1-yl]phenyl]-2-(trifluoromethyl)pyridine-3-carboxamide

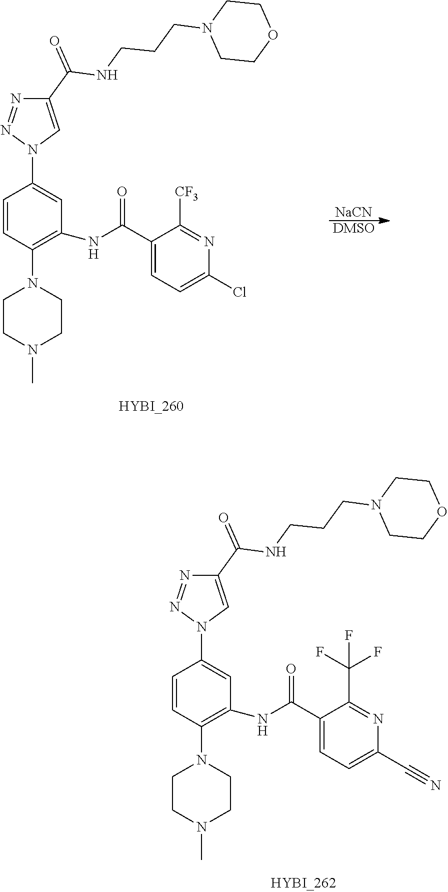

To a solution of HYBI_260 (150 mg, 235.82 umol, 1 eq) in DMSO (3 mL) was added NaCN (23.12 mg, 471.65 umol, 2 eq). The mixture was stirred at 90° C. for 12 hr. The residue was diluted with H₂O (50 mL), and the mixture was extracted with DCM (30 mL×2). The combined organic phase was washed with brine (20 mL), dried over anhydrous Na₂SO₄, filtered and concentrated. The residue was purified by twice prep-HPLC (column: Xtimate C18 150*40 mm*5 um; mobile phase: [water(0.05% HCl)-ACN]; B %: 1%-30%, 10 min) and (column: Phenomenex Gemini-NX 80*40 mm*3 um; mobile phase: [water(0.05% NH₃H₂O)-ACN]; B %: 32%-62%, 8 min). HYBI_262 (16 mg, 25.53 umol, 10.83% yield, 100% purity) was obtained as a white solid.

¹H NMR (400 MHz, CDCl₃)^{6H}=9.12 (s, 1H), 8.93 (d, J=2.4 Hz, 1H), 8.65-8.45 (m, 2H), 8.24 (d, J=8.0 Hz, 1H), 8.05 (d, J=8.0 Hz, 1H), 7.64 (dd, J=2.8, 8.8 Hz, 1H), 7.45 (d, J=8.8 Hz, 1H), 3.87-3.82 (m, 4H), 3.65-3.57 (m, 2H), 2.98-2.87 (m, 4H), 2.68-2.37 (m, 10H), 2.33 (s, 3H), 1.89-1.76 (m, 2H).

HPLC Rₜ=3.351 min in 8 min chromatography, Ultimate C18 3*50 mm 3 um, purity 100%.

LCMS Rₜ=1.295 min in 2 min chromatography, ChromCore 120 C18 3 um 3.0*30 mm, purity 100%, MS ESI calcd. for 626.27 [M+H]⁺ 627.27, found 627.4.

### Example 40: N5-[2-(4-methylpiperazin-1-yl)-5-[4-(3-morpholinopropylcarbamoyl)triazol-1-yl]phenyl]-6-(trifluoromethyl)pyridine-2,5-dicarboxamide

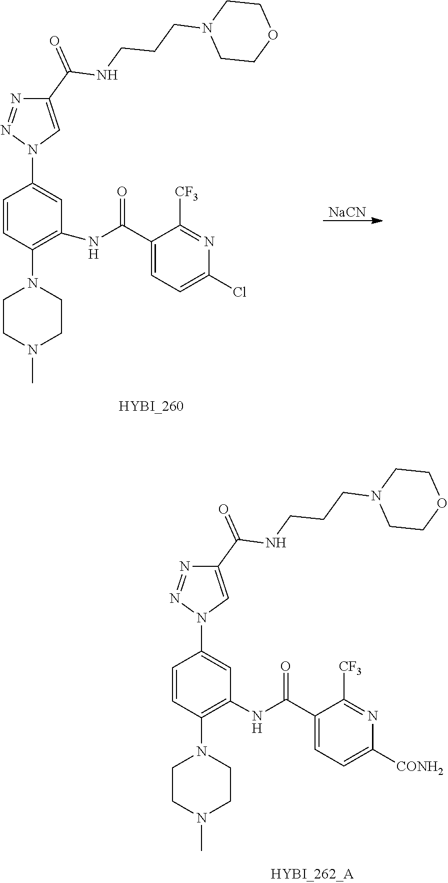 [SMILES: CN1CCN(c2ccc(-n3cc(C(=O)NCCCN4CCOCC4)nn3)cc2NC(=O)c2ccc(Cl)nc2C(F)(F)F)CC1 ; "NaCN" ; CN1CCN(c2ccc(-n3cc(C(=O)NCCCN4CCOCC4)nn3)cc2NC(=O)c2ccc(C(N)=O)nc2C(F)(F)F)CC1]

To a mixture of HYBI_260 (100.00 mg, 0.16 mmol) in DMSO (2 mL) was added NaCN (70 mg, 1.43 mmol), 1,4-diazabicyclo[2.2.2]octane (8.82 mg, 0.079 mmol) and H₂O (0.2 mL), and the mixture was stirred at 100° C. for 1 h. The residue was diluted with H₂O (50 mL), and the mixture was extracted with EtOAc (30 mL×2). The combined organic phase was washed with brine (20 mL), dried over anhydrous Na₂SO₄, filtered and concentrated to give the crude product. The crude product was purified by prep-HPLC (column: Phenomenex Gemini-NX C18 75×30 mm×3 um; mobile phase: [water(10 mM NH₄HCO₃)-ACN]; B %: 25%-45%, 7 min) to give HYBI_262_A (30 mg, 46.54 umol, 29.60% yield) as a white solid.

¹H NMR (DMSO-d₆, 400 MHz) δ_{H}=10.02 (s, 1H), 9.20 (s, 1H), 8.83 (br t, J=5.6 Hz, 1H), 8.53 (d, J=2.0 Hz, 1H), 8.40 (s, 2H), 8.13 (br s, 1H), 7.98 (br s, 1H), 7.76 (dd, J=8.8, 2.4 Hz, 1H), 7.38 (d, J=8.8 Hz, 1H), 3.62 (m, 4H), 3.37 (br s, 2H), 2.95 (br s, 4H), 2.47-2.50 (m, 4H), 2.33-2.42 (m, 6H), 2.22 (s, 3H), 1.67-1.76 (m, 2H).

HPLC Rₜ=1.915 min in 8 min chromatography, purity 99.56%.

LCMS Rₜ=1.776 min in 7 min chromatography, Xtimate C18, 3 m, 2.1×30 mm, purity 98.97%, MS ESI calcd. for 644.28 [M+H]⁺ 645.28, found 645.5.

### Example 41. 6-amino-N-(2-(4-methylpiperazin-1-yl)-5-(4-((3-morpholinopropyl)carbamoyl)-1H-1,2,3-triazol-1-yl)phenyl)-2-(trifluoromethyl)nicotinamide

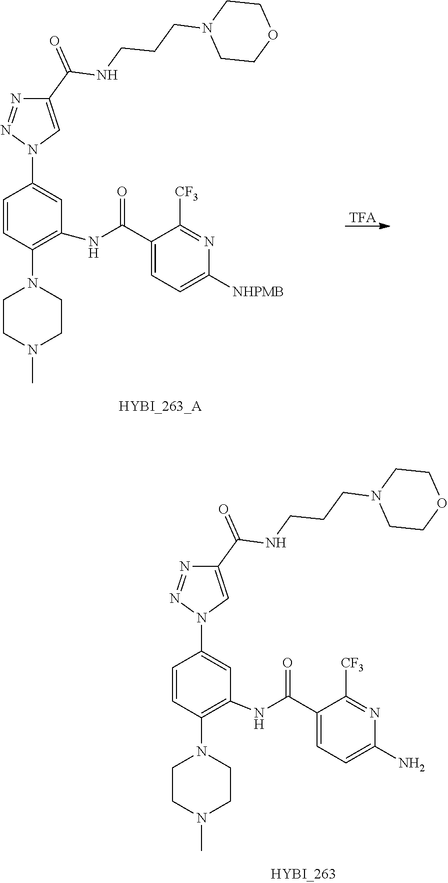

A mixture of HYBI_263_A (20 mg, 27.14 umol, 1 eq) and TFA (1.54 g, 13.51 mmol, 1 mL, 497.55 eq) was stirred at 50° C. for 1 h. The reaction mixture was concentrated directly. Water (2 mL) was added to the reaction mixture. The reaction mixture was then adjusted to pH˜9 with aq. NaOH (1 N) and concentrated to dryness. The residue was purified by prep-HPLC (column: Phenomenex Gemini-NX C18 75*30 mm*3 um; mobile phase: [water(10 mM NH₄HCO₃)-ACN]; B %: 24%-44%, 7 min). HYBI_263 (6 mg, 9.35 umol, 17.23% yield, 96.12% purity) was obtained as a white solid.

¹H NMR (DMSO-d₆, 400 MHz) δ_{H}=9.25 (s, 1H), 9.17 (s, 1H), 8.85-8.76 (m, 1H), 8.28 (s, 1H), 7.65 (dd, J=2.8, 8.8 Hz, 2H), 7.44 (d, J=8.8 Hz, 1H), 6.47 (s, 2H), 6.33 (s, 1H), 3.61 (t, J=4.4 Hz, 4H), 3.40-3.33 (m, 2H), 2.93 (t, J=4.4 Hz, 4H), 2.54-2.51 (m, 4H), 2.40-2.34 (m, 6H), 2.24 (s, 3H), 1.77-1.65 (m, 2H).

HPLC Rₜ=3.179 min in 8 min chromatography, purity 96.13%.

LCMS Rₜ=1.487 min in 4 min chromatography, purity 97.32%, MS ESI calcd. for 616.28, (M+H)*617.28, found 617.3.

### Example 42. 6-((4-methoxybenzyl)amino)-N-(2-(4-methylpiperazin-1-yl)-5-(4-((3-morpholinopropyl)carbamoyl)-1H-1,2,3-triazol-1-yl)phenyl)-2-(trifluoromethyl)nicotinamide

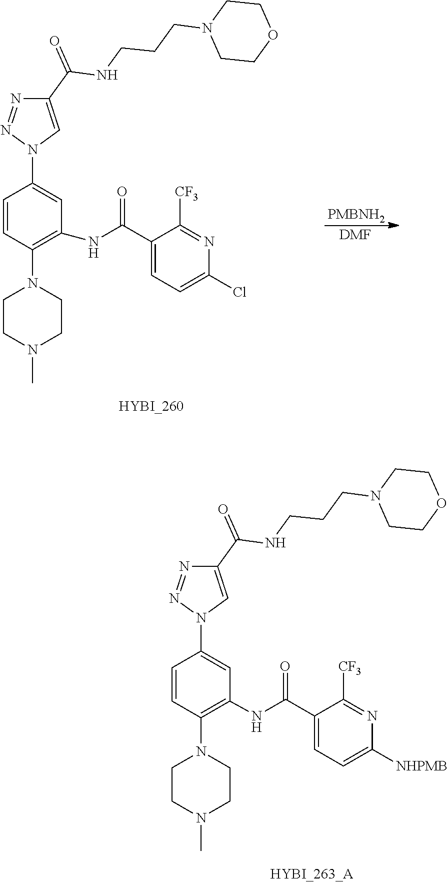

To a solution of HYBI_260 (150 mg, 235.82 umol, 1 eq) in DMF (2 mL) was added PMBNH₂ (32.35 mg, 235.82 umol, 30.52 uL, 1 eq). The mixture was stirred at 80° C. for 16 h. Water (20 mL) was added to the residue. The resulting mixture was extracted with EtOAc (20 mL*3). The combined organic phase was washed with brine (20 mL), dried over anhydrous Na₂SO₄, filtered and concentrated. The residue was purified by prep-HPLC (column: Phenomenex Gemini-NX C18 75*30 mm*3 um; mobile phase: [water(10 mM NH₄HCO₃)-ACN]; B %: 40%-70%, 10 min). HYBI_263_A (238 mg, 49.73 umol, 14.66% yield, 96.42% purity) was obtained as a white solid.

¹H NMR (DMSO-d₆, 400 MHz) δ_{H}=9.43 (s, 1H), 9.17 (s, 1H), 8.80 (t, J=5.6 Hz, 1H), 8.55 (d, J=2 Hz, 1H), 7.89 (t, J=6 Hz, 1H), 7.74-7.65 (m, 2H), 7.38 (d, J=8.4 Hz, 1H), 7.31 (d, J=8.4 Hz, 2H), 6.90 (d, J=8.4 Hz, 2H), 6.79 (d, J=8.8 Hz, 1H), 4.45 (d, J=5.6 Hz, 2H), 3.72 (s, 3H), 3.60 (t, J=4.4 Hz, 4H), 3.42-3.34 (m, 2H), 2.96-2.85 (m, 4H), 2.48-2.42 (m, 4H), 2.41-2.31 (m, 6H), 2.21 (s, 3H), 1.75-1.65 (m, 2H).

HPLC Rₜ=4.490 min in 8 min chromatography, purity 96.42%.

LCMS Rₜ=2.158 min in 4 min chromatography, purity 97.13%, MS ESI calcd. for 736.34, [M+H]⁺ 737.34, found 737.4.

### Example 43. N-(2-(4-methylpiperazin-1-yl)-5-(4-((3-morpholinopropyl)carbamoyl)-1H-1,2,3-triazol-1-yl)phenyl)-6-(methylthio)-2-(trifluoromethyl)nicotinamide

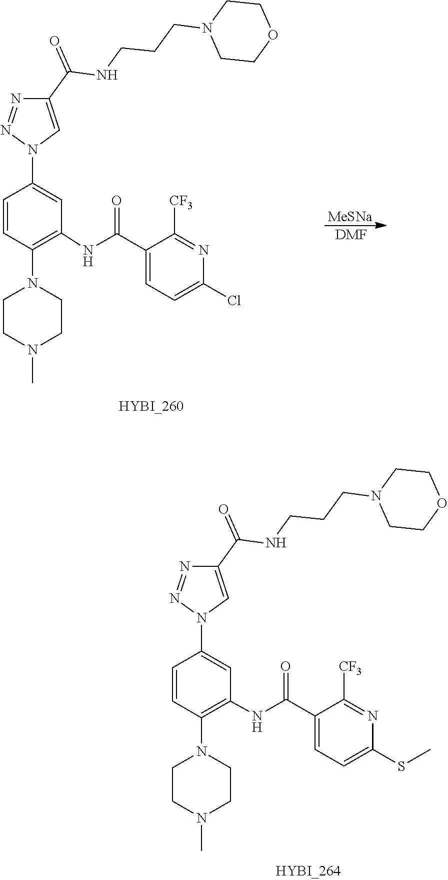

To a solution of HYBI_260 (50 mg, 78.61 umol, 1 eq) in DMF (1 mL) was added NaSMe (55 mg, 786.08 umol, 50.09 uL, 10 eq). The mixture was stirred at 40° C. for 16 hrs. The mixture was quenched with water (10 mL). The mixture was extracted with DCM (10 mL×3). The organic layer was washed with water (10 mL×3) and brine (10 mL). The organic layer was dried over anhydrous Na₂SO₄, filtered and concentrated. The mixture was purified with prep-HPLC (column: Phenomenex Gemini-NX C18 75*30 mm*3 um; mobile phase: [water (0.05% NH₃H₂O+10 mM NH₄HCO₃)-ACN]; B %: 28%-68%, 11 min). HYBI_264 (17.6 mg, 26.18 umol, 33.31% yield, 96.35% purity) was obtained as a white solid.

¹H NMR (DMSO-d₆, 400 MHz) δH=9.84 (s, 1H) 9.19 (s, 1H) 8.77-8.87 (s, 1H) 8.51 (s, 1H) 8.03 (d, J=8.0 Hz, 1H) 7.76 (dd, J=16.4, 8.0 Hz, 2H) 7.38 (d, J=8.4 Hz, 1H) 3.55-3.67 (m, 4H) 3.29 (s, 2H) 2.94 (s, 4H) 2.68 (s, 3H) 2.62 (s, 4H) 2.32-2.39 (m, 6H) 2.22 (s, 3H) 1.71 (t, J=7.2 Hz, 2H).

HPLC Rₜ=4.040 min in 8 min chromatography, Ultimate XB-C18 3.0*50 mm, 3 um, purity 98.01%.

LCMS Rₜ=2.000 min in 4 min chromatography, Chromolith Flash RP-18.5 um, 3.0*25 mm, purity 97.78%, MS ESI calcd. for 647.26 [M+H]⁺ 648.26, found 648.3.

### Example 44. N-[2-(4-methylpiperazin-1-yl)-5-[4-(3-morpholinopropylcarbamoyl)triazol-1-yl]phenyl]-6-methylsulfonyl-2-(trifluoromethyl)pyridine-3-carboxamide

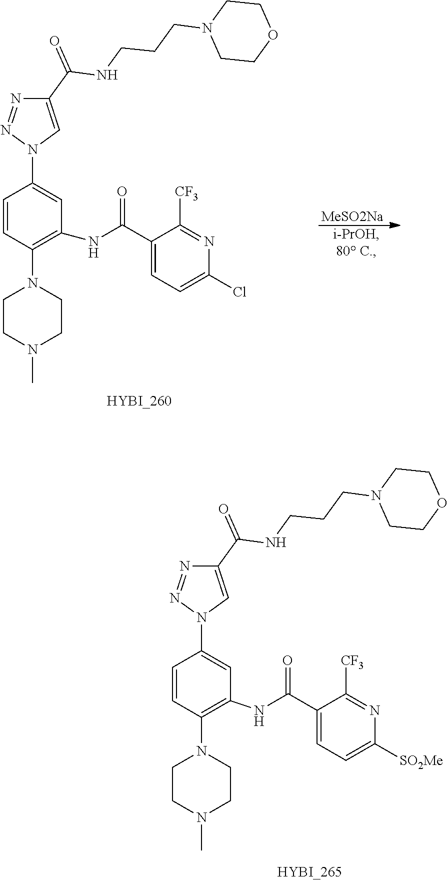 [SMILES: "MeSO2Na" ; CN1CCN(c2ccc(-n3cc(C(=O)NCCCN4CCOCC4)nn3)cc2NC(=O)c2ccc(Cl)nc2C(F)(F)F)CC1 ; "i-PrOH,80° C.," ; CN1CCN(c2ccc(-n3cc(C(=O)NCCCN4CCOCC4)nn3)cc2NC(=O)c2ccc(S(C)(=O)=O)nc2C(F)(F)F)CC1]

To a solution of HYBI_260 (50.0 mg, 78.6 umol) in i-PrOH (2.00 mL) was added sodium methanesulfinate (24.1 mg, 236 umol) and the mixture was stirred at 80° C. for 12 hours under N₂. The mixture was concentrated under vacuum to get a residue. The residue was added into H₂O (10 mL) and the mixture was extracted with DCM (3×10 mL). The combined organic phase was washed with brine (10 mL), dried over anhydrous Na₂SO₄, filtered and concentrated under vacuum to give a crude. The crude was purified by prep-HPLC (column: Phenomenex Gemini-NX 80*40 mm*3 um; mobile phase: [water(0.05% NH3H₂O)-ACN]; B %: 33%-63%, 8 min) to give HYBI_265 (17.3 mg, 25.5 umol, 32.4% yield, 100% purity) as an off-white solid.

¹H NMR (DMSO 400 MHz) δ_{H}=10.12 (s, 1H), 9.20 (s, 1H), 8.83 (t, J=5.6 Hz, 1H), 8.61 (d, J=8.0 Hz, 1H), 8.57-8.47 (m, 2H), 7.76 (dd, J=2.4, 8.4 Hz, 1H), 7.38 (d, J=8.8 Hz, 1H), 3.61 (br t, J=4.0 Hz, 4H), 3.43 (s, 3H), 3.39-3.33 (m, 8H), 3.03-2.82 (m, 4H), 2.40-2.30 (m, 4H), 2.24 (s, 3H), 1.75-1.66 (m, 2H).

LCMS: R, =0.681 min in 1.5 min chromatography, 5-95AB, Agilent Pursult 5 C18 20*2.0 mm, purity 92.5%, LCMS ESI calcd. for C₂₉H₃₇F₃N₉O₅S [M+H]⁺ 680.25, found 680.3.

HPLC: R, =2.84 min in 8 min chromatography, 10-80CD, Xbridge Shield RP18 5 um 2.1*50 mm, purity 100%.

### Example 45. 5-amino-N-(2-(4-methylpiperazin-1-yl)-5-(4-((3-morpholinopropyl)carbamoyl)-1H-1,2,3-triazol-1-yl)phenyl)pyrimidine-2-carboxamide

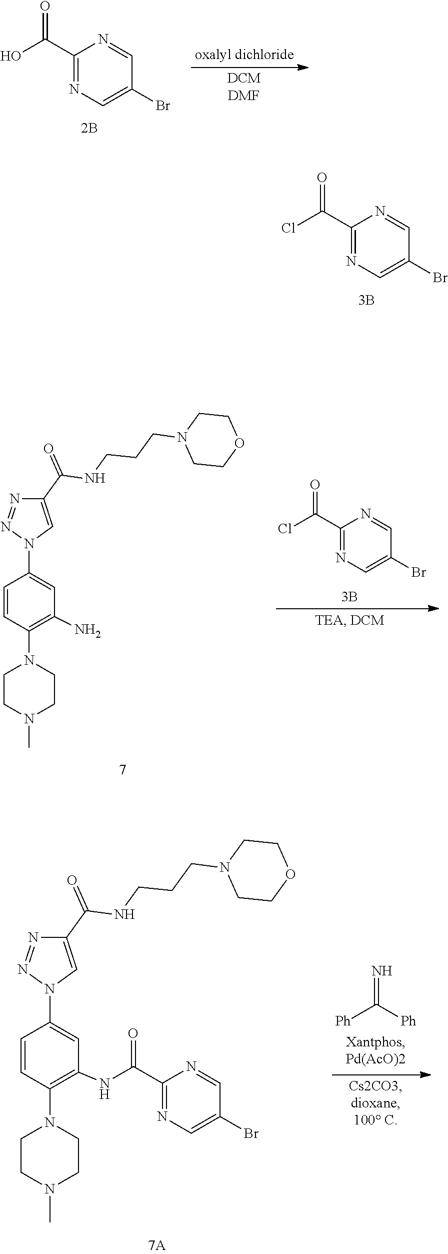 [SMILES: O=C(O)c1ncc(Br)cn1 ; O=C(Cl)c1ncc(Br)cn1 ; CN1CCN(c2ccc(-n3cc(C(=O)NCCCN4CCOCC4)nn3)cc2N)CC1 ; O=C(Cl)c1ncc(Br)cn1 ; CN1CCN(c2ccc(-n3cc(C(=O)NCCCN4CCOCC4)nn3)cc2NC(=O)c2ncc(Br)cn2)CC1 ; N=C(c1ccccc1)c1ccccc1]

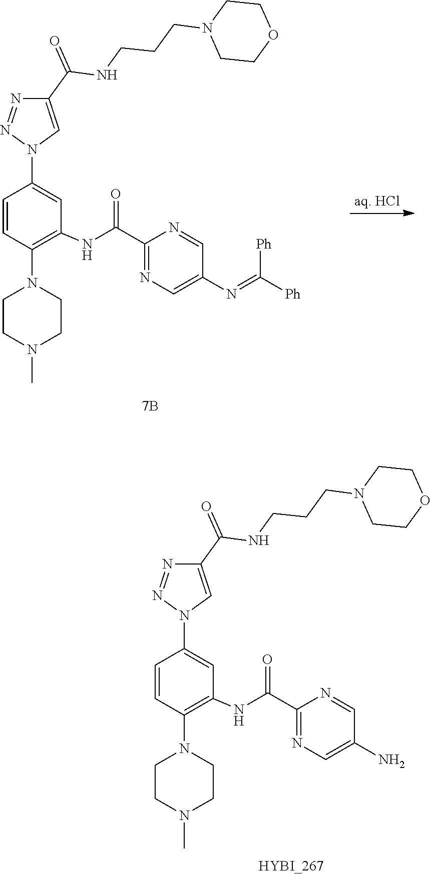 [SMILES: CN1CCN(c2ccc(-n3cc(C(=O)NCCCN4CCOCC4)nn3)cc2NC(=O)c2ncc(N=C(c3ccccc3)c3ccccc3)cn2)CC1 ; CN1CCN(c2ccc(-n3cc(C(=O)NCCCN4CCOCC4)nn3)cc2NC(=O)c2ncc(N)cn2)CC1]

Note: The preparation method of compound 7 can be found in Example 1 above.

### Step 1: 5-bromopyrimidine-2-carbonyl chloride (Compound 3B)

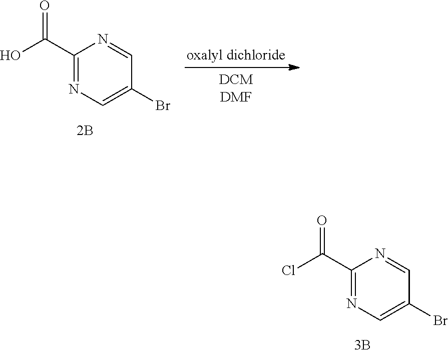 [SMILES: O=C(O)c1ncc(Br)cn1 ; O=C(Cl)c1ncc(Br)cn1]

To a solution of compound 2B (420 mg, 2.07 mmol, 1 eq) and DMF (15.12 mg, 206.90 umol, 15.92 uL, 0.1 eq) in DCM (4 mL) was added oxalyl dichloride (1.31 g, 10.35 mmol, 905.59 uL, 5 eq) dropwise at 0° C. The reaction mixture was stirred at 25° C. for 20 min. The mixture was concentrated to remove DCM. The crude product was used in the next step without further purification. Compound 3B (450 mg, 2.03 mmol, 98.22% yield) was obtained as a white solid.

### Step 2: 5-bromo-N-(2-(4-methylpiperazin-1-yl)-5-(4-((3-morpholinopropyl)carbamoyl)-1H-1,2,3-triazol-1-yl)phenyl)pyrimidine-2-carboxamide (Compound 7A)

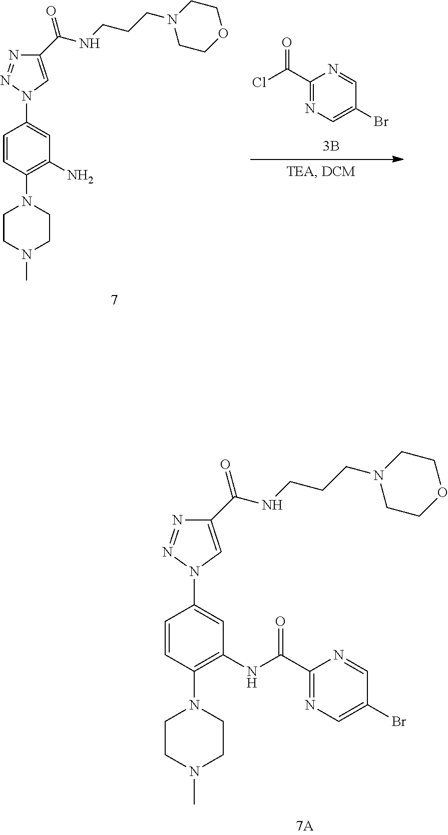 [SMILES: CN1CCN(c2ccc(-n3cc(C(=O)NCCCN4CCOCC4)nn3)cc2N)CC1 ; O=C(Cl)c1ncc(Br)cn1 ; CN1CCN(c2ccc(-n3cc(C(=O)NCCCN4CCOCC4)nn3)cc2NC(=O)c2ncc(Br)cn2)CC1]

To a mixture of compound 7 (622.03 mg, 1.45 mmol, 1 eq) and compound 3B (450 mg, 2.03 mmol, 1.4 eq) in DCM (6 mL) was added TEA (734.40 mg, 7.26 mmol, 1.01 mL, 5 eq) dropwise at −10° C. The reaction mixture was stirred at 25° C. for 20 min. The mixture was concentrated to remove DCM. The residue was purified by prep-HPLC [column: Xtimate C18 150*40 mm*10 um; mobile phase: [water (10 mM NH4HCO3)-ACN]; B %: 20%-50%, 10 min]. Compound 7A (150 mg, 228.85 umol, 15.77% yield, 93.6% purity) was obtained as yellow solid.

LCMS Rₜ=1.571 min in 4 min chromatography, XBridge Shield RP18, 5 um, 2.1*50 mm, purity 93.6%, MS ESI calcd. for 612.19 [M+H]⁺ 613.19, found 613.2.

### Step 3: 5-((diphenylmethylene)amino)-N-(2-(4-methylpiperazin-1-yl)-5-(4-((3-morpholinopropyl)carbamoyl)-1H-1,2,3-triazol-1-yl)phenyl)pyrimidine-2-carboxamide (Compound 7B)

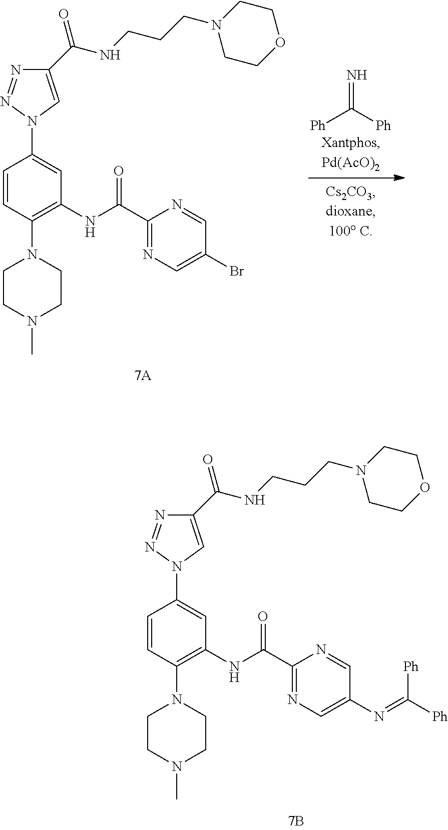 [SMILES: CN1CCN(c2ccc(-n3cc(C(=O)NCCCN4CCOCC4)nn3)cc2NC(=O)c2ncc(Br)cn2)CC1 ; N=C(c1ccccc1)c1ccccc1 ; CN1CCN(c2ccc(-n3cc(C(=O)NCCCN4CCOCC4)nn3)cc2NC(=O)c2ncc(N=C(c3ccccc3)c3ccccc3)cn2)CC1]

To a solution of compound 7A (100 mg, 163.00 umol, 1 eq) and diphenylmethanimine (44.31 mg, 244.50 umol, 41.03 uL, 1.5 eq) in 1,4-dioxane (1.5 mL) was added Pd(AcO)₂ (3.66 mg, 16.30 umol, 0.1 eq), Xantphos (14.15 mg, 24.45 umol, 0.15 eq) and Cs₂CO₃ (106.22 mg, 325.99 umol, 2 eq). The mixture was degassed and purged with N₂ for 3 times. The mixture was stirred at 100° C. for 12 hr under N₂ atmosphere. Water (15 mL) was added to the reaction mixture. The reaction mixture was extracted with DCM (20 mL*3). The combined organic phase was washed with brine dried over anhydrous Na₂SO₄, filtered and concentrated. The residue was purified by flash silica gel chromatography (Eluent of 0-12% MeOH/DCM). Compound 7B (63 mg, 72.90 umol, 44.72% yield, 82.6% purity) was obtained as a yellow oil.

LCMS Rₜ=1.177 min in 2.5 min chromatography, purity 82.6%, MS ESI calcd. for 713.36 [M+H]⁺ 714.36, found 714.4.

### Step 4: 5-amino-N-(2-(4-methylpiperazin-1-yl)-5-(4-((3-morpholinopropyl)carbamoyl)-1H-1,2,3-triazol-1-yl)phenyl)pyrimidine-2-carboxamide (HYBI_267)

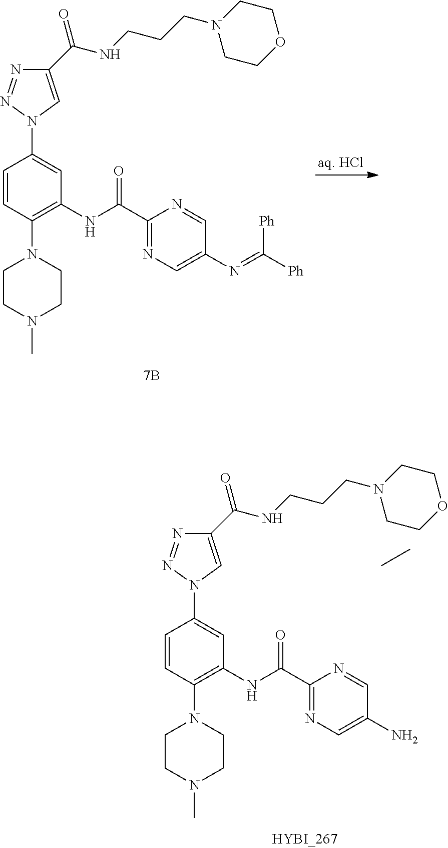

To a solution of compound 7B (63 mg, 88.26 umol, 1 eq) in THF (2 mL) was added HCl (12 M, 73.55 uL, 10 eq). The mixture was stirred at 25° C. for 3 hr. Water (10 mL) was added to the mixture. The aqueous phase was adjusted to pH=8 with solid NaHCO₃. The mixture was extracted with DCM (10 mL*3). The combined organic layers were concentrated to dryness. The residue was purified by prep-HPLC [column: Phenomenex Gemini-NX C18 75*30 mm*3 um; mobile phase: [water(10 mM NH4HCO3)-ACN]; B %: 10%-40%, 8 min]. HYBI_267 (10.7 mg, 19.47 umol, 22.06% yield, 100% purity) was obtained as a white solid.

¹H NMR (DMSO-d₆, 400 MHz) δ_{H}=10.83 (s, 1H), 9.18 (s, 1H), 9.02 (d, J=2.4 Hz, 1H), 8.83 (t, J=5.6 Hz, 1H), 8.26 (s, 2H), 7.61 (dd, J=2.4 Hz, J=8.4 Hz 1H), 7.44 (d, J=8.4 Hz, 1H), 6.39 (s, 2H), 3.61 (t, J=4.8 Hz, 4H), 3.40-3.30 (m, 2H), 2.94 (t, J=4.8 Hz, 4H), 2.70-2.55 (m, 4H), 2.40-2.33 (m, 6H), 2.29 (s, 3H), 1.71 (t, J=6.8 Hz, 2H).

HPLC Rₜ=2.922 min in 8 min chromatography, purity 100%.

LCMS Rₜ=1.311 min in 4 min chromatography, purity 98.86%, MS ESI calcd. for 549.29 [M+H]⁺ 550.29, found 550.3.

### Example 46. 1-(3-(4-chloro-2-(trifluoromethyl)benzamido)-4-(4-methylpiperazin-1-yl)phenyl)-N-(3-morpholinopropyl)-1H-1,2,3-triazole-4-carboxamide

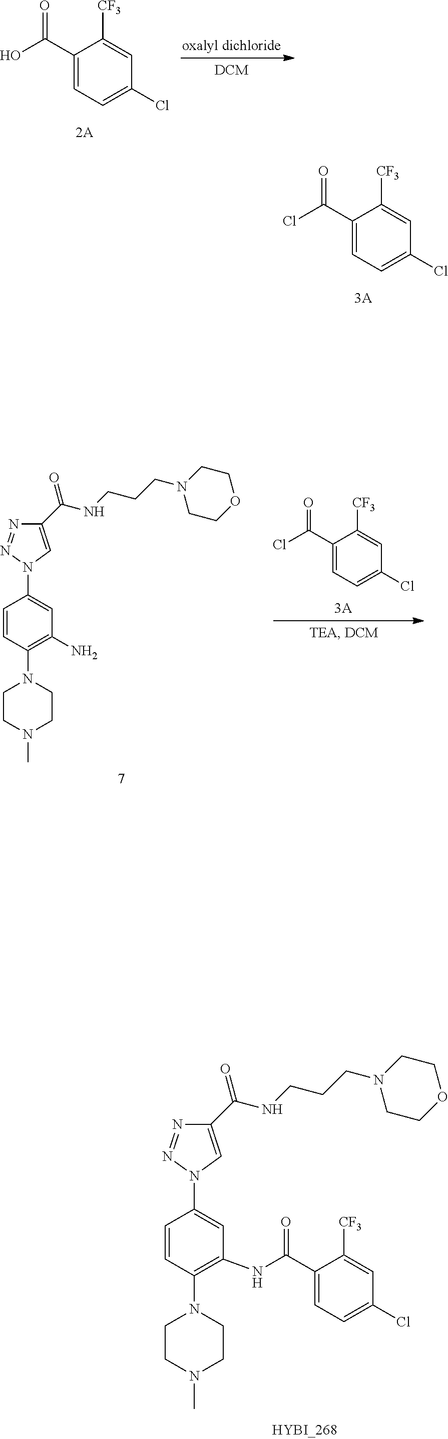 [SMILES: O=C(O)c1ccc(Cl)cc1C(F)(F)F ; O=C(Cl)c1ccc(Cl)cc1C(F)(F)F ; CN1CCN(c2ccc(-n3cc(C(=O)NCCCN4CCOCC4)nn3)cc2N)CC1 ; O=C(Cl)c1ccc(Cl)cc1C(F)(F)F ; CN1CCN(c2ccc(-n3cc(C(=O)NCCCN4CCOCC4)nn3)cc2NC(=O)c2ccc(Cl)cc2C(F)(F)F)CC1]

Note: The preparation method of compound 7 can be found in Example 1 above.

### Step 1: 4-chloro-2-(trifluoromethyl)benzoyl chloride (Compound 3A)

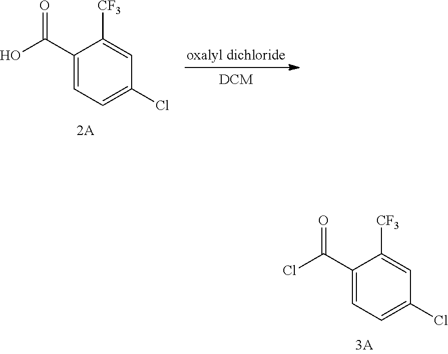 [SMILES: O=C(O)c1ccc(Cl)cc1C(F)(F)F ; O=C(Cl)c1ccc(Cl)cc1C(F)(F)F]

To a solution of compound 2A (500 mg, 2.23 mmol, 1 eq) and DMF (16.27 mg, 222.65 umol, 17.13 uL, 0.1 eq) in DCM (5 mL) was added oxalyl dichloride (1.41 g, 11.13 mmol, 974.53 uL, 5 eq) dropwise at 0° C. The reaction mixture was stirred at 25° C. for 20 min. The mixture was concentrated to remove DCM. The product was used in the next step without further purification. Compound 3A (540 mg, crude) was obtained as yellow oil.

### Step 2: 1-(3-(4-chloro-2-(trifluoromethyl)benzamido)-4-(4-methylpiperazin-1-yl)phenyl)-N-(3-morpholinopropyl)-1H-1,2,3-triazole-4-carboxamide (HYBI_268)

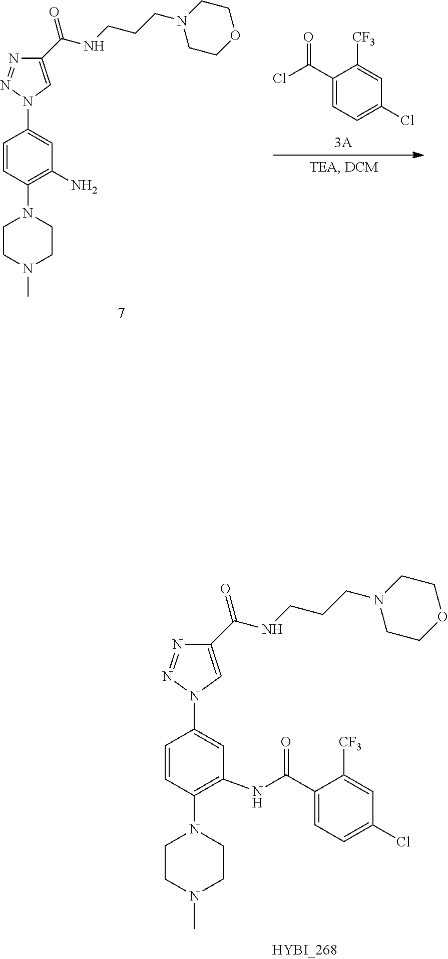 [SMILES: CN1CCN(c2ccc(-n3cc(C(=O)NCCCN4CCOCC4)nn3)cc2N)CC1 ; O=C(Cl)c1ccc(Cl)cc1C(F)(F)F ; CN1CCN(c2ccc(-n3cc(C(=O)NCCCN4CCOCC4)nn3)cc2NC(=O)c2ccc(Cl)cc2C(F)(F)F)CC1]

To a mixture of compound 7 (680.18 mg, 1.59 mmol, 1 eq) and compound 3A (540 mg, 2.22 mmol, 1.4 eq) in DCM (7 mL) was added TEA (803.05 mg, 7.94 mmol, 1.10 mL, 5 eq) at −10° C. The reaction mixture was stirred at 25° C. for 20 min. The mixture was concentrated to remove DCM. The residue was purified by prep-HPLC column: Phenomenex luna 30*30 mm*10 um+YMC AQ 100*30*10 um; mobile phase: [water(0.05% HCl)-ACN]; B %: 0%-30%, 30 min and was further separated by prep-HPLC column: Phenomenex Gemini-NX C18 75*30 mm*3 um; mobile phase: [water(10 mM NH4HCO3)-ACN]; B %: 35%-60%, 8 min. HYBI_268 (4.7 mg, 7.22 umol, 4.55e-1% yield, 97.58% purity) was obtained as a white solid.

¹H NMR (CD₃CN, 400 MHz) δ_{H}=9.00 (s, 1H), 8.89 (d, J=2.4 Hz, 1H), 8.62 (s, 1H), 8.51-8.42 (m, 1H), 7.89 (d, J=1.6 Hz, 1H), 7.80 (dd, J=1.6, 8.0 Hz 1H), 7.72 (d, J=8.0 Hz 1H), 7.55 (dd, J=2.8, 8.8 Hz 1H), 7.45 (d, J=8 Hz, 1H), 3.72 (t, J=4.8 Hz, 4H), 3.48 (q, J=6.4 Hz, 2H), 2.91 (t, J=4.8 Hz, 4H), 2.51-2.40 (m, 10H), 2.22 (s, 3H), 1.81-1.72 (m, 2H).

HPLC Rₜ=4.158 min in 8 min chromatography, purity 97.59%.

LCMS Rₜ=2.089 min in 4 min chromatography, purity 99.45%, MS ESI calcd. for 634.24 [M+H]⁺ 635.3, found 635.3.

### Example 47. 1-(3-(3,5-bis(trifluoromethyl)benzamido)-4-(4-methylpiperazin-1-yl)phenyl)-N-(3-morpholinopropyl)-1H-1,2,3-triazole-4-carboxamide

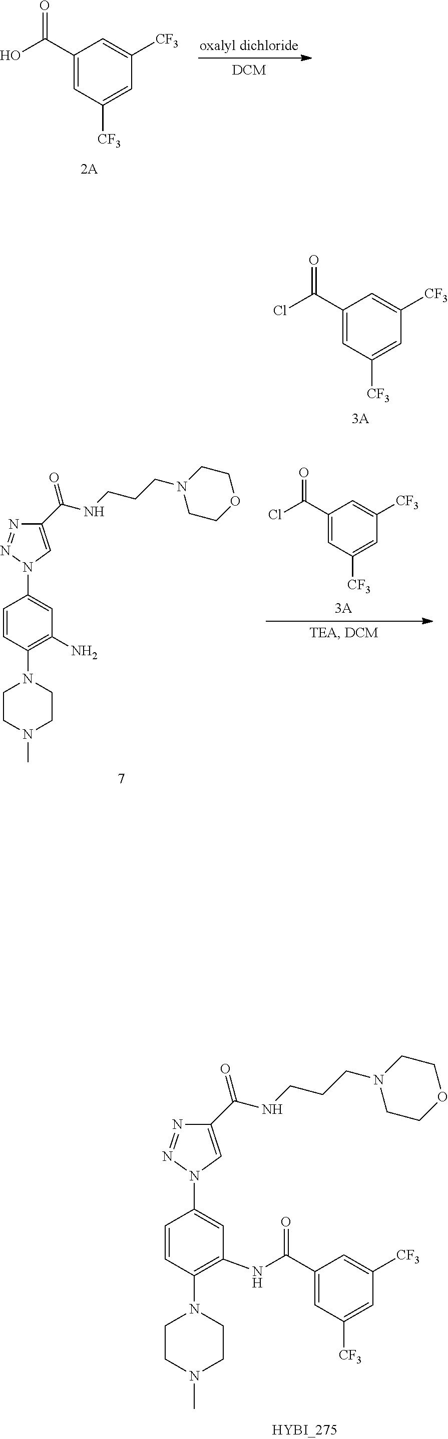

Note: The preparation method of compound 7 can be found in Example 1 above.

### Step 1: 3,5-bis(trifluoromethyl)benzoyl chloride (Compound 3A)

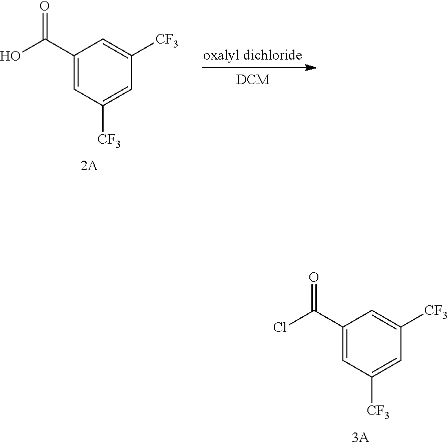 [SMILES: O=C(O)c1cc(C(F)(F)F)cc(C(F)(F)F)c1 ; O=C(Cl)c1cc(C(F)(F)F)cc(C(F)(F)F)c1]

To a solution of compound 2A (100 mg, 387.42 umol, 1 eq) and DMF (2.83 mg, 38.74 umol, 2.98 uL, 0.1 eq) in DCM (1.5 mL) was added oxalyl dichloride (245.88 mg, 1.94 mmol, 169.57 uL, 5 eq) dropwise at 0° C. The reaction mixture was stirred at 25° C. for 20 mins. The mixture was concentrated to remove DCM. The product was used in the next step without further purification. Compound 3A (100 mg, 361.58 umol, 93.33% yield,) was obtained as yellow oil.

### Step 2: 1-(3-(3,5-bis(trifluoromethyl)benzamido)-4-(4-methylpiperazin-1-yl)phenyl)-N-(3-morpholinopropyl)-1H-1,2,3-triazole-4-carboxamide (HYBI_275)

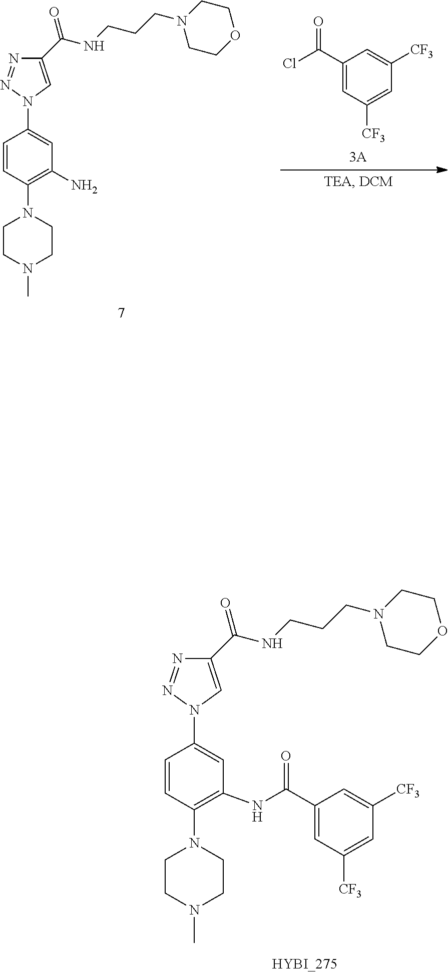 [SMILES: CN1CCN(c2ccc(-n3cc(C(=O)NCCCN4CCOCC4)nn3)cc2N)CC1 ; O=C(Cl)c1cc(C(F)(F)F)cc(C(F)(F)F)c1 ; CN1CCN(c2ccc(-n3cc(C(=O)NCCCN4CCOCC4)nn3)cc2NC(=O)c2cc(C(F)(F)F)cc(C(F)(F)F)c2)CC1]

To a mixture of compound 7 (110.68 mg, 258.27 umol, 1 eq) and compound 3A (100 mg, 361.58 umol, 65.36 uL, 1.4 eq) in DCM (1 mL) was added TEA (130.67 mg, 1.29 mmol, 179.74 uL, 5 eq) at −10° C. The reaction mixture was stirred at 25° C. for 20 min. The reaction mixture was concentrated. The residue was purified by prep-HPLC column: Phenomenex Gemini-NX C18 75*30 mm*3 um; mobile phase: [water(10 mM NH4HCO3)-ACN]; B %: 40%-70%, 10 min. HYBI_275 (36.6 mg, 52.56 umol, 20.35% yield, 96.04% purity) was obtained as a white solid.

¹H NMR (DMSO, 400 MHz) δ_{H}=10.22 (s, 1H), 9.21 (s, 1H), 8.84 (t, J=5.6 Hz, 1H), 8.61-8.56 (m, 3H), 8.43 (s, 1H), 7.77 (dd, J=2.8, 8.8 Hz, 1H), 7.43 (d, J=8.8 Hz, 1H), 3.61 (t, J=4.8 Hz, 4H), 3.38-3.34 (m, 2H), 2.97 (t, J=4.2 Hz, 4H), 2.49-2.45 (m, 4H), 2.37 (t, J=6.8 Hz, 6H), 2.22 (s, 3H), 1.75-1.67 (m, 2H).

HPLC Rₜ=4.410 min in 8 min chromatography, purity 96.04%.

LCMS Rₜ=2.263 min in 4 min chromatography, purity 93.74%, MS ESI calcd. for 668.27 [M+H]⁺ 669.3, found 669.3.

### Example 48: 1-[3-[(2-chloro-4-methyl-5-nitro-benzoyl)amino]-4-(4-methylpiperazin-1-yl)phenyl]-N-(3-morpholinopropyl)triazole-4-carboxamide

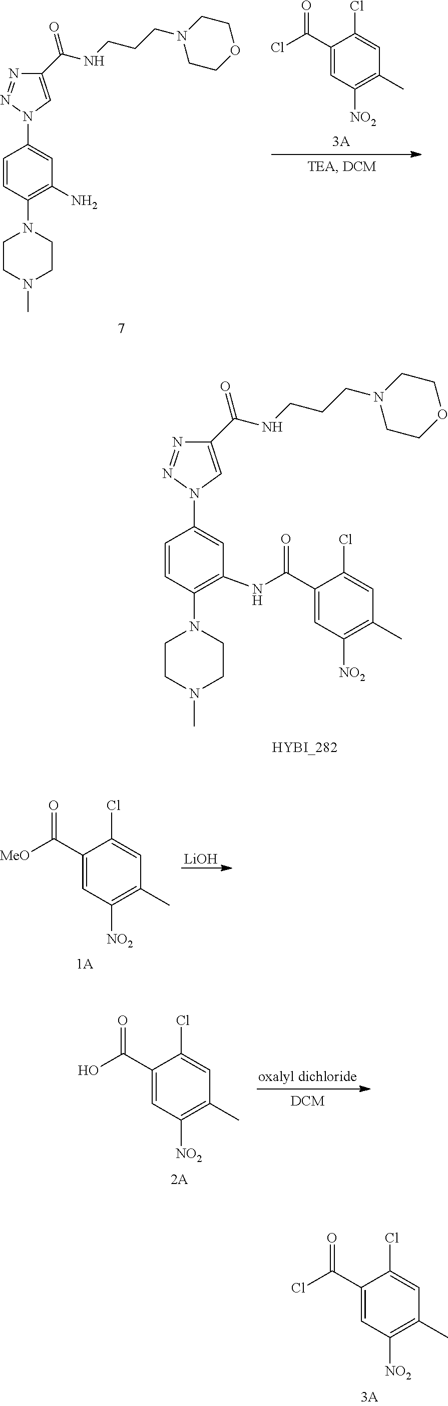

Note: The preparation method of compound 7 can be found in Example 1 above.

### Step 1: 2-chloro-4-methyl-5-nitro-benzoic acid (Compound 2A)

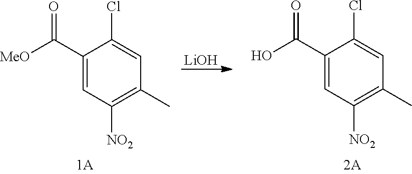

To a mixture of compound 1A (600 mg, 2.61 mmol,) in THF (2 mL) and H₂O (8 mL) was added LiOH·H₂O (548.26 mg, 13.07 mmol), and the mixture was stirred at 70° C. for 1 h. The mixture was diluted with H₂O (50 mL) and acidified with 1 N HCl to pH˜4. The mixture was extracted with EtOAc (50 mL×2). The combined organic phase was washed with water (20 mL) and brine (20 mL), dried over anhydrous Na₂SO₄, filtered and concentrated to give the crude product. The crude compound 2A (560 mg, 2.60 mmol, 99.41% yield) as a yellow solid, which was used into the next step without further purification.

### Step 2: 2-chloro-4-methyl-5-nitro-benzoyl chloride (Compound 3A)

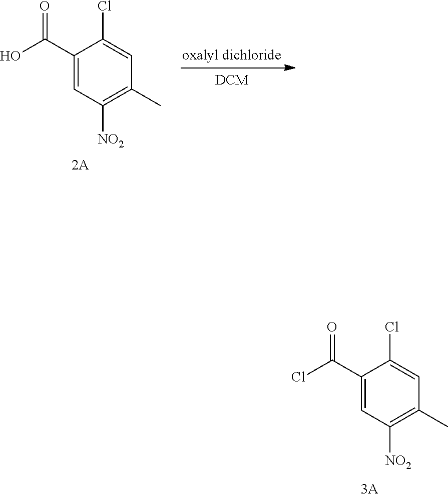

To a mixture of compound 2A (480 mg, 2.23 mmol) in DCM (5 mL) was added DMF (one drop) and oxalyl dichloride (1.41 g, 11.13 mmol, 0.97 mL) at 0° C., and the mixture was stirred at 20° C. for 30 min. The mixture was concentrated to give the residue. The crude product compound 3A (521 mg, 2.23 mmol, 99.99% yield) was obtained as a yellow oil, which was used into the next step without further purification.

### Step 3: 1-[3-[(2-chloro-4-methyl-5-nitro-benzoyl)amino]-4-(4-methylpiperazin-1-yl)phenyl]-N-(3-morpholinopropyl)triazole-4-carboxamide (HYBI_282)

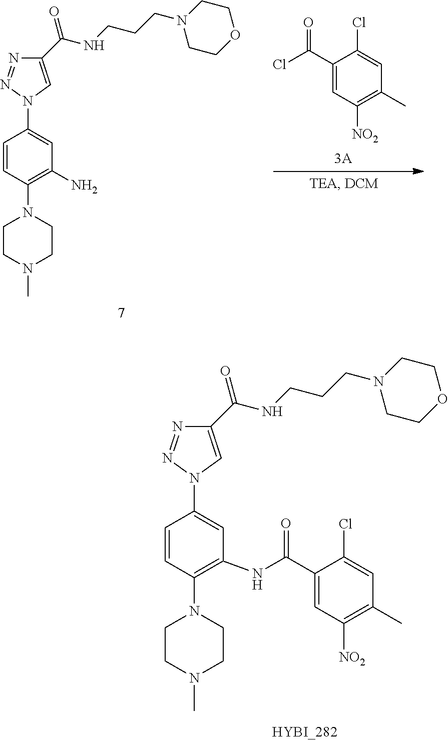 [SMILES: CN1CCN(c2ccc(-n3cc(C(=O)NCCCN4CCOCC4)nn3)cc2N)CC1 ; Cc1cc(Cl)c(C(=O)Cl)cc1[N+](=O)[O-] ; Cc1cc(Cl)c(C(=O)Nc2cc(-n3cc(C(=O)NCCCN4CCOCC4)nn3)ccc2N2CCN(C)CC2)cc1[N+](=O)[O-]]

To a mixture of compound 7 (600 mg, 1.40 mmol) and compound 3A (458.76 mg, 1.96 mmol,) in DCM (20 mL) at −10° C. was added TEA (1.42 g, 14.00 mmol, 1.95 mL), and the mixture was stirred at 20° C. for 30 min. The residue was diluted with H₂O (100 mL), and the mixture was extracted with DCM (50 mL×2). The combined organic phase was washed with water (20 mL), dried over anhydrous Na₂SO₄, filtered and concentrated to give the crude product. The crude product was purified by reversed-phase HPLC (column: Phenomenex Gemini-NX C18 75×30 mm×3 um; mobile phase: [water(10 mM NH₄HCO₃)-ACN]; B %: 36%-56%, 7 min) to give HYBI_282 (40 mg, 63.89 umol, 40.00% yield) as a white solid.

¹H NMR (DMSO-d₆, 400 MHz) δ_{H}=9.96 (s, 1H) 9.22 (s, 1H) 8.83 (br t, J=5.6 Hz, 1H) 8.59 (br s, 1H) 8.30-8.38 (m, 1H) 7.80-7.88 (m, 1H) 7.72-7.78 (m, 1H) 7.33-7.45 (m, 1H) 3.61 (t, J=4.4 Hz, 4H) 3.35-3.40 (m, 2H) 2.90-3.00 (m, 4H) 2.59 (s, 3H) 2.53 (br s, 4H) 2.32-2.42 (m, 6H) 2.18-2.25 (m, 3H) 1.66-1.76 (m, 2H).

HPLC Rₜ=3.933 min in 8 min chromatography, purity 98.02%.

LCMS Rₜ=1.959 min in 4 min chromatography, purity 96.18%, MS ESI calcd. for 626.25 [M+H]⁺ 626.25, found 626.3.

### Example 49: 1-(3-(5-amino-2-chloro-4-methylbenzamido)-4-(4-methylpiperazin-1-yl)phenyl)-N-(3-morpholinopropyl)-1H-1,2,3-triazole-4-carboxamide

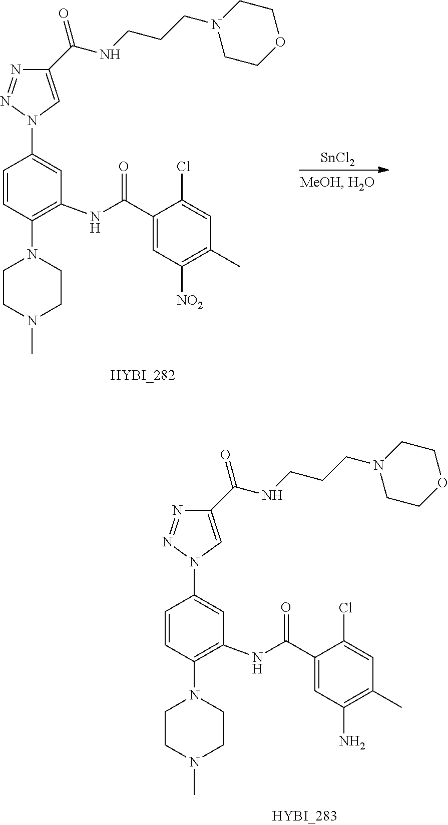 [SMILES: Cc1cc(Cl)c(C(=O)Nc2cc(-n3cc(C(=O)NCCCN4CCOCC4)nn3)ccc2N2CCN(C)CC2)cc1[N+](=O)[O-] ; "SnCl2" ; Cc1cc(Cl)c(C(=O)Nc2cc(-n3cc(C(=O)NCCCN4CCOCC4)nn3)ccc2N2CCN(C)CC2)cc1N]

To a solution of HYBI_282 (200 mg, 319.44 umol, 1 eq) in MeOH (1.5 mL) and H₂O (0.5 mL) was added dichlorotin (181.71 mg, 958.31 umol, 24.86 uL, 3 eq). The mixture was stirred at 68° C. for 2 hr. The mixture was adjusted with saturated aqueous NaHCO₃ to pH˜8. The mixture was filtered and the filtrate was concentrated. The residue was purified by prep-HPLC (column: Phenomenex Gemini-NX C18 75*30 mm*3 um; mobile phase: [water(10 mM NH4HCO3)-ACN]; B %: 30%-50%, 7 min). Compound HYBI_283 (46.5 mg, 77.50 umol, 24.26% yield, 99.35% purity) was obtained as a white solid.

¹H NMR (DMSO-d₆, 400 MHz) δ_{H}=9.48 (s, 1H), 9.22-9.16 (m, 1H), 8.88-8.75 (m, 2H), 7.68 (dd, J=2.4, 8.8 Hz, 1H), 7.47 (d, J=8.8 Hz, 1H), 7.14 (s, 1H), 6.95 (s, 1H), 5.30 (s, 2H), 3.61 (t, J=4.4 Hz, 4H), 3.38-3.35 (m, 2H), 2.91 (t, J=4.4 Hz, 4H), 2.48-2.43 (m, 3H), 2.41-2.33 (m, 6H), 2.26-2.19 (m, 4H), 2.10 (s, 3H), 1.76-1.67 (m, 2H).

HPLC Rₜ=3.702 min in 8 min chromatography, purity 99.35%.

LCMS Rₜ=1.802 min in 4 min chromatography, purity 97.81%, MS ESI calcd. for 595.28, [M+H]⁺ 596.28, found 596.4.

### Example 51. 6-chloro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)-4-(trifluoromethyl)nicotinamide

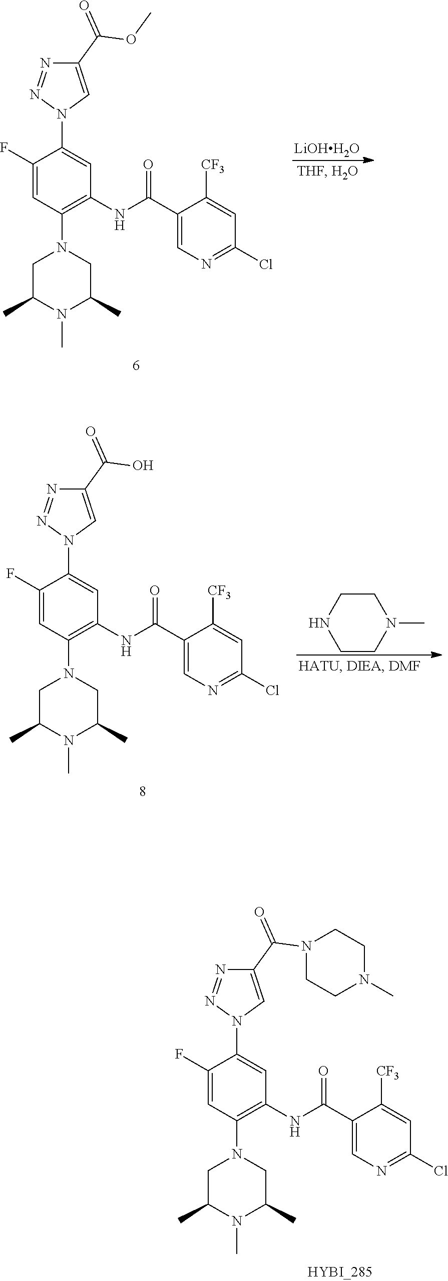

### Step 1: 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylic acid (Compound 8)

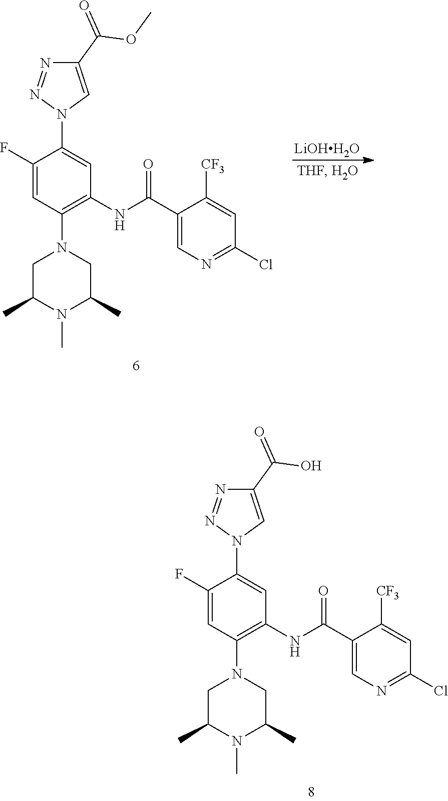 [SMILES: COC(=O)c1cn(-c2cc(NC(=O)c3cnc(Cl)cc3C(F)(F)F)c(N3C[C@@H](C)N(C)[C@@H](C)C3)cc2F)nn1 ; "LiOH·H2O" ; C[C@@H]1CN(c2cc(F)c(-n3cc(C(=O)O)nn3)cc2NC(=O)c2cnc(Cl)cc2C(F)(F)F)C[C@H](C)N1C]

To a mixture of compound 6 (70 mg, 122.82 umol, 1 eq) in THF (3.5 mL) and H₂O (0.35 mL) was added LiOH·H₂O (10 mg, 245.64 umol, 2 eq). The mixture was stirred at 25° C. for 2 hrs. The mixture was acidified with 2N HCl to pH=5. The mixture was concentrated to dryness. Compound 8 (68.28 mg, 122.83 umol, 100.00% yield) was obtained as a white solid.

### Step 2: 6-chloro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)-4-(trifluoromethyl)nicotinamide (HYBI_285)

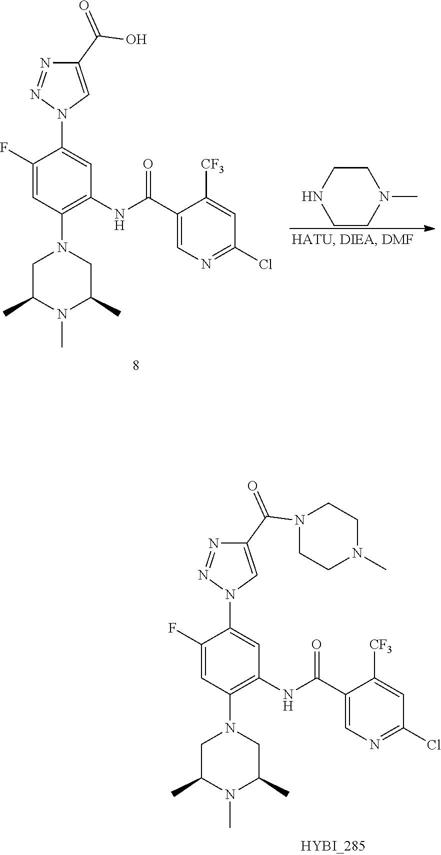

To a mixture of compound 8 (88 mg, 157.92 umol, 1 eq) and 1-methylpiperazine (23.73 mg, 237 umol, 26.28 uL, 1.5 eq) in DMF (4 mL) was added DIEA (61 mg, 473.76 umol, 82.52 uL, 3 eq). HATU (90 mg, 236.88 umol, 1.5 eq) was added into the mixture. The mixture was stirred at 25° C. for 2 hrs. The mixture was concentrated to dryness. The mixture was purified with prep-HPLC (column: Phenomenex Gemini-NX C18 75*30 mm*3 um; mobile phase: [water(0.05% NH₃H₂O+10 mM NH₄HCO₃)-ACN]; B %: 32%-62%, 10 min and column: Phenomenex Gemini NX C18 150*40 mm*5 um; mobile phase: [water(0.05% HCl)-ACN]; B %: 5%-35%, 10 min). HYBI_285 (12.6 mg, 19.52 umol, 12.36% yield, 98.87% purity) was obtained as a white solid.

¹H NMR (DMSO-d₆, 400 MHz) δ_{H}=10.70-11.17 (m, 1H), 10.10-10.30 (m, 1H), 8.81-9.08 (m, 2H), 8.30-8.53 (m, 1H), 8.06 (s, 1H), 7.39-7.66 (m, 1H), 4.42-5.17 (m, 2H), 3.84-4.08 (m, 1H), 3.42-3.55 (m, 4H), 3.11 (s, 6H), 2.83 (s, 6H), 2.72-2.64 (m, 1H), 1.23-1.53 (m, 6H).

HPLC Rₜ=2.231 min in 8 min chromatography, purity 98.87%.

LCMS Rₜ=1.374 min in 4 min chromatography, Xtimate C18.3 um, 2.1*30 mm, purity 100.00%, MS ESI calcd. for 637.23 [M+H]⁺ 638.23, found 638.5.

### Example 52. 6-chloro-N-(4-fluoro-5-(4-((1-methylpiperidin-4-yl)carbamoyl)-1H-1,2,3-triazol-1-yl)-2-((3S,5R)-3,4,5-trimethylpiperazin-1-yl phenyl)-4-(trifluoromethyl)nicotinamide

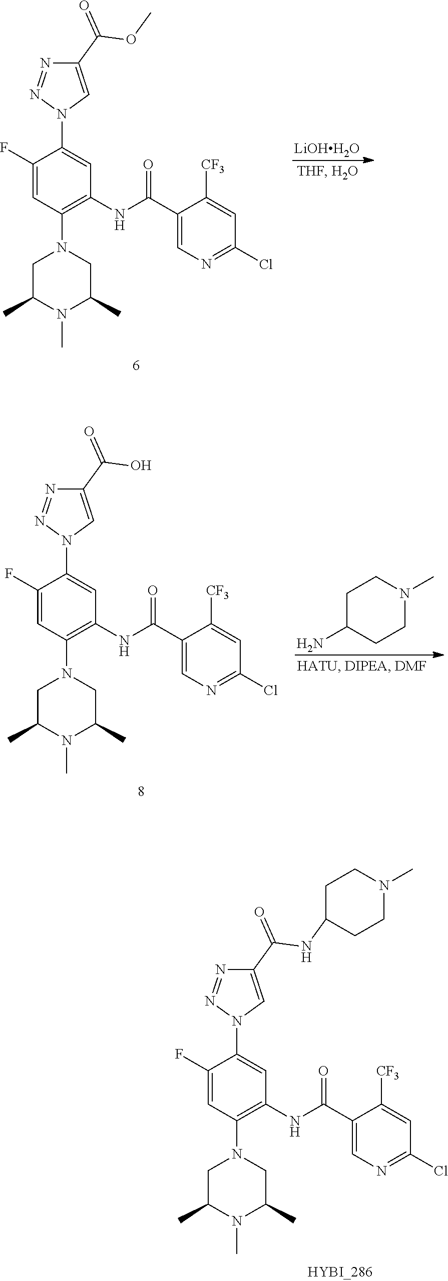

### Step 1: 1-(5-(6-chloro-4-(trifluoromethyl)nicotinamido)-2-fluoro-4-((3S,5R)-3,4,5-trimethylpiperazin-1-yl)phenyl)-1H-1,2,3-triazole-4-carboxylic acid (Compound 8)

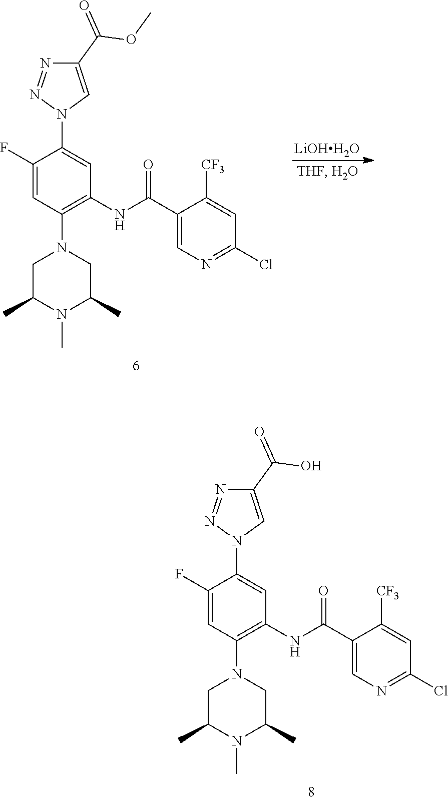 [SMILES: COC(=O)c1cn(-c2cc(NC(=O)c3cnc(Cl)cc3C(F)(F)F)c(N3C[C@@H](C)N(C)[C@@H](C)C3)cc2F)nn1 ; "LiOH·H2O" ; C[C@@H]1CN(c2cc(F)c(-n3cc(C(=O)O)nn3)cc2NC(=O)c2cnc(Cl)cc2C(F)(F)F)C[C@H](C)N1C]

To a mixture of compound 6 (90 mg, 157.91 umol, 1 eq) in THF (4.5 mL) and H₂O (0.45 mL) was added LiOH·H₂O (13 mg, 315.82 umol, 2 eq). The mixture was stirred at 25° C. for 2 hrs. The mixture was acidified with 2N HCl to pH=5. The mixture was concentrated to dryness. Compound 8 (87.79 mg, 157.92 umol, 100.00% yield) was obtained as a white solid.

### Step 2: 6-chloro-N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-4-(trifluoromethyl)nicotinamide (HYBI_286)

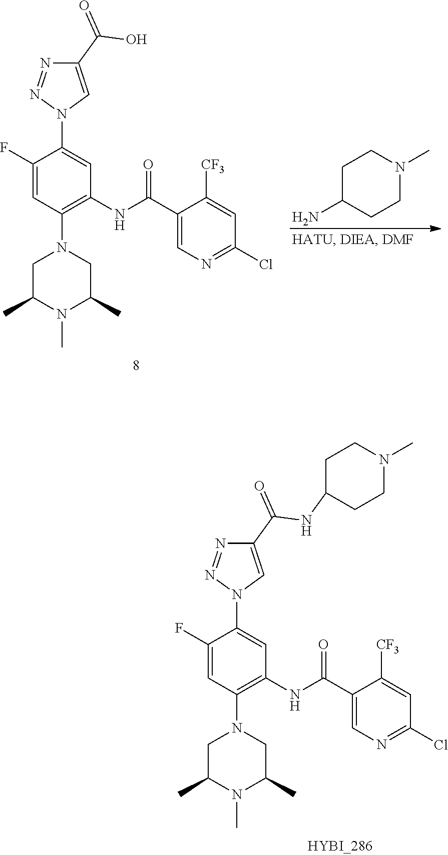

To a mixture of compound 8 (88 mg, 157.92 umol, 1 eq) and 1-methylpiperidin-4-amine (27 mg, 236.88 umol, 1.5 eq) in DMF (4 mL) was added DIEA (61 mg, 473.76 umol, 82.52 uL, 3 eq). HATU (90.07 mg, 236.88 umol, 1.5 eq) was added into the mixture. The mixture was stirred at 25° C. for 2 hrs. The mixture was concentrated to dryness. The mixture was purified by prep-HPLC (column: Phenomenex Gemini-NX C18 75*30 mm*3 um; mobile phase: [water(0.05% NH₃H₂O+10 mM NH₄HCO₃)-ACN]; B %: 34%-74%, 10 min and column: Phenomenex Gemini NX C18 150*40 mm*5 um; mobile phase: [water(0.05% HCl)-ACN]; B %: 0%-30%, 10 min). HYBI_286 (9.0 mg, 13.70 umol, 8.68% yield, 99.29% purity) was obtained as a white solid.

¹H NMR (DMSO-d₆, 400 MHz) δ_{H}=10.07-10.75 (m, 2H), 8.83-9.10 (m, 2H), 8.29-8.63 (m, 2H), 8.00-8.10 (m, 1H), 7.39-7.63 (m, 1H), 3.88-4.35 (m, 1.5H), 3.22-3.62 (m, 8H), 3.01-3.10 (s, 1.5H), 2.65-2.89 (m, 6H), 2.01-2.17 (m, 4H), 1.27-1.50 (m, 6H).

HPLC Rₜ=2.340 min in 8 min chromatography, purity 99.27%.

LCMS Rₜ=1.399 min in 4 min chromatography, Xtimate C18.3 um, 2.1*30 mm, purity 100.00%, MS ESI calcd. for 651.25 [M+H]⁺ 652.25, found 652.5.

### Example 53. N-(5-(4-(diethylcarbamoyl)-1H-1,2,3-triazol-1-yl)-4-fluoro-2-((3S,5R)-3,4,5-trimethylpiperazin-1-yl)phenyl)-6-fluoro-4-(trifluoromethyl)nicotinamide

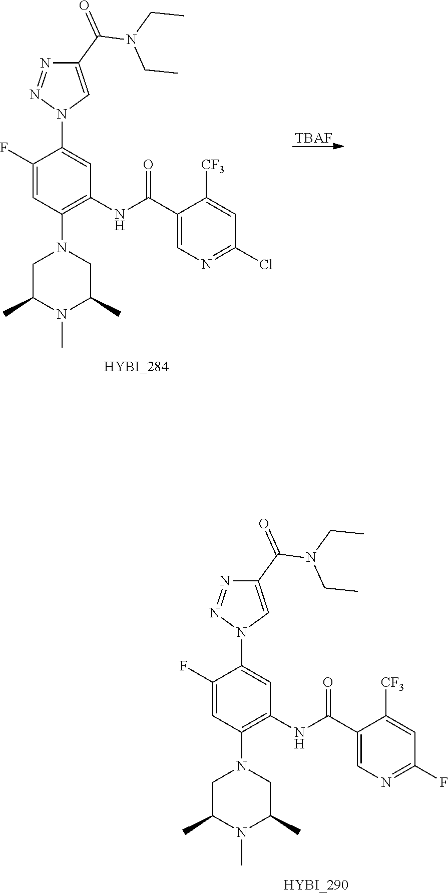 [SMILES: CCN(CC)C(=O)c1cn(-c2cc(NC(=O)c3cnc(Cl)cc3C(F)(F)F)c(N3C[C@@H](C)N(C)[C@@H](C)C3)cc2F)nn1 ; "TBAF" ; CCN(CC)C(=O)c1cn(-c2cc(NC(=O)c3cnc(F)cc3C(F)(F)F)c(N3C[C@@H](C)N(C)[C@@H](C)C3)cc2F)nn1]

To a mixture of HYBI_284 (120 mg, 196.39 umol, 1 eq) in DMSO (1 mL) was added TBAF·3H₂O (62 mg, 196.39 umol, 1 eq). The mixture was stirred at 100° C. for 1 hr. The mixture was concentrated to dryness. The mixture was purified with prep-HPLC (column: Phenomenex Gemini-NX C18 75*30 mm*3 um; mobile phase: [water(0.04% NH₃H₂O 10 mM NH₄HCO₃)-ACN]; B %: 35%-65%, 10 min). HYBI_290 (10.1 mg, 16.99 umol, 8.65% yield, 100% purity) was obtained as a white solid.

¹H NMR (DMSO-d₆, 400 MHz) δ_{H}=10.15 (s, 1H), 8.93 (s, 1H), 8.70 (s, 1H), 8.25 (d, J=8.0 Hz, 1H), 7.88 (s, 1H), 7.31 (d, J=12.0 Hz, 1H), 3.68-3.84 (m, 2H), 3.44-3.52 (m, 2H), 3.30 (s, 2H), 3.13 (d, J=11.2 Hz, 2H), 2.35-2.44 (m, 2H), 2.19 (s, 3H), 1.24 (t, J=6.8 Hz, 3H), 1.16 (t, J=6.8 Hz, 3H), 1.03 (d, J=6.0 Hz, 6H).

HPLC Rₜ=3.862 min in 8 min chromatography, purity 100%.

LCMS Rₜ=2.231 min in 4 min chromatography, purity 100%, MS ESI calcd. for 594.25 [M+H]⁺ 595.25, found 595.4.

### Example 54. N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-methoxy-4-(trifluoromethyl)nicotinamide

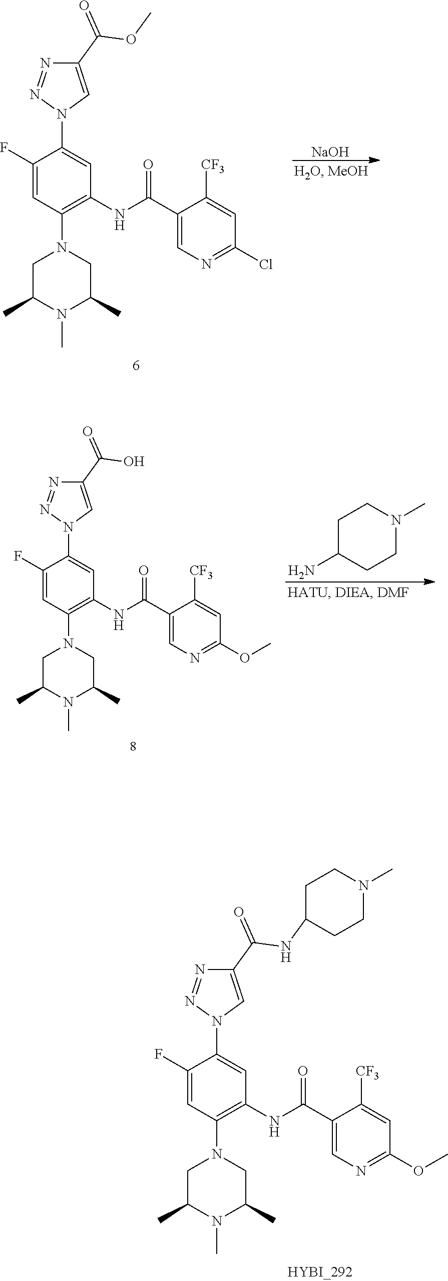

### Step 1: 1-(2-fluoro-5-(6-methoxy-4-(trifluoromethyl)nicotinamido)-4-((3S,5R)-3,4,5-trimethylpiperazin-1-yl)phenyl)-1H-1,2,3-triazole-4-carboxylic acid (Compound 8)

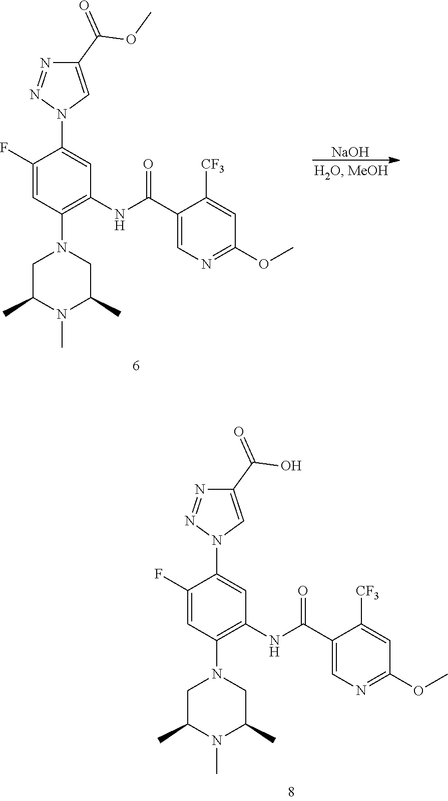 [SMILES: COC(=O)c1cn(-c2cc(NC(=O)c3cnc(OC)cc3C(F)(F)F)c(N3C[C@@H](C)N(C)[C@@H](C)C3)cc2F)nn1 ; "NaOH" ; COc1cc(C(F)(F)F)c(C(=O)Nc2cc(-n3cc(C(=O)O)nn3)c(F)cc2N2C[C@@H](C)N(C)[C@@H](C)C2)cn1]

To a mixture of compound 6 (70 mg, 122.82 umol, 1 eq) in MeOH (4 mL) was added a solution of NaOH (25 mg, 614.10 umol, 5 eq) in H₂O (1 mL). The mixture was stirred at 60° C. for 2 hrs. The mixture was acidified with 2N HCl to pH=5. The mixture was concentrated to dryness. The mixture was used directly to the next step without purification. Compound 8 (67.73 mg, 122.81 umol, 100.00% yield) was obtained as a brown solid.

### Step 2: N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-methoxy-4-(trifluoromethyl)nicotinamide (HYBI_292)

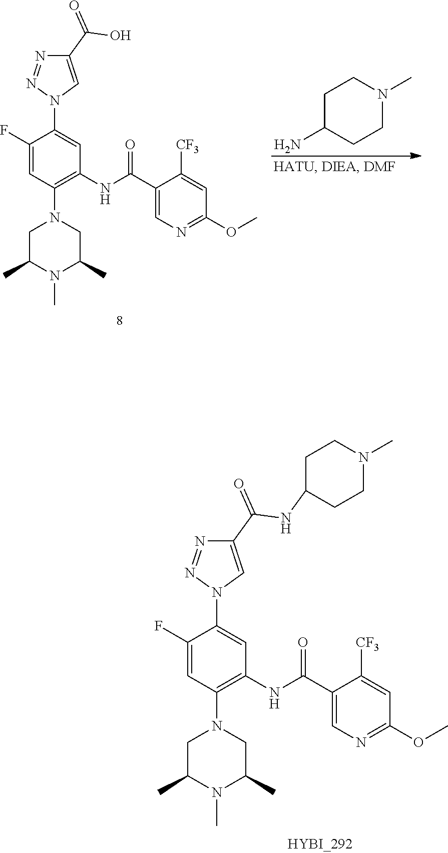

To a mixture of compound 8 (67.73 mg, 122.81 umol, 1 eq) and 1-methylpiperidin-4-amine (21.04 mg, 184.22 umol, 1.5 eq) in DMF (3 mL) was added DIEA (48 mg, 368.44 umol, 64.17 uL, 3 eq). HATU (70 mg, 184.22 umol, 1.5 eq) was added into the mixture. The mixture was stirred at 25° C. for 2 hrs. The mixture was concentrated to dryness. The mixture was purified with perp-HPLC (column: Phenomenex Gemini-NX C18 75*30 mm*3 um; mobile phase: [water(0.05% NH₃H₂O+10 mM NH₄HCO₃)-ACN]; B %: 29%-69%, 10 min) and chiral SFC (column: DAICEL CHIRALCEL OD (250 mm*30 mm, 10 um); mobile phase: [0.1% NH₃H₂O ETOH]; B %: 30%-30%, min). HYBI_292 (23.3 mg, 34.77 umol, 28.31% yield, 96.66% purity) was obtained as a white solid.

¹H NMR (DMSO-d₆, 400 MHz) δ_{H}=9.99 (s, 1H), 8.98 (d, J=1.6 Hz, 1H), 8.60 (s, 1H), 8.51 (d, J=8.0 Hz, 1H), 8.16 (d, J=8.0 Hz, 1H), 7.29-7.35 (m, 2H), 4.00 (s, 3H), 3.74-3.83 (m, 1H), 3.12 (d, J=10.0 Hz, 2H), 2.77 (d, J=11.2 Hz, 2H), 2.35-2.47 (m, 4H), 2.18 (d, J=11.2 Hz, 6H), 1.91-2.00 (m, 2H), 1.65-1.78 (m, 4H), 1.03 (d, J=6.0 Hz, 6H).

HPLC Rₜ=2.381 min in 8 min chromatography, purity 96.66%.

LCMS Rₜ=2.243 min in 4 min chromatography, purity 100.00%, MS ESI calcd. for 647.3 [M+H]⁺ 648.3, found 648.3.

### Example 55. N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-methoxy-4-(trifluoromethyl)nicotinamide

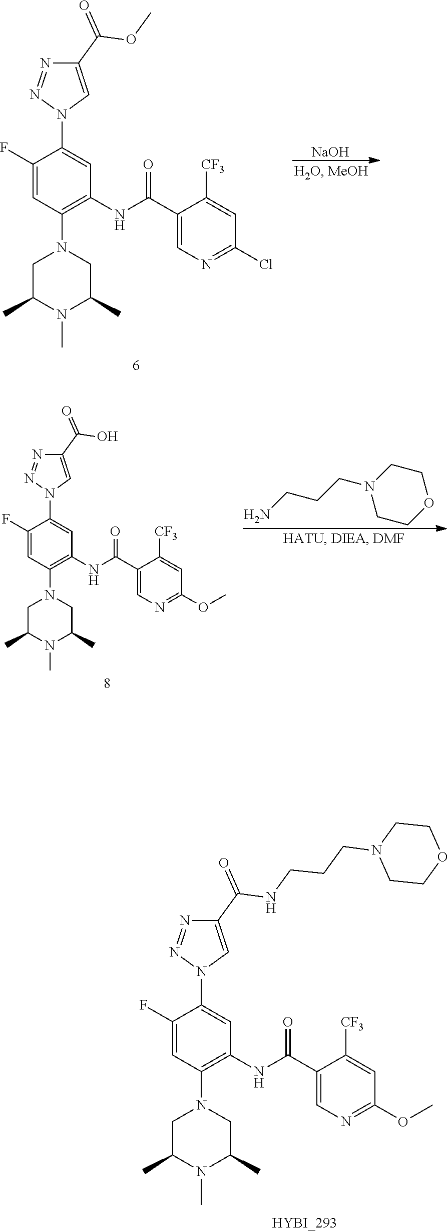 [SMILES: COC(=O)c1cn(-c2cc(NC(=O)c3cnc(Cl)cc3C(F)(F)F)c(N3C[C@@H](C)N(C)[C@@H](C)C3)cc2F)nn1 ; "NaOH" ; COc1cc(C(F)(F)F)c(C(=O)Nc2cc(-n3cc(C(=O)O)nn3)c(F)cc2N2C[C@@H](C)N(C)[C@@H](C)C2)cn1 ; NCCCN1CCOCC1 ; COc1cc(C(F)(F)F)c(C(=O)Nc2cc(-n3cc(C(=O)NCCCN4CCOCC4)nn3)c(F)cc2N2C[C@@H](C)N(C)[C@@H](C)C2)cn1]

### Step 1:1-(2-fluoro-5-(6-methoxy-4-(trifluoromethyl)nicotinamido)-4-((3S,5R)-3,4,5-trimethylpiperazin-1-yl)phenyl)-1H-1,2,3-triazole-4-carboxylic acid (Compound 8)

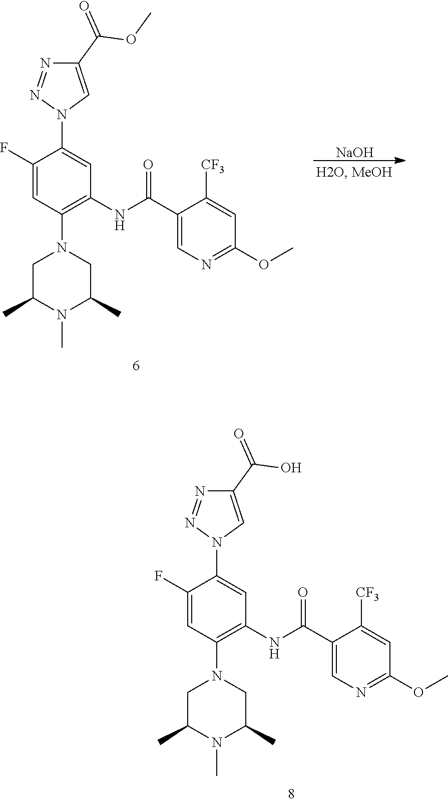 [SMILES: COC(=O)c1cn(-c2cc(NC(=O)c3cnc(OC)cc3C(F)(F)F)c(N3C[C@@H](C)N(C)[C@@H](C)C3)cc2F)nn1 ; "NaOH" ; COc1cc(C(F)(F)F)c(C(=O)Nc2cc(-n3cc(C(=O)O)nn3)c(F)cc2N2C[C@@H](C)N(C)[C@@H](C)C2)cn1]

To a mixture of compound 6 (100 mg, 175.46 umol, 1 eq) in MeOH (5 mL) was added a solution of NaOH (35 mg, 877.29 umol, 5 eq) in H₂O (1.25 mL). The mixture was stirred at 60° C. for 2 hrs. The mixture was acidified with 2N HCl to pH=5. The mixture was concentrated to dryness. The mixture was used directly to the next step without purification. Compound 8 (97 mg, 175.45 umol, 100.00% yield) was obtained as a brown solid.

### Step 2: N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-methoxy-4-(trifluoromethyl)nicotinamide (HYBI_293)

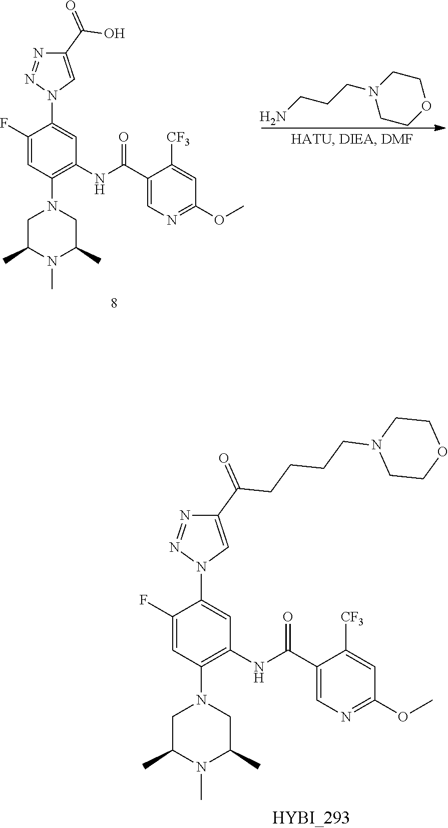 [SMILES: COc1cc(C(F)(F)F)c(C(=O)Nc2cc(-n3cc(C(=O)O)nn3)c(F)cc2N2C[C@@H](C)N(C)[C@@H](C)C2)cn1 ; NCCCN1CCOCC1 ; COc1cc(C(F)(F)F)c(C(=O)Nc2cc(-n3cc(C(=O)CCCCN4CCOCC4)nn3)c(F)cc2N2C[C@@H](C)N(C)[C@@H](C)C2)cn1]

To a mixture of compound 8 (96.76 mg, 175.45 umol, 1 eq) and 3-morpholinopropan-1-amine (38 mg, 263.18 umol, 38.45 uL, 1.5 eq) in DMF (4 mL) was added DIEA (68 mg, 526.35 umol, 91.68 uL, 3 eq), HATU (100 mg, 263.18 umol, 1.5 eq) was added into the mixture. The mixture was purged and degassed with N₂ for 3 times, and stirred at 25° C. for 2 hrs under N₂ atmosphere. The mixture was concentrated to dryness. The mixture was purified with perp-HPLC (column: Phenomenex Gemini-NX C18 75*30 mm*3 um; mobile phase: [water(0.04% NH₃H₂O 10 mM NH₄HCO₃)-ACN]; B %: 35%-55%, 8 min) and chiral SFC (column: DAICEL CHIRALCEL OJ (250 mm*30 mm, 10 um); mobile phase: [0.1% NH₃H₂O ETOH]; B %: 21%-21%, min). HYBI_293 (29.9 mg, 43.70 umol, 24.91% yield, 99.04% purity) was obtained as a white solid.

¹H NMR (DMSO-d₆, 400 MHz) δ_{H}=9.85-10.13 (m, 1H), 8.96 (d, J=1.6 Hz, 1H), 8.84 (t, J=5.6 Hz, 1H), 8.60 (s, 1H), 8.17 (d, J=8.0 Hz, 1H), 7.27-7.38 (m, 2H), 4.00 (s, 3H), 3.61 (t, J=4.4 Hz, 4H), 3.13 (d, J=10.0 Hz, 2H), 2.49-2.47 (m, 4H), 2.35-2.41 (m, 8H), 2.20 (s, 3H), 1.71 (t, J=6.8 Hz, 2H), 1.03 (d, J=6.0 Hz, 6H).

HPLC Rₜ=3.974 min in 8 min chromatography, purity 99.04%.

LCMS Rₜ=2.035 min in 4 min chromatography, purity 100.00%, MS ESI calcd. for 677.31 [M+H]⁺ 678.31, found 678.3.

### Example 56. 4,6-dichloro-N-[5-[4-(diethylcarbamoyl)triazol-1-yl]-4-fluoro-2-[(3R,5S)-3,4,5-trimethylpiperazin-1-yl]phenyl]pyridine-3-carboxamide

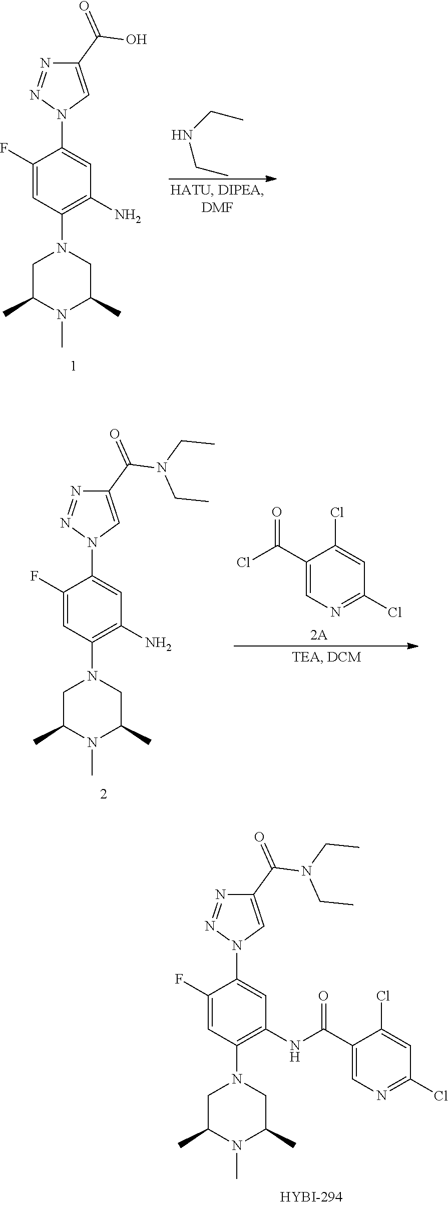

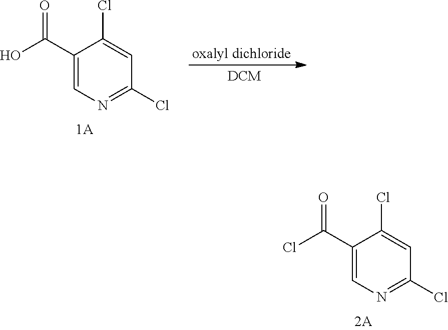 [SMILES: O=C(O)c1cnc(Cl)cc1Cl ; O=C(Cl)c1cnc(Cl)cc1Cl]

Note: The preparation method of compound 1 can be found in Example 57 above.

### Step 1: 1-(5-amino-2-fluoro-4-((3S,5R)-3,4,5-trimethylpiperazin-1-yl)phenyl)-N,N-diethyl-1H-1,2,3-triazole-4-carboxamide (Compound 2)

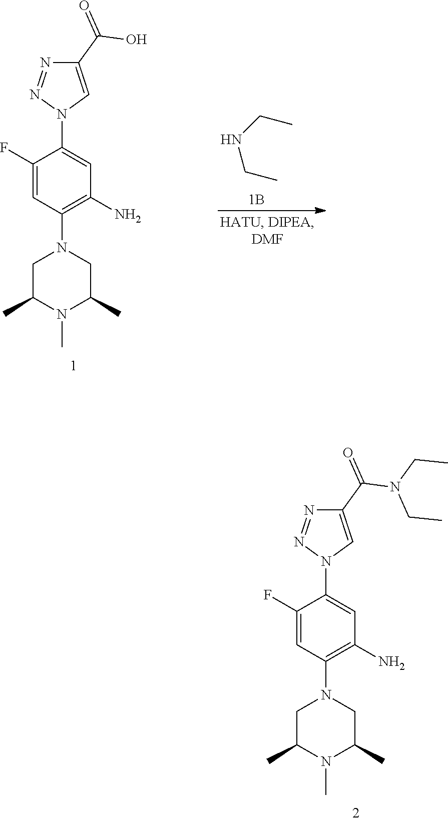

To a mixture of compound 1 (240 mg, 688.91 umol, 1 eq), compound 1B (50.38 mg, 688.91 umol, 70.96 uL, 1 eq) and DIEA (267.11 mg, 2.07 mmol, 359.99 uL, 3 eq) in DMF (3 mL) was added HATU (392.92 mg, 1.03 mmol, 1.5 eq). The reaction mixture was stirred at 20° C. for 2 hr. Water (10 mL) was added to the reaction mixture. The resulting mixture was extracted with DCM (20 mL*3). The combined organic phase was washed with brine (10 mL*2), dried over anhydrous Na₂SO₄, filtered and concentrated. The residue was purified by flash silica gel chromatography (eluent of 0-10% MeOH/DCM). Compound 2 (290 mg, 639.66 umol, 92.85% yield, 89% purity) as a yellow solid.

### Step 2: 4,6-dichloropyridine-3-carbonyl chloride (Compound 2A)

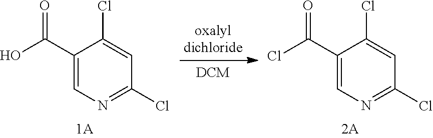 [SMILES: O=C(O)c1cnc(Cl)cc1Cl ; O=C(Cl)c1cnc(Cl)cc1Cl]

To a mixture of compound 1A (60 mg, 0.31 mmol) and DMF (one drop) in DCM (1 mL) was added oxalyl dichloride (198.33 mg, 1.56 mmol, 0.14 mL) at 0° C., and the mixture was stirred at 20° C. for 30 min. The mixture was concentrated to give the residue. The crude compound 2A (60 mg, 285.11 umol, 91.23% yield) was obtained as a yellow oil, which was used into the next step without further purification.

### Step 3: 4,6-dichloro-N-[5-[4-(diethylcarbamoyl)triazol-1-yl]-4-fluoro-2-[(3R,5S)-3,4,5-trimethylpiperazin-1-yl]phenyl]pyridine-3-carboxamide (HYBI_294)

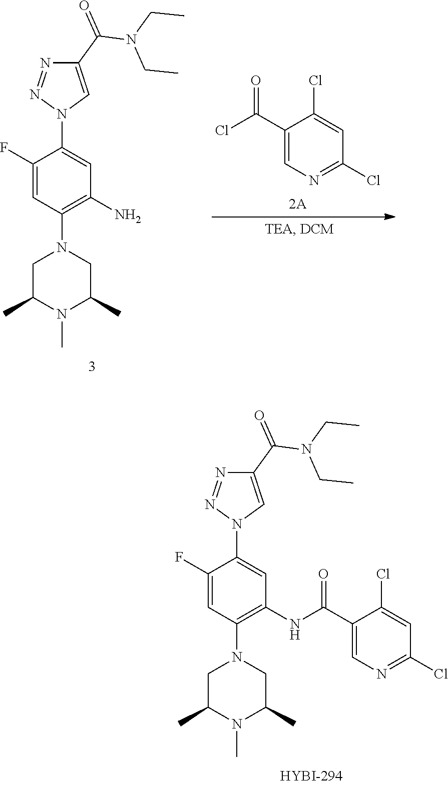 [SMILES: CCN(CC)C(=O)c1cn(-c2cc(N)c(N3C[C@@H](C)N(C)[C@@H](C)C3)cc2F)nn1 ; O=C(Cl)c1cnc(Cl)cc1Cl ; CCN(CC)C(=O)c1cn(-c2cc(NC(=O)c3cnc(Cl)cc3Cl)c(N3C[C@@H](C)N(C)[C@@H](C)C3)cc2F)nn1]

To a mixture of compound 3 (100 mg, 0.25 mmol) and compound 2A (57.37 mg, 0.27 mmol) in DCM (2 mL) at −10° C. was added TEA (125.39 mg, 1.24 mmol, 0.17 mL). The mixture was stirred at 20° C. for 30 min. The residue was diluted with H₂O (100 mL), and the mixture was extracted with DCM (50 mL×2). The combined organic phase was washed with water (20 mL), dried over anhydrous Na₂SO₄, filtered and concentrated to give the crude product. The crude product was purified by prep-HPLC (column: Phenomenex Gemini-NX C18 75×30 mm×3 um; mobile phase: [water(10 mM NH₄HCO₃)-ACN]; B %: 40%-90%, 12 min) to give HYBI-294 (30 mg, 51.95 umol, 20.96% yield) as a white solid.

¹H NMR (DMSO-d₆, 400 MHz) δ_{H}=10.14 (s, 1H), 8.94 (d, J=1.6 Hz, 1H), 8.65 (s, 1H), 8.30 (d, J=8.0 Hz, 1H), 8.01 (s, 1H), 7.33 (d, J=12.4 Hz, 1H), 3.72-3.80 (m, 2H), 3.48 (m, 2H), 3.33 (br s, 4H), 3.14 (br d, J=10.8 Hz, 2H), 2.20 (s, 3H), 1.25 (br t, J=6.8 Hz, 3H), 1.17 (br t, J=7.2 Hz, 3H), 1.04 (d, J=6.0 Hz, 6H).

HPLC Rₜ=3.348 min in 8 min chromatography, purity 97.8%.

LCMS Rₜ=2.148 min in 4 min chromatography, purity 92.09%, MS ESI calcd. for 576.19 [M+H]⁺ 577.19, found 577.1.

### Example 57. 4,6-dichloro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)nicotinamide

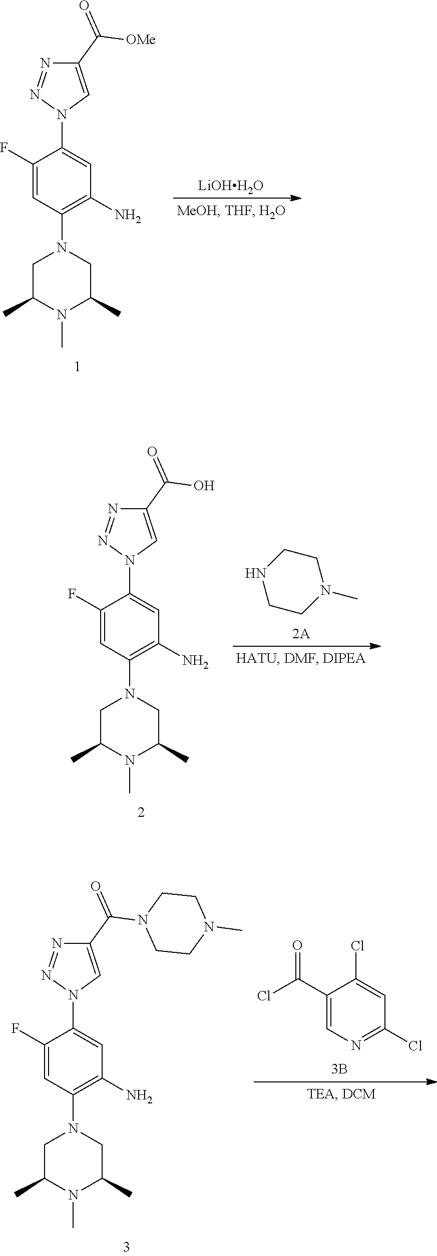

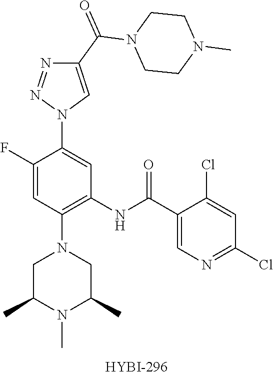 [SMILES: C[C@@H]1CN(c2cc(F)c(-n3cc(C(=O)N4CCN(C)CC4)nn3)cc2NC(=O)c2cnc(Cl)cc2Cl)C[C@H](C)N1C]

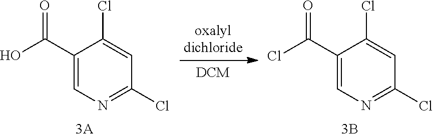 [SMILES: O=C(O)c1cnc(Cl)cc1Cl ; O=C(Cl)c1cnc(Cl)cc1Cl]

Note: The preparation method of compound 1 can be found in Example 52 above.

### Step 1:1-(5-amino-2-fluoro-4-((3S,5R)-3,4,5-trimethylpiperazin-1-yl)phenyl)-1H-1,2,3-triazole-4-carboxylic acid (Compound 2)

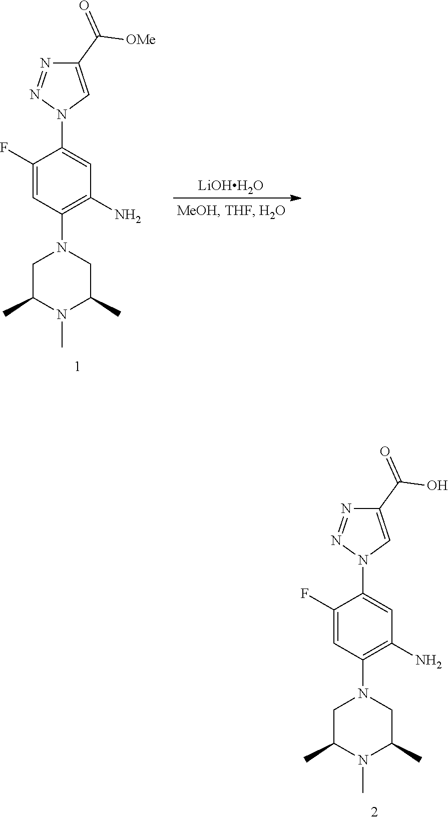 [SMILES: COC(=O)c1cn(-c2cc(N)c(N3C[C@@H](C)N(C)[C@@H](C)C3)cc2F)nn1 ; "LiOH·H2O" ; C[C@@H]1CN(c2cc(F)c(-n3cc(C(=O)O)nn3)cc2N)C[C@H](C)N1C]

To a mixture of compound 1 (250 mg, 689.84 umol, 1 eq) in THF (3 mL) and H₂O (1 mL) was added LiOH·H₂O (86.84 mg, 2.07 mmol, 3 eq). The reaction mixture was stirred at 20° C. for 2 hr. The reaction mixture was concentrated directly. The resulting mixture was then adjusted to pH˜5 by aq. HCl and concentrated to dryness. The product was used in the next step without further purification. Compound 2 (240 mg, 688.91 umol, 99.87% yield) was obtained as a yellow solid.

### Step 2:1-(5-amino-2-fluoro-4-((3S,5R)-3,4,5-trimethylpiperazin-1-yl)phenyl)-1H-1,2,3-triazol-4-yl)(4-methylpiperazin-1-yl)methanone (Compound 3)

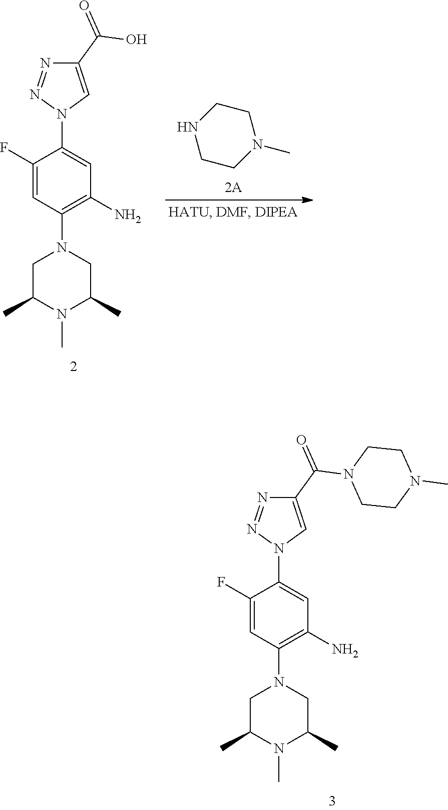

To a mixture of compound 2 (240 mg, 688.91 umol, 1 eq), compound 2A (69.00 mg, 688.91 umol, 76.41 uL, 1 eq) and DIEA (267.11 mg, 2.07 mmol, 359.99 uL, 3 eq) in DMF (3 mL) was added HATU (392.92 mg, 1.03 mmol, 1.5 eq). The reaction mixture was stirred at 20° C. for 2 hr. Water (10 mL) was added to the reaction mixture. The resulting mixture was extracted with DCM (10 mL*3). The combined organic phase was washed with brine (10 mL*2 mL), dried over anhydrous Na₂SO₄, filtered and concentrated. The residue was purified by flash silica gel chromatography (eluent of 0-12% MeOH/DCM). Compound 3 (300 mg, 634.12 umol, 92.05% yield, 91% purity) was obtained as a yellow solid.

LCMS Rₜ=1.401 min in 4 min chromatography, purity 91.06%, MS ESI calcd. for 430.26 [M+H]⁺ 431.26, found 431.2.

### Step 3: 4,6-dichloronicotinoyl chloride (Compound 3B)

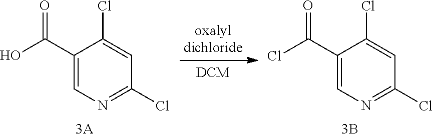 [SMILES: O=C(O)c1cnc(Cl)cc1Cl ; O=C(Cl)c1cnc(Cl)cc1Cl]

To a mixture of compound 3A (70 mg, 364.58 umol, 1 eq) in DCM (1 mL) and DMF (one drop) was added oxalyl dichloride (231.38 mg, 1.82 mmol, 159.57 uL, 5 eq) at 0° C. The mixture was stirred at 20° C. for 30 min. The reaction mixture was concentrated directly. The product was used in the next step without further purification. Compound 3B (70 mg, 332.63 umol, 91.23% yield) was obtained as a white oil.

### Step 4: 4,6-dichloro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)nicotinamide (Compound HYBI_296)

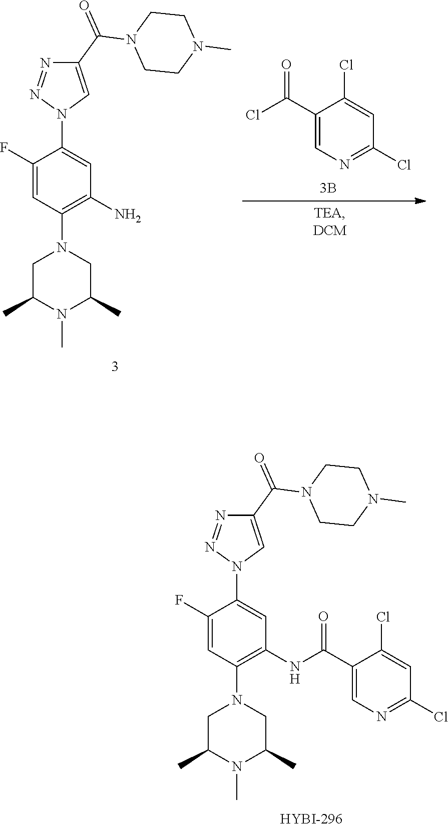 [SMILES: C[C@@H]1CN(c2cc(F)c(-n3cc(C(=O)N4CCN(C)CC4)nn3)cc2N)C[C@H](C)N1C ; O=C(Cl)c1cnc(Cl)cc1Cl ; C[C@@H]1CN(c2cc(F)c(-n3cc(C(=O)N4CCN(C)CC4)nn3)cc2NC(=O)c2cnc(Cl)cc2Cl)C[C@H](C)N1C]

To a solution of compound 3 (102.29 mg, 237.59 umol, 1 eq), compound 3B (70 mg, 332.63 umol, 1.4 eq) in DCM (1.5 mL) was added TEA (120.21 mg, 1.19 mmol, 165.35 uL, 5 eq) at −10° C. The mixture was stirred at 25° C. for 30 min. Water (10 mL) was added to the reaction mixture. The resulting mixture was extracted with DCM (20 mL*3). The combined organic phase was washed with brine (10 mL*2 mL), dried over anhydrous Na₂SO₄, filtered and concentrated. The residue was purified by prep-HPLC (column: Phenomenex Gemini-NX C18 75*30 mm*3 um; mobile phase: [water(10 mM NH₄HCO₃)-ACN]; B %: 35%-50%, 6 min). Compound HYBI_296 (19.8 mg, 31.73 umol, 13.36% yield, 96.88% purity) was obtained as a white solid.

¹H NMR (DMSO-d₆, 400 MHz) δ_{H}=10.16-9.98 (m, 1H), 8.97-8.92 (m, 1H), 8.67-8.61 (m, 1H), 8.28 (d, J=8 Hz, 1H), 7.99 (s, 1H), 7.32 (d, J=12 Hz, 1H), 3.97 (s, 2H), 3.66 (s, 2H), 3.13 (d, J=10.8 Hz, 2H), 2.57-2.52 (m, 2H), 2.38 (t, J=4.8 Hz, 6H), 2.20 (d, J=8.8 Hz, 6H), 1.03 (d, J=6 Hz, 6H).

HPLC Rₜ=3.699 min in 8 min chromatography, purity 96.88%.

LCMS Rₜ=1.854 min in 4 min chromatography, purity 98.41%, MS ESI calcd. for 603.20, [M+H]⁺ 604.20, found 604.2.

### Example 58. 4-chloro-N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-(trifluoromethyl)nicotinamide

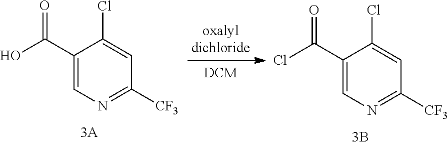 [SMILES: O=C(O)c1cnc(C(F)(F)F)cc1Cl ; O=C(Cl)c1cnc(C(F)(F)F)cc1Cl]

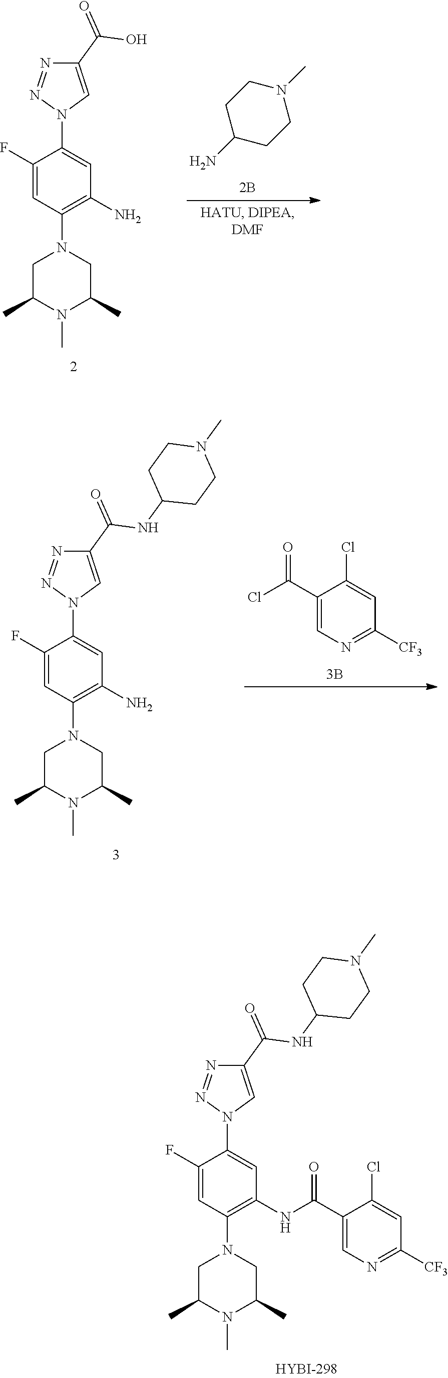

Note: The preparation method of compound 2 can be found in Example 57 above.

### Step 1: 4-chloro-6-(trifluoromethyl)nicotinoyl chloride (Compound 3)

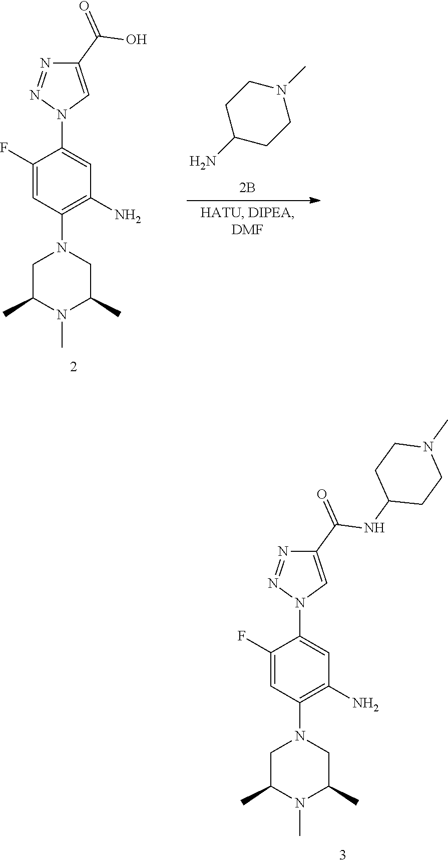

To a mixture of compound 2 (240 mg, 688.91 umol, 1 eq), compound 2B (78.67 mg, 688.91 umol, 1 eq) and DIEA (267.11 mg, 2.07 mmol, 359.99 uL, 3 eq) in DMF (3 mL) was added HATU (392.92 mg, 1.03 mmol, 1.5 eq). The reaction mixture was stirred at 20° C. for 2 hr. Water (10 mL) was added to the reaction mixture. The resulting mixture was extracted with DCM (20 mL*3). The combined organic phase was washed with brine (10 mL*2), dried over anhydrous Na₂SO₄, filtered and concentrated. The residue was purified by flash silica gel chromatography (Ieluent of 0-50% MeOH/DCM). Compound 3 (320 mg, 611.86 umol, 88.81% yield, 85% purity) was obtained as yellow solid.

LCMS Rₜ=1.593 min in 4 min chromatography, purity 85.06%, MS ESI calcd. for 444.28 [M+H]⁺ 445.28, found 445.3.

### Step 2: 4-chloro-6-(trifluoromethyl)nicotinoyl chloride (Compound 3B)

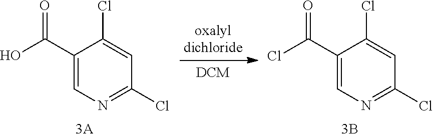 [SMILES: O=C(O)c1cnc(Cl)cc1Cl ; O=C(Cl)c1cnc(Cl)cc1Cl]

To a solution of compound 3A (150 mg, 665.03 umol, 1 eq) and DMF (4.86 mg, 66.50 umol, 5.12 uL, 0.1 eq) in DCM (2 mL) was added oxalyl dichloride (422.06 mg, 3.33 mmol, 291.08 uL, 5 eq) dropwise at 0° C. The reaction mixture was stirred at 25° C. for 20 min. The mixture was concentrated to remove DCM. The product was used in the next step without further purification. Compound 3B (160 mg, 655.74 umol, 98.60% yield) was obtained as yellow oil.

### Step 3: 4-chloro-N-(4-fluoro-5-(4-((1-methylpiperidin-4-yl)carbamoyl)-1H-1,2,3-triazol-1-yl)-2-((3S,5R)-3,4,5-trimethylpiperazin-1-yl)phenyl)-6-(trifluoromethyl)nicotinamide (HYBI_298)

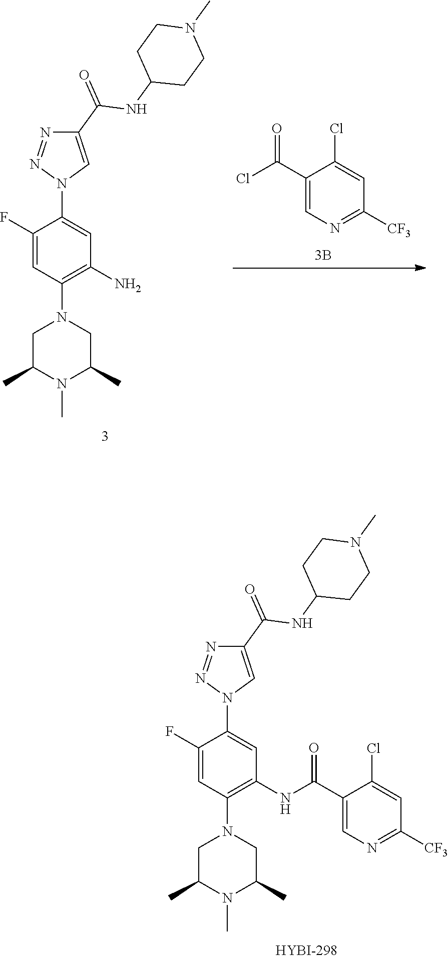 [SMILES: C[C@@H]1CN(c2cc(F)c(-n3cc(C(=O)NC4CCN(C)CC4)nn3)cc2N)C[C@H](C)N1C ; O=C(Cl)c1cnc(C(F)(F)F)cc1Cl ; C[C@@H]1CN(c2cc(F)c(-n3cc(C(=O)NC4CCN(C)CC4)nn3)cc2NC(=O)c2cnc(C(F)(F)F)cc2Cl)C[C@H](C)N1C]

To a mixture of compound 3 (210 mg, 472.39 umol, 1 eq) and compound 3B (160 mg, 655.74 umol, 1.39 eq) in DCM (2 mL) was added TEA (239.00 mg, 2.36 mmol, 328.75 uL, 5 eq) at −10° C. The reaction mixture was stirred at 25° C. for 20 min. The mixture was concentrated to remove DCM. The residue was purified by prep-HPLC column: Phenomenex Gemini-NX C18 75*30 mm*3 um; mobile phase: [water(10 mM NH4HCO3)-ACN]; B %: 25%-60%, 10 min. HYBI_298 (8.8 mg, 13.29 umol, 2.81% yield, 98.49% purity) was obtained as a white solid.

¹H NMR (DMSO, 400 MHz) δ_{H}=10.24 (s, 1H), 9.01-8.95 (m, 2H), 8.51 (d, J=8.4 Hz, 1H), 8.37-8.29 (m, 2H), 7.33 (d, J=12.4 Hz, 1H), 3.83-3.70 (m, 1H), 3.14 (d, J=10.8 Hz, 2H), 2.76 (d, J=11.2 Hz, 2H), 2.57-2.53 (m, 2H), 2.43-2.35 (m, 2H), 2.17 (d, J=12.4 Hz, 6H), 1.99-1.89 (m, 2H), 1.76-1.65 (m, 4H), 1.03 (d, J=6.0 Hz, 6H).

HPLC Rₜ=4.422 min in 8 min chromatography, purity 98.49%.

LCMS Rₜ=2.282 min in 4 min chromatography, purity 97.53%, MS ESI calcd. for 651.25 [M+H]⁺ 652.3, found 652.3.

### Example 59. 4,6-dichloro-N-[4-fluoro-5-[4-(3-morpholinopropylcarbamoyl)triazol-1-yl]-2-[(3R,5S)-3,4,5-trimethylpiperazin-1-yl]phenyl]-5-methyl-pyridine-3-carboxamide

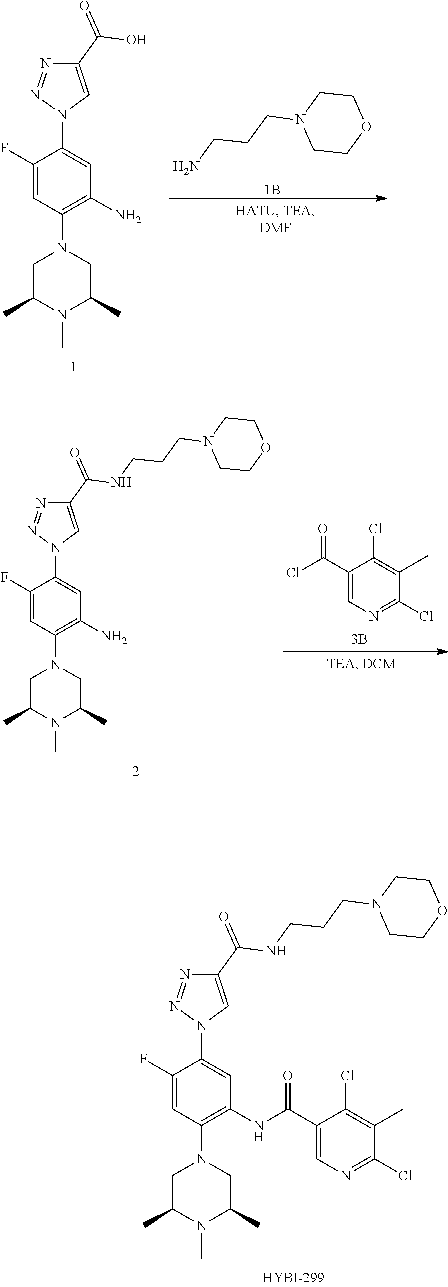 [SMILES: C[C@@H]1CN(c2cc(F)c(-n3cc(C(=O)O)nn3)cc2N)C[C@H](C)N1C ; NCCCN1CCOCC1 ; "1B" ; C[C@@H]1CN(c2cc(F)c(-n3cc(C(=O)NCCCN4CCOCC4)nn3)cc2N)C[C@H](C)N1C ; Cc1c(Cl)ncc(C(=O)Cl)c1Cl ; Cc1c(Cl)ncc(C(=O)Nc2cc(-n3cc(C(=O)NCCCN4CCOCC4)nn3)c(F)cc2N2C[C@@H](C)N(C)[C@@H](C)C2)c1Cl]

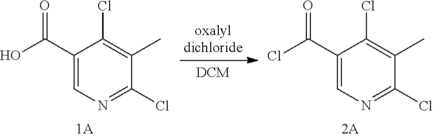

Note: The preparation method of compound 1 can be found in Example 57 above.

### Step 1: 4,6-dichloro-5-methyl-pyridine-3-carbonyl chloride (Compound 2)

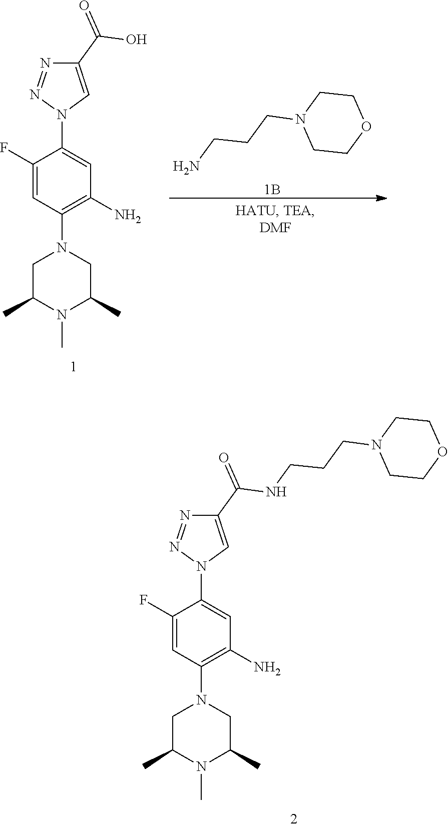 [SMILES: C[C@@H]1CN(c2cc(F)c(-n3cc(C(=O)O)nn3)cc2N)C[C@H](C)N1C ; NCCCN1CCOCC1 ; "1B" ; C[C@@H]1CN(c2cc(F)c(-n3cc(C(=O)NCCCN4CCOCC4)nn3)cc2N)C[C@H](C)N1C]

To a solution of compound 1 (140 mg, 401.87 umol, 1 eq), compound 1B (57.95 mg, 401.87 umol, 58.72 uL, 1 eq) and DIEA (155.81 mg, 1.21 mmol, 209.99 uL, 3 eq) in DMF (1 mL) was added HATU (229.20 mg, 602.80 umol, 1.5 eq). The mixture was stirred at 20° C. for 2 h. Water (10 mL) was added to the reaction mixture. The resulting mixture was extracted with DCM (20 mL*3). The combined organic phase was washed with brine (10 mL*2), dried over anhydrous Na₂SO₄, filtered and concentrated. The residue was purified by flash silica gel chromatography (eluent of 0-11% MeOH/DCM). The crude compound 2 (300 mg, 632.15 umol, 78.65% yield) was obtained as a yellow solid.

LCMS Rₜ=1.460 min in 4 min chromatography, purity 73.449%, MS ESI calcd. for 474.29 [M+H]⁺ 475.29, found 475.3.

### Step 2: 4,6-dichloro-5-methyl-pyridine-3-carbonyl chloride (Compound 2A)

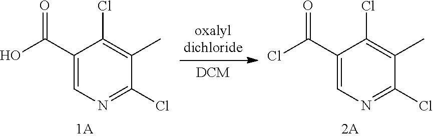

To a mixture of compound 1A (60 mg, 0.29 mmol) and DMF (one drop) in DCM (1 mL) was added oxalyl dichloride (184.83 mg, 1.46 mmol, 0.13 mL) at 0° C., and the mixture was stirred at 20° C. for 30 min. The mixture was concentrated to give the residue. The crude compound 2A (60 mg, 267.29 umol, 91.78% yield) was obtained as a yellow oil, which was used into the next step without further purification.

### Step 3: 4,6-dichloro-N-[4-fluoro-5-[4-(3-morpholinopropylcarbamoyl)triazol-1-yl]-2-[(3R,5S)-3,4,5-trimethylpiperazin-1-yl]phenyl]-5-methyl-pyridine-3-carboxamide (HYBI_299)

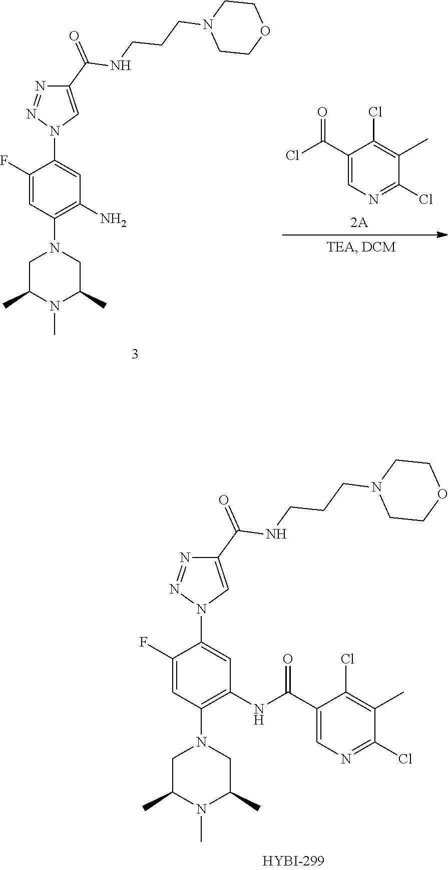 [SMILES: C[C@@H]1CN(c2cc(F)c(-n3cc(C(=O)NCCCN4CCOCC4)nn3)cc2N)C[C@H](C)N1C ; Cc1c(Cl)ncc(C(=O)Cl)c1Cl ; Cc1c(Cl)ncc(C(=O)Nc2cc(-n3cc(C(=O)NCCCN4CCOCC4)nn3)c(F)cc2N2C[C@@H](C)N(C)[C@@H](C)C2)c1Cl]

To a mixture of compound 3 (100 mg, 0.21 mmol) and compound 2A (56.76 mg, 0.25 mmol) in DCM (2 mL) at −10° C. was added TEA (106.61 mg, 1.05 mmol, 0.15 mL). The mixture was stirred at 20° C. for 30 min. The residue was diluted with H₂O (100 mL), and the mixture was extracted with DCM (50 mL×2). The combined organic phase was washed with water (20 mL), dried over anhydrous Na₂SO₄, filtered and concentrated to give the crude product. The crude product was purified by prep-HPLC (column: Phenomenex Gemini-NX C18 75×30 mm×3 um; mobile phase: [water(10 mM NH₄HCO₃)-ACN]; B %: 35%-55%, 7 min) to give HYBI-299 (10 mg, 15.09 umol, 7.16% yield) as a white solid.

¹H NMR (DMSO-d₆, 400 MHz) δ_{H}=10.09 (br s, 1H), 8.97 (d, J=1.6 Hz, 1H), 8.86 (t, J=5.6 Hz, 1H), 8.47 (s, 1H), 8.23-8.30 (m, 1H), 7.31 (d, J=12.4 Hz, 1H), 3.60 (m, 4H), 3.37 (s, 2H), 3.32 (br s, 4H), 3.14 (br d, J=10.4 Hz, 2H), 2.52 (br s, 3H), 2.34-2.38 (m, 6H), 2.19 (s, 3H), 1.65-1.75 (m, 2H), 1.03 (d, J=6.00 Hz, 6H).

HPLC Rₜ=2.379 min in 8 min chromatography, purity 96.6%.

LCMS Rₜ=1.624 min in 4 min chromatography, purity 97.27%, MS ESI calcd. for 661.25 [M+H]⁺ 662.25, found 662.3.

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### Biological Examples

### Example B-1: Enzyme assay of inhibition in leukemia cell lines

Procedure: MV-411 cells were seeded into 384 well plates at 2000 cells/well density in 50 μL total volume, according to plate map and were allowed to naturally sediment by waiting about 30 min on a Clean Bench. Next, plated cells were centrifuged for 1 min at 1000 rpm and the excess cells were transferred into the flasks for further culture. Cells in the assay plates were incubated (at least 4 hrs.) at 37° C., 5% CO₂ followed by adding the compounds as the plate map indicated. The tests were performed in duplicates with treatment of compounds at 10 pts 3-fold titration in 384 well plates. Taxol was used as positive control while DMSO as negative control. To rule out edge effect, the wells on the edge were not seeded and therefore one 384 well plate holds 13 compounds. Cells viability was measured 72h after incubation with compounds using CellTiterGlo (promega) viability assay according to manufacturer's instruction to check the ATP production in each well.

Experiments on anti-proliferative activity against leukemia cells were conducted with some of the compounds of the invention. Table 4 shows the results of evaluation of the anti-proliferative activity of some of the compounds disclosed herein against acute leukemia cells, wherein MV-411 is human acute monocytic leukemia cells.

TABLE 4
Anti-proliferative activity of some of the compounds
of the invention against leukemia cells.

| Huya No. | Compound No. | GI₅₀ μM (MV-411) | |
|---|---|---|---|
| 065A | 1 | 1.38 | |
| 065 | 2 | >200 | |
| 063A | 3 | 0.298 | |
| 063 | 4 | >200 | |
| 070A | 5 | 7.26 | |
| 070 | 6 | >200 | |
| 067A | 7 | 0.4 | |
| 067 | 8 | >200 | |
| 064A | 9 | 0.241 | |
| 064 | 10 | >200 | |
| HYBI-200 | 11 | 2.14, | (2.01) |
| HYBI-201 | 12 | 60.14, | (88.75) |
| HYBI-202 | 13 | 21.81, | (23.72) |
| HYBI-203 | 14 | 25.31, | (21.37) |
| HYBI-204 | 15 | >237.29 | |
| HYBI-205 | 16 | 51.89 | |
| HYBI-206 | 17 | 36.14, | (78.57) |
| HYBI-207 | 18 | 42.72 | |
| HYBI-208 | 19 | 0.91, | (0.94) |
| HYBI-209 | 20 | 2.86, | (3.19) |
| HYBI-210 | 21 | 2.5, | (2.33) |
| HYBI-212A | 22 | 39.66 | |
| HYBI-213_A | 23 | 196.14 | |
| HYBI-215 | 24 | >200 | |
| HYBI-215A | 25 | 145.89 | |
| HYBI-219 | 26 | 75.24, | (61.72) |
| HYBI-221 | 27 | 18.53 | |
| HYBI-222A | 28 | >200 | |
| HYBI-224 | 29 | 18.38 | |
| HYBI-227-A | 30 | 12.21 | |
| HYBI-229 | 31 | 105.63 | |
| HYBI-236 | 32 | 1.24 | |
| HYBI-238-A | 33 | 35.29 | |
| HYBI-256 | 34 | 0.37 | |
| HYBI-257 | 35 | 2.42 | |
| HYBI-257B | 36 | 28.24 | |
| HYBI-260 | 37 | 44.29, | (46.25) |
| HYBI-261 | 38 | 67.93 | |
| HYBI-262 | 39 | 20.15 | |
| HYBI-262_A | 40 | >200 | |
| HYBI-263 | 41 | 108.44 | |
| HYBI-263-A | 42 | 3.17 | |
| HYBI-264 | 43 | 19.91 | |
| HYBI-265 | 44 | 49.94 | |
| HYBI-267 | 45 | 11.22 | |
| HYBI-268 | 46 | 22.43 | |
| HYBI-275 | 47 | 3.4, | (8.97) |
| HYBI-282 | 48 | 2.76, | (1.19) |
| HYBI-283 | 49 | 27.70 | |
| HYBI-284 | 50 | 0.49 | |
| HYBI-285 | 51 | 1.14 | |
| HYBI-286 | 52 | 1.22 | |
| HYBI-290 | 53 | 0.90 | |
| HYBI-292 | 54 | 14.34 | |
| HYBI-293 | 55 | 27.78 | |
| HYBI-294 | 56 | 1.17 | |
| HYBI-296 | 57 | 0.74 | |
| HYBI-298 | 58 | 0.68 | |
| HYBI-299 | 59 | >204.18 | (poor solubility) |

| | |
|---|---|
| DDO-2083 | 17.7 |

ND indicates not detected.

### Example B-2: Enzyme assay of inhibition against MLL1-WDR5 protein-protein interactions

WDR5 TR-FRET Assay Procedure: Stock compounds were transferred to the assay plate by Echo Liquid Handler. Reactions were performed in the assay buffer (1×PBS, 300 mM NaCl, 0.5 mM TCEP, 0.1% CHAPS) containing 5 nM WDR5 protein, 10 nM peptide (Ac-ARTEVHLRKS-[Ahx-Ahx][C]-Alexa Fluor 488-NH2) and 0.25 nM Tb-anti His antibody (Tb-Ab) in 384-well white plate (PerkinElmer), with a final volume of 20 μl. Stock compounds were incubated with WDR5 protein for 30 min at room temperature. Plates were covered, protected from light and incubated for 60 min at room temperature, after adding the peptide and Tb-Ab. EnVision Multimode Plate Reader (PerkinElmer) was used for the TR-FRET assay with excitation wavelength at 340 nm and emission wavelength at 495 and 520 nm. The ratio of the 520/495 wavelengths were used to assess the degree of the FRET signal. IC₅₀ was calculated by fitting the inhibition data using XLfit software to sigmoidal dose-response model. Table 5 shows the results of the WDR5 TR-FRET assay, for some of the compounds disclosed herein.

TABLE 5
MLL1-WDR5 PPI inhibitory activity of representative
compounds disclosed herein.

| COMPOUND_ID | IC50 (nM) |
|---|---|
| HYBI-063 | 51.31 |
| HYBI-063A | >10000 |
| HYBI-064 | 9.12 |
| HYBI-064A | >10000 |
| HYBI-065 | 16.65 |
| HYBI-065A | ND* |
| HYBI-067 | 21.17 |
| HYBI-067A | >10000 |
| HYBI-070 | 7.23 |
| HYBI-070A | 7271.71 |
| HYBI-200 | 4231.63 |
| HYBI-201 | >10000 |
| HYBI-202 | >10000 |
| HYBI-203 | >10000 |
| HYBI-204 | >10000 |
| HYBI-205 | >10000 |
| HYBI-206 | >10000 |
| HYBI-207 | >10000 |
| HYBI-208 | >10000 |
| HYBI-209 | >10000 |
| HYBI-210 | 6570.93 |
| HYBI-212A | >10000 |
| HYBI-213_A | >10000 |
| HYBI-215 | >10000 |
| HYBI-215A | >10000 |
| HYBI-219 | >10000 |
| HYBI-221 | >10000 |
| HYBI-222A | >10000 |
| HYBI-224 | >10000 |
| HYBI-227-A | >10000 |
| HYBI-229 | >10000 |
| HYBI-236 | >10000 |
| HYBI-238-A | >10000 |
| HYBI-256 | >10000 |
| HYBI-257 | >10000 |
| HYBI-257B | >10000 |
| HYBI-260 | 1957.17 |
| HYBI-261 | >10000 |
| HYBI-262 | 1146.99 |
| HYBI-262_A | ND* |
| HYBI-263 | ND* |
| HYBI-263-A | >10000 |
| HYBI-264 | >10000 |
| HYBI-265 | >10000 |
| HYBI-267 | >10000 |
| HYBI-268 | 458.12 |
| HYBI-275 | 3515.45 |
| HYBI-282 | >10000 |
| HYBI-283 | 4368.03 |
| HYBI-284 | 1269.85 |
| HYBI-285 | 326.44 |
| HYBI-286 | 286.57 |
| HYBI-290 | 535.17 |
| HYBI-292 | 617.70 |
| HYBI-293 | 2822.05 |
| HYBI-294 | 1155.30 |
| HYBI-296 | 2174.58 |
| HYBI-298 | >10000 |
| HYBI-299 | >10000 |

ND* = Not Determined

### Example B-3: hERG Assay Results

Procedure: Compounds were prepared and diluted with DMSO to make 0.2 mM and 0.02 mM solution. Reference compound was diluted with DMSO to make 8-point 4-fold serial dilution, starting at 0.2 mM. One μl of compounds/high control/low control was transferred to the assay plate according to the plate map. Next, and by following the plate map, 100 μl of membrane stocks was dispensed into the plate followed by adding 100 μl of radio ligand. Plates were then sealed and were incubated at RT for 1 hours. In the meantime, the Unifilter-96 GF/C filter plates were soaked with 50 μL of 0.5% BSA per well for at least 0.5 hour at room temperature. When binding assays were completed, the reaction mixture was filtered through GF/C plates using Perkin Elmer Filtermate Harvester, and then each plate was washed for 4 times with cold wash buffer. Next, the filter plates were dried for 1 hr at 50 degrees and the bottom of the filter plate wells were sealed using Perkin Elmer Unifilter-96 backing seal tape. Next, 50 μl of Perkin Elmer Microscint 20 cocktail was added. The top of the filter plate was sealed with Perkin Elmer TopSeal-A sealing film. Using Perkin Elmer MicroBeta2 Reader count 3H trapped on filter. Finally, the data was analyzed with GraphPad Prism 5. The “Inhibition [% Control]” was calculated using the equation: % Inh=(1-Background subtracted Assay value/Background subtracted HC value)*100.

The compounds of the disclosure were tested in several hERG assays, the results of which are listed in Table 6.

TABLE 6

| | | | | Max Dose | % Inh at |
|---|---|---|---|---|---|
| Huya No. | Cmpd. No. | IC₅₀ (nM) | Ki (nM) | (nM) | Max dose |
| 063A | 3 | >10,000 | NA | 10,000 | 3.31 |
| 067A | 7 | >10,000 | NA | 10,000 | 11.75 |
| 064A | 9 | >10,000 | NA | 10,000 | 6.39 |
| | Dofetilide | 2.53 | 1.43 | 10,000 | 99.86 |

Furthermore, 6-chloro-4-(trifluoromethyl)-nicotinamide analogs were tested in the hERG channel assay and found to be essentially inactive, with IC₅₀>10.0 μM. These hERG assay results for the compounds of this disclosure are encouraging as the selectivity ratios (IC₅₀hERG/EC₅₀ MV-411) are quite high, ˜25- to 42-fold selectivity, so potential cardiotoxicity issues should be minimal.

The compounds disclosed herein have strong inhibitory activity against MLL1-WDR5 protein-protein interaction, can reduce the MLL1 catalytic activity of MLL1 at cellular level, downregulate the expression of Hox and Meis-1 genes and induce apoptosis of leukemia cells. Also, the phenyl triazole compounds of the invention exhibit good water solubility and pharmaceutical safety, and can be used for treating leukemia.

It is understood that the examples and embodiments described herein are for illustrative purposes only and that various modifications or changes in light thereof will be suggested by persons skilled in the art and are to be included within the spirit and purview of this application and scope of the appended claims. All publications, patents, and patent applications cited herein are hereby incorporated by reference in their entirety for all purposes.

1. A compound having the structure of Formula (I), or a pharmaceutically acceptable salt or solvate thereof: 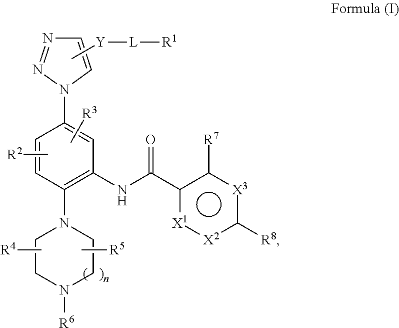
wherein,
Y is absent, —O—, —S—, —C(O)—, —CH₂O—, —NR¹⁰—, —C(O)NR¹¹— or —NR¹²C(O)—, wherein R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, —C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶, wherein
R¹³ is hydrogen, C₁-C₄ alkyl, C₁-C₄ haloalkyl, substituted or unsubstituted phenyl,
R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring,
or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X¹, X², and X³ is independently N or CR⁹, wherein one of X¹, X², or X³ is N;
each R⁷, R⁸, and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

2. The compound of claim 1, wherein n is 1 or 2.

3. The compound of claim 1, wherein L is —(CH₂)ₘ—,
wherein m is an integer from 1-6.

4. The compound of claim 3, wherein m is 1, 2, 3, or 4.

5. The compound of claim 1, wherein the compound has the structure of Formula (II), or a pharmaceutically acceptable salt or solvate thereof:

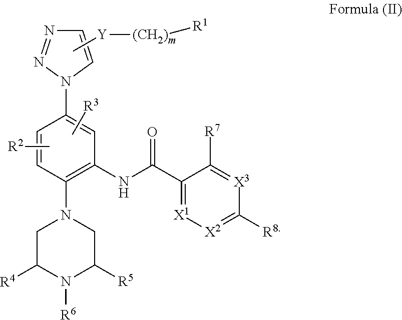

6. The compound of claim 1, wherein X¹ is N; and X² and X³ are each independently CR⁹.

7. The compound of claim 1, wherein X² is N; and X¹ and X³ are each independently CR⁹.

8. The compound of claim 1, wherein X³ is N; and X¹ and X² are each independently CR⁹.

9. The compound of claim 1, wherein X¹ is N; and X² and X³ are CR⁹.

10. The compound of claim 1, wherein X¹ and X² are N; and X³ is CR⁹.

11. The compound of claim 1, wherein X¹, X², and X³ are each N.

12. The compound of claim 5, wherein the compound has the structure of Formula (IIIA), or a pharmaceutically acceptable salt or solvate thereof:

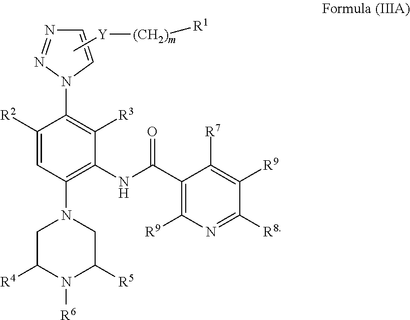

13. The compound of claim 12, wherein each R⁹ is independently hydrogen, halogen, C₁-C₆ alkyl, C₁-C₆ alkoxy, trifluoromethyl, difluoromethyl, trifluoromethoxy, difluoromethoxy, amino, nitro, or cyano.

14. The compound of claim 12, wherein each R⁹ is independently hydrogen, chloro, fluoro, bromo, amino, cyano, methyl, methoxy, trifluoromethyl, difluoromethyl, or trifluoromethyl.

15. The compound of claim 1, wherein each R⁷ and R⁸ is independently hydrogen, halogen, C₁-C₆ alkyl, C₁-C₆ alkoxy, trifluoromethyl, difluoromethyl, trifluoromethoxy, difluoromethoxy, nitro or cyano.

16. The compound of claim 1, wherein R⁷ is trifluoromethyl, difluoromethyl, trifluoromethoxy, or difluoromethoxy; and R⁸ is chloro, fluoro, or bromo.

17. The compound of claim 5, wherein the compound has the structure of Formula (IV), or a pharmaceutically acceptable salt or solvate thereof:

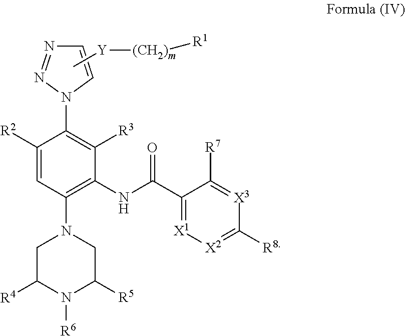

18. The compound of claim 17, wherein Y is absent.

19. The compound of claim 1, wherein Y is —O—, —S—, —C(O)—, —CH₂O—, —NR¹¹—, —C(O)NR¹¹— or —NR¹²C(O)—.

20. The compound of claim 19, wherein Y is —O— or —NR¹⁰—, wherein R¹⁰ is hydrogen or C₁-C₄ alkyl.

21. The compound of claim 19, wherein Y is —C(O)NR¹¹—, wherein R¹¹ is hydrogen or C₁-C₄ alkyl.

22. The compound of claim 1, wherein R¹ is hydrogen, amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring.

23. The compound of claim 22, wherein R¹ is substituted or unsubstituted nitrogen- or oxygen-containing 3 to 7 membered heterocyclic ring.

24. The compound of claim 22, wherein the 3-7 membered heterocyclic ring is piperidine, piperazine, or morpholine.

25. The compound of claim 1, wherein R¹ is —NR¹³COR¹⁴, —C(O)NR¹⁵R¹⁶ or —NR¹⁵R¹⁶.

26. The compound of claim 25, wherein R¹ is —NR¹⁵R¹⁶, wherein R¹⁵ and R¹⁶ are bonded to form a nitrogen- or oxygen-containing 3 to 7 membered heterocyclic ring.

27. The compound of claim 1, wherein R⁴ and R⁵ are each independently hydrogen or C₁-C₆ alkyl.

28. The compound of claim 27, wherein R⁴ and R⁵ are each methyl.

29. The compound of claim 27, wherein R⁴ and R⁵ are each hydrogen.

30. The compound of claim 1, wherein R⁴ is hydrogen and R⁵ is C₁-C₆ alkyl.

31. The compound of claim 1, wherein R⁴ is C₁-C₆ alkyl and R⁵ is hydrogen.

32. The compound of claim 1, wherein R⁶ is hydrogen or C₁-C₆ alkyl.

33. The compound of claim 32, wherein R⁶ is methyl.

34. The compound of claim 1, wherein R² is halogen or hydrogen; and R³ is hydrogen.

35. A compound having the structure of Formula (V), or a pharmaceutically acceptable salt or solvate thereof: 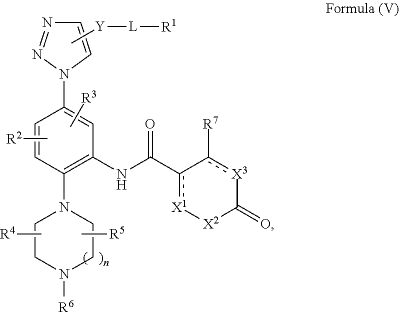
wherein;
Y is absent, —O—, —S—, —C(O)—, —CH₂O—, —NR¹⁰—, —C(O)NR¹¹— or —NR¹²C(O)—, wherein R¹⁰, R¹¹, and R¹² each independently is hydrogen, C₁-C₄ alkyl, C₁-C₄ haloalkyl, or substituted or unsubstituted phenyl, substituted with one, two or three halogen, amino, cyano, hydroxyl, trifluoro, —C₁-C₄ alkyl, C₁-C₄ alkoxy, carboxyl, or imidazolyl;
L is absent or a substituted or unsubstituted C₁-C₆ alkylene linker;
R¹ is hydrogen, amino, hydroxyl, thiol, carboxyl, cyano, C₁-C₄ alkyl, C₁-C₆ alkoxy, substituted or unsubstituted phenyl, substituted or unsubstituted nitrogen- or oxygen-containing 3 to 7 membered heterocyclic ring, —NR¹³COR¹⁴, —C(O)NR¹⁵R¹⁶ or —NR¹⁵R¹⁶ wherein
R¹³ is hydrogen, C₁-C₄ alkyl, C₁-C₄ haloalkyl, substituted or unsubstituted phenyl,
R¹⁴ is amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring,
R¹⁵ and R¹⁶ are each independently is hydrogen, C₁-C₄ alkyl, substituted or unsubstituted phenyl, substituted or unsubstituted nitrogen- or oxygen-containing 3 to 7 membered heterocyclic ring,
or R¹⁵ and R¹⁶ are bonded to form a nitrogen- or oxygen-containing 3 to 7 membered heterocyclic ring, wherein the substituent is halogen, C₁-C₄ alkyl, C₁-C₄ alkoxy, amino, hydroxyl, thiol, carboxyl, cyano, trifluoromethyl or imidazolyl;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each X⁴, X⁵, and X⁶ is independently NR^{9A} or CR⁹; wherein one of X⁴, X⁵, or X⁶ is NR^{9A};
each R^{9A} is independently hydrogen or C₁-C₆ alkyl;
each R⁷ and R⁹ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano; and
n is an integer from 0-2.

36. The compound of claim 35, wherein n is 1 or 2.

37. The compound of claim 35, wherein L is —(CH₂)ₘ, wherein m is an integer from 1-6.

38. The compound of claim 35, wherein X² is NH; and X¹ and X³ are each independently CR⁹.

39. The compound of claim 35, wherein the compound has the structure of Formula (VI), or a pharmaceutically acceptable salt or solvate thereof:

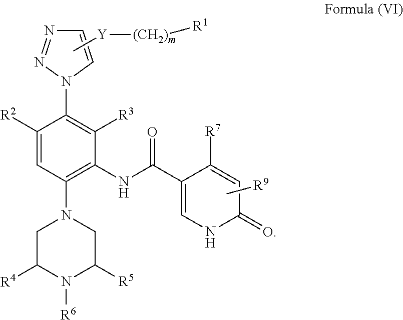

40. The compound of claim 39, wherein each R⁷ and R⁹ is independently hydrogen, halogen, C₁-C₆ alkyl, C₁-C₆ alkoxy, trifluoromethyl, difluoromethyl, trifluoromethoxy, difluoromethoxy, amino, nitro, or cyano.

41. The compound of claim 35, wherein Y is absent.

42. The compound of claim 35, wherein Y is —O—, —S—, —C(O)—, —CH₂O—, —NR¹⁰—, —C(O)NR¹¹— or —NR¹²C(O)—.

43. The compound of claim 42, wherein Y is —O— or —NR¹—, wherein R¹⁰ is hydrogen or C₁-C₄ alkyl.

44. The compound of claim 42, wherein Y is —C(O)NR¹¹—, wherein R¹¹ is hydrogen or C₁-C₄ alkyl.

45. The compound of claim 35, wherein R¹ is hydrogen, amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring.

46. The compound of claim 35, wherein R¹ is —NR¹⁵R¹⁶, wherein R¹⁵ and R¹⁶ are bonded to form a nitrogen- or oxygen-containing 3 to 7 membered heterocyclic ring.

47. The compound of claim 35, wherein R⁴ and R⁵ are each independently hydrogen or C₁-C₆ alkyl.

48. The compound of claim 47, wherein R⁴ and R⁵ are each methyl.

49. The compound of claim 47, wherein R⁴ and R⁵ are each hydrogen.

50. The compound of claim 35, wherein R⁴ is hydrogen and R⁵ is C₁-C₆ alkyl.

51. The compound of claim 35, wherein R⁴ is C₁-C₆ alkyl and R⁵ is hydrogen.

52. The compound of claim 35, wherein R⁶ is hydrogen or C₁-C₆ alkyl.

53. The compound of claim 35, wherein R² is halogen or hydrogen; and R³ is hydrogen.

54. The compound of claim 1, wherein the compound is selected from a compound in Table 1, 2, or 3, or a pharmaceutically acceptable salt thereof.

55. A pharmaceutical composition comprising a compound of claim 1, or a pharmaceutically acceptable salt or solvate thereof, and a pharmaceutically acceptable excipient.

56. A method for the treatment or prevention of acute leukemia in a patient in need thereof, comprising administering to the patient a therapeutically acceptable dose of the compound of claim 1, or the pharmaceutical composition of claim 55.

57. The method of claim 56, wherein the acute leukemia is acute leukemia with MLL1 gene rearrangement.

58. A compound having the following structure: 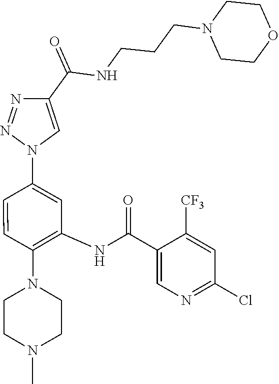 [SMILES: CN1CCN(c2ccc(-n3cc(C(=O)NCCCN4CCOCC4)nn3)cc2NC(=O)c2cnc(Cl)cc2C(F)(F)F)CC1]
or a pharmaceutically acceptable salt or solvate thereof.

59. A compound having the following structure: 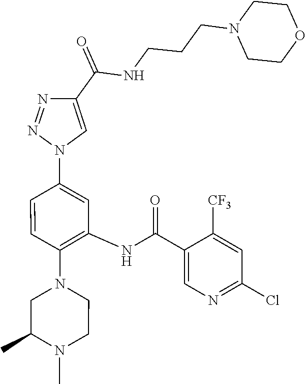 [SMILES: C[C@H]1CN(c2ccc(-n3cc(C(=O)NCCCN4CCOCC4)nn3)cc2NC(=O)c2cnc(Cl)cc2C(F)(F)F)CCN1C]
or a pharmaceutically acceptable salt or solvate thereof.

60. A compound having the following structure: 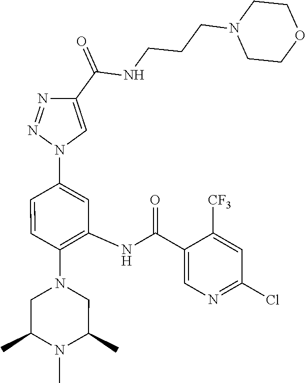 [SMILES: C[C@@H]1CN(c2ccc(-n3cc(C(=O)NCCCN4CCOCC4)nn3)cc2NC(=O)c2cnc(Cl)cc2C(F)(F)F)C[C@H](C)N1C]
or a pharmaceutically acceptable salt or solvate thereof.

61. A compound having the following structure: 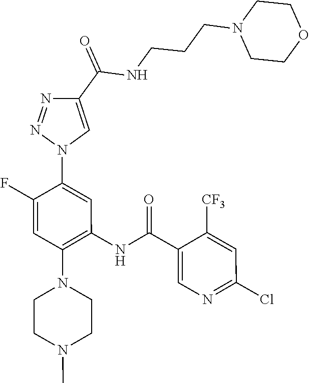 [SMILES: CN1CCN(c2cc(F)c(-n3cc(C(=O)NCCCN4CCOCC4)nn3)cc2NC(=O)c2cnc(Cl)cc2C(F)(F)F)CC1]
or a pharmaceutically acceptable salt or solvate thereof.

62. A compound having the following structure: 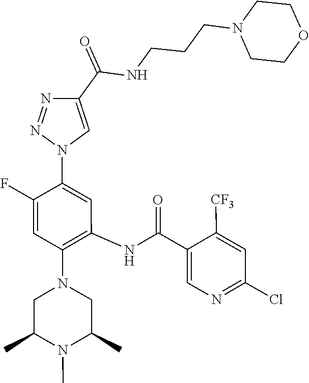 [SMILES: C[C@@H]1CN(c2cc(F)c(-n3cc(C(=O)NCCCN4CCOCC4)nn3)cc2NC(=O)c2cnc(Cl)cc2C(F)(F)F)C[C@H](C)N1C]
or a pharmaceutically acceptable salt or solvate thereof.
